SEQUENCE LISTING (508 sequences)

SEQ ID NO: 1 - protein, 6 aa, synthetic construct
SDYAWN

SEQ ID NO: 2 - protein, 5 aa, synthetic construct
NYGMN

SEQ ID NO: 3 - protein, 5 aa, synthetic construct
NYGMN

SEQ ID NO: 4 - protein, 5 aa, synthetic construct
NYGMN

SEQ ID NO: 5 - protein, 5 aa, synthetic construct
NYGMN

SEQ ID NO: 6 - protein, 5 aa, synthetic construct
DYGMA

SEQ ID NO: 7 - protein, 5 aa, synthetic construct
GYTMN

SEQ ID NO: 8 - protein, 5 aa, synthetic construct
RFWMS

SEQ ID NO: 9 - protein, 5 aa, synthetic construct
DYGMA

SEQ ID NO: 10 - protein, 5 aa, synthetic construct
DYGMA

SEQ ID NO: 11 - protein, 5 aa, synthetic construct
RFWMS

SEQ ID NO: 12 - protein, 5 aa, synthetic construct
RYWMS

SEQ ID NO: 13 - protein, 5 aa, synthetic construct
RYWMS

SEQ ID NO: 14 - protein, 5 aa, synthetic construct
RYWMT

SEQ ID NO: 15 - protein, 5 aa, synthetic construct
RFWMS

SEQ ID NO: 16 - protein, 5 aa, synthetic construct
RYWMS

SEQ ID NO: 17 - protein, 5 aa, synthetic construct
GYTMN

SEQ ID NO: 18 - protein, 5 aa, synthetic construct
RFWMS

SEQ ID NO: 19 - protein, 5 aa, synthetic construct
RYWMS

SEQ ID NO: 20 - protein, 5 aa, synthetic construct
RYWMS

SEQ ID NO: 21 - protein, 5 aa, synthetic construct
DFGMA

SEQ ID NO: 22 - protein, 16 aa, synthetic construct
YISYSGSTSYNPSLKS

SEQ ID NO: 23 - protein, 17 aa, synthetic construct
WINTYSGEPTYVDDFKG

SEQ ID NO: 24 - protein, 17 aa, synthetic construct
WINTNTGEPTYSEEFKG

SEQ ID NO: 25 - protein, 17 aa, synthetic construct
WINTYTGEPTYADDFKG

SEQ ID NO: 26 - protein, 17 aa, synthetic construct
WINTYTGEPTYADDFKG

SEQ ID NO: 27 - protein, 17 aa, synthetic construct
FISNLAYSVYYADTVTG

SEQ ID NO: 28 - protein, 17 aa, synthetic construct
LISPYSGGTNYNQRFKG

SEQ ID NO: 29 - protein, 17 aa, synthetic construct
EISPDSNTIDLTPSLKD

SEQ ID NO: 30 - protein, 17 aa, synthetic construct
FISNLAYSVYYADTVTG

SEQ ID NO: 31 - protein, 17 aa, synthetic construct
FISNLAYSVYYADTVTG

SEQ ID NO: 32 - protein, 17 aa, synthetic construct
EISPNSSTIHQTPSLKD

SEQ ID NO: 33 - protein, 17 aa, synthetic construct
EINPDSSTIDLTPSLKD

SEQ ID NO: 34 - protein, 17 aa, synthetic construct
EISPDSSTIDLTPSLKD

SEQ ID NO: 35 - protein, 17 aa, synthetic construct
EINPHSSTITYTPSLKD

SEQ ID NO: 36 - protein, 17 aa, synthetic construct
EINPDSSTITYTPSLKD

SEQ ID NO: 37 - protein, 17 aa, synthetic construct
EISPDSSTINYTASLKD

SEQ ID NO: 38 - protein, 17 aa, synthetic construct
LISPYSGGTNYNQKFKG

SEQ ID NO: 39 - protein, 17 aa, synthetic construct
EISPNSSTIHQTPSLKD

SEQ ID NO: 40 - protein, 17 aa, synthetic construct
EINPDSSTIDLTPSLKD

SEQ ID NO: 41 - protein, 17 aa, synthetic construct
EISPDSNTIDLTPSLKD

SEQ ID NO: 42 - protein, 17 aa, synthetic construct
FISNLAYNIYYSDTVTG

SEQ ID NO: 43 - protein, 14 aa, synthetic construct
EGGRGGTEGYYFDY

SEQ ID NO: 44 - protein, 8 aa, synthetic construct
GTVVGFDY

SEQ ID NO: 45 - protein, 8 aa, synthetic construct
SSYYAMDY

SEQ ID NO: 46 - protein, 5 aa, synthetic construct
YAMDY

SEQ ID NO: 47 - protein, 9 aa, synthetic construct
WDDYDGFAY

SEQ ID NO: 48 - protein, 10 aa, synthetic construct
DRGLRESMDY

SEQ ID NO: 49 - protein, 14 aa, synthetic construct
SGFITTGVPTNFDV

SEQ ID NO: 50 - protein, 6 aa, synthetic construct
PYYGYY

SEQ ID NO: 51 - protein, 10 aa, synthetic construct
DRGLRESMDY

SEQ ID NO: 52 - protein, 10 aa, synthetic construct
DRGLRETMDY

SEQ ID NO: 53 - protein, 6 aa, synthetic construct
PYYGYY

SEQ ID NO: 54 - protein, 6 aa, synthetic construct
PYHGYY

SEQ ID NO: 55 - protein, 6 aa, synthetic construct
PYHGYY

SEQ ID NO: 56 - protein, 6 aa, synthetic construct
PYYGYY

SEQ ID NO: 57 - protein, 6 aa, synthetic construct
PYYGYY

SEQ ID NO: 58 - protein, 6 aa, synthetic construct
PYYGYY

SEQ ID NO: 59 - protein, 14 aa, synthetic construct
SGFITTGVATNFDV

SEQ ID NO: 60 - protein, 6 aa, synthetic construct
PYYGYY

SEQ ID NO: 61 - protein, 6 aa, synthetic construct
PYYGYY

SEQ ID NO: 62 - protein, 6 aa, synthetic construct
PYYGYY

SEQ ID NO: 63 - protein, 10 aa, synthetic construct
DRGLRETMDY

SEQ ID NO: 64 - protein, 30 aa, synthetic construct
DVQLQESGPGLVKPSQSLSLTCTVTGYSIT

SEQ ID NO: 65 - protein, 30 aa, synthetic construct
QIQLVQSGPELKKPGETVKISCKASGYIFT

SEQ ID NO: 66 - protein, 30 aa, synthetic construct
QIQLVQSGPELKKPGETVKISCKASGYTFT

SEQ ID NO: 67 - protein, 30 aa, synthetic construct; identical to SEQ ID NO: 66

SEQ ID NO: 68 - protein, 30 aa, synthetic construct; identical to SEQ ID NO: 66

SEQ ID NO: 69 - protein, 30 aa, synthetic construct
EVKLVESGGGLVQPGGSRKLSCAASGFTFS

SEQ ID NO: 70 - protein, 30 aa, synthetic construct
EVQLQQSGPELVKPGASMKISCKASGYAFT

SEQ ID NO: 71 - protein, 30 aa, synthetic construct
EVKLLESGGGLVQPGGSLKLSCAASGFDFS

SEQ ID NO: 72 - protein, 30 aa, synthetic construct; identical to SEQ ID NO: 69

SEQ ID NO: 73 - protein, 30 aa, synthetic construct; identical to SEQ ID NO: 69

SEQ ID NO: 74 - protein, 30 aa, synthetic construct
EVNLLESGGGLVQPGGSLKLSCEASGFDFS

SEQ ID NO: 75 - protein, 30 aa, synthetic construct; identical to SEQ ID NO: 71

SEQ ID NO: 76 - protein, 30 aa, synthetic construct; identical to SEQ ID NO: 71

SEQ ID NO: 77 - protein, 30 aa, synthetic construct; identical to SEQ ID NO: 71

SEQ ID NO: 78 - protein, 30 aa, synthetic construct
EVKLLESGGDLVQPGGSLKLSCAASGFDFS

SEQ ID NO: 79 - protein, 30 aa, synthetic construct; identical to SEQ ID NO: 71

SEQ ID NO: 80 - protein, 30 aa, synthetic construct
EVQLQQSGPELVKPGASMKMSCKASGYSFT

SEQ ID NO: 81 - protein, 30 aa, synthetic construct; identical to SEQ ID NO: 74

SEQ ID NO: 82 - protein, 30 aa, synthetic construct; identical to SEQ ID NO: 71

SEQ ID NO: 83 - protein, 30 aa, synthetic construct; identical to SEQ ID NO: 71

SEQ ID NO: 84 - protein, 30 aa, synthetic construct
EVKLVESGGGLVQSGGSRKLSCAASGFTFS

SEQ ID NO: 85 - protein, 14 aa, synthetic construct
WIRQFPGNKLEWMG

SEQ ID NO: 86 - protein, 14 aa, synthetic construct
WVKQAPGKGLKWMG

SEQ ID NO: 87 - protein, 14 aa, synthetic construct
WVKQAPGKGLKWMG

SEQ ID NO: 88 - protein, 14 aa, synthetic construct
WVKQAPGKGLKWMG

SEQ ID NO: 89 - protein, 14 aa, synthetic construct
WVKQAPGKGLKWMG

SEQ ID NO: 90 - protein, 14 aa, synthetic construct
WFRQAPGKGPEWVA

SEQ ID NO: 91 - protein, 14 aa, synthetic construct
WVKQSHGKNLEWIG

SEQ ID NO: 92 - protein, 14 aa, synthetic construct
WVRQAPGKGLEWIG

SEQ ID NO: 93 - protein, 14 aa, synthetic construct
WFRQAPGKGPEWVA

SEQ ID NO: 94 - protein, 14 aa, synthetic construct
WFRQAPGKGPEWVA

SEQ ID NO: 95 - protein, 14 aa, synthetic construct
WVRQTPGEGLEWIG

SEQ ID NO: 96 - protein, 14 aa, synthetic construct
WVRQAPGKGLEWIG

SEQ ID NO: 97 - protein, 14 aa, synthetic construct
WVRQAPGKGLEWIG

SEQ ID NO: 98 - protein, 14 aa, synthetic construct
WVRQAPGKGLEWIG

SEQ ID NO: 99 - protein, 14 aa, synthetic construct
WVRQAPGKGLEWIG

SEQ ID NO: 100 - protein, 14 aa, synthetic construct
WVRQAPGKGLEWIG

SEQ ID NO: 101 - protein, 14 aa, synthetic construct
WVKQSHGKNLEWIG

SEQ ID NO: 102 - protein, 14 aa, synthetic construct
WVRQTPGEGLEWIG

SEQ ID NO: 103 - protein, 14 aa, synthetic construct
WVRQAPGKGLEWIG

SEQ ID NO: 104 - protein, 14 aa, synthetic construct
WVRQAPGKGLEWIG

SEQ ID NO: 105 - protein, 14 aa, synthetic construct
WVRQAPGKGPEWVA

SEQ ID NO: 106 - protein, 32 aa, synthetic construct
RISITRDASKNQFFLQLNSVTTEDTATYYCAR

SEQ ID NO: 107 - protein, 32 aa, synthetic construct
RFVFSLETSASTAYLQINNLKNEDMATYFCAR

SEQ ID NO: 108 - protein, 32 aa, synthetic construct
RFAFSLETSASTAYLQINNLKNEDTATYFCGR

SEQ ID NO: 109 - protein, 32 aa, synthetic construct
RFAFSLETSASTAYLQINNLKNEDMATYFCAR

SEQ ID NO: 110 - protein, 32 aa, synthetic construct
RFAFSLETSASTAYLQINNLKNEDTATYFCAR

SEQ ID NO: 111 - protein, 32 aa, synthetic construct
RFTISRENAKNTLFLEMSSLRSEDTAIYYCTR

SEQ ID NO: 112 - protein, 32 aa, synthetic construct
KATFTVDKSSSTAYMELLSLTSEDSGVYYCAR

SEQ ID NO: 113 - protein, 32 aa, synthetic construct
NFIISRDNAKNALFLQMSKVRSEDTALYYCTR

SEQ ID NO: 114 - protein, 32 aa, synthetic construct
RFTISRENAKNTLYLEMSSLRSEDTAIYYCTR

SEQ ID NO: 115 - protein, 32 aa, synthetic construct
RFTISRENAKNTLYLEMNSLRSEDTAIYYCTR

SEQ ID NO: 116 - protein, 32 aa, synthetic construct
RFIISRDNAKNTLFLQMTKVTSDDSALYYCAR

SEQ ID NO: 117 - protein, 32 aa, synthetic construct
NFIISRDNAKNALYLQMSKVKSEDTALYYCTR

SEQ ID NO: 118 - protein, 32 aa, synthetic construct; identical to SEQ ID NO: 117

SEQ ID NO: 119 - protein, 32 aa, synthetic construct
KFIISRDNAKNTLYLQMSKVSSEDTALYYCAR

SEQ ID NO: 120 - protein, 32 aa, synthetic construct
KFIISRDNAKSSLYLQMSKVTSEDTALYYCAR

SEQ ID NO: 121 - protein, 32 aa, synthetic construct
KFNISRDNAKNTLYLQMSKVRSEDTALYYCAR

SEQ ID NO: 122 - protein, 32 aa, synthetic construct
KATLTVDKSSSTAYMEVFSLTSEDSGVYYCAR

SEQ ID NO: 123 - protein, 32 aa, synthetic construct
KFFISRDNAKSTLFLQMTNVTSEDSALYYCAR

SEQ ID NO: 124 - protein, 32 aa, synthetic construct
NFIISRDNAKNALYLQMSKVRSEDTALYYCTR

SEQ ID NO: 125 - protein, 32 aa, synthetic construct
NFIISRDNAKNALYLQMSKLRSEDTALYYCTR

SEQ ID NO: 126 - protein, 32 aa, synthetic construct
RFTISRENAKNTLYLEMSSLRSEDTAVYYCTR

SEQ ID NO: 127 - protein, 11 aa, synthetic construct
WGQGTTLTVSS

SEQ ID NO: 128 - protein, 11 aa, synthetic construct
WGQGTTLTVSS

SEQ ID NO: 129 - protein, 11 aa, synthetic construct
WGQGTSVTVSS

SEQ ID NO: 130 - protein, 11 aa, synthetic construct
WGQGTSVTVSS

SEQ ID NO: 131 - protein, 11 aa, synthetic construct
WGQGTLVTVSA

SEQ ID NO: 132 - protein, 11 aa, synthetic construct
WGQGTSVTVSS

SEQ ID NO: 133 - protein, 11 aa, synthetic construct
WGAGTTVTVSS

SEQ ID NO: 134 - protein, 11 aa, synthetic construct
WGQGTLVTVSA

SEQ ID NO: 135 - protein, 11 aa, synthetic construct
WGQGTSVTVSS

SEQ ID NO: 136 - protein, 11 aa, synthetic construct
WGQGTSVTVSS

SEQ ID NO: 137 - protein, 11 aa, synthetic construct
WGHGTLVTVSA

SEQ ID NO: 138 - protein, 11 aa, synthetic construct
WGQGTLVTVSA

SEQ ID NO: 139 - protein, 11 aa, synthetic construct
WGQGTLVTVSA

SEQ ID NO: 140 - protein, 11 aa, synthetic construct
WGQGTLVTVSA

SEQ ID NO: 141 - protein, 11 aa, synthetic construct
WGQGTLVTVSA

SEQ ID NO: 142 - protein, 11 aa, synthetic construct
WGQGTLVTVSE

SEQ ID NO: 143 - protein, 11 aa, synthetic construct
WGAGTTVTVSS

SEQ ID NO: 144 - protein, 11 aa, synthetic construct
WGHGTLVTVSA

SEQ ID NO: 145 - protein, 11 aa, synthetic construct
WGQGTLVTVSA

SEQ ID NO: 146 - protein, 11 aa, synthetic construct
WGQGTLVTVSA

SEQ ID NO: 147 - protein, 11 aa, synthetic construct
WGQGTSVTVSS

SEQ ID NO: 148 - protein, 123 aa, synthetic construct
DVQLQESGPGLVKPSQSLSLTCTVTGYSITSDYAWNWIRQFPGNKLEWMGYISYSGSTSYNPSLKSRISITRDASKNQFFLQLNSVTTEDTATYYCAREGGRGGTEGYYFDYWGQGTTLTVSS

SEQ ID NO: 149 - protein, 117 aa, synthetic construct
QIQLVQSGPELKKPGETVKISCKASGYIFTNYGMNWVKQAPGKGLKWMGWINTYSGEPTYVDDFKGRFVFSLETSASTAYLQINNLKNEDMATYFCARGTVVGFDYWGQGTTLTVSS

SEQ ID NO: 150 - protein, 117 aa, synthetic construct
QIQLVQSGPELKKPGETVKISCKASGYTFTNYGMNWVKQAPGKGLKWMGWINTNTGEPTYSEEFKGRFAFSLETSASTAYLQINNLKNEDTATYFCGRSSYYAMDYWGQGTSVTVSS

SEQ ID NO: 151 - protein, 114 aa, synthetic construct
QIQLVQSGPELKKPGETVKISCKASGYTFTNYGMNWVKQAPGKGLKWMGWINTYTGEPTYADDFKGRFAFSLETSASTAYLQINNLKNEDMATYFCARYAMDYWGQGTSVTVSS

SEQ ID NO: 152 - protein, 118 aa, synthetic construct
QIQLVQSGPELKKPGETVKISCKASGYTFTNYGMNWVKQAPGKGLKWMGWINTYTGEPTYADDFKGRFAFSLETSASTAYLQINNLKNEDTATYFCARWDDYDGFAYWGQGTLVTVSA

SEQ ID NO: 153 - protein, 119 aa, synthetic construct
EVKLVESGGGLVQPGGSRKLSCAASGFTFSDYGMAWFRQAPGKGPEWVAFISNLAYSVYYADTVTGRFTISRENAKNTLFLEMSSLRSEDTAIYYCTRDRGLRESMDYWGQGTSVTVSS

SEQ ID NO: 154 - protein, 123 aa, synthetic construct
EVQLQQSGPELVKPGASMKISCKASGYAFTGYTMNWVKQSHGKNLEWIGLISPYSGGTNYNQRFKGKATFTVDKSSSTAYMELLSLTSEDSGVYYCARSGFITTGVPTNFDVWGAGTTVTVSS

SEQ ID NO: 155 - protein, 115 aa, synthetic construct
EVKLLESGGGLVQPGGSLKLSCAASGFDFSRFWMSWVRQAPGKGLEWIGEISPDSNTIDLTPSLKDNFIISRDNAKNALFLQMSKVRSEDTALYYCTRPYYGYYWGQGTLVTVSA

SEQ ID NO: 156 - protein, 119 aa, synthetic construct
EVKLVESGGGLVQPGGSRKLSCAASGFTFSDYGMAWFRQAPGKGPEWVAFISNLAYSVYYADTVTGRFTISRENAKNTLYLEMSSLRSEDTAIYYCTRDRGLRESMDYWGQGTSVTVSS

SEQ ID NO: 157 - protein, 119 aa, synthetic construct
EVKLVESGGGLVQPGGSRKLSCAASGFTFSDYGMAWFRQAPGKGPEWVAFISNLAYSVYYADTVTGRFTISRENAKNTLYLEMNSLRSEDTAIYYCTRDRGLRETMDYWGQGTSVTVSS

SEQ ID NO: 158 - protein, 115 aa, synthetic construct
EVNLLESGGGLVQPGGSLKLSCEASGFDFSRFWMSWVRQTPGEGLEWIGEISPNSSTIHQTPSLKDRFIISRDNAKNTLFLQMTKVTSDDSALYYCARPYYGYYWGHGTLVTVSA

SEQ ID NO: 159 - protein, 115 aa, synthetic construct
EVKLLESGGGLVQPGGSLKLSCAASGFDFSRYWMSWVRQAPGKGLEWIGEINPDSSTIDLTPSLKDNFIISRDNAKNALYLQMSKVKSEDTALYYCTRPYHGYYWGQGTLVTVSA

SEQ ID NO: 160 - protein, 115 aa, synthetic construct
EVKLLESGGGLVQPGGSLKLSCAASGFDFSRYWMSWVRQAPGKGLEWIGEISPDSSTIDLTPSLKDNFIISRDNAKNALYLQMSKVKSEDTALYYCTRPYHGYYWGQGTLVTVSA

SEQ ID NO: 161 - protein, 115 aa, synthetic construct
EVKLLESGGGLVQPGGSLKLSCAASGFDFSRYWMTWVRQAPGKGLEWIGEINPHSSTITYTPSLKDKFIISRDNAKNTLYLQMSKVSSEDTALYYCARPYYGYYWGQGTLVTVSA

SEQ ID NO: 162 - protein, 115 aa, synthetic construct
EVKLLESGGDLVQPGGSLKLSCAASGFDFSRFWMSWVRQAPGKGLEWIGEINPDSSTITYTPSLKDKFIISRDNAKSSLYLQMSKVTSEDTALYYCARPYYGYYWGQGTLVTVSA

SEQ ID NO: 163 - protein, 115 aa, synthetic construct
EVKLLESGGGLVQPGGSLKLSCAASGFDFSRYWMSWVRQAPGKGLEWIGEISPDSSTINYTASLKDKFNISRDNAKNTLYLQMSKVRSEDTALYYCARPYYGYYWGQGTLVTVSE

SEQ ID NO: 164 - protein, 123 aa, synthetic construct
EVQLQQSGPELVKPGASMKMSCKASGYSFTGYTMNWVKQSHGKNLEWIGLISPYSGGTNYNQKFKGKATLTVDKSSSTAYMEVFSLTSEDSGVYYCARSGFITTGVATNFDVWGAGTTVTVSS

SEQ ID NO: 165 - protein, 115 aa, synthetic construct
EVNLLESGGGLVQPGGSLKLSCEASGFDFSRFWMSWVRQTPGEGLEWIGEISPNSSTIHQTPSLKDKFFISRDNAKSTLFLQMTNVTSEDSALYYCARPYYGYYWGHGTLVTVSA

SEQ ID NO: 166 - protein, 115 aa, synthetic construct
EVKLLESGGGLVQPGGSLKLSCAASGFDFSRYWMSWVRQAPGKGLEWIGEINPDSSTIDLTPSLKDNFIISRDNAKNALYLQMSKVRSEDTALYYCTRPYYGYYWGQGTLVTVSA

SEQ ID NO: 167 - protein, 115 aa, synthetic construct
EVKLLESGGGLVQPGGSLKLSCAASGFDFSRYWMSWVRQAPGKGLEWIGEISPDSNTIDLTPSLKDNFIISRDNAKNALYLQMSKLRSEDTALYYCTRPYYGYYWGQGTLVTVSA

SEQ ID NO: 168 - protein, 119 aa, synthetic construct
EVKLVESGGGLVQSGGSRKLSCAASGFTFSDFGMAWVRQAPGKGPEWVAFISNLAYNIYYSDTVTGRFTISRENAKNTLYLEMSSLRSEDTAVYYCTRDRGLRETMDYWGQGTSVTVSS

SEQ ID NO: 169 - protein, 15 aa, synthetic construct
RASESVDSYGNSFMH

SEQ ID NO: 170 - protein, 16 aa, synthetic construct
KSSQSLLDSDGKTYLN

SEQ ID NO: 171 - protein, 16 aa, synthetic construct
KSSQSLLDSDGKTYLN

SEQ ID NO: 172 - protein, 14 aa, synthetic construct
RSSTGAVTTSNYAN

SEQ ID NO: 173 - protein, 11 aa, synthetic construct
RASQSISNNLH

SEQ ID NO: 174 - protein, 16 aa, synthetic construct
RSSKSLLHNNGNTYLC

SEQ ID NO: 175 - protein, 11 aa, synthetic construct
KASQDVSTAVA

SEQ ID NO: 176 - protein, 17 aa, synthetic construct
RSSQSLFNSTNQKNYLT

SEQ ID NO: 177 - protein, 16 aa, synthetic construct
GSSKSLLHKNGNTYLC

SEQ ID NO: 178 - protein, 16 aa, synthetic construct
RSSKSLLHNNGNTYLC

SEQ ID NO: 179 - protein, 17 aa, synthetic construct
KSSQSLLSSINQKNYLT

SEQ ID NO: 180 - protein, 17 aa, synthetic construct
RSSQSLLNSTNQKNYLT

SEQ ID NO: 181 - protein, 17 aa, synthetic construct
RSSQSLLNSTNQKNYLT

SEQ ID NO: 182 - protein, 17 aa, synthetic construct
KSSQSLLNSGNQKIYLT

SEQ ID NO: 183 - protein, 17 aa, synthetic construct
KSSQSLLNSTNQKNYLT

SEQ ID NO: 184 - protein, 17 aa, synthetic construct
KSSQSLFNSGNQKNYLT

SEQ ID NO: 185 - protein, 11 aa, synthetic construct
KASQDVSTAVA

SEQ ID NO: 186 - protein, 17 aa, synthetic construct
KSSQSLLNSINQKNYLT

SEQ ID NO: 187 - protein, 17 aa, synthetic construct
RSSQSLLNSTNQKNYLT

SEQ ID NO: 188 - protein, 17 aa, synthetic construct
RSSQSLLNSTNQKNYLT

SEQ ID NO: 189 - protein, 16 aa, synthetic construct
RSNKSLLHSNGNAYLS

SEQ ID NO: 190 - protein, 7 aa, synthetic construct
LASNLES

SEQ ID NO: 191 - protein, 7 aa, synthetic construct
LVSKLDS

SEQ ID NO: 192 - protein, 7 aa, synthetic construct
LVSKLDS

SEQ ID NO: 193 - protein, 7 aa, synthetic construct
GTSNRAP

SEQ ID NO: 194 - protein, 7 aa, synthetic construct
YASQSIS

SEQ ID NO: 195 - protein, 7 aa, synthetic construct
RMSDLAS

SEQ ID NO: 196 - protein, 7 aa, synthetic construct
SASYRYT

SEQ ID NO: 197 - protein, 7 aa, synthetic construct
WASSRES

SEQ ID NO: 198 - protein, 7 aa, synthetic construct
RMSNLAS

SEQ ID NO: 199 - protein, 7 aa, synthetic construct
RMSNLAS

SEQ ID NO: 200 - protein, 7 aa, synthetic construct
WATSRES

SEQ ID NO: 201 - protein, 7 aa, synthetic construct
WASTRES

SEQ ID NO: 202 - protein, 7 aa, synthetic construct
WASTKES

SEQ ID NO: 203 - protein, 7 aa, synthetic construct
WASIRES

SEQ ID NO: 204 - protein, 7 aa, synthetic construct
WASTRDS

SEQ ID NO: 205 - protein, 7 aa, synthetic construct
WAYTRES

SEQ ID NO: 206 - protein, 7 aa, synthetic construct
SASNRYT

SEQ ID NO: 207 - protein, 7 aa, synthetic construct
WATFRES

SEQ ID NO: 208 - protein, 7 aa, synthetic construct
WASTRES

SEQ ID NO: 209 - protein, 7 aa, synthetic construct
WASTRES

SEQ ID NO: 210 - protein, 7 aa, synthetic construct
RVSDLAS

SEQ ID NO: 211 - protein, 9 aa, synthetic construct
QQNNEDPLT

SEQ ID NO: 212 - protein, 9 aa, synthetic construct
WQGTHFPQT

SEQ ID NO: 213 - protein, 9 aa, synthetic construct
WQATHFPQT

SEQ ID NO: 214 - protein, 9 aa, synthetic construct
ALWYSTHYV

SEQ ID NO: 215 - protein, 9 aa, synthetic construct
QQSNSWPLT

SEQ ID NO: 216 - protein, 9 aa, synthetic construct
MQHLEYPYT

SEQ ID NO: 217 - protein, 9 aa, synthetic construct
QQHYTTPRT

SEQ ID NO: 218 - protein, 9 aa, synthetic construct
QNDYTSPFT

SEQ ID NO: 219 - protein, 9 aa, synthetic construct
MQHLEYPYT

SEQ ID NO: 220 - protein, 9 aa, synthetic construct
MQHLEYPYT

SEQ ID NO: 221 - protein, 9 aa, synthetic construct
HNDYSYPFT

SEQ ID NO: 222 - protein, 9 aa, synthetic construct
QNDYTSPFT

SEQ ID NO: 223 - protein, 9 aa, synthetic construct
QNDYTSPFT

SEQ ID NO: 224 - protein, 9 aa, synthetic construct
QNDYSSPFT

SEQ ID NO: 225 - protein, 9 aa, synthetic construct
HNDYISPFT

SEQ ID NO: 226 - protein, 9 aa, synthetic construct
HNDYTSPFT

SEQ ID NO: 227 - protein, 9 aa, synthetic construct
QQHYSTPRT

SEQ ID NO: 228 - protein, 9 aa, synthetic construct
HNDYSYPFT

SEQ ID NO: 229 - protein, 9 aa, synthetic construct
QNDYTSPFT

SEQ ID NO: 230 - protein, 9 aa, synthetic construct
QNDYTSPFT

SEQ ID NO: 231 - protein, 9 aa, synthetic construct
MQHLEFPYT

SEQ ID NO: 232 - protein, 23 aa, synthetic construct
NIVLTQSPASLAVSLGQRATISC

SEQ ID NO: 233 - protein, 23 aa, synthetic construct
DVVMTQTPLTLSVTIGQPASISC

SEQ ID NO: 234 - protein, 23 aa, synthetic construct
DVVMTQTPLTLSVTIGQPASISC

SEQ ID NO: 235 - protein, 22 aa, synthetic construct
QAVVTQESALTTSPGGTVILTC

SEQ ID NO: 236 - protein, 23 aa, synthetic construct
DIVLTQSPATLSVTPGDSVSLSC

SEQ ID NO: 237 - protein, 23 aa, synthetic construct
DIVMTQAAPSVPVTPGESVSISC

SEQ ID NO: 238 - protein, 23 aa, synthetic construct
DIVMTQSHKFMSTSVGDGVNITC

SEQ ID NO: 239 - protein, 23 aa, synthetic construct
DIVMTQSPSSLTVTAGEKVTMNC

SEQ ID NO: 240 - protein, 23 aa, synthetic construct
DIVMTQAAPSVPVTPGESVSISC

SEQ ID NO: 241 - protein, 23 aa, synthetic construct
DIVMTQAAPSVPVTPGESVSISC

SEQ ID NO: 242 - protein, 23 aa, synthetic construct
DIVMTQSPSSLTVTAGEKVTMSC

SEQ ID NO: 243 - protein, 23 aa, synthetic construct
DIVMTQSPSSLTVTAGEKVTMNC

SEQ ID NO: 244 - protein, 23 aa, synthetic construct
DIVMTQSPSSLTVTAGEKVTMNC

SEQ ID NO: 245 - protein, 23 aa, synthetic construct
DIVMTQSPSSLTVTAGEKVTMSC

SEQ ID NO: 246 - protein, 23 aa, synthetic construct
DIVMTQSPSSLTVTAGKEVTMSC

SEQ ID NO: 247 - protein, 23 aa, synthetic construct
DIVMTQSPSSLTVTAGEKVTMSC

SEQ ID NO: 248 - protein, 23 aa, synthetic construct
DIVMTQSHKFMSTSVGDRVSITC

SEQ ID NO: 249 - protein, 23 aa, synthetic construct
DIVMTQSPSSLTVTAGEKVTMSC

SEQ ID NO: 250 - protein, 23 aa, synthetic construct
DIVMTQSPSSLTVTAGEKVTMNC

SEQ ID NO: 251 - protein, 23 aa, synthetic construct
DIVMTQSPSSLTVTAGEKVTMNC

SEQ ID NO: 252 - protein, 23 aa, synthetic construct
DIVMTQAAPSVPVTPGESVSISC

SEQ ID NO: 253 - protein, 15 aa, synthetic construct
WYQQKPGQPPKLLIY

SEQ ID NO: 254 - protein, 15 aa, synthetic construct
WLLQRPGQSPKRLIY

SEQ ID NO: 255 - protein, 15 aa, synthetic construct
WLLQRPGQSPKRLIY

SEQ ID NO: 256 - protein, 15 aa, synthetic construct
WVQEKPDHLFTGLIG

SEQ ID NO: 257 - protein, 15 aa, synthetic construct
WYQQKSHESPRLLIK

SEQ ID NO: 258 - protein, 15 aa, synthetic construct
WFLQRPGQSPQLLIY

SEQ ID NO: 259 - protein, 15 aa, synthetic construct
WYQQKPGQSPRLLIY

SEQ ID NO: 260 - protein, 15 aa, synthetic construct
WYQQKPGQPPKLLIY

SEQ ID NO: 261 - protein, 15 aa, synthetic construct
WFLQRPGQSPQLLIY

SEQ ID NO: 262 - protein, 15 aa, synthetic construct
WFLQRPGQSPQLLIY

SEQ ID NO: 263 - protein, 15 aa, synthetic construct
WYQQKPGQPPKLLIY

SEQ ID NO: 264 - protein, 15 aa, synthetic construct
WYQQKPGQPPKLLIY

SEQ ID NO: 265 - protein, 15 aa, synthetic construct
WYQQKPGQPPKLLIY

SEQ ID NO: 266 - protein, 15 aa, synthetic construct
WYQQKPGQPPKLLIY

SEQ ID NO: 267 - protein, 15 aa, synthetic construct
WYQQKPGQSPKLLIY

SEQ ID NO: 268 - protein, 15 aa, synthetic construct
WYQQKPGQPPKLLIY

SEQ ID NO: 269 - protein, 15 aa, synthetic construct
WYQQRPGQSPKLLIY

SEQ ID NO: 270 - protein, 15 aa, synthetic construct
WYQQKPGQPPKLLIY

SEQ ID NO: 271 - protein, 15 aa, synthetic construct
WYQQRPGQPPKLLIY

SEQ ID NO: 272 - protein, 15 aa, synthetic construct
WYQQKPGQPPKLLIY

SEQ ID NO: 273 - protein, 15 aa, synthetic construct
WFLQRPGQSPQLLIY

SEQ ID NO: 274 - protein, 32 aa, synthetic construct
GVPARFSGSGSRTDFTLTIDPVEADDAATYYC

SEQ ID NO: 275 - protein, 32 aa, synthetic construct
GVPDRFTGSGSGTDFTLKISRVEAEDLGVYYC

SEQ ID NO: 276 - protein, 32 aa, synthetic construct; identical to SEQ ID NO: 275

SEQ ID NO: 277 - protein, 32 aa, synthetic construct
GVPVRFSGSLIGDKAALTITGAQTEDDAMYFC

SEQ ID NO: 278 - protein, 32 aa, synthetic construct
GIPSRFSGSGSGTDFTLSINSVETEDFGMYFC

SEQ ID NO: 279 - protein, 32 aa, synthetic construct
GVPDRFSGSGSGTAFTLRISRVEAEDVGVYYC

SEQ ID NO: 280 - protein, 32 aa, synthetic construct
GVPDRFTGSGSGTDFTFTISSMQAEDLAVYYC

SEQ ID NO: 281 - protein, 32 aa, synthetic construct
GVPDRFTGSGSGTDFTLTISTVQTEDLAVYYC

SEQ ID NO: 282 - protein, 32 aa, synthetic construct
GVPDRFSGSGSGTAFTLRISSVEAEDVGIYYC

SEQ ID NO: 283 - protein, 32 aa, synthetic construct; identical to SEQ ID NO: 279

SEQ ID NO: 284 - protein, 32 aa, synthetic construct
GVPDRFTGSGSGTDFTLTISSLQAEDLAVYYC

SEQ ID NO: 285 - protein, 32 aa, synthetic construct
GVPDRFTGSGSGTDFTLTINTVQAEDLAIYYC

SEQ ID NO: 286 - protein, 32 aa, synthetic construct
GVPDRFTGSGSGTDFTLTINTVQAEDLAVYYC

SEQ ID NO: 287 - protein, 32 aa, synthetic construct
GVPDRFTGSGSGTDFTLTISSVQAEDLAVYYC

SEQ ID NO: 288 - protein, 32 aa, synthetic construct
GVPDRFTGSGSGTDFTLTISSVQAEDLAIYYC

SEQ ID NO: 289 - protein, 32 aa, synthetic construct
GVPDRFTGSGSGTDFTLTIHGVQAEDLAVYYC

SEQ ID NO: 290 - protein, 32 aa, synthetic construct
GVPDRFTGSGSGTDFTFTISSMQAEDLAIYYC

SEQ ID NO: 291 - protein, 32 aa, synthetic construct
GVPDRFTGSGSGTDFTLTISGLQAEDLAVYYC

SEQ ID NO: 292 - protein, 32 aa, synthetic construct
GVPDRFTGSGSGTDFTLTISTVQAEDLAVYYC

SEQ ID NO: 293 - protein, 32 aa, synthetic construct
GVPDRFTGSGSGSDFTLTISTVQAEDLAVYYC

SEQ ID NO: 294 - protein, 32 aa, synthetic construct
GVPDRFSGSGSGTAFTLRISRVEAEDVGIYYC

SEQ ID NO: 295 - protein, 10 aa, synthetic construct
FGAGTKLELK

SEQ ID NO: 296 - protein, 10 aa, synthetic construct
FGGGTKLEIK

SEQ ID NO: 297 - protein, 10 aa, synthetic construct
FGGGTKLEIK

SEQ ID NO: 298 - protein, 10 aa, synthetic construct
FGGGTKVTVL

SEQ ID NO: 299 - protein, 10 aa, synthetic construct
FGAGTKLELK

SEQ ID NO: 300 - protein, 10 aa, synthetic construct
FGGGTKLEIK

SEQ ID NO: 301 - protein, 10 aa, synthetic construct
FGGGTKLEIK

SEQ ID NO: 302 - protein, 10 aa, synthetic construct
FGSGTRLEIK

SEQ ID NO: 303 - protein, 10 aa, synthetic construct
FGGGTKLEIK

SEQ ID NO: 304 - protein, 10 aa, synthetic construct
FGGGTKLEIK

SEQ ID NO: 305 - protein, 10 aa, synthetic construct
FGSGTRLEIK

SEQ ID NO: 306 - protein, 10 aa, synthetic construct
FGSGTKLEIK

SEQ ID NO: 307 - protein, 10 aa, synthetic construct
FGSGTKLEIK

SEQ ID NO: 308 - protein, 10 aa, synthetic construct
FGSGTKLEIK

SEQ ID NO: 309 - protein, 10 aa, synthetic construct
FGSGTKLEIK

SEQ ID NO: 310 - protein, 10 aa, synthetic construct
FGSGTKLEMK

SEQ ID NO: 311 - protein, 10 aa, synthetic construct
FGGGTKLEIK

SEQ ID NO: 312 - protein, 10 aa, synthetic construct
FGSGTRLEIK

SEQ ID NO: 313 - protein, 10 aa, synthetic construct
FGSGTKLEIK

SEQ ID NO: 314 - protein, 10 aa, synthetic construct
FGSGTKLEIK

SEQ ID NO: 315 - protein, 10 aa, synthetic construct
FGGGTKLEIK

SEQ ID NO: 316 - protein, 111 aa, synthetic construct
NIVLTQSPASLAVSLGQRATISCRASESVDSYGNSFMHWYQQKPGQPPKLLIYLASNLESGVPARFSGSGSRTDFTLTIDPVEADDAATYYCQQNNEDPLTFGAGTKLELK

SEQ ID NO: 317 - protein, 112 aa, synthetic construct
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 318 - protein, 112 aa, synthetic construct
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQATHFPQTFGGGTKLEIK

SEQ ID NO: 319 - protein, 109 aa, synthetic construct
QAVVTQESALTTSPGGTVILTCRSSTGAVTTSNYANWVQEKPDHLFTGLIGGTSNRAPGVPVRFSGSLIGDKAALTITGAQTEDDAMYFCALWYSTHYVFGGGTKVTVL

SEQ ID NO: 320 - protein, 107 aa, synthetic construct
DIVLTQSPATLSVTPGDSVSLSCRASQSISNNLHWYQQKSHESPRLLIKYASQSISGIPSRFSGSGSGTDFTLSINSVETEDFGMYFCQQSNSWPLTFGAGTKLELK

SEQ ID NO: 321 - protein, 112 aa, synthetic construct
DIVMTQAAPSVPVTPGESVSISCRSSKSLLHNNGNTYLCWFLQRPGQSPQLLIYRMSDLASGVPDRFSGSGSGTAFTLRISRVEAEDVGVYYCMQHLEYPYTFGGGTKLEIK

SEQ ID NO: 322 - protein, 107 aa, synthetic construct
DIVMTQSHKFMSTSVGDGVNITCKASQDVSTAVAWYQQKPGQSPRLLIYSASYRYTGVPDRFTGSGSGTDFTFTISSMQAEDLAVYYCQQHYTTPRTFGGGTKLEIK

SEQ ID NO: 323 - protein, 113 aa, synthetic construct
DIVMTQSPSSLTVTAGEKVTMNCRSSQSLFNSTNQKNYLTWYQQKPGQPPKLLIYWASSRESGVPDRFTGSGSGTDFTLTISTVQTEDLAVYYCQNDYTSPFTFGSGTRLEIK

SEQ ID NO: 324 - protein, 112 aa, synthetic construct
DIVMTQAAPSVPVTPGESVSISCGSSKSLLHKNGNTYLCWFLQRPGQSPQLLIYRMSNLASGVPDRFSGSGSGTAFTLRISSVEAEDVGIYYCMQHLEYPYTFGGGTKLEIK

SEQ ID NO: 325 - protein, 112 aa, synthetic construct
DIVMTQAAPSVPVTPGESVSISCRSSKSLLHNNGNTYLCWFLQRPGQSPQLLIYRMSNLASGVPDRFSGSGSGTAFTLRISRVEAEDVGVYYCMQHLEYPYTFGGGTKLEIK

SEQ ID NO: 326 - protein, 113 aa, synthetic construct
DIVMTQSPSSLTVTAGEKVTMSCKSSQSLLSSINQKNYLTWYQQKPGQPPKLLIYWATSRESGVPDRFTGSGSGTDFTLTISSLQAEDLAVYYCHNDYSYPFTFGSGTRLEIK

SEQ ID NO: 327 - protein, 113 aa, synthetic construct
DIVMTQSPSSLTVTAGEKVTMNCRSSQSLLNSTNQKNYLTWYQQKPGQPPKLLIYWASTRESGVPDRFTGSGSGTDFTLTINTVQAEDLAIYYCQNDYTSPFTFGSGTKLEIK

SEQ ID NO: 328 - protein, 113 aa, synthetic construct
DIVMTQSPSSLTVTAGEKVTMNCRSSQSLLNSTNQKNYLTWYQQKPGQPPKLLIYWASTKESGVPDRFTGSGSGTDFTLTINTVQAEDLAVYYCQNDYTSPFTFGSGTKLEIK

SEQ ID NO: 329 - protein, 113 aa, synthetic construct
DIVMTQSPSSLTVTAGEKVTMSCKSSQSLLNSGNQKIYLTWYQQKPGQPPKLLIYWASIRESGVPDRFTGSGSGTDFTLTISSVQAEDLAVYYCQNDYSSPFTFGSGTKLEIK

SEQ ID NO: 330 - protein, 113 aa, synthetic construct
DIVMTQSPSSLTVTAGKEVTMSCKSSQSLLNSTNQKNYLTWYQQKPGQSPKLLIYWASTRDSGVPDRFTGSGSGTDFTLTISSVQAEDLAIYYCHNDYISPFTFGSGTKLEIK

SEQ ID NO: 331 - protein, 113 aa, synthetic construct
DIVMTQSPSSLTVTAGEKVTMSCKSSQSLFNSGNQKNYLTWYQQKPGQPPKLLIYWAYTRESGVPDRFTGSGSGTDFTLTIHGVQAEDLAVYYCHNDYTSPFTFGSGTKLEMK

SEQ ID NO: 332 - protein, 107 aa, synthetic construct
DIVMTQSHKFMSTSVGDRVSITCKASQDVSTAVAWYQQRPGQSPKLLIYSASNRYTGVPDRFTGSGSGTDFTFTISSMQAEDLAIYYCQQHYSTPRTFGGGTKLEIK

SEQ ID NO: 333 - protein, 113 aa, synthetic construct
DIVMTQSPSSLTVTAGEKVTMSCKSSQSLLNSINQKNYLTWYQQKPGQPPKLLIYWATFRESGVPDRFTGSGSGTDFTLTISGLQAEDLAVYYCHNDYSYPFTFGSGTRLEIK

SEQ ID NO: 334 - protein, 113 aa, synthetic construct
DIVMTQSPSSLTVTAGEKVTMNCRSSQSLLNSTNQKNYLTWYQQRPGQPPKLLIYWASTRESGVPDRFTGSGSGTDFTLTISTVQAEDLAVYYCQNDYTSPFTFGSGTKLEIK

SEQ ID NO: 335 - protein, 113 aa, synthetic construct
DIVMTQSPSSLTVTAGEKVTMNCRSSQSLLNSTNQKNYLTWYQQKPGQPPKLLIYWASTRESGVPDRFTGSGSGSDFTLTISTVQAEDLAVYYCQNDYTSPFTFGSGTKLEIK

SEQ ID NO: 336 - protein, 112 aa, synthetic construct
DIVMTQAAPSVPVTPGESVSISCRSNKSLLHSNGNAYLSWFLQRPGQSPQLLIYRVSDLASGVPDRFSGSGSGTAFTLRISRVEAEDVGIYYCMQHLEFPYTFGGGTKLEIK

SEQ ID NO: 337 - protein, 30 aa, synthetic construct
QVQLVQSGSELKKPGASVKVSCKASGYTFT

SEQ ID NO: 338 - protein, 14 aa, synthetic construct
WVRQAPGQGLEWMG

SEQ ID NO: 339 - protein, 32 aa, synthetic construct
RFVFSLDTSVSTAYLQISSLKNEDMAVYFCAR

SEQ ID NO: 340 - protein, 11 aa, synthetic construct
WGQGTLVTVSS

SEQ ID NO: 341 - protein, 30 aa, synthetic construct
EVQLLESGGGLVQPGGSLRLSCAASGFTFS

SEQ ID NO: 342 - protein, 14 aa, synthetic construct
WVRQAPGKGLEWVS

SEQ ID NO: 343 - protein, 32 aa, synthetic construct
RFTISRDNSKNTLYLQMNSLRAEDTAVYYCAK

SEQ ID NO: 344 - protein, 11 aa, synthetic construct
WGQGTLVTVSS

SEQ ID NO: 345 - protein, 22 aa, synthetic construct
EAVVTQEPSLTVSPGGTVTLTC

SEQ ID NO: 346 - protein, 15 aa, synthetic construct
WVQQKPGQAFTGLIG

SEQ ID NO: 347 - protein, 32 aa, synthetic construct
GTPARFSGSLLGDKAALTLLGAQPEDEAEYFC

SEQ ID NO: 348 - protein, 10 aa, synthetic construct
FGGGTKLTVL

SEQ ID NO: 349 - protein, 23 aa, synthetic construct
DIVMTQSPDSLAVSLGERATMNC

SEQ ID NO: 350 - protein, 15 aa, synthetic construct
WYQQKPGQPPKLLIY

SEQ ID NO: 351 - protein, 32 aa, synthetic construct
GVPDRFSGSGSGTDFTLTISSLQAEDVAVYYC

SEQ ID NO: 352 - protein, 10 aa, synthetic construct
FGQGTRLEIK

SEQ ID NO: 353 - protein, 114 aa, synthetic construct
QVQLVQSGSELKKPGASVKVSCKASGYTFTNYGMNWVRQAPGQGLEWMGWINTYTGEPTYADDFKGRFVFSLDTSVSTAYLQISSLKNEDMAVYFCARYAMDYWGQGTLVTVSS

SEQ ID NO: 354 - protein, 109 aa, synthetic construct
EAVVTQEPSLTVSPGGTVTLTCRSSTGAVTTSNYANWVQQKPGQAFTGLIGGTSNRAPGTPARFSGSLLGDKAALTLLGAQPEDEAEYFCALWYSTHYVFGGGTKLTVL

SEQ ID NO: 355 - protein, 115 aa, synthetic construct
EVQLLESGGGLVQPGGSLRLSCAASGFTFSRFWMSWVRQAPGKGLEWVSEISPDSNTIDLTPSLKDRFTISRDNSKNTLYLQMNSLRAEDTAVYYCAKPYYGYYWGQGTLVTVSS

SEQ ID NO: 356 - protein, 113 aa, synthetic construct
DIVMTQSPDSLAVSLGERATMNCRSSQSLFSSTNQKNYLTWYQQKPGQPPKLLIYWASSRESGVPDRFSGSGSGTDFTLTISSLQAEDVAVYYCQNDYTSPFTFGQGTRLEIK

SEQ ID NO: 357 - protein, 5 aa, synthetic construct
  VARIANT 2: note = Y can be replaced by W or F
NYGMN

SEQ ID NO: 358 - protein, 17 aa, synthetic construct
  VARIANT 1: note = W can be replaced by Y or F
  VARIANT 3: note = N can be replaced by Q
WINTYTGEPTYADDFKG

SEQ ID NO: 359 - protein, 5 aa, synthetic construct
YAMDY

SEQ ID NO: 360 - protein, 14 aa, synthetic construct
  VARIANT 12: note = Y can be replaced by W or F
RSSTGAVTTSNYAN

SEQ ID NO: 361 - protein, 7 aa, synthetic construct
GTSNRAP

SEQ ID NO: 362 - protein, 9 aa, synthetic construct
  VARIANT 3: note = Y can be replaced by W or F
  VARIANT 8: note = Y can be replaced by W or F
ALYYSTHYV

SEQ ID NO: 363 - protein, 5 aa, synthetic construct
  VARIANT 3: note = Y can be replaced by W or F
RFYMS

SEQ ID NO: 364 - protein, 17 aa, synthetic construct
  VARIANT 1: note = E can be replaced by D
EISPDSNTIDLTPSLKD

SEQ ID NO: 365 - protein, 6 aa, synthetic construct
  VARIANT 5: note = Y can be replaced by W or F
PYYGYY

SEQ ID NO: 366 - protein, 17 aa, synthetic construct
  VARIANT 15: note = Y can be replaced by W or F
RSSQSLFSSTNQKNYLT

SEQ ID NO: 367 - protein, 7 aa, synthetic construct
WASSRES

SEQ ID NO: 368 - protein, 9 aa, synthetic construct
QNDYTSPFT

SEQ ID NO: 369 - protein, 17 aa, synthetic construct
RSSQSLFSSTNQKNYLT

SEQ ID NO: 370 - protein, 5 aa, synthetic construct
NHHIN

SEQ ID NO: 371 - protein, 5 aa, synthetic construct
NHHIN

SEQ ID NO: 372 - protein, 5 aa, synthetic construct
NNAMT

SEQ ID NO: 373 - protein, 5 aa, synthetic construct
RYGVS

SEQ ID NO: 374 - protein, 5 aa, synthetic construct
YYAMG

SEQ ID NO: 375 - protein, 5 aa, synthetic construct
HYAMG

SEQ ID NO: 376 - protein, 5 aa, synthetic construct
NNHMG

SEQ ID NO: 377 - protein, 5 aa, synthetic construct
SYWMI

SEQ ID NO: 378 - protein, 5 aa, synthetic construct
DYAVF

SEQ ID NO: 379 - protein, 5 aa, synthetic construct
KYAVN

SEQ ID NO: 380 - protein, 5 aa, synthetic construct
NYAVN

SEQ ID NO: 381 - protein, 5 aa, synthetic construct
NYAVY

SEQ ID NO: 382 - protein, 5 aa, synthetic construct
DYNFQ

SEQ ID NO: 383 - protein, 5 aa, synthetic construct
NYYMN

SEQ ID NO: 384 - protein, 5 aa, synthetic construct
SYYMN

SEQ ID NO: 385 - protein, 5 aa, synthetic construct
RYYMS

SEQ ID NO: 386 - protein, 5 aa, synthetic construct
RYVMH

SEQ ID NO: 387 - protein, 5 aa, synthetic construct
DYIML

SEQ ID NO: 388 - protein, 5 aa, synthetic construct
GYFMN

SEQ ID NO: 389 - protein, 5 aa, synthetic construct
HYSIG

SEQ ID NO: 390 - protein, 16 aa, synthetic construct
IISASGSAYYANWAKG

SEQ ID NO: 391 - protein, 16 aa, synthetic construct
IISNGGSAYYANWAKG

SEQ ID NO: 392 - protein, 16 aa, synthetic construct
YIWRGGETYYASWAKG

SEQ ID NO: 393 - protein, 17 aa, synthetic construct
YIYIGDGSTYYASWVNG

SEQ ID NO: 394 - protein, 16 aa, synthetic construct
IIDTNNVTYYASWAKG

SEQ ID NO: 395 - protein, 16 aa, synthetic construct
YITFGGIAYYANWAKG

SEQ ID NO: 396 - protein, 18 aa, synthetic construct
FINGNGVGSAFYANWAKG

SEQ ID NO: 397 - protein, 16 aa, synthetic construct
GISTIDNTYYASWAKG

SEQ ID NO: 398 - protein, 16 aa, synthetic construct
GISTIDNTYYATWAKG

SEQ ID NO: 399 - protein, 16 aa, synthetic construct
GISTIDNTYYSTWAKG

SEQ ID NO: 400 - protein, 16 aa, synthetic construct
FISSIGSTYYASWAKG

SEQ ID NO: 401 - protein, 16 aa, synthetic construct
FISSVGTTFYASWAKG

SEQ ID NO: 402 - protein, 16 aa, synthetic construct
FISSVGTTFYATWAKG

SEQ ID NO: 403 - protein, 16 aa, synthetic construct
FIDAVGDTYYARWAKG

SEQ ID NO: 404 - protein, 16 aa, synthetic construct
FIDAVGNTYYARWAKG

SEQ ID NO: 405 - protein, 16 aa, synthetic construct
FIDAVGSTYYARWAKG

SEQ ID NO: 406 - protein, 16 aa, synthetic construct
FISSVGTKFYASWAKG

SEQ ID NO: 407 - protein, 16 aa, synthetic construct
FISSVGTTYYAPWAKG

SEQ ID NO: 408 - protein, 16 aa, synthetic construct
TIGIGTDTYYATWAIG

SEQ ID NO: 409 - protein, 16 aa, synthetic construct
FINTGGSAYYASWAKG

SEQ ID NO: 410 - protein, 16 aa, synthetic construct
FIDTGGSAYFASWAKG

SEQ ID NO: 411 - protein, 16 aa, synthetic construct
SITTGGKGYYASWAKG

SEQ ID NO: 412 - protein, 17 aa, synthetic construct
YINPYNHDTKYNEKFKG

SEQ ID NO: 413 - protein, 17 aa, synthetic construct
NINPYYGSTSYNLKFKG

SEQ ID NO: 414 - protein, 17 aa, synthetic construct
WINTYTGEPTYVDDFKG

SEQ ID NO: 415 - protein, 17 aa, synthetic construct
RINHYNGDTFYNQKFKG

SEQ ID NO: 416 - protein, 17 aa, synthetic construct
WINTYTGEPTYSDDFKG

SEQ ID NO: 417 - protein, 17 aa, synthetic construct
CISTSDGSTYYADSVKG

SEQ ID NO: 418 - protein, 6 aa, synthetic construct
ESWMDL

SEQ ID NO: 419 - protein, 11 aa, synthetic construct
SYLSSGWGVDL

SEQ ID NO: 420 - protein, 5 aa, synthetic construct
GGLDL

SEQ ID NO: 421 - protein, 8 aa, synthetic construct
YADFAYNL

SEQ ID NO: 422 - protein, 9 aa, synthetic construct
GPGYGCFDL

SEQ ID NO: 423 - protein, 4 aa, synthetic construct
NFDT

SEQ ID NO: 424 - protein, 4 aa, synthetic construct
NFDI

SEQ ID NO: 425 - protein, 5 aa, synthetic construct
WNLDI

SEQ ID NO: 426 - protein, 5 aa, synthetic construct
WNTDI

SEQ ID NO: 427 - protein, 5 aa, synthetic construct
WNMDI

SEQ ID NO: 428 - protein, 8 aa, synthetic construct
DLYGGDSV

SEQ ID NO: 429 - protein, 8 aa, synthetic construct
DLYGGDSI

SEQ ID NO: 430 - protein, 8 aa, synthetic construct
DLYGGDNI

SEQ ID NO: 431 - protein, 9 aa, synthetic construct
FAVVAGLNI

SEQ ID NO: 432 - protein, 9 aa, synthetic construct
FAVVGGLNI

SEQ ID NO: 433 - protein, 9 aa, synthetic construct
WDYLTDFDI

SEQ ID NO: 434 - protein, 9 aa, synthetic construct
LDGYYAMDY

SEQ ID NO: 435 - protein, 11 aa, synthetic construct
DGNEKYGRFAY

SEQ ID NO: 436 - protein, 14 aa, synthetic construct
YGDYYGNSYRGFDY

SEQ ID NO: 437 - protein, 13 aa, synthetic construct
AEIHYDYDDAMDY

SEQ ID NO: 438 - protein, 14 aa, synthetic construct
YGDYYGSSYRGFDY

SEQ ID NO: 439 - protein, 12 aa, synthetic construct
GKGSRCHGDLDA

SEQ ID NO: 440 - protein, 13 aa, synthetic construct
QASQSVGGNNDLA

SEQ ID NO: 441 - protein, 13 aa, synthetic construct
QSSQSVGNNNDLA

SEQ ID NO: 442 - protein, 12 aa, synthetic construct
QSSQRVYNNWLS

SEQ ID NO: 443 - protein, 11 aa, synthetic construct
QASESIDNALA

SEQ ID NO: 444 - protein, 13 aa, synthetic construct
QSSQSVYNYNWLS

SEQ ID NO: 445 - protein, 13 aa, synthetic construct
QASQSVYSDNRLA

SEQ ID NO: 446 - protein, 13 aa, synthetic construct
QSSQSVYNNNLLA

SEQ ID NO: 447 - protein, 11 aa, synthetic construct
QANETIGNALA

SEQ ID NO: 448 - protein, 13 aa, synthetic construct
QSSQSVGSNNRLS

SEQ ID NO: 449 - protein, 13 aa, synthetic construct
QSSHSVGSNNRLS

SEQ ID NO: 450 - protein, 13 aa, synthetic construct
QSSQSVGNNKRLS

SEQ ID NO: 451 - protein, 13 aa, synthetic construct
QSSQTIWDNNWLA

SEQ ID NO: 452 - protein, 13 aa, synthetic construct
QSSQSIWDNNWLA

SEQ ID NO: 453 - protein, 13 aa, synthetic construct
HSSHSIYDNNWLS

SEQ ID NO: 454 - protein, 13 aa, synthetic construct
QSSESVWQNNRLS

SEQ ID NO: 455 - protein, 13 aa, synthetic construct
QASQSVWQNNRLS

SEQ ID NO: 456 - protein, 13 aa, synthetic construct
QSSQSVYNNNWLS

SEQ ID NO: 457 - protein, 12 aa, synthetic construct
QSSESVYSDWLS

SEQ ID NO: 458 - protein, 12 aa, synthetic construct
QSSQSVYSDWLS

SEQ ID NO: 459 - protein, 13 aa, synthetic construct
QASESVYNNNFLS

SEQ ID NO: 460 - protein, 11 aa, synthetic construct
SASQAISNYLN

SEQ ID NO: 461 - protein, 17 aa, synthetic construct
KSSQSLLYSSNQKNYLA

SEQ ID NO: 462 - protein, 11 aa, synthetic construct
KASQDVGTVVA

SEQ ID NO: 463 - protein, 7 aa, synthetic construct
KASNLAS

SEQ ID NO: 464 - protein, 7 aa, synthetic construct
RTSNLAP

SEQ ID NO: 465 - protein, 7 aa, synthetic construct
EASKLAS

SEQ ID NO: 466 - protein, 7 aa, synthetic construct
LASTLVP

SEQ ID NO: 467 - protein, 7 aa, synthetic construct
SVSTLAS

SEQ ID NO: 468 - protein, 7 aa, synthetic construct
RASTLAS

SEQ ID NO: 469 - protein, 7 aa, synthetic construct
STSTLAS

SEQ ID NO: 470 - protein, 7 aa, synthetic construct
LASTLAS

SEQ ID NO: 471 - protein, 7 aa, synthetic construct
GASNLAS

SEQ ID NO: 472 - protein, 7 aa, synthetic construct
GAFNLAS

SEQ ID NO: 473 - protein, 7 aa, synthetic construct
LASKLAS

SEQ ID NO: 474 - protein, 7 aa, synthetic construct
SASTLAS

SEQ ID NO: 475 - protein, 7 aa, synthetic construct
LASKLAT

SEQ ID NO: 476 - protein, 7 aa, synthetic construct
TASTLAS

SEQ ID NO: 477 - protein, 7 aa, synthetic construct
QASKLAS

SEQ ID NO: 478 - protein, 7 aa, synthetic construct
YASTLAS

SEQ ID NO: 479 - protein, 7 aa, synthetic construct
YTSSLYS

SEQ ID NO: 480 - protein, 7 aa, synthetic construct
WASTRHT

SEQ ID NO: 481 - protein, 10 aa, synthetic construct
QGYYPGWIRA

SEQ ID NO: 482 - protein, 10 aa, synthetic construct
LAYYPDWIRG

SEQ ID NO: 483 - protein, 13 aa, synthetic construct
LGTYDCSSADCGA

SEQ ID NO: 484 - protein, 12 aa, synthetic construct
QQGYSISNVDNV

SEQ ID NO: 485 - protein, 13 aa, synthetic construct
LGIYDCVSADCYG

SEQ ID NO: 486 - protein, 11 aa, synthetic construct
LGEYSDSGEDT

SEQ ID NO: 487 - protein, 10 aa, synthetic construct
QAFYNDDMWV

SEQ ID NO: 488 - protein, 13 aa, synthetic construct
LGSYNCADGDCNA

SEQ ID NO: 489 - protein, 13 aa, synthetic construct
LGSYNCANGDCNA

SEQ ID NO: 490 - protein, 13 aa, synthetic construct
LGSYNCADGDCNT

SEQ ID NO: 491 - protein, 13 aa, synthetic construct
LGSYNCAEGDCNA

SEQ ID NO: 492 - protein, 10 aa, synthetic construct
AGTYSDNIYS

SEQ ID NO: 493 - protein, 10 aa, synthetic construct
AGTYSDNVYT

SEQ ID NO: 494 - protein, 10 aa, synthetic construct
AGTYSYNIYT

SEQ ID NO: 495 - protein, 10 aa, synthetic construct
AGTYTYNIYT

SEQ ID NO: 496 - protein, 10 aa, synthetic construct
AGTYSDNIYT

SEQ ID NO: 497 - protein, 12 aa, synthetic construct
AGLKAWNDDDDG

SEQ ID NO: 498 - protein, 12 aa, synthetic construct
AGLRAWNDDDDG

SEQ ID NO: 499 - protein, 10 aa, synthetic construct
AGDYSVGIYT

SEQ ID NO: 500 - protein, 10 aa, synthetic construct
AGDYSVGIYA

SEQ ID NO: 501 - protein, 12 aa, synthetic construct
AGVKSDSTDGIA

SEQ ID NO: 502 - protein, 9 aa, synthetic construct
QQYIKLPRT

SEQ ID NO: 503 - protein, 8 aa, synthetic construct
QQYYSYPT

SEQ ID NO: 504 - protein, 9 aa, synthetic construct
QQYSSYPLT

SEQ ID NO: 505 - protein, 9 aa, synthetic construct
QQYYNYPLT

SEQ ID NO: 506 - protein, 9 aa, synthetic construct
QQYSRYPLT

SEQ ID NO: 507 - protein, 9 aa, synthetic construct
QQYSSFPLT

SEQ ID NO: 508 - protein, 29 aa, synthetic construct
GAPGAYPGAPAPGVYPGPPSGPGAYPSSG

(19) United States
(12) Patent Application Publication
(10) Pub. No.: US 2026/0034233 A1
(43) Pub. Date: Feb. 5, 2026

(54) ANTI-GALECTIN 3 ANTIBODIES AND THEIR USE IN EPILEPSY AND RELATED DISEASES

(71) Applicant: SUNMED THERAPEUTIC LIMITED, Hong Kong (CN)

(72) Inventor: Dongxu SUN, Shenzhen (CN)

(21) Appl. No.: 19/347,462

(22) Filed: Oct. 1, 2025

Related U.S. Application Data
(63) Continuation of application No. PCT/CN2025/081169, filed on Mar. 7, 2025.

(30) Foreign Application Priority Data
Mar. 8, 2024 (WO) PCT/CN2024/080726

(51) Int. Cl. A61K 47/68; A61K 9/00; A61K 39/00; A61P 25/08; C07K 16/28

(52) U.S. Cl. CPC A61K 47/6849; A61K 9/0019; A61P 25/08; C07K 16/2851; A61K 2039/505; A61K 2039/545; C07K 2317/24; C07K 2317/33; C07K 2317/565

(57) ABSTRACT

Provided herein are antibodies that target Galectin-3. Such antibodies are used in methods of treating epilepsy and related neurological disorders, such as Alzheimer's disease (AD) and Parkinson's disease (PD).

### CROSS REFERENCE TO RELATED APPLICATIONS

This application is a continuation of International Application No. PCT/CN2025/081169, filed Mar. 7, 2025, which claims priority to International Application No. PCT/CN2024/080726, filed Mar. 8, 2024, the disclosure of which is herein incorporated by reference in its entirety for all purposes.

### SEQUENCE LISTING

The instant application contains a Sequence Listing which has been submitted via Patent Center and is hereby incorporated by reference in its entirety. Said .xml copy, created on Sep. 29, 2025, is named 112130-1526877-000100US, and is 452,679 bytes in size.

### BACKGROUND

Epilepsy is a neurological disease characterized by recurrent spontaneous seizures. Despite the efficacy of current anti-epileptic drugs, almost 30% of patients with epilepsy are refractory to medical treatment, have progressive cognitive impairment, and may require neurosurgical resection of the epileptic focus to alleviate seizure recurrence. Although the exact cellular and molecular mechanisms of epileptogenesis are not clear, it is postulated that focal or systemic unregulated inflammatory processes lead to aberrant neural connectivity and the hyper-excitable neuronal network, which mediate the onset of epilepsy. Epileptogenesis is associated with an increased and persistent inflammatory state in the microenvironment of neural tissues, which can lead to the production of cytokines by glial cells and neurons. A safe and efficient treatment of epilepsy and/or related neurological disorders is highly needed.

### SUMMARY

In one aspect, provided herein is an antibody or a binding fragment thereof that binds to Galectin-3, wherein the antibody comprises: a heavy chain variable (V_{H}) region comprising: a HCDR1 amino acid sequence at least 70% identical to any one of SEQ ID NOS: 1-21 and 370-389, a HCDR2 amino acid sequence at least 70% identical to any one of SEQ ID NOS: 22-42 and 390-417, and/or a HCDR3 amino acid sequence at least 70% identical to any one of SEQ ID NOS: 43-63 and 418-439, and/or a light chain variable (V_{L}) region comprising: a LCDR1 amino acid sequence at least 70% identical to any one of SEQ ID NOS: 169-189 and 440-462, a LCDR2 amino acid sequence at least 70% identical to any one of SEQ ID NOS: 190-210 and 463-480, and/or a LCDR3 amino acid sequence at least 70% identical to any one of SEQ ID NOS: 211-231 and 481-507. In some embodiments, the Galectin-3 is human Galectin-3.

In some embodiments, the antibody comprises: a heavy chain variable region comprising a HCDR1 amino acid sequence at least 70% identical to SEQ ID NO: 1, a HCDR2 amino acid sequence at least 70% identical to SEQ ID NO: 22, and a HCDR3 amino acid sequence at least 70% identical to SEQ ID NO: 43; and a light chain variable region comprising a LCDR1 amino acid sequence at least 70% identical to SEQ ID NO: 169, a LCDR2 amino acid sequence at least 70% identical to SEQ ID NO: 190, and a LCDR3 amino acid sequence at least 70% identical to SEQ ID NO: 211, or
a heavy chain variable region comprising a HCDR1 amino acid sequence at least 70% identical to SEQ ID NO: 2, a HCDR2 amino acid sequence at least 70% identical to SEQ ID NO: 23, and a HCDR3 amino acid sequence at least 70% identical to SEQ ID NO: 44; and a light chain variable region comprising a LCDR1 amino acid sequence at least 70% identical to SEQ ID NO: 170, a LCDR2 amino acid sequence at least 70% identical to SEQ ID NO: 191, and a LCDR3 amino acid sequence at least 70% identical to SEQ ID NO: 212, or
a heavy chain variable region comprising a HCDR1 amino acid sequence at least 70% identical to SEQ ID NO: 3, a HCDR2 amino acid sequence at least 70% identical to SEQ ID NO: 24, and a HCDR3 amino acid sequence at least 70% identical to SEQ ID NO: 45; and a light chain variable region comprising a LCDR1 amino acid sequence at least 70% identical to SEQ ID NO: 171, a LCDR2 amino acid sequence at least 70% identical to SEQ ID NO: 192, and a LCDR3 amino acid sequence at least 70% identical to SEQ ID NO: 213, or
a heavy chain variable region comprising a HCDR1 amino acid sequence at least 70% identical to SEQ ID NO: 4, a HCDR2 amino acid sequence at least 70% identical to SEQ ID NO: 25, and a HCDR3 amino acid sequence at least 70% identical to SEQ ID NO: 46; and a light chain variable region comprising a LCDR1 amino acid sequence at least 70% identical to SEQ ID NO: 172, a LCDR2 amino acid sequence at least 70% identical to SEQ ID NO: 193, and a LCDR3 amino acid sequence at least 70% identical to SEQ ID NO: 214, or
a heavy chain variable region comprising a HCDR1 amino acid sequence at least 70% identical to SEQ ID NO: 5, a HCDR2 amino acid sequence at least 70% identical to SEQ ID NO: 26, and a HCDR3 amino acid sequence at least 70% identical to SEQ ID NO: 47; and a light chain variable region comprising a LCDR1 amino acid sequence at least 70% identical to SEQ ID NO: 173, a LCDR2 amino acid sequence at least 70% identical to SEQ ID NO: 194, and a LCDR3 amino acid sequence at least 70% identical to SEQ ID NO: 215, or
a heavy chain variable region comprising a HCDR1 amino acid sequence at least 70% identical to SEQ ID NO: 6, a HCDR2 amino acid sequence at least 70% identical to SEQ ID NO: 27, and a HCDR3 amino acid sequence at least 70% identical to SEQ ID NO: 48; and a light chain variable region comprising a LCDR1 amino acid sequence at least 70% identical to SEQ ID NO: 174, a LCDR2 amino acid sequence at least 70% identical to SEQ ID NO: 195, and a LCDR3 amino acid sequence at least 70% identical to SEQ ID NO: 216, or
a heavy chain variable region comprising a HCDR1 amino acid sequence at least 70% identical to SEQ ID NO: 7, a HCDR2 amino acid sequence at least 70% identical to SEQ ID NO: 28, and a HCDR3 amino acid sequence at least 70% identical to SEQ ID NO: 49; and a light chain variable region comprising a LCDR1 amino acid sequence at least 70% identical to SEQ ID NO: 175, a LCDR2 amino acid sequence at least 70% identical to SEQ ID NO: 196, and a LCDR3 amino acid sequence at least 70% identical to SEQ ID NO: 217, or
a heavy chain variable region comprising a HCDR1 amino acid sequence at least 70% identical to SEQ ID NO: 8, a HCDR2 amino acid sequence at least 70% identical to SEQ ID NO: 29, and a HCDR3 amino acid sequence at least 70% identical to SEQ ID NO: 50; and a light chain variable region comprising a LCDR1 amino acid sequence at least 70% identical to SEQ ID NO: 176, a LCDR2 amino acid sequence at least 70% identical to SEQ ID NO: 197, and a LCDR3 amino acid sequence at least 70% identical to SEQ ID NO: 218, or
a heavy chain variable region comprising a HCDR1 amino acid sequence at least 70% identical to SEQ ID NO: 9, a HCDR2 amino acid sequence at least 70% identical to SEQ ID NO: 30, and a HCDR3 amino acid sequence at least 70% identical to SEQ ID NO: 51; and a light chain variable region comprising a LCDR1 amino acid sequence at least 70% identical to SEQ ID NO: 177, a LCDR2 amino acid sequence at least 70% identical to SEQ ID NO: 198, and a LCDR3 amino acid sequence at least 70% identical to SEQ ID NO: 219, or
a heavy chain variable region comprising a HCDR1 amino acid sequence at least 70% identical to SEQ ID NO: 10, a HCDR2 amino acid sequence at least 70% identical to SEQ ID NO: 31, and a HCDR3 amino acid sequence at least 70% identical to SEQ ID NO: 52; and a light chain variable region comprising a LCDR1 amino acid sequence at least 70% identical to SEQ ID NO: 178, a LCDR2 amino acid sequence at least 70% identical to SEQ ID NO: 199, and a LCDR3 amino acid sequence at least 70% identical to SEQ ID NO: 220, or
a heavy chain variable region comprising a HCDR1 amino acid sequence at least 70% identical to SEQ ID NO: 11, a HCDR2 amino acid sequence at least 70% identical to SEQ ID NO: 32, and a HCDR3 amino acid sequence at least 70% identical to SEQ ID NO: 53; and a light chain variable region comprising a LCDR1 amino acid sequence at least 70% identical to SEQ ID NO: 179, a LCDR2 amino acid sequence at least 70% identical to SEQ ID NO: 200, and a LCDR3 amino acid sequence at least 70% identical to SEQ ID NO: 221, or
a heavy chain variable region comprising a HCDR1 amino acid sequence at least 70% identical to SEQ ID NO: 12, a HCDR2 amino acid sequence at least 70% identical to SEQ ID NO: 33, and a HCDR3 amino acid sequence at least 70% identical to SEQ ID NO: 54; and a light chain variable region comprising a LCDR1 amino acid sequence at least 70% identical to SEQ ID NO: 180, a LCDR2 amino acid sequence at least 70% identical to SEQ ID NO: 201, and a LCDR3 amino acid sequence at least 70% identical to SEQ ID NO: 222, or
a heavy chain variable region comprising a HCDR1 amino acid sequence at least 70% identical to SEQ ID NO: 13, a HCDR2 amino acid sequence at least 70% identical to SEQ ID NO: 34, and a HCDR3 amino acid sequence at least 70% identical to SEQ ID NO: 55; and a light chain variable region comprising a LCDR1 amino acid sequence at least 70% identical to SEQ ID NO: 181, a LCDR2 amino acid sequence at least 70% identical to SEQ ID NO: 202, and a LCDR3 amino acid sequence at least 70% identical to SEQ ID NO: 223, or
a heavy chain variable region comprising a HCDR1 amino acid sequence at least 70% identical to SEQ ID NO: 14, a HCDR2 amino acid sequence at least 70% identical to SEQ ID NO: 35, and a HCDR3 amino acid sequence at least 70% identical to SEQ ID NO: 56; and a light chain variable region comprising a LCDR1 amino acid sequence at least 70% identical to SEQ ID NO: 182, a LCDR2 amino acid sequence at least 70% identical to SEQ ID NO: 203, and a LCDR3 amino acid sequence at least 70% identical to SEQ ID NO: 224, or
a heavy chain variable region comprising a HCDR1 amino acid sequence at least 70% identical to SEQ ID NO: 15, a HCDR2 amino acid sequence at least 70% identical to SEQ ID NO: 36, and a HCDR3 amino acid sequence at least 70% identical to SEQ ID NO: 57; and a light chain variable region comprising a LCDR1 amino acid sequence at least 70% identical to SEQ ID NO: 183, a LCDR2 amino acid sequence at least 70% identical to SEQ ID NO: 204, and a LCDR3 amino acid sequence at least 70% identical to SEQ ID NO: 225, or
a heavy chain variable region comprising a HCDR1 amino acid sequence at least 70% identical to SEQ ID NO: 16, a HCDR2 amino acid sequence at least 70% identical to SEQ ID NO: 37, and a HCDR3 amino acid sequence at least 70% identical to SEQ ID NO: 58; and a light chain variable region comprising a LCDR1 amino acid sequence at least 70% identical to SEQ ID NO: 184, a LCDR2 amino acid sequence at least 70% identical to SEQ ID NO: 205, and a LCDR3 amino acid sequence at least 70% identical to SEQ ID NO: 226, or
a heavy chain variable region comprising a HCDR1 amino acid sequence at least 70% identical to SEQ ID NO: 17, a HCDR2 amino acid sequence at least 70% identical to SEQ ID NO: 38, and a HCDR3 amino acid sequence at least 70% identical to SEQ ID NO: 59; and a light chain variable region comprising a LCDR1 amino acid sequence at least 70% identical to SEQ ID NO: 185, a LCDR2 amino acid sequence at least 70% identical to SEQ ID NO: 206, and a LCDR3 amino acid sequence at least 70% identical to SEQ ID NO: 227, or
a heavy chain variable region comprising a HCDR1 amino acid sequence at least 70% identical to SEQ ID NO: 18, a HCDR2 amino acid sequence at least 70% identical to SEQ ID NO: 39, and a HCDR3 amino acid sequence at least 70% identical to SEQ ID NO: 60; and a light chain variable region comprising a LCDR1 amino acid sequence at least 70% identical to SEQ ID NO: 186, a LCDR2 amino acid sequence at least 70% identical to SEQ ID NO: 207, and a LCDR3 amino acid sequence at least 70% identical to SEQ ID NO: 228, or
a heavy chain variable region comprising a HCDR1 amino acid sequence at least 70% identical to SEQ ID NO: 19, a HCDR2 amino acid sequence at least 70% identical to SEQ ID NO: 40, and a HCDR3 amino acid sequence at least 70% identical to SEQ ID NO: 61; and a light chain variable region comprising a LCDR1 amino acid sequence at least 70% identical to SEQ ID NO: 187, a LCDR2 amino acid sequence at least 70% identical to SEQ ID NO: 208, and a LCDR3 amino acid sequence at least 70% identical to SEQ ID NO: 229, or
a heavy chain variable region comprising a HCDR1 amino acid sequence at least 70% identical to SEQ ID NO: 20, a HCDR2 amino acid sequence at least 70% identical to SEQ ID NO: 41, and a HCDR3 amino acid sequence at least 70% identical to SEQ ID NO: 62; and a light chain variable region comprising a LCDR1 amino acid sequence at least 70% identical to SEQ ID NO: 188, a LCDR2 amino acid sequence at least 70% identical to SEQ ID NO: 209, and a LCDR3 amino acid sequence at least 70% identical to SEQ ID NO: 230, or
a heavy chain variable region comprising a HCDR1 amino acid sequence at least 70% identical to SEQ ID NO: 21, a HCDR2 amino acid sequence at least 70% identical to SEQ ID NO: 42, and a HCDR3 amino acid sequence at least 70% identical to SEQ ID NO: 63; and a light chain variable region comprising a LCDR1 amino acid sequence at least 70% identical to SEQ ID NO: 189, a LCDR2 amino acid sequence at least 70% identical to SEQ ID NO: 210, and a LCDR3 amino acid sequence at least 70% identical to SEQ ID NO: 231.

In some embodiments, the antibody comprises: a heavy chain variable region comprising a HCDR1 amino acid sequence at least 70% identical to SEQ ID NO: 370, a HCDR2 amino acid sequence at least 70% identical to SEQ ID NO: 390, and a HCDR3 amino acid sequence at least 70% identical to SEQ ID NO: 418; and a light chain variable region comprising a LCDR1 amino acid sequence at least 70% identical to SEQ ID NO: 440, a LCDR2 amino acid sequence at least 70% identical to SEQ ID NO: 463, and a LCDR3 amino acid sequence at least 70% identical to SEQ ID NO: 481, or
a heavy chain variable region comprising a HCDR1 amino acid sequence at least 70% identical to SEQ ID NO: 371, a HCDR2 amino acid sequence at least 70% identical to SEQ ID NO: 391, and a HCDR3 amino acid sequence at least 70% identical to SEQ ID NO: 418; and a light chain variable region comprising a LCDR1 amino acid sequence at least 70% identical to SEQ ID NO: 441, a LCDR2 amino acid sequence at least 70% identical to SEQ ID NO: 464, and a LCDR3 amino acid sequence at least 70% identical to SEQ ID NO: 482, or
a heavy chain variable region comprising a HCDR1 amino acid sequence at least 70% identical to SEQ ID NO: 372, a HCDR2 amino acid sequence at least 70% identical to SEQ ID NO: 392, and a HCDR3 amino acid sequence at least 70% identical to DNL; and a light chain variable region comprising a LCDR1 amino acid sequence at least 70% identical to SEQ ID NO: 442, a LCDR2 amino acid sequence at least 70% identical to SEQ ID NO: 465, and a LCDR3 amino acid sequence at least 70% identical to SEQ ID NO: 483, or
a heavy chain variable region comprising a HCDR1 amino acid sequence at least 70% identical to SEQ ID NO: 373, a HCDR2 amino acid sequence at least 70% identical to SEQ ID NO: 393, and a HCDR3 amino acid sequence at least 70% identical to SEQ ID NO: 419; and a light chain variable region comprising a LCDR1 amino acid sequence at least 70% identical to SEQ ID NO: 443, a LCDR2 amino acid sequence at least 70% identical to SEQ ID NO: 466, and a LCDR3 amino acid sequence at least 70% identical to SEQ ID NO: 484, or
a heavy chain variable region comprising a HCDR1 amino acid sequence at least 70% identical to SEQ ID NO: 374, a HCDR2 amino acid sequence at least 70% identical to SEQ ID NO: 394, and a HCDR3 amino acid sequence at least 70% identical to SEQ ID NO: 420; and a light chain variable region comprising a LCDR1 amino acid sequence at least 70% identical to SEQ ID NO: 444, a LCDR2 amino acid sequence at least 70% identical to SEQ ID NO: 467, and a LCDR3 amino acid sequence at least 70% identical to SEQ ID NO: 485, or
a heavy chain variable region comprising a HCDR1 amino acid sequence at least 70% identical to SEQ ID NO: 375, a HCDR2 amino acid sequence at least 70% identical to SEQ ID NO: 395, and a HCDR3 amino acid sequence at least 70% identical to SEQ ID NO: 421; and a light chain variable region comprising a LCDR1 amino acid sequence at least 70% identical to SEQ ID NO: 445, a LCDR2 amino acid sequence at least 70% identical to SEQ ID NO: 468, and a LCDR3 amino acid sequence at least 70% identical to SEQ ID NO: 486, or
a heavy chain variable region comprising a HCDR1 amino acid sequence at least 70% identical to SEQ ID NO: 376, a HCDR2 amino acid sequence at least 70% identical to SEQ ID NO: 396, and a HCDR3 amino acid sequence at least 70% identical to SEQ ID NO: 422; and a light chain variable region comprising a LCDR1 amino acid sequence at least 70% identical to SEQ ID NO: 446, a LCDR2 amino acid sequence at least 70% identical to SEQ ID NO: 469, and a LCDR3 amino acid sequence at least 70% identical to SEQ ID NO: 487, or
a heavy chain variable region comprising a HCDR1 amino acid sequence at least 70% identical to SEQ ID NO: 373, a HCDR2 amino acid sequence at least 70% identical to SEQ ID NO: 393, and a HCDR3 amino acid sequence at least 70% identical to SEQ ID NO: 419; and a light chain variable region comprising a LCDR1 amino acid sequence at least 70% identical to SEQ ID NO: 447, a LCDR2 amino acid sequence at least 70% identical to SEQ ID NO: 470, and a LCDR3 amino acid sequence at least 70% identical to SEQ ID NO: 484, or
a heavy chain variable region comprising a HCDR1 amino acid sequence at least 70% identical to SEQ ID NO: 377, a HCDR2 amino acid sequence at least 70% identical to SEQ ID NO: 397, and a HCDR3 amino acid sequence at least 70% identical to SEQ ID NO: 423; and a light chain variable region comprising a LCDR1 amino acid sequence at least 70% identical to SEQ ID NO: 448, a LCDR2 amino acid sequence at least 70% identical to SEQ ID NO: 471, and a LCDR3 amino acid sequence at least 70% identical to SEQ ID NO: 488, or
a heavy chain variable region comprising a HCDR1 amino acid sequence at least 70% identical to SEQ ID NO: 377, a HCDR2 amino acid sequence at least 70% identical to SEQ ID NO: 397, and a HCDR3 amino acid sequence at least 70% identical to SEQ ID NO: 423; and a light chain variable region comprising a LCDR1 amino acid sequence at least 70% identical to SEQ ID NO: 449, a LCDR2 amino acid sequence at least 70% identical to SEQ ID NO: 472, and a LCDR3 amino acid sequence at least 70% identical to SEQ ID NO: 488, or
a heavy chain variable region comprising a HCDR1 amino acid sequence at least 70% identical to SEQ ID NO: 377, a HCDR2 amino acid sequence at least 70% identical to SEQ ID NO: 397, and a HCDR3 amino acid sequence at least 70% identical to SEQ ID NO: 423; and a light chain variable region comprising a LCDR1 amino acid sequence at least 70% identical to SEQ ID NO: 450, a LCDR2 amino acid sequence at least 70% identical to SEQ ID NO: 471, and a LCDR3 amino acid sequence at least 70% identical to SEQ ID NO: 488, or
a heavy chain variable region comprising a HCDR1 amino acid sequence at least 70% identical to SEQ ID NO: 377, a HCDR2 amino acid sequence at least 70% identical to SEQ ID NO: 398, and a HCDR3 amino acid sequence at least 70% identical to SEQ ID NO: 423; and a light chain variable region comprising a LCDR1 amino acid sequence at least 70% identical to SEQ ID NO: 448, a LCDR2 amino acid sequence at least 70% identical to SEQ ID NO: 471, and a LCDR3 amino acid sequence at least 70% identical to SEQ ID NO: 488, or
a heavy chain variable region comprising a HCDR1 amino acid sequence at least 70% identical to SEQ ID NO: 377, a HCDR2 amino acid sequence at least 70% identical to SEQ ID NO: 397, and a HCDR3 amino acid sequence at least 70% identical to SEQ ID NO: 424; and a light chain variable region comprising a LCDR1 amino acid sequence at least 70% identical to SEQ ID NO: 448, a LCDR2 amino acid sequence at least 70% identical to SEQ ID NO: 472, and a LCDR3 amino acid sequence at least 70% identical to SEQ ID NO: 489, or
a heavy chain variable region comprising a HCDR1 amino acid sequence at least 70% identical to SEQ ID NO: 377, a HCDR2 amino acid sequence at least 70% identical to SEQ ID NO: 397, and a HCDR3 amino acid sequence at least 70% identical to SEQ ID NO: 423; and a light chain variable region comprising a LCDR1 amino acid sequence at least 70% identical to SEQ ID NO: 448, a LCDR2 amino acid sequence at least 70% identical to SEQ ID NO: 472, and a LCDR3 amino acid sequence at least 70% identical to SEQ ID NO: 488, or
a heavy chain variable region comprising a HCDR1 amino acid sequence at least 70% identical to SEQ ID NO: 377, a HCDR2 amino acid sequence at least 70% identical to SEQ ID NO: 397, and a HCDR3 amino acid sequence at least 70% identical to SEQ ID NO: 423; and a light chain variable region comprising a LCDR1 amino acid sequence at least 70% identical to SEQ ID NO: 448, a LCDR2 amino acid sequence at least 70% identical to SEQ ID NO: 471, and a LCDR3 amino acid sequence at least 70% identical to SEQ ID NO: 490, or
a heavy chain variable region comprising a HCDR1 amino acid sequence at least 70% identical to SEQ ID NO: 377, a HCDR2 amino acid sequence at least 70% identical to SEQ ID NO: 398, and a HCDR3 amino acid sequence at least 70% identical to SEQ ID NO: 423; and a light chain variable region comprising a LCDR1 amino acid sequence at least 70% identical to SEQ ID NO: 448, a LCDR2 amino acid sequence at least 70% identical to SEQ ID NO: 471, and a LCDR3 amino acid sequence at least 70% identical to SEQ ID NO: 491, or
a heavy chain variable region comprising a HCDR1 amino acid sequence at least 70% identical to SEQ ID NO: 377, a HCDR2 amino acid sequence at least 70% identical to SEQ ID NO: 399, and a HCDR3 amino acid sequence at least 70% identical to SEQ ID NO: 424; and a light chain variable region comprising a LCDR1 amino acid sequence at least 70% identical to SEQ ID NO: 448, a LCDR2 amino acid sequence at least 70% identical to SEQ ID NO: 472, and a LCDR3 amino acid sequence at least 70% identical to SEQ ID NO: 489, or
a heavy chain variable region comprising a HCDR1 amino acid sequence at least 70% identical to SEQ ID NO: 377, a HCDR2 amino acid sequence at least 70% identical to SEQ ID NO: 397, and a HCDR3 amino acid sequence at least 70% identical to SEQ ID NO: 424; and a light chain variable region comprising a LCDR1 amino acid sequence at least 70% identical to SEQ ID NO: 448, a LCDR2 amino acid sequence at least 70% identical to SEQ ID NO: 471, and a LCDR3 amino acid sequence at least 70% identical to SEQ ID NO: 488, or
a heavy chain variable region comprising a HCDR1 amino acid sequence at least 70% identical to SEQ ID NO: 378, a HCDR2 amino acid sequence at least 70% identical to SEQ ID NO: 400, and a HCDR3 amino acid sequence at least 70% identical to SEQ ID NO: 425; and a light chain variable region comprising a LCDR1 amino acid sequence at least 70% identical to SEQ ID NO: 451, a LCDR2 amino acid sequence at least 70% identical to SEQ ID NO: 473, and a LCDR3 amino acid sequence at least 70% identical to SEQ ID NO: 492, or
a heavy chain variable region comprising a HCDR1 amino acid sequence at least 70% identical to SEQ ID NO: 378, a HCDR2 amino acid sequence at least 70% identical to SEQ ID NO: 401, and a HCDR3 amino acid sequence at least 70% identical to SEQ ID NO: 425; and a light chain variable region comprising a LCDR1 amino acid sequence at least 70% identical to SEQ ID NO: 452, a LCDR2 amino acid sequence at least 70% identical to SEQ ID NO: 473, and a LCDR3 amino acid sequence at least 70% identical to SEQ ID NO: 493, or
a heavy chain variable region comprising a HCDR1 amino acid sequence at least 70% identical to SEQ ID NO: 378, a HCDR2 amino acid sequence at least 70% identical to SEQ ID NO: 402, and a HCDR3 amino acid sequence at least 70% identical to SEQ ID NO: 425; and a light chain variable region comprising a LCDR1 amino acid sequence at least 70% identical to SEQ ID NO: 452, a LCDR2 amino acid sequence at least 70% identical to SEQ ID NO: 473, and a LCDR3 amino acid sequence at least 70% identical to SEQ ID NO: 493, or
a heavy chain variable region comprising a HCDR1 amino acid sequence at least 70% identical to SEQ ID NO: 379, a HCDR2 amino acid sequence at least 70% identical to SEQ ID NO: 403, and a HCDR3 amino acid sequence at least 70% identical to SEQ ID NO: 426; and a light chain variable region comprising a LCDR1 amino acid sequence at least 70% identical to SEQ ID NO: 453, a LCDR2 amino acid sequence at least 70% identical to SEQ ID NO: 470, and a LCDR3 amino acid sequence at least 70% identical to SEQ ID NO: 494, or
a heavy chain variable region comprising a HCDR1 amino acid sequence at least 70% identical to SEQ ID NO: 380, a HCDR2 amino acid sequence at least 70% identical to SEQ ID NO: 403, and a HCDR3 amino acid sequence at least 70% identical to SEQ ID NO: 426; and a light chain variable region comprising a LCDR1 amino acid sequence at least 70% identical to SEQ ID NO: 453, a LCDR2 amino acid sequence at least 70% identical to SEQ ID NO: 470, and a LCDR3 amino acid sequence at least 70% identical to SEQ ID NO: 494, or
a heavy chain variable region comprising a HCDR1 amino acid sequence at least 70% identical to SEQ ID NO: 380, a HCDR2 amino acid sequence at least 70% identical to SEQ ID NO: 403, and a HCDR3 amino acid sequence at least 70% identical to SEQ ID NO: 426; and a light chain variable region comprising a LCDR1 amino acid sequence at least 70% identical to SEQ ID NO: 453, a LCDR2 amino acid sequence at least 70% identical to SEQ ID NO: 474, and a LCDR3 amino acid sequence at least 70% identical to SEQ ID NO: 494, or
a heavy chain variable region comprising a HCDR1 amino acid sequence at least 70% identical to SEQ ID NO: 381, a HCDR2 amino acid sequence at least 70% identical to SEQ ID NO: 404, and a HCDR3 amino acid sequence at least 70% identical to SEQ ID NO: 426; and a light chain variable region comprising a LCDR1 amino acid sequence at least 70% identical to SEQ ID NO: 453, a LCDR2 amino acid sequence at least 70% identical to SEQ ID NO: 470, and a LCDR3 amino acid sequence at least 70% identical to SEQ ID NO: 494, or
a heavy chain variable region comprising a HCDR1 amino acid sequence at least 70% identical to SEQ ID NO: 381, a HCDR2 amino acid sequence at least 70% identical to SEQ ID NO: 404, and a HCDR3 amino acid sequence at least 70% identical to SEQ ID NO: 426; and a light chain variable region comprising a LCDR1 amino acid sequence at least 70% identical to SEQ ID NO: 453, a LCDR2 amino acid sequence at least 70% identical to SEQ ID NO: 470, and a LCDR3 amino acid sequence at least 70% identical to SEQ ID NO: 495, or
a heavy chain variable region comprising a HCDR1 amino acid sequence at least 70% identical to SEQ ID NO: 381, a HCDR2 amino acid sequence at least 70% identical to SEQ ID NO: 405, and a HCDR3 amino acid sequence at least 70% identical to SEQ ID NO: 426; and a light chain variable region comprising a LCDR1 amino acid sequence at least 70% identical to SEQ ID NO: 453, a LCDR2 amino acid sequence at least 70% identical to SEQ ID NO: 470, and a LCDR3 amino acid sequence at least 70% identical to SEQ ID NO: 494, or
a heavy chain variable region comprising a HCDR1 amino acid sequence at least 70% identical to SEQ ID NO: 378, a HCDR2 amino acid sequence at least 70% identical to SEQ ID NO: 406, and a HCDR3 amino acid sequence at least 70% identical to SEQ ID NO: 427; and a light chain variable region comprising a LCDR1 amino acid sequence at least 70% identical to SEQ ID NO: 452, a LCDR2 amino acid sequence at least 70% identical to SEQ ID NO: 470, and a LCDR3 amino acid sequence at least 70% identical to SEQ ID NO: 496, or
a heavy chain variable region comprising a HCDR1 amino acid sequence at least 70% identical to SEQ ID NO: 378, a HCDR2 amino acid sequence at least 70% identical to SEQ ID NO: 407, and a HCDR3 amino acid sequence at least 70% identical to SEQ ID NO: 425; and a light chain variable region comprising a LCDR1 amino acid sequence at least 70% identical to SEQ ID NO: 452, a LCDR2 amino acid sequence at least 70% identical to SEQ ID NO: 475, and a LCDR3 amino acid sequence at least 70% identical to SEQ ID NO: 496, or
a heavy chain variable region comprising a HCDR1 amino acid sequence at least 70% identical to SEQ ID NO: 382, a HCDR2 amino acid sequence at least 70% identical to SEQ ID NO: 408, and a HCDR3 amino acid sequence at least 70% identical to SEQ ID NO: 428; and a light chain variable region comprising a LCDR1 amino acid sequence at least 70% identical to SEQ ID NO: 454, a LCDR2 amino acid sequence at least 70% identical to SEQ ID NO: 471, and a LCDR3 amino acid sequence at least 70% identical to SEQ ID NO: 497, or
a heavy chain variable region comprising a HCDR1 amino acid sequence at least 70% identical to SEQ ID NO: 382, a HCDR2 amino acid sequence at least 70% identical to SEQ ID NO: 408, and a HCDR3 amino acid sequence at least 70% identical to SEQ ID NO: 429; and a light chain variable region comprising a LCDR1 amino acid sequence at least 70% identical to SEQ ID NO: 455, a LCDR2 amino acid sequence at least 70% identical to SEQ ID NO: 476, and a LCDR3 amino acid sequence at least 70% identical to SEQ ID NO: 498, or
a heavy chain variable region comprising a HCDR1 amino acid sequence at least 70% identical to SEQ ID NO: 382, a HCDR2 amino acid sequence at least 70% identical to SEQ ID NO: 408, and a HCDR3 amino acid sequence at least 70% identical to SEQ ID NO: 429; and a light chain variable region comprising a LCDR1 amino acid sequence at least 70% identical to SEQ ID NO: 454, a LCDR2 amino acid sequence at least 70% identical to SEQ ID NO: 468, and a LCDR3 amino acid sequence at least 70% identical to SEQ ID NO: 497, or
a heavy chain variable region comprising a HCDR1 amino acid sequence at least 70% identical to SEQ ID NO: 382, a HCDR2 amino acid sequence at least 70% identical to SEQ ID NO: 408, and a HCDR3 amino acid sequence at least 70% identical to SEQ ID NO: 430; and a light chain variable region comprising a LCDR1 amino acid sequence at least 70% identical to SEQ ID NO: 454, a LCDR2 amino acid sequence at least 70% identical to SEQ ID NO: 477, and a LCDR3 amino acid sequence at least 70% identical to SEQ ID NO: 497, or
a heavy chain variable region comprising a HCDR1 amino acid sequence at least 70% identical to SEQ ID NO: 383, a HCDR2 amino acid sequence at least 70% identical to SEQ ID NO: 409, and a HCDR3 amino acid sequence at least 70% identical to SEQ ID NO: 431; and a light chain variable region comprising a LCDR1 amino acid sequence at least 70% identical to SEQ ID NO: 456, a LCDR2 amino acid sequence at least 70% identical to SEQ ID NO: 474, and a LCDR3 amino acid sequence at least 70% identical to SEQ ID NO: 499, or
a heavy chain variable region comprising a HCDR1 amino acid sequence at least 70% identical to SEQ ID NO: 383, a HCDR2 amino acid sequence at least 70% identical to SEQ ID NO: 409, and a HCDR3 amino acid sequence at least 70% identical to SEQ ID NO: 431; and a light chain variable region comprising a LCDR1 amino acid sequence at least 70% identical to SEQ ID NO: 456, a LCDR2 amino acid sequence at least 70% identical to SEQ ID NO: 478, and a LCDR3 amino acid sequence at least 70% identical to SEQ ID NO: 499, or
a heavy chain variable region comprising a HCDR1 amino acid sequence at least 70% identical to SEQ ID NO: 384, a HCDR2 amino acid sequence at least 70% identical to SEQ ID NO: 410, and a HCDR3 amino acid sequence at least 70% identical to SEQ ID NO: 431; and a light chain variable region comprising a LCDR1 amino acid sequence at least 70% identical to SEQ ID NO: 457, a LCDR2 amino acid sequence at least 70% identical to SEQ ID NO: 474, and a LCDR3 amino acid sequence at least 70% identical to SEQ ID NO: 500, or
a heavy chain variable region comprising a HCDR1 amino acid sequence at least 70% identical to SEQ ID NO: 384, a HCDR2 amino acid sequence at least 70% identical to SEQ ID NO: 410, and a HCDR3 amino acid sequence at least 70% identical to SEQ ID NO: 432; and a light chain variable region comprising a LCDR1 amino acid sequence at least 70% identical to SEQ ID NO: 458, a LCDR2 amino acid sequence at least 70% identical to SEQ ID NO: 474, and a LCDR3 amino acid sequence at least 70% identical to SEQ ID NO: 500, or
a heavy chain variable region comprising a HCDR1 amino acid sequence at least 70% identical to SEQ ID NO: 385, a HCDR2 amino acid sequence at least 70% identical to SEQ ID NO: 411, and a HCDR3 amino acid sequence at least 70% identical to SEQ ID NO: 433; and a light chain variable region comprising a LCDR1 amino acid sequence at least 70% identical to SEQ ID NO: 459, a LCDR2 amino acid sequence at least 70% identical to SEQ ID NO: 470, and a LCDR3 amino acid sequence at least 70% identical to SEQ ID NO: 501.

In some embodiments, the antibody comprises: a heavy chain variable region comprising a HCDR1 amino acid sequence at least 70% identical to SEQ ID NO: 386, a HCDR2 amino acid sequence at least 70% identical to SEQ ID NO: 412, and a HCDR3 amino acid sequence at least 70% identical to SEQ ID NO: 434; and a light chain variable region comprising a LCDR1 amino acid sequence at least 70% identical to SEQ ID NO: 460, a LCDR2 amino acid sequence at least 70% identical to SEQ ID NO: 479, and a LCDR3 amino acid sequence at least 70% identical to SEQ ID NO: 502, or
a heavy chain variable region comprising a HCDR1 amino acid sequence at least 70% identical to SEQ ID NO: 387, a HCDR2 amino acid sequence at least 70% identical to SEQ ID NO: 413, and a HCDR3 amino acid sequence at least 70% identical to SEQ ID NO: 435; and a light chain variable region comprising a LCDR1 amino acid sequence at least 70% identical to SEQ ID NO: 461, a LCDR2 amino acid sequence at least 70% identical to SEQ ID NO: 201, and a LCDR3 amino acid sequence at least 70% identical to SEQ ID NO: 503, or
a heavy chain variable region comprising a HCDR1 amino acid sequence at least 70% identical to SEQ ID NO: 4, a HCDR2 amino acid sequence at least 70% identical to SEQ ID NO: 414, and a HCDR3 amino acid sequence at least 70% identical to SEQ ID NO: 436; and a light chain variable region comprising a LCDR1 amino acid sequence at least 70% identical to SEQ ID NO: 462, a LCDR2 amino acid sequence at least 70% identical to SEQ ID NO: 480, and a LCDR3 amino acid sequence at least 70% identical to SEQ ID NO: 504, or
a heavy chain variable region comprising a HCDR1 amino acid sequence at least 70% identical to SEQ ID NO: 388, a HCDR2 amino acid sequence at least 70% identical to SEQ ID NO: 415, and a HCDR3 amino acid sequence at least 70% identical to SEQ ID NO: 437; and a light chain variable region comprising a LCDR1 amino acid sequence at least 70% identical to SEQ ID NO: 461, a LCDR2 amino acid sequence at least 70% identical to SEQ ID NO: 201, and a LCDR3 amino acid sequence at least 70% identical to SEQ ID NO: 505, or
a heavy chain variable region comprising a HCDR1 amino acid sequence at least 70% identical to SEQ ID NO: 4, a HCDR2 amino acid sequence at least 70% identical to SEQ ID NO: 416, and a HCDR3 amino acid sequence at least 70% identical to SEQ ID NO: 438; and a light chain variable region comprising a LCDR1 amino acid sequence at least 70% identical to SEQ ID NO: 462, a LCDR2 amino acid sequence at least 70% identical to SEQ ID NO: 480, and a LCDR3 amino acid sequence at least 70% identical to SEQ ID NO: 506, or
a heavy chain variable region comprising a HCDR1 amino acid sequence at least 70% identical to SEQ ID NO: 4, a HCDR2 amino acid sequence at least 70% identical to SEQ ID NO: 414, and a HCDR3 amino acid sequence at least 70% identical to SEQ ID NO: 436; and a light chain variable region comprising a LCDR1 amino acid sequence at least 70% identical to SEQ ID NO: 462, a LCDR2 amino acid sequence at least 70% identical to SEQ ID NO: 480, and a LCDR3 amino acid sequence at least 70% identical to SEQ ID NO: 507.

In some embodiments, the antibody comprises: a heavy chain variable region comprising a HCDR1 amino acid sequence at least 70% identical to SEQ ID NO: 389, a HCDR2 amino acid sequence at least 70% identical to SEQ ID NO: 417, and a HCDR3 amino acid sequence at least 70% identical to SEQ ID NO: 439.

In some embodiments, the antibody comprises all six CDRs of an antibody selected from the group consisting of 1104SBC1068-033, 1104SBC1068-335, 1104SBC1068-365, 1104SBC1068-374, 1104SBC1068-378, 1220SBC1068-022, 1220SBC1068-035, 1220SBC1068-041, 1220SBC1068-056, 1220SBC1068-058, 1220SBC1068-063, 1220SBC1068-097, 1220SBC1068-098, 1220SBC1068-099, 1220SBC1068-129, 1220SBC1068-164, 1220SBC1068-186, 1220SBC1068-197, 1220SBC1068-210, 1220SBC1068-274 and 1220SBC1068-281.

In some embodiments, the antibody or the binding fragment thereof comprises: a V_{H} region comprising a V_{H} amino acid sequence at least 70% identity to any one of SEQ ID NOS: 148-168, 353, and 355; and/or a V_{L} region comprising a V_{L} amino acid sequence at least 70% identity to any one of SEQ ID NOS: 316-336, 354, and 356.

In some embodiments, the antibody or the binding fragment thereof comprises: a V_{H} region comprising a V_{H} amino acid sequence at least 70% identity to SEQ ID NO: 148, and a V_{L} region comprising a V_{L} amino acid sequence at least 70% identity to SEQ ID NO: 316; or a V_{H} region comprising a V_{H} amino acid sequence at least 70% identity to SEQ ID NO: 149, and a V_{L} region comprising a V_{L} amino acid sequence at least 70% identity to SEQ ID NO: 317; or a V_{H} region comprising a V_{H} amino acid sequence at least 70% identity to SEQ ID NO: 150, and a V_{L} region comprising a V_{L} amino acid sequence at least 70% identity to SEQ ID NO: 318; or a V_{H} region comprising a V_{H} amino acid sequence at least 70% identity to SEQ ID NO: 151, and a V_{L} region comprising a V_{L} amino acid sequence at least 70% identity to SEQ ID NO: 319; or a V_{H} region comprising a V_{H} amino acid sequence at least 70% identity to SEQ ID NO: 152, and a V_{L} region comprising a V_{L} amino acid sequence at least 70% identity to SEQ ID NO: 320; or a V_{H} region comprising a V_{H} amino acid sequence at least 70% identity to SEQ ID NO: 153, and a V_{L} region comprising a V_{L} amino acid sequence at least 70% identity to SEQ ID NO: 321; or a V_{H} region comprising a V_{H} amino acid sequence at least 70% identity to SEQ ID NO: 154, and a V_{L} region comprising a V_{L} amino acid sequence at least 70% identity to SEQ ID NO: 322; or
a V_{H} region comprising a V_{H} amino acid sequence at least 70% identity to SEQ ID NO: 155, and a V_{L} region comprising a V_{L} amino acid sequence at least 70% identity to SEQ ID NO: 323; or
a V_{H} region comprising a V_{H} amino acid sequence at least 70% identity to SEQ ID NO: 156, and a V_{L} region comprising a V_{L} amino acid sequence at least 70% identity to SEQ ID NO: 324; or
a V_{H} region comprising a V_{H} amino acid sequence at least 70% identity to SEQ ID NO: 157, and a V_{L} region comprising a V_{L} amino acid sequence at least 70% identity to SEQ ID NO: 325; or
a V_{H} region comprising a V_{H} amino acid sequence at least 70% identity to SEQ ID NO: 158, and a V_{L} region comprising a V_{L} amino acid sequence at least 70% identity to SEQ ID NO: 326; or
a V_{H} region comprising a V_{H} amino acid sequence at least 70% identity to SEQ ID NO: 159, and a V_{L} region comprising a V_{L} amino acid sequence at least 70% identity to SEQ ID NO: 327; or
a V_{H} region comprising a V_{H} amino acid sequence at least 70% identity to SEQ ID NO: 160, and a V_{L} region comprising a V_{L} amino acid sequence at least 70% identity to SEQ ID NO: 328; or
a V_{H} region comprising a V_{H} amino acid sequence at least 70% identity to SEQ ID NO: 161, and a V_{L} region comprising a V_{L} amino acid sequence at least 70% identity to SEQ ID NO: 329; or
a V_{H} region comprising a V_{H} amino acid sequence at least 70% identity to SEQ ID NO: 162, and a V_{L} region comprising a V_{L} amino acid sequence at least 70% identity to SEQ ID NO: 330; or
a V_{H} region comprising a V_{H} amino acid sequence at least 70% identity to SEQ ID NO: 163, and a V_{L} region comprising a V_{L} amino acid sequence at least 70% identity to SEQ ID NO: 331; or
a V_{H} region comprising a V_{H} amino acid sequence at least 70% identity to SEQ ID NO: 164, and a V_{L} region comprising a V_{L} amino acid sequence at least 70% identity to SEQ ID NO: 332; or
a V_{H} region comprising a V_{H} amino acid sequence at least 70% identity to SEQ ID NO: 165, and a V_{L} region comprising a V_{L} amino acid sequence at least 70% identity to SEQ ID NO: 333; or
a V_{H} region comprising a V_{H} amino acid sequence at least 70% identity to SEQ ID NO: 166, and a V_{L} region comprising a V_{L} amino acid sequence at least 70% identity to SEQ ID NO: 334; or
a V_{H} region comprising a V_{H} amino acid sequence at least 70% identity to SEQ ID NO: 167, and a V_{L} region comprising a V_{L} amino acid sequence at least 70% identity to SEQ ID NO: 335; or
a V_{H} region comprising a V_{H} amino acid sequence at least 70% identity to SEQ ID NO: 168, and a V_{L} region comprising a V_{L} amino acid sequence at least 70% identity to SEQ ID NO: 336; or
a V_{H} region comprising a V_{H} amino acid sequence at least 70% identity to SEQ ID NO: 353, and a V_{L} region comprising a V_{L} amino acid sequence at least 70% identity to SEQ ID NO: 354; or
a V_{H} region comprising a V_{H} amino acid sequence at least 70% identity to SEQ ID NO: 355, and a V_{L} region comprising a V_{L} amino acid sequence at least 70% identity to SEQ ID NO: 356.

In some embodiments, the antibody or the binding fragment thereof comprises both the V_{H} and V_{L} of an antibody selected from the group consisting of 1104SBC1068-033, 1104SBC1068-335, 1104SBC1068-365, 1104SBC1068-374, 1104SBC1068-378, 1220SBC1068-022, 1220SBC1068-035, 1220SBC1068-041, 1220SBC1068-056, 1220SBC1068-058, 1220SBC1068-063, 1220SBC1068-097, 1220SBC1068-098, 1220SBC1068-099, 1220SBC1068-129, 1220SBC1068-164, 1220SBC1068-186, 1220SBC1068-197, 1220SBC1068-210, 1220SBC1068-274, 1220SBC1068-281, SIF-001, SIF-002, and a variant thereof.

In some embodiments, the antibody is a humanized, chimeric, or human antibody.

In yet another aspect, provided herein is a polypeptide comprising a V_{H} sequence and/or a V_{L} sequence of an antibody described above. In some embodiments, provided herein is a polynucleotide encoding the polypeptide. In some embodiments, provided herein is an expression vector comprising the polynucleotide. In some embodiments, provided herein is a cell that comprises the expression vector of claim 11.

In some embodiments, provided herein is a kit that comprises an antibody, a polypeptide, a polynucleotide, an expression vector, and/or a cell of described above.

In some embodiments, provided herein is a pharmaceutical composition comprising an antibody described above and a pharmaceutically acceptable carrier.

In yet another aspect, provided herein is a method of treating epilepsy or a related neurological disorder (e.g., Alzheimer's disease (AD) or Parkinson's disease (PD)) in a subject in need thereof, comprising administering the subject an effective amount of the pharmaceutical composition above. In some embodiments, the effective amount is about 5-100 mg/kg of the antibody or the immunoconjugate per the subject's body weight. In some embodiments, the effective amount is about 5, 10, 20, 30, 40, 50, 60, 70, 80, 90, and 100 mg/kg of the antibody or the immunoconjugate per the subject's body weight or an amount within a range defined by any of the two values above. In some embodiments, the pharmaceutical composition is administered intravenously into the subject. In some embodiments, the pharmaceutical composition is administered more than once, such as two, three, four, or more times. In some embodiments, each administration is at least 7 days apart. In some embodiments, each administration is at least 14 days apart. In some embodiments, each administration is at least four weeks apart.

In some embodiments, the method for treating epilepsy, an inflammatory or fibrotic disease, or a related neurological disorder (e.g., Alzheimer's disease (AD) or Parkinson's disease (PD)) in a subject in need thereof, comprising administering to the subject an antibody comprising a means for binding to Galectin-3, for example, a human Galectin-3. In some embodiments, the neurological disorder is Alzheimer's disease (AD) or Parkinson's disease (PD).

In some embodiments, the antibody is administered in combination with an additional therapeutic agent. In some embodiments, the antibody is administered intravenously into the subject. In some embodiments, the administration dosage of the antibody is between about 5-100 mg/kg per the subject's body weight. In some embodiments, the administration dosage of the antibody is about 5, 10, 20, 30, 40, 50, 60, 70, 80, 90, and 100 mg/kg of the antibody or the immunoconjugate per the subject's body weight or a dosage within a range defined by any of the two values above.

In some embodiments, provided herein is an antibody or a binding fragment thereof for use in the treatment of epilepsy or a related neurological disorder (e.g., Alzheimer's disease (AD) or Parkinson's disease (PD)) in a subject, wherein the antibody comprises: a heavy chain variable (V_{H}) region comprising:
a HCDR1 amino acid sequence at least 70% identical to any one of SEQ ID NOS: 1-21 and 481-507, a HCDR2 amino acid sequence at least 70% identical to any one of SEQ ID NOS: 22-42 and 390-417, and/or a HCDR3 amino acid sequence at least 70% identical to any one of SEQ ID NOS: 43-63 and 418-439, and/or
a light chain variable (V_{L}) region comprising:
a LCDR1 amino acid sequence at least 70% identical to any one of SEQ ID NOS: 169-189 and 440-462, a LCDR2 amino acid sequence at least 70% identical to any one of SEQ ID NOS: 190-210 and 463-480, and/or a LCDR3 amino acid sequence at least 70% identical to any one of SEQ ID NOS: 211-231 and 481-507.

In some embodiments, provided herein is an antibody or a binding fragment thereof that binds to Galectin-3, wherein the antibody comprises: a heavy chain variable region comprising:
a HCDR1 comprising a sequence NYGMN (SEQ ID NO: 4), or a variant HCDR1 in which 1, 2, or 3 amino acids are substituted relative to the sequence;
a HCDR2 comprising a sequence WINTYTGEPTYADDFKG (SEQ ID NO: 25), or a variant HCDR2 in which 1, 2, or 3 amino acids are substituted relative to the sequence; and
a HCDR3 comprising a sequence YAMDY (SEQ ID NO: 46), or a variant HCDR3 in which 1, 2, or 3 amino acids are substituted relative to the sequence; and
a light chain variable region comprising:
a LCDR1 comprising a sequence RSSTGAVTTSNYAN (SEQ ID NO: 172), or a variant LCDR1 in which 1 amino acid is substituted relative to the sequence;
a LCDR2 comprising a sequence GTSNRAP (SEQ ID NO: 193), or variant LCDR2 in which 1 amino acid is substituted relative to the sequence; and
a LCDR3 comprising a sequence ALWYSTHYV (SEQ ID NO: 214), or a variant LCDR3 in which 1 amino acid is substituted relative to the sequence, optionally, the substitution in HCDR1 is in any of the residue #1, and #3-#5, wherein the substitution in HCDR2 is in any of the residue #2, #4-#17, wherein the substitution in HCDR3 is in any of the residues #2-#4, wherein the substitution in LCDR1 is in any of the residues #1-#11 and #13-#14, and/or wherein the substitution in LCDR3 is in any of the residues #1-#2, #4-#7, and #9.

In some embodiments, the amino acid (“aa”) at position #2 residue in HCDR1 of the antibody above is Y, W, or F,
wherein the aa at position #1 residue in HCDR2 is W, Y, or F, and aa at position #3 residue in HCDR1 is N or Q,
wherein the aa at position #1 residue in HCDR3 is Y,
wherein the aa at position #12 residue in LCDR1 is Y, W, or F,
wherein the aa at position #3 in LCDR3 is Y, W, or F, and aa at position #8 residue in LCDR3 is Y, W, or F.

In some embodiments, disclosed herein is an antibody or a binding fragment thereof that binds to Galectin-3, wherein the antibody comprises: a heavy chain variable region comprising:
a HCDR1 comprising a sequence RFWMS (SEQ ID NO: 8), or a variant HCDR1 in which 1, 2, or 3 amino acids are substituted relative to the sequence;
a HCDR2 comprising a sequence EISPDSNTIDLTPSLKD (SEQ ID NO: 29), or a variant HCDR2 in which 1, 2, or 3 amino acids are substituted relative to the sequence; and
a HCDR3 comprising a sequence PYYGYY (SEQ ID NO: 50), or a variant HCDR3 in which 1, 2, or 3 amino acids are substituted relative to the sequence; and
a light chain variable region comprising:
a LCDR1 comprising a sequence RSSQSLFNSTNQKNYLT (SEQ ID NO: 176) or RSSQSLFSSTNQKNYLT (SEQ ID NO: 369), or a variant LCDR1 in which 1 amino acid is substituted relative to the sequence;
a LCDR2 comprising a sequence WASSRES (SEQ ID NO: 197), or variant LCDR2 in which 1 amino acid is substituted relative to the sequence; and
a LCDR3 comprising a sequence QNDYTSPFT (SEQ ID NO: 218), or a variant LCDR3 in which 1 amino acid is substituted relative to the sequence.

In some embodiments, the substitution in HCDR1 in the antibody above is in any of the residue #1-2, and #4-#5, the substitution in HCDR2 is in any of the residue #2-#17, the substitution in HCDR3 is in any of the residues #1-#4 and #6, the substitution in LCDR1 is in any of the residues #1-#14 and #16-#17, and the substitution in LCDR3 is in any of the residues #1-#2, #4-#9.

In some embodiments, the aa at position #3 residue in HCDR1 is Y, W, or F, the aa at position #1 residue in HCDR2 is E or D, the aa at position #5 residue in HCDR3 is Y, W, or F, the aa at position #15 residue in LCDR1 is Y, W, or F, and/or the aa at position #3 in LCDR3 is Y.

In some embodiments, provided herein is an antibody competes for binding to GAL-3 with an antibody disclosed herein.

In some embodiments, provided herein is an antibody or a binding fragment thereof that binds to Galectin-3, wherein the antibody comprises:
a HCDR1 comprising a sequence of NX₂GMN (SEQ ID NO:357), wherein X₂ is Y, W, or F, wherein the HCDR1 has zero or one aa substitution in rest of the residues relative to the HCDR1 sequence,
a HCDR2 comprising a sequence of X₁IX₃TYTGEPTYADDFKG (SEQ ID NO: 358), Where X₁ is W, Y, or F, and wherein X₃ is N or Q, wherein the HCDR2 has zero, one, two, three, or four aa substitutions in rest of the residues relative to the HCDR2 sequence,
a HCDR3 comprising a sequence of YAMDY (SEQ ID NO:359), wherein the HCDR3 has zero, one, or two aa substitutions relative to the HCDR3 sequence,
a LCDR1 comprising a sequence of RSSTGAVTTSNX₁₂AN (SEQ ID NO:360), wherein X₁₂ is Y, W, or F, wherein the LCDR1 has zero, one, two, three, or four aa substitutions in the rest of the residues relative to the LCDR1 sequence,
a LCDR2 comprising a sequence of GTSNRAP (SEQ ID NO: 361), wherein the LCDR2 has zero, one, two, or three aa substitutions in the rest of the residues relative to the LCDR2 sequence, and
a LCDR3 comprising a sequence of ALX₃YSTHX₈V (SEQ ID NO: 362), wherein X₃ is Y, W, or F, wherein X₈ is Y, W, or F, wherein the LCDR3 has zero, one, two aa substitutions in the rest of the residues relative to the LCDR3 sequence.

In some embodiments, provided herein is an antibody or a binding fragment thereof that binds to Galectin-3, wherein the antibody comprises:
a HCDR1 comprising a sequence of NX₂GMN (SEQ ID NO:357), wherein X₂ is Y, W, or F,
a HCDR2 comprising a sequence of X₁IX₃TYTGEPTYADDFKG (SEQ ID NO: 358), Where X₁ is W, Y, or F, and wherein X₃ is N or Q,
a HCDR3 comprising a sequence of YAMDY (SEQ ID NO:359),
a LCDR1 comprising a sequence of RSSTGAVTTSNX₁₂AN (SEQ ID NO:360), wherein X₁₂ is Y, W, or F,
a LCDR2 comprising a sequence of GTSNRAP (SEQ ID NO: 361), and
a LCDR3 comprising a sequence of ALX₃YSTHX₈V (SEQ ID NO: 362), wherein X₃ is Y, W, or F, wherein X₈ is Y, W, or F;
wherein
the HCDR1 has zero or one aa substitution in rest of the residues relative to the HCDR1 sequence,
the HCDR2 has zero, one, two, three, or four aa substitutions in rest of the residues relative to the HCDR2 sequence,
the HCDR3 has zero, one, or two aa substitutions relative to the HCDR3 sequence,
the LCDR1 has zero, one, two, three, or four aa substitutions in the rest of the residues relative to the LCDR1 sequence,
the LCDR2 has zero, one, two, or three aa substitutions in the rest of the residues relative to the LCDR2 sequence, and/or
the LCDR3 has zero, one, two aa substitutions in the rest of the residues relative to the LCDR3 sequence.

In some embodiments, provided herein is an antibody or a binding fragment thereof that binds to Galectin-3, wherein the antibody comprises:
a HCDR1 comprising a sequence of RFX₃MS (SEQ ID NO: 363), wherein X₃ is Y, W, or F, and wherein the HCDR1 has zero, one, two aa substitutions in the rest of the residues relative to the HCDR1 sequence,
a HCDR2 comprising X₁ISPDSNTIDLTPSLKD (SEQ ID NO: 364), wherein X₁ is E or D, and wherein the HCDR2 has zero, one, two, three, or four aa substitutions in the rest of the residues relative to the HCDR2 sequence,
a HCDR3 comprising PYYGX₅Y (SEQ ID NO: 365), wherein X₅ is Y, W, or F, wherein the HCDR3 has zero, one, two aa substitutions in the rest of the residues relative to the HCDR3 sequence,
a LCDR1 comprising RSSQSLFSSTNQKNX₁₅LT (SEQ ID NO: 366), wherein X₁₅ is Y, W, or F, wherein the LCDR1 has zero, one, two, three, or four aa substitutions in the rest of the residues relative to the LCDR1 sequence,
a LCDR2 comprising WASSRES (SEQ ID NO: 367), wherein the LCDR2 has zero, one, two, or three aa substitutions relative to the LCDR2 sequence, and/or
a LCDR3 comprising QNDYTSPFT (SEQ ID NO: 368), wherein the LCDR3 has zero, one, two, three, or four aa substitutions in the rest of the residues relative to the LCDR3 sequence.

In some embodiments, provided herein is an antibody or a binding fragment thereof that binds to Galectin-3, wherein the antibody comprises:
a HCDR1 comprising a sequence of RFX₃MS (SEQ ID NO: 363), wherein X₃ is Y, W, or F,
a HCDR2 comprising X₁ISPDSNTIDLTPSLKD (SEQ ID NO: 364), wherein X₁ is E or D,
a HCDR3 comprising PYYGX₅Y (SEQ ID NO: 365), wherein X₅ is Y, W, or F,
a LCDR1 comprising RSSQSLFSSTNQKNX₁₅LT (SEQ ID NO: 366), wherein X₁₅ is Y, W, or F,
a LCDR2 comprising WASSRES (SEQ ID NO: 367), and
a LCDR3 comprising QNDYTSPFT (SEQ ID NO: 368);
and wherein
the HCDR1 has zero, one, two aa substitutions in the rest of the residues relative to the HCDR1 sequence,
the HCDR2 has zero, one, two, three, or four aa substitutions in the rest of the residues relative to the HCDR2 sequence,
the HCDR3 has zero, one, two aa substitutions in the rest of the residues relative to the HCDR3 sequence,
the LCDR1 has zero, one, two, three, or four aa substitutions in the rest of the residues relative to the LCDR1 sequence,
the LCDR2 has zero, one, two, or three aa substitutions relative to the LCDR2 sequence, and/or
the LCDR3 has zero, one, two, three, or four aa substitutions in the rest of the residues relative to the LCDR3 sequence.

In some embodiments, provided herein is an antibody or a binding fragment thereof that binds to Galectin-3, wherein the antibody comprises a HCDR1 comprising a sequence of NYGMN (SEQ ID NO: 4), a HCDR2 comprising a sequence of WINTYTGEPTYADDFKG (SEQ ID NO: 25), a HCDR3 comprising a sequence of YAMDY (SEQ ID NO: 46), a LCDR1 comprising a sequence of RSSTGAVTTSNYAN (SEQ ID NO: 172), a LCDR2 comprising a sequence of GTSNRAP (SEQ ID NO: 193), and a LCDR3 comprising a sequence of ALWYSTHYV (SEQ ID NO: 214).

In some embodiments, provided herein is an antibody comprises a V_{H} region comprising a sequence of SEQ ID NO: 353, and a V_{L} region comprising a sequence of SEQ ID NO: 354.

In some embodiments, provided herein is an antibody or a binding fragment thereof that binds to Galectin-3, wherein the antibody comprises a HCDR1 comprising a sequence of RFWMS (SEQ ID NO: 8), a HCDR2 comprising a sequence of EISPDSNTIDLTPSLKD (SEQ ID NO: 29), a HCDR3 comprising a sequence of PYYGYY (SEQ ID NO: 50), a LCDR1 comprising a sequence of RSSQSLFNSTNQKNYLT (SEQ ID NO: 176) or RSSQSLFSSTNQKNYLT (SEQ ID NO: 369), a LCDR2 comprising a sequence of WASSRES (SEQ ID NO: 197), and a LCDR3 comprising a sequence of QNDYTSPFT (SEQ ID NO: 218).

In some embodiments, provided herein is an antibody comprises a V_{H} region comprising a sequence of SEQ ID NO: 355, and a V_{L} region comprising a sequence of SEQ ID NO: 356.

As disclosed herein, any of the embodiments described above can be combined with any other embodiments disclosed above, provided that the resulting combined embodiment remains operable and feasible. Such resulting combined embodiments are also within the scope of the disclosure.

### BRIEF DESCRIPTION OF THE DRAWINGS

FIGS. 1A-1D depict a kinetics analysis of in vitro cross-species reactivity of SIF001 against GAL-3 in (A) human, (B) mouse, (C) rat, and (D) monkey samples.

FIG. 2 depicts the effect of SIF001 on TNFα release in microglia BV2 cells stimulated with LPS.

FIG. 3 depicts the effect of SIF001 on epilepsy model in microglia BV2 cells stimulated by KA via cell immunofluorescence assay.

FIG. 4A depicts a study schema of the evaluation SIF001 therapeutic effects in epilepsy model in C57BL/6J mouse stimulated by KA. FIGS. 4B-4E depict the effects of SIF001 in epilepsy model in C57BL/6J mouse stimulated by KA, as evaluated in (B) seizure occurring rate, (C) seizure frequency, (D) seizure severity, and (E) seizure duration.

FIG. 5 depicts the effects of SIF001 on the ionized calcium-binding adaptor molecule 1 (Iba1) in hippocampus in epilepsy model of C57BL/6J mouse stimulated by KA.

FIG. 6 depicts the effects of SIF001 on the Glial fibrillary acidic protein (GFAP) in hippocampus in epilepsy model of C57BL/6J mouse stimulated by KA.

FIG. 7 depicts the alanine scanning of SIF001 CDRs. (A) Alanine scanning of SIF001 heavy chain CDRs (SEQ ID Nos: 4, 25, and 46) indicates that the amino acid residues: Y32 and G33 in HCDR1, W50 and N52 in HCDR2; and Y99 in HCDR3 (respectively underlined in the CDR sequences in the table) are essential to the antibody SIF001 binding to the antigen GAL-3. (B) Alanine scanning of SIF001 light chain CDRs (SEQ ID Nos: 172, 193, and 214) indicates that the amino acid residues: Y34 in LCDR1, and W93 and Y98 in LCDR3 (respectively underlined in the CDR sequences in the table) are essential to the antibody SIF001 binding to the antigen GAL-3.

FIG. 8 depicts the antigen binding effects of anti-GAL-3 antibodies derived from SIF001 with one amino acid substitution in the CDR regions. (A) indicates that a Y32W, Y32F, W50T, W50F, or N53Q mutation in the HCDR regions (SEQ ID Nos: 358-359) of SIF001 heavy chain does not impact antibody binding to the antigen GAL-3. (B) indicates that a Y34W, Y34F, W93Y, W93F, Y98W, or Y98F mutation in the LCDR regions (SEQ ID Nos: 360-362) of SIF001 light chain does not impact antibody binding to the antigen GAL-3.

FIG. 9 depicts the alanine scanning of SIF002 CDRs. (A) Alanine scanning of SIF002 heavy chain CDRs (SEQ ID Nos: 8, 29, and 50) indicates that the amino acid residues: W33 in HCDR1, E50 in HCDR2; and Y103 in HCDR3 (respectively underlined in the CDR sequences in the table) are essential to the antibody SIF002 binding to the antigen GAL-3. (B) Alanine scanning of SIF002 light chain CDRs (SEQ ID Nos: 369, 197, and 218) indicates that the amino acid residues: Y38 in LCDR1, and D97 in LCDR3 (respectively underlined in the CDR sequences in the table) are essential to the antibody SIF002 binding to the antigen GAL-3.

FIG. 10 depicts the antigen binding effects of anti-GAL-3 antibodies derived from SIF002 with one amino acid substitution in the CDR regions. (A) indicates that a W33Y, W33F, E50D, Y103W, or Y103F mutation in the HCDR regions (SEQ ID Nos: 363-365) of SIF002 heavy chain does not impact antibody binding to the antigen GAL-3. (B) indicates that a Y38W, or Y38F mutation in the LCDR regions (SEQ ID Nos: 366-368) of SIF002 light chain does not impact antibody binding to the antigen GAL-3.

FIG. 11 depicts the antibody SIF001 prevents seizure occurring in a dose-dependent manner in mice, as measured in seizure frequency (A), seizure severity (B), and seizure duration (C).

FIG. 12 depicts SEC of the Gal3-e-Fab complex.

FIG. 13 depicts SDS-PAGE of Gal3-e-Fab complex.

FIG. 14 depicts local density map of Gal3-e, a Galectin-3 epitope sequence (SEQ ID NO: 508).

FIG. 15 depicts interface between Gal3-e and Fab.

FIG. 16 depicts interactions between Gal3-e (SEQ ID NO: 508) and Fab of the SIF001 antibody with HCDR1 (SEQ ID NO: 2), HCDR2 (SEQ ID NO: 25), HCDR3 (SEQ ID NO: 46), LCDR1 (SEQ ID NO: 172), LCDR2 (SEQ ID NO: 193), and LCDR3 (SEQ ID NO: 214). Bold amino acids of the Gal3-e sequence indicate these residues are essential for Fab binding. The boxed sequence of the Gal3-e sequence indicates these residues are traced in the density map. The trace lines represent weak interactions; the bolded trace lines represent stronger interactions, and the dashed trace lines represent potential interactions.

FIG. 17 depicts study design and dosing regimen of the mouse study to evaluate the therapeutic effects of mSIF001 in the Parkinson's disease mimic mouse model.

FIG. 18 depicts Galectin-3 intrinsically promotes oligomerization of Alpha synuclein (a-Synuclein).

FIG. 19 depicts stereotactic injection of a-Synuclein oligomers leads to locomotor dysfunction.

FIG. 20 depicts treatment with mSIF001 significantly improved locomotor function as compared to the isotype control antibody treatment using rotarod.

FIG. 21 depicts treatment with SF001 Ab significantly reduced activated microglia probed with IBA-1 Ab (B) and aggregated α-Synuclein (C) in the PD mimic model in the selected region of substantia nigra in mouse brains (A).

FIG. 22 depicts study design and dosing regimen of the mouse study to evaluate the therapeutic effects of mSIF001 in the Alzheimer's disease mimic mouse model.

FIG. 23 depicts Galectin-3 intrinsically promotes oligomerization of Aβ₄₂, as characterized by dot blot using a conformational oligomer specific antibody A11 (A) and an Aβ₄₂ sequence-dependent antibody 6E10 (B). Gal-3 oligomerized Aβ₄₂ peptide into high molecular weight oligomers in a dose dependent manner as characterized by Western blot probed with an Aβ sequence-dependent antibody 6E10 (C).

FIG. 24 depicts Galectin-3 intrinsically promotes oligomerization of Phospho Tau (396) (B) but not normal Tau (A).

FIG. 25 depicts Galectin-3 intrinsically promotes oligomerization of APOE4 (A) but not APOE3 (B).

FIG. 26 depicts SIF001Ab dose-dependently dissolves Aβ42 oligomers induced by Gal-3 (A), while an iso-type control antibody had no effect (B).

FIG. 27 depicts the cognitive deficit of APP/PS1 mice in terms of latency to reach the platform (A) and number of crosses in the Morris water maze (B) as compared to age matched wild type mice.

FIG. 28 depicts dose dependent efficacy of SIF001 in Alzheimer's mouse model APP/PS1 on hippocampal dependent spatial memory (Morris water maze training) in terms of latency to reach the platform (A) and number of crosses (B).

FIG. 29 depicts the efficacy of SIF001 treatment in patient 1 during the epilepsy investigator-initiated trial (IIT).

FIG. 30 depicts the efficacy of SIF001 treatment in patient 2 during the epilepsy investigator-initiated trial (IIT).

FIG. 31 depicts the efficacy of SIF001 treatment in patient 3 during the epilepsy investigator-initiated trial (IIT).

FIG. 32 depicts the efficacy of SIF001 treatment in patient 4 during the epilepsy investigator-initiated trial (IIT).

FIG. 33 depicts the efficacy of SIF001 treatment in patient 5 during the epilepsy investigator-initiated trial (IIT), as measured by daily seizure frequency before and after SIF001 treatment.

FIG. 34 depicts the efficacy of SIF001 treatment in patient 5 during the epilepsy investigator-initiated trial (IIT), as measured by hourly seizure frequency during sleep through electroencephalogram (EEG) before and after SIF001 treatment.

FIG. 35 depicts a schematic drawing of LSA epitope binning workflow.

### DETAILED DESCRIPTION

### I. Introduction

The present disclosure provides compositions and methods for treating epilepsy and/or related diseases such as neurological disorders (e.g., AD). In particular, disclosed herein are anti-Galectin-3 antibodies and the use of these antibodies for epilepsy and/or related disease treatment.

Galectin-3 (GAL-3) plays a diverse role in biological processes including cell adhesion, proliferation, migration, apoptosis, tumor progression, and inflammation, especially in neuroinflammation. The inventor has discovered that anti-GAL-3 antibodies can significantly decrease pro-inflammatory cytokine (e.g., tumor necrosis factor-α (TNFα)), in LPS-stimulated microglia cells, and inhibit microglial activation in kainic acid (KA)-stimulated microglia BV2 cells, concluding that targeting GAL-3 with anti-GAL-3 antibodies may offer therapeutic benefits in epilepsy and related neurological disorders by suppressing neuroinflammation and reducing microglial activation.

### II. Definitions

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the present disclosure. For purposes of the present disclosure, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Numeric ranges recited within the specification are inclusive of the numbers within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range (e.g., 1 to 5 includes 1, 1.5, 2, 2.75, 3, 3.80, 4, and 5).

Also, the words “comprise,” “comprising,” “contains,” “containing,” “include,” “including,” and “includes,” when used in this specification and in the following claims, are intended to specify the presence of stated features, integers, components, or steps, but they do not preclude the presence or addition of one or more other features, integers, components, steps, acts, or groups.

The term “amino acid” refers to any monomeric unit that can be incorporated into a peptide, polypeptide, or protein. Amino acids include naturally occurring a-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an 1-amino acid and the corresponding d-amino acid).

The terms “identity,” “substantial identity,” “similarity,” “substantial similarity,” “homology” and the related terms and expressions used in the context of describing amino acid sequences refer to a sequence that has at least 60% sequence identity to a reference sequence. Examples include at least: 60%, 65%, 70%, 75%, 80%, 85%, 86%, 87%, 88%, 89%, 90%, 91%, 92%, 93%, 94%, 95%, 96%, 97%, 98%, or 99%, sequence identity, as compared to a reference sequence using the programs for comparison of amino acid sequences, such as BLAST using standard parameters. For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default (standard) program parameters can be used, or alternative parameters can be designated. The sequence comparison algorithm then calculates the percent sequence identities for the test sequences relative to the reference sequence, based on the program parameters. A “comparison window” includes reference to a segment of any one of the number of contiguous positions (from 20 to 600, usually about 50 to about 200, more commonly about 100 to about 150), in which a sequence may be compared to a reference sequence of the same number of contiguous positions after the two sequences are optimally aligned. Methods of alignment of sequences for comparison are well-known. Optimal alignment of sequences for comparison may be conducted, for example, by the local homology algorithm of Smith and Waterman, 1981, by the homology alignment algorithm of Needleman and Wunsch, 1970, by the search for similarity method of Pearson and Lipman, 1988, by computerized implementations of these algorithms (for example, BLAST), or by manual alignment and visual inspection.

Algorithms that are suitable for determining percent sequence identity and sequence similarity include BLAST and BLAST 2.0 algorithms, which are described in Altschul et al., 1990, and Altschul et al., 1977, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (Henikoff and Henikoff, 1989). The BLAST algorithm also performs a statistical analysis of the similarity between two sequences (Karlin and Altschul, 1993).

As disclosed herein, when referring to a range defined by “greater than a first minimum value,” such range includes any subrange defined by a second minimum value that is greater than the first minimum value. This is to say, all values equal to or greater than the first minimum value are encompassed within the broader range. For example, when stating “at least 70%,” this phrase inherently includes all percentages equal to or greater than 70%. It is equivalent to explicitly listing each percentage value, as follows: “at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.”

As used herein, the term “antibody” means an isolated or recombinant binding agent that comprises the necessary variable region sequences to specifically bind an antigenic epitope. Therefore, an “antibody” as used herein is any form of an antibody of any class or subclass or fragment thereof that exhibits the desired biological activity, e.g., binding a specific target antigen. Thus, it is used in the broadest sense and specifically covers a monoclonal antibody (including full-length monoclonal antibodies), human antibodies, chimeric antibodies, nanobodies, diabodies, multispecific antibodies (e.g., bispecific antibodies), antibody fragments, antigen-binding fragments including but not limited to scFv, Fab, and the like so long as they exhibit the desired biological activity.

“Antibody fragments” or “antigen-binding fragments” comprise a portion of an intact antibody, for example, the antigen-binding or variable region of the intact antibody. Examples of antibody fragments include Fab, Fab′, F(ab′)₂, and Fv fragments; diabodies; linear antibodies (e.g., Zapata et al., Protein Eng. 8 (10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′) 2 fragment with two antigen combining sites and is still capable of cross-linking antigen.

As used herein, “V-region” or “variable region” refers to an antibody variable region domain comprising the segments of Framework 1, CDR1, Framework 2, CDR2, and Framework 3, including CDR3 and Framework 4. The heavy chain V-region, V_{H}, is a consequence of the rearrangement of a V-gene (HV), a D-gene (HD), and a J-gene (HJ), in what is termed V(D)J recombination during B-cell differentiation. The light chain V-region, V_{L}, is a consequence of the rearrangement of a V-gene (LV) and a J-gene (LJ).

As used herein, “complementarity-determining region (CDR)” refers to the three hypervariable regions (HVRs) in each chain that interrupt the four “framework” regions established by the light and heavy chain variable regions. The CDRs are the primary contributors to binding to an epitope of an antigen. The CDRs of each chain are referred to as CDR1, CDR2, and CDR3, numbered sequentially starting from the N-terminus, and are also identified by the chain in which the CDR is located. Thus, a V_{H} CDR3 (HCDR3) is in the variable domain of the heavy chain of the antibody in which it is found, whereas a V_{L} CDR3 (LCDR3) is the CDR3 from the variable domain of the light chain of the antibody in which it is located. The term “CDR” is used interchangeably with “HVR” when referring to CDR sequences.

The amino acid sequences of the CDRs and framework regions can be determined using various well-known definitions in the art, e.g., Kabat, Chothia, international ImMunoGeneTics database (IMGT), and AbM (see, e.g., Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, Structural repertoire of the human V_{H} segments J. Mol. Biol. 227, 799-817; Al-Lazikani et al., J. Mol. Biol. 1997, 273 (4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29 (1): 207-9 (2001); MacCallum et al., Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin et al., Methods Enzymol., 203, 121-153, (1991); Pedersen et al., Immunomethods, 1, 126, (1992); and Rees et al., In Sternberg M. J. E. (ed.), Protein Structure Prediction. Oxford University Press, Oxford, 141-172 1996). Reference to CDRs as determined by Kabat numbering is based, for example, on Kabat et al., Sequences of Proteins of Immunological Interest, 5th Ed. Public Health Service, National Institute of Health, Bethesda, MD (1991)). Chothia CDRs are determined as defined by Chothia (see, e.g., Chothia and Lesk J. Mol. Biol. 196:901-917 (1987)).

CDRs in this disclosure are defined by Kabat. As known in the art, numbering and placement of the CDRs can differ depending on the numbering system employed. It is understood that disclosure of a variable heavy and/or variable light sequence includes the disclosure of the associated CDRs, regardless of the numbering system employed.

An “Fc region” refers to the constant region of an antibody excluding the first constant region immunoglobulin domain. Thus, e.g., for human immunoglobulins, “Fc” refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 and Cγ3 and the hinge between Cγ1 and Cγ2. It is understood in the art that the boundaries of the Fc region may vary, however, the human IgG heavy chain Fc region is usually defined to comprise residues C226 or P230 to its carboxyl-terminus, using the numbering according to the EU index as in Kabat et al. (1991, NIH Publication 91-3242, National Technical Information Service, Springfield, Va.). The term “Fc region” may refer to this region in isolation or this region in the context of an antibody or antibody fragment. “Fc region” includes naturally occurring allelic variants of the Fc region as well as modified Fc regions, e.g., that are modified to modulate effector function or other properties such as pharmacokinetics, stability or production properties of an antibody. Fc regions also include variants that do not exhibit alterations in biological function. For example, one or more amino acids can be deleted from the N-terminus or C-terminus of the Fc region of an immunoglobulin without substantial loss of biological function. Such variants can be selected according to general rules known in the art to have minimal effect on activity (see, e.g., Bowie et al., Science 247:306-1310, 1990). For example, for IgG4 antibodies, a single amino acid substitution (S228P according to Kabat numbering; designated IgG4Pro) may be introduced to abolish the heterogeneity observed in recombinant IgG4 antibodies (see, e.g., Angal et al., Mol Immunol 30:105-108, 1993).

An “EC₅₀” as used herein refers to the half-maximal effective concentration, which is the concentration of an antibody that induces a response (signal generated in engagement assay) halfway between the baseline and maximum after a specified exposure time. In some embodiments, the “fold over EC50” is determined by dividing the EC50 of a reference antibody by the EC₅₀ of the test antibody.

The term “equilibrium dissociation constant” abbreviated (K_{D}), refers to the dissociation rate constant (k_{d}, time⁻¹) divided by the association rate constant (kₐ, time⁻¹ M⁻¹). Equilibrium dissociation constants can be measured using any method. Thus, in some embodiments antibodies of the present disclosure have a K_{D} of less than about 50 nM, typically less than about 25 nM, or less than 10 nM, e.g., less than about 5 nM or than about 1 nM and often less than about 10 nM as determined by surface plasmon resonance analysis using a biosensor system such as a Biacore® system performed at 37° C. In some embodiments, an antibody of the present disclosure has a K_{D} of less than 5×10⁻⁵ M, less than 10⁻⁵ M, less than 5×10⁻⁶ M, less than 10⁻⁶M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M or lower as measured as a bivalent antibody. In the context of the present invention, an “improved” K_{D} refers to a lower K_{D}. In some embodiments, an antibody of the present disclosure has a K_{D} of less than 5×10⁻⁵ M, less than 10⁻⁵ M, less than 5×10⁻⁶ M, less than 10⁻⁶ M, less than 5×10⁻⁷M, less than 10⁻⁷M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴M, less than 10⁻¹⁴ M, less than 5×10⁻¹⁵ M, or less than 10⁻¹⁵ M or lower as measured as a monovalent antibody, such as a monovalent Fab. In some embodiments, an anti-GAL-3 antibody of the present disclosure has K_{D} less than 100 pM, e.g., or less than 75 pM, e.g., in the range of 1 to 100 pM, when measured by surface plasmon resonance analysis using a biosensor system such as a Biacore® system performed at 37° C. In some embodiments, an anti-GAL-3 antibody of the present disclosure has K_{D} of greater than 100 pM, e.g., in the range of 100-1000 pM or 500-1000 pM when measured by surface plasmon resonance analysis using a biosensor system such as a Biacore® system performed at 37° C.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, or combinations thereof. The terms also include, but is not limited to, single- and double-stranded forms of DNA. In addition, a polynucleotide, e.g., a cDNA or mRNA, may include cither or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, the substitution of one or more of the naturally occurring nucleotides with an analog, internucleotide modifications such as uncharged linkages (e.g., methyl phosphonates, phosphotriesters, phosphoramidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The terms “vector” and “expression vector” refer to a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter. The term “promoter” is used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators).

An “expression cassette” refers to a nucleic acid construct that, when introduced into a host cell, results in transcription and/or translation of an RNA or polypeptide, respectively.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “promoter,” as used herein, refers to a polynucleotide sequence capable of driving transcription of a coding sequence in a cell. Thus, promoters can include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription.

The phrase “percent identical” or equivalents used in the context of two nucleic acids or polypeptides, refers to a sequence that has at least a specified level of identity, e.g., at least 50% sequence identity with a reference sequence (e.g., any SEQ ID NO included herein). Alternatively, percent identity can be any integer from 50% to 100%. Some embodiments include at least: 50%, 55%, 60%, 65%, 70%, 75%, 80%, 85%, 90%, 91%, 92%, 93%, 94%, 95%, 96%, 97%, 98%, or 99%, compared to a reference sequence using the programs described herein, e.g., BLAST using standard parameters, as described below.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the numbers of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site.

A “substitution” denotes the replacement of one or more amino acids or nucleotides by different amino acids or nucleotides, respectively.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody or fragment thereof” refers to one or more nucleic acid molecules encoding antibody heavy or light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Thus, a host cell is a recombinant host cell and includes the primary transformed cell and progeny derived therefrom without regard to the number of passages.

A polypeptide “variant” is a polypeptide that typically differs from one or more polypeptide sequences specifically disclosed herein in one or more substitutions, deletions, additions, and/or insertions.

A “therapeutic agent” refers to an agent that when administered to a patient suffering from a disease, in a therapeutically effective dose, will cure, or at least partially arrest the symptoms of the disease and complications associated with the disease.

A “variant” of a reference antibody refers to an antibody that typically differs from the reference antibody in one or more substitutions, deletions, additions, and/or insertions in the amino acid sequence of the heavy and/or light chain.

As used herein, the term “internalize,” or “internalization” refer to the phenomenon that an antibody molecule crosses the cell membrane and reaches the cytoplasm and/or the nucleus.

The terms “therapy,” “treatment,” and “amelioration” refer to any reduction in the severity of symptoms. For example, in the case of treating cancer, treatment can refer to reducing the number of cancer cells or growth rate or cell death of non-cancer cells, etc. As used herein, the terms “treat” and “prevent” are not intended to be absolute terms. Treatment and prevention can refer to any delay in onset, amelioration of symptoms, improvement in patient survival, increase in survival time or rate, etc. Treatment and prevention can be complete (no detectable symptoms remaining) or partial, such that symptoms are less frequent of severe than in a patient without the treatment described herein. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” “therapeutically effective amount,” etc. refer to that amount of the therapeutic agent sufficient to ameliorate a disorder, as described above. For example, for the given parameter, a therapeutically effective amount will show an increase or decrease of therapeutic effect at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “pharmaceutically acceptable” is used synonymously with physiologically acceptable and pharmacologically acceptable. A pharmaceutical composition will generally comprise agents for buffering and preservation in storage, and can include buffers and carriers for appropriate delivery, depending on the route of administration.

The terms “dose” and “dosage” are used interchangeably herein. A dose refers to the amount of active ingredient given to an individual at each administration. The dose will vary depending on a number of factors, including frequency of administration; size and tolerance of the individual; severity of the condition; risk of side effects; the route of administration; and the imaging modality of the detectable moiety (if present). One of skill in the art will recognize that the dose can be modified depending on the above factors or based on therapeutic progress. The term “dosage form” refers to the particular format of the pharmaceutical and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, dogs, cats, and other mammalian species. The term does not necessarily indicate that the subject has been diagnosed with a particular disease but typically refers to an individual under medical supervision. A patient can be an individual that is seeking treatment, monitoring, adjustment or modification of an existing therapeutic regimen, etc.

### III. Detailed Description of the Embodiments

The present disclosure is directed to compositions and methods to prevent and treat metabolic epilepsy and/or related neurological disorders through targeting Galectin-3 with anti-GAL-3 antibodies.

### Galectin-3 (GAL-3)

The galectin family comprises at least fifteen β-galactoside-binding lectins, playing pivotal roles in growth and development, and contributing to the advancement of several diseases. Galectin-3 (GAL-3), a monomer but can form a multimer (dimer or pentamer) at higher concentrations^{[17]}, is one of the most studied members of the galectin family^{[2,18]}.

All galectins, including GAL-3, bind to β-galactoside, but they exhibit slight variations in their carbohydrate-binding abilities. Typically, galectins show a preference for N-acetyllactosamine, binding it significantly stronger than lactose. N-glycans rich in N-acetyllactosamine serve as effective ligands for most galectins. Notably, GAL-3's interaction with the TF-disaccharide found in O-glycans is distinct from that of galectin-1, displaying a much higher affinity in isothermal titration calorimetry (ITC) assays^{[22]}. These differences in binding properties among galectins can be attributed to their unique three-dimensional structures.^{[22-24]}

GAL-3 interacts with both intracellular and extracellular molecules. Unlike other galectins, GAL-3 is secreted without a conventional signal peptide^{[25]}, existing within the cytosol and the extracellular matrix (ECM)^{[26, 27]}. Known extracellular ligands include ECM and cell surface glycoproteins like laminin^{[28, 29]}, fibronectin^{[30]}, CD29^{[31]}, CD66^{[32]}, α1β1 integrin^{[30]}, and Mac-2 binding protein^{[33]}. Intracellularly, GAL-3 binds to ligands such as gemin 4^{[34]}, Bcl-2^{[34]}, nucling^{[35]}, synexin^{[36]}, and β-catenin^{[37, 38]} through various protein-carbohydrate and protein-protein interactions.

GAL-3 functions in various biological capacities, both inside cells, within the nucleus or cytoplasm, and outside, on the cell surface or in the extracellular space^{[3,4,17]}. It connects with β-galactose-rich glycoconjugates or glycolipids on the cell membrane, influencing key cellular processes such as proliferation, apoptosis, adhesion, invasion, angiogenesis, and metastasis. These functions are critical during normal development and also play a role in the progression of diseases associated with chronic inflammation, such as cancer, fibrosis, and type 2 diabetes, etc. Studies have also shown that galectins, a family of 15 glycan-binding proteins that have been conserved throughout evolution, in modulating neuroinflammation and potentially influencing neurodegeneration^{[41, 42]}

Galectin-3 (GAL-3) plays a diverse role in biological processes including cell adhesion, proliferation, migration, apoptosis, tumor progression, and inflammation^{[43-45]}. It is also involved in the modulation of both the innate and adaptive immune systems. The function of GAL-3, whether pro-inflammatory or anti-inflammatory, is influenced by several factors including the specific brain region, the nature of the injury, and the stage of the disease^{[45]}.

In normal brain tissue, GAL-3 is constitutively expressed across a wide range of neuronal tissues, including neurons and glial cells in various brain regions^{[46]}. Detailed studies, particularly those using immunohistochemistry techniques in adult rats, have mapped the presence of GAL-3 in numerous brain areas such as parts of the telencephalon (including certain areas of the cerebral cortex, olfactory region, amygdaloid nucleus, stria terminalis, and the vascular organ of the lamina terminalis), the diencephalon (encompassing the thalamus and hypothalamus), as well as in the brain stem and cerebellum (including the inferior colliculus, lateral parabrachial nucleus, pontine nucleus, cochlear nucleus, and the fibers of the cerebellar peduncles).

Under neuroinflammatory conditions within the central nervous system, GAL-3 expression is in glia, astrocytes, and oligodendrocytes. However, it is primarily expressed by microglia and astrocytes when studied in vitro^{[47]}. This distribution and expression pattern highlight the potential roles of GAL-3 in normal brain function and its reactive upregulation in response to neuroinflammatory conditions, pointing to its significance in both the healthy and diseased states of the nervous system.

Microglia, in their activated state, undergo proliferation, morphological changes, migrate to damaged sites, and produce cytokines^{[48]}, acting as primary effectors in CNS inflammation^{[49]}. Their functional plasticity allows them to exhibit dual phenotypes—proinflammatory M1 and anti-inflammatory M2—enabling them to adapt to various microenvironments and maintain tissue homeostasis^{[50]}.

GAL-3's involvement extends to regulating microglial polarization, influenced by the context of brain damage, through lectin-glycan interactions^{[42]}. Its expression and activity, which can be upregulated by factors like IFN-γ, play a significant role in the proinflammatory response by activating the MI phenotype and promoting cytokine production through pathways like JAK/STAT^{[51]}. However, GAL-3's function in neuroinflammation is complex, displaying both protective and detrimental effects depending on the disease context, stages, and severity^{[52-54]}. For instance, in conditions like traumatic brain injury, GAL-3 exacerbates proinflammatory responses^{[55]}, whereas in autoimmune diseases like EAE (Experimental Autoimmune Encephalomyelitis), it can contribute to neuroprotection by facilitating debris clearance and promoting regeneration and remyelination (56). This dual nature underscores the nuanced role of GAL-3 in neuroinflammatory processes, offering potential therapeutic targets for modulating microglial activity in neurological diseases.

Several GAL-3 inhibitors or antagonists, either small-molecule carbohydrates or large-molecule products, are being developed by private and public companies for therapeutic applications in various diseases.^{[57]}

The terms “GAL-3 inhibitor” or “GAL-3 antagonist” or “GAL-3 blocker” or the like include any substance that decreases the expression, ligand binding (e.g., binding to GAL-3), or any biological activity of GAL-3 (e.g., regulating microglial polarization), that would elicit a biological or medical response of a tissue, system, subject or patient that is being sought by the administrator (such as a researcher, doctor or veterinarian) which includes any measurable alleviation of the signs, symptoms and/or clinical indicia of epilepsy and related neurological disorders, and/or the prevention, slowing or halting of progression of epilepsy and related neurological disorders. GAL-3 inhibitors include both small-molecule carbohydrates or large-molecule natural or unnatural products. Several Gal-3 inhibitors or antagonists, including small-molecule inhibitors and large molecule products, are undergoing clinical development in various diseases associated with Gal-3. A summary of the small molecule and large molecule therapies are presented in Table 1 and Table 2, respectively.

Anti-Gal-3 inhibitors that have been explored further in nonclinical and clinical development of neurological diseases include, for example, TD139, a small molecule Gal-3 inhibitor as described in Hirani, Nikhil et al. “Target inhibition of galectin-3 by inhaled TD139 in patients with idiopathic pulmonary fibrosis.” The European respiratory journal vol. 57, 5 2002559. 27 May 2021, and TB006, an anti-Gal-3 antibody as described in Rasool, S., Patel, P., Johansson, J., Voloboueva, L., Lee, S., Sun, J., Lan, X., Ahmed, T. and Sun, D. (2022), Potential Reversal of Alzheimer's Disease pathology by Antibody TB006 Targeting Galectin-3, the Root Cause of Oligomerization of Amyloid Proteins. Alzheimer's Dement., 18: e060393; each of which is hereby incorporated by reference in its entirety.

### Small-Molecular GAL-3 Targeted Inhibitors

Small molecule anti-GAL-3 inhibitors offer several benefits compared to the large molecule counterparts, including the ability to create a compact structure with desired traits like suitable polar surface area and biostability which enabled the oral formulation development, that can be manufactured consistently on a large scale and is straightforward to characterize, and facilitate pharmacokinetic studies. Nonetheless, synthetic molecules may pose toxicity risks at elevated dosages. Table 1 lists a summary of clinical trials of small molecule Gal-3 inhibitors.

TABLE 1
Overview of Clinical Trials of Small Molecule Gal-3 Inhibitors

| Sponsor | Compound | Indication | Phase | Trial# | Status |
|---|---|---|---|---|---|
| Galecto Bio | GB0139 | IPF | 2 | NCT03832946 | Active, not rec (n = 426) |
| | TD139 | IPF | 1/2a | NCT02257177 | Completed (n = 60) |
| | TD139 | COVID-19 | 1, 2 | NCT04473053 | Active, not rec (n = 200) |
| | GB1211 | Healthy Adults | 1 | NCT03809052 | Completed (n = 78) |
| | | NASH | 1b | NCT04607655 | Withdrawn |
| | | Hepatic Impairment | 1 | NCT05009680 | Active, not rec (n = 54) |
| | | NSCLC | 1 | NCT05009680 | Recruiting (n = 102) |

Galecto Bio, Galecto Biotech; IPF, Idiopathic pulmonary fibrosis; NASH, Nonalcoholic steatohepatitis; NSCLC, Non-small cell lung cancer; not rec, not recruiting.

### Large-Molecular GAL-3 Targeted Inhibitors

Pectins, sourced or modified from plants, represent large molecule antagonists of GAL-3 that have undergone clinical examination for a range of conditions, as documented on www.clinicaltrials.gov (retrieved on 20 Feb. 2023). However, these pectins do not target a specific galectin and exhibit weak binding to GAL-3, with affinities ranging 2.6 to 10 μM. Table 2 lists a summary of clinical trials of large molecule Gal-3 inhibitors.

TABLE 2
Overview of Clinical Trials of Large Molecule Gal-3 Inhibitors

| Sponsor | Compound | Indication | Phase | Trial# | Status |
|---|---|---|---|---|---|
| TrueBinding | TB006 | Healthy Adults | 1 | NCT04920786 | Unknown |
| | | AD | 1b/2a | NCT05074498 | Completed (n = 154) |
| | | COVID-19 | 1 | NCT04801056 | Withdrawn |
| | | Acute ischemic stroke | 2 | NCT05156827 | Withdrawn |
| | | Autism spectrum disorder | 2 | NCT06500637 | Recruiting |
| EcoNugenics | MCP | Prostatic neoplasm | 2 | NCT01681823 | Completed (n = 60) |
| MGH | MCP | Osteoarthritis | 3 | NCT02800629 | Unknown |
| La Jolla | GCS-100 | CKD | 1 | NCT01717248 | Completed (n = 29) |
| | | CLL | 2 | NCT00514696 | Completed (n = 12) |
| | | CKD | 2a | NCT01843790 | Completed (n = 120) |
| | | | 2a | NCT02155673 | Completed (n = 92) |
| | | | 2a | NCT02333955 | Withdrawn |
| | | Multiple myeloma | 1/2 | NCT00609817 | Terminated |
| | | B cell lymphoma | 1/2 | NCT00776802 | Withdrawn |
| Gal Thera | GM-CT-01 | Solid tumors | 1 | NCT00054977 | Completed (n = 40) |
| | GM-CT-01 + | Colorectal Cancer | 2 | NCT00110721 | Terminated |
| | 5-FU | | | | |
| | GM-CT-01 | Colorectal Cancer | 2 | NCT00388700 | Withdrawn |
| | in comb | | | | |
| | GM-CT-01 | Metastatic melanoma | 2 | NCT01723813 | Terminated |
| | | Bile duct and Gall | 2 | NCT00386516 | Withdrawn |
| | | Bladder cancer | | | |
| | GR-MD-02 | NASH and fibrosis | 1 | NCT01899859 | Completed (n = 31) |
| | | NASH and fibrosis | 2 | NCT02421094 | Completed (n = 30) |
| | | NASH cirrhosis and | 2 | NCT02462967 | Completed (n = 162) |
| | | Portal hypertension | | | |
| | | NASH cirrhosis and | 2b/3 | NCT04365868 | Recruiting |
| | | Esophageal varices | | | |
| | | Psoriasis | 2 | NCT02407041 | Completed (n = 5) |
| Prov Med | GR-MD-02 + | Metastatic melanoma | 1 | NCT02117362 | Completed (n = 8) |
| | Ipilumumab | | | | |
| | GR-MD-02 + | Melanoma, NSCLC | 1 | NCT02575404 | Active, not rec |
| | Pembrolizumab | | | | |
| | GR-MD-02 + | Metastatic melanoma | 2 | NCT04987996 | Suspended |
| | Pembrolizumab | | | | |
| BioXyTran | Prolectin-M | COVID-19 | N/A | NCT04512027 | Completed (n = 10) |
| | | COVID-19 | 1b/2a | NCT05733780 | Not yet recruiting |
| | | COVID-19 | 3 | NCT05096052 | Not yet recruiting |

AD, Alheimer’s disease; MCP, Modified citrus pectin; MGH, Massachusetts General Hospital; CKD, Chronic Kidney disease; CLL, Chronic lymphocytic leukemia; Gal Thera, Galectin Therapeutics; GM-CT- 01 in comb, GM-CT-01 in combination with 5-flourouracil, Avastin, and Leucovorin; Prov Med, Providence Portland Medical Center; NSCLC, Non-small cell lung cancer; not rec, not recruiting; NA, Not applicable.

In some embodiments, the anti-GAL-3 antibodies disclosed herein can be used in combination with any of the aforementioned small- or large-molecule GAL-3 targeted inhibitors, for treating epilepsy or a neurological disorder in a subject in need thereof. In some embodiments, the anti-GAL-3 antibodies disclosed herein can be used to treat patients who have not responded to treatment with any of the aforementioned small- or large-molecule GAL-3 targeted inhibitors.

### GAL-3 Targeted Inhibitors for Treatment of Neurological Disorders

Recently, anti-GAL-3 inhibitors for the treatment of neurological disorders such as Alzheimer's disease (AD) have been studied in clinical trials as disclosed on https://clinicaltrials.gov/(NCT05074498, NCT05476783, NCT05156827).

### Anti-GAL-3 Antibodies

As disclosed herein, the GAL-3 inhibitor can be an anti-GAL-3 antibody or antigen-binding fragment thereof that binds specifically to GAL-3 (e.g., human GAL-3) or any soluble fragment thereof (e.g., monoclonal antibodies (e.g., fully human monoclonal antibodies), polyclonal antibodies, bispecific antibodies, Fab antibody fragments, F(ab)2 antibody fragments, Fv antibody fragments (e.g., V_{H} or V_{L}), single chain Fv antibody fragments, dsFv antibody fragments, humanized antibodies or chimeric antibodies.

In some instances, the anti-GAL-3 antibody specifically binds to human Galectin-3. In other instances, the anti-GAL-3 antibody binds to both human Galectin-3 and Galectin-3 from other species such as mouse, rat, monkey, etc. As disclosed herein, the anti-GAL-3 antibody can be any humanized, chimeric, or human antibody that binds to Galectin-3. In some embodiments, the anti-GAL-3 antibody is generated from other species but humanized to bind human Galectin-3. In some embodiments, the anti-GAL-3 antibody is a chimeric antibody. In some embodiments, the anti-GAL-3 antibody is a human antibody.

In some embodiments, the anti-GAL-3 antibody comprises a heavy chain variable (V_{H}) region comprising a heavy chain CDR1 (HCDR1), HCDR2, and HCDR3 and a light chain variable (V_{L}) region comprising light chain CDR1 (LCDR1), LCDR2, and LCDR3. In some embodiments, the V_{H} region comprises a HCDR1 amino acid sequence at least 70%, at least 75%, at least 80%, at least 85%, at least 90%, or at least 95% identical to any one of SEQ ID NOS: 1-21 and 481-507, a HCDR2 amino acid sequence at least 70%, at least 75%, at least 80%, at least 85%, at least 90%, or at least 95% identical to any one of SEQ ID NOS: 22-42 and 390-417, and/or a HCDR3 amino acid sequence at least 70%, at least 75%, at least 80%, at least 85%, at least 90%, or at least 95% identical to any one of SEQ ID NOS: 43-63 and 418-439. In some embodiments, the V_{L} region comprises a LCDR1 amino acid sequence at least 70%, at least 75%, at least 80%, at least 85%, at least 90%, or at least 95% identical to any one of SEQ ID NOS: 169-189 and 440-462, a LCDR2 amino acid sequence at least 70%, at least 75%, at least 80%, at least 85%, at least 90%, or at least 95% identical to any one of SEQ ID NOS: 190-210 and 463-480, and/or a LCDR3 amino acid sequence at least 70%, at least 75%, at least 80%, at least 85%, at least 90%, or at least 95% identical to any one of SEQ ID NOS: 211-231 and 481-507. The exemplary heavy chain CDRs and light chain CDRs are listed in Tables 3 and 4, respectively.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 1, a HCDR2 amino acid sequence at least 70%, at least 75%, at least 80%, at least 85%, at least 90%, or at least 95% identical to SEQ ID NO: 22, and a HCDR3 amino acid sequence at least 70%, at least 75%, at least 80%, at least 85%, at least 90%, or at least 95% identical to SEQ ID NO: 43; and a light chain variable region comprising a LCDR1 amino acid sequence at least 70%, at least 75%, at least 80%, at least 85%, at least 90%, or at least 95% identical to SEQ ID NO: 169, a LCDR2 amino acid sequence at least 70%, at least 75%, at least 80%, at least 85%, at least 90%, or at least 95% identical to SEQ ID NO: 190, and a LCDR3 amino acid sequence at least 70%, at least 75%, at least 80%, at least 85%, at least 90%, or at least 95% identical to SEQ ID NO: 211. In a particular embodiment, the anti-GAL-3 antibody comprises a HCDR1 amino acid sequence of SEQ ID NO: 1, a HCDR2 amino acid sequence of SEQ ID NO: 22, a HCDR3 amino acid sequence of SEQ ID NO: 43, a LCDR1 amino acid sequence of SEQ ID NO: 169, a LCDR2 amino acid sequence of SEQ ID NO: 190, and a LCDR3 amino acid sequence of SEQ ID NO: 211.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 2, a HCDR2 amino acid sequence at least 70%, at least 75%, at least 80%, at least 85%, at least 90%, or at least 95% identical to SEQ ID NO: 23, and a HCDR3 amino acid sequence at least 70%, at least 75%, at least 80%, at least 85%, at least 90%, or at least 95% identical to SEQ ID NO: 44; and a light chain variable region comprising a LCDR1 amino acid sequence at least 70%, at least 75%, at least 80%, at least 85%, at least 90%, or at least 95% identical to SEQ ID NO: 170, a LCDR2 amino acid sequence at least 70%, at least 75%, at least 80%, at least 85%, at least 90%, or at least 95% identical to SEQ ID NO: 191, and a LCDR3 amino acid sequence at least 70%, at least 75%, at least 80%, at least 85%, at least 90%, or at least 95% identical to SEQ ID NO: 212. In a particular embodiment, the anti-GAL-3 antibody comprises a HCDR1 amino acid sequence of SEQ ID NO: 2, a HCDR2 amino acid sequence of SEQ ID NO: 23, a HCDR3 amino acid sequence of SEQ ID NO: 44, a LCDR1 amino acid sequence of SEQ ID NO: 170, a LCDR2 amino acid sequence of SEQ ID NO: 191, and a LCDR3 amino acid sequence of SEQ ID NO: 212.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 3, a HCDR2 amino acid sequence at least 70%, at least 75%, at least 80%, at least 85%, at least 90%, or at least 95% identical to SEQ ID NO: 24, and a HCDR3 amino acid sequence at least 70%, at least 75%, at least 80%, at least 85%, at least 90%, or at least 95% identical to SEQ ID NO: 45; and a light chain variable region comprising a LCDR1 amino acid sequence at least 70%, at least 75%, at least 80%, at least 85%, at least 90%, or at least 95% identical to SEQ ID NO: 171, a LCDR2 amino acid sequence at least 70%, at least 75%, at least 80%, at least 85%, at least 90%, or at least 95% identical to SEQ ID NO: 192, and a LCDR3 amino acid sequence at least 70%, at least 75%, at least 80%, at least 85%, at least 90%, or at least 95% identical to SEQ ID NO: 213. In a particular embodiment, the anti-GAL-3 antibody comprises a HCDR1 amino acid sequence of SEQ ID NO: 3, a HCDR2 amino acid sequence of SEQ ID NO: 24, a HCDR3 amino acid sequence of SEQ ID NO: 45, a LCDR1 amino acid sequence of SEQ ID NO: 171, a LCDR2 amino acid sequence of SEQ ID NO: 192, and a LCDR3 amino acid sequence of SEQ ID NO: 213.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 4, a HCDR2 amino acid sequence at least 70%, at least 75%, at least 80%, at least 85%, at least 90%, or at least 95% identical to SEQ ID NO: 25, and a HCDR3 amino acid sequence at least 70%, at least 75%, at least 80%, at least 85%, at least 90%, or at least 95% identical to SEQ ID NO: 46; and a light chain variable region comprising a LCDR1 amino acid sequence at least 70%, at least 75%, at least 80%, at least 85%, at least 90%, or at least 95% identical to SEQ ID NO: 172, a LCDR2 amino acid sequence at least 70%, at least 75%, at least 80%, at least 85%, at least 90%, or at least 95% identical to SEQ ID NO: 193, and a LCDR3 amino acid sequence at least 70%, at least 75%, at least 80%, at least 85%, at least 90%, or at least 95% identical to SEQ ID NO: 214. In a particular embodiment, the anti-GAL-3 antibody comprises a HCDR1 amino acid sequence of SEQ ID NO: 4, a HCDR2 amino acid sequence of SEQ ID NO: 25, a HCDR3 amino acid sequence of SEQ ID NO: 46, a LCDR1 amino acid sequence of SEQ ID NO: 172, a LCDR2 amino acid sequence of SEQ ID NO: 193, and a LCDR3 amino acid sequence of SEQ ID NO: 214.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 5, a HCDR2 amino acid sequence at least 70%, at least 75%, at least 80%, at least 85%, at least 90%, or at least 95% identical to SEQ ID NO: 26, and a HCDR3 amino acid sequence at least 70%, at least 75%, at least 80%, at least 85%, at least 90%, or at least 95% identical to SEQ ID NO: 47; and a light chain variable region comprising a LCDR1 amino acid sequence at least 70%, at least 75%, at least 80%, at least 85%, at least 90%, or at least 95% identical to SEQ ID NO: 173, a LCDR2 amino acid sequence at least 70%, at least 75%, at least 80%, at least 85%, at least 90%, or at least 95% identical to SEQ ID NO: 194, and a LCDR3 amino acid sequence at least 70%, at least 75%, at least 80%, at least 85%, at least 90%, or at least 95% identical to SEQ ID NO: 215. In a particular embodiment, the anti-GAL-3 antibody comprises a HCDR1 amino acid sequence of SEQ ID NO: 5, a HCDR2 amino acid sequence of SEQ ID NO: 26, a HCDR3 amino acid sequence of SEQ ID NO: 47, a LCDR1 amino acid sequence of SEQ ID NO: 173, a LCDR2 amino acid sequence of SEQ ID NO: 194, and a LCDR3 amino acid sequence of SEQ ID NO: 215.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 7, a HCDR2 amino acid sequence at least 70%, at least 75%, at least 80%, at least 85%, at least 90%, or at least 95% identical to SEQ ID NO: 28, and a HCDR3 amino acid sequence at least 70%, at least 75%, at least 80%, at least 85%, at least 90%, or at least 95% identical to SEQ ID NO: 49; and a light chain variable region comprising a LCDR1 amino acid sequence at least 70%, at least 75%, at least 80%, at least 85%, at least 90%, or at least 95% identical to SEQ ID NO: 174, a LCDR2 amino acid sequence at least 70%, at least 75%, at least 80%, at least 85%, at least 90%, or at least 95% identical to SEQ ID NO: 195, and a LCDR3 amino acid sequence at least 70%, at least 75%, at least 80%, at least 85%, at least 90%, or at least 95% identical to SEQ ID NO: 216. In a particular embodiment, the anti-GAL-3 antibody comprises a HCDR1 amino acid sequence of SEQ ID NO: 6, a HCDR2 amino acid sequence of SEQ ID NO: 27, a HCDR3 amino acid sequence of SEQ ID NO: 48, a LCDR1 amino acid sequence of SEQ ID NO: 174, a LCDR2 amino acid sequence of SEQ ID NO: 195, and a LCDR3 amino acid sequence of SEQ ID NO: 216.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 7, a HCDR2 amino acid sequence at least 70%, at least 75%, at least 80%, at least 85%, at least 90%, or at least 95% identical to SEQ ID NO: 28, and a HCDR3 amino acid sequence at least 70%, at least 75%, at least 80%, at least 85%, at least 90%, or at least 95% identical to SEQ ID NO: 49; and a light chain variable region comprising a LCDR1 amino acid sequence at least 70%, at least 75%, at least 80%, at least 85%, at least 90%, or at least 95% identical to SEQ ID NO: 175, a LCDR2 amino acid sequence at least 70%, at least 75%, at least 80%, at least 85%, at least 90%, or at least 95% identical to SEQ ID NO: 196, and a LCDR3 amino acid sequence at least 70%, at least 75%, at least 80%, at least 85%, at least 90%, or at least 95% identical to SEQ ID NO: 217. In a particular embodiment, the anti-GAL-3 antibody comprises a HCDR1 amino acid sequence of SEQ ID NO: 7, a HCDR2 amino acid sequence of SEQ ID NO: 28, a HCDR3 amino acid sequence of SEQ ID NO: 49, a LCDR1 amino acid sequence of SEQ ID NO: 175, a LCDR2 amino acid sequence of SEQ ID NO: 196, and a LCDR3 amino acid sequence of SEQ ID NO: 217.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 8, a HCDR2 amino acid sequence at least 70%, at least 75%, at least 80%, at least 85%, at least 90%, or at least 95% identical to SEQ ID NO: 29, and a HCDR3 amino acid sequence at least 70%, at least 75%, at least 80%, at least 85%, at least 90%, or at least 95% identical to SEQ ID NO: 50; and a light chain variable region comprising a LCDR1 amino acid sequence at least 70%, at least 75%, at least 80%, at least 85%, at least 90%, or at least 95% identical to SEQ ID NO: 176 or 369, a LCDR2 amino acid sequence at least 70%, at least 75%, at least 80%, at least 85%, at least 90%, or at least 95% identical to SEQ ID NO: 197, and a LCDR3 amino acid sequence at least 70%, at least 75%, at least 80%, at least 85%, at least 90%, or at least 95% identical to SEQ ID NO: 218. In a particular embodiment, the anti-GAL-3 antibody comprises a HCDR1 amino acid sequence of SEQ ID NO: 8, a HCDR2 amino acid sequence of SEQ ID NO: 29, a HCDR3 amino acid sequence of SEQ ID NO: 50, a LCDR1 amino acid sequence of SEQ ID NO: 176 or 369, a LCDR2 amino acid sequence of SEQ ID NO: 197, and a LCDR3 amino acid sequence of SEQ ID NO: 218.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 9, a HCDR2 amino acid sequence at least 70%, at least 75%, at least 80%, at least 85%, at least 90%, or at least 95% identical to SEQ ID NO: 80, and a HCDR3 amino acid sequence at least 70%, at least 75%, at least 80%, at least 85%, at least 90%, or at least 95% identical to SEQ ID NO: 51; and a light chain variable region comprising a LCDR1 amino acid sequence at least 70%, at least 75%, at least 80%, at least 85%, at least 90%, or at least 95% identical to SEQ ID NO: 177, a LCDR2 amino acid sequence at least 70%, at least 75%, at least 80%, at least 85%, at least 90%, or at least 95% identical to SEQ ID NO: 198, and a LCDR3 amino acid sequence at least 70%, at least 75%, at least 80%, at least 85%, at least 90%, or at least 95% identical to SEQ ID NO: 219. In a particular embodiment, the anti-GAL-3 antibody comprises a HCDR1 amino acid sequence of SEQ ID NO: 9, a HCDR2 amino acid sequence of SEQ ID NO: 30, a HCDR3 amino acid sequence of SEQ ID NO: 51, a LCDR1 amino acid sequence of SEQ ID NO: 177, a LCDR2 amino acid sequence of SEQ ID NO: 198, and a LCDR3 amino acid sequence of SEQ ID NO: 219.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 10, a HCDR2 amino acid sequence at least 70%, at least 75%, at least 80%, at least 85%, at least 90%, or at least 95% identical to SEQ ID NO: 31, and a HCDR3 amino acid sequence at least 70%, at least 75%, at least 80%, at least 85%, at least 90%, or at least 95% identical to SEQ ID NO: 52; and a light chain variable region comprising a LCDR1 amino acid sequence at least 70%, at least 75%, at least 80%, at least 85%, at least 90%, or at least 95% identical to SEQ ID NO: 178, a LCDR2 amino acid sequence at least 70%, at least 75%, at least 80%, at least 85%, at least 90%, or at least 95% identical to SEQ ID NO: 199, and a LCDR3 amino acid sequence at least 70%, at least 75%, at least 80%, at least 85%, at least 90%, or at least 95% identical to SEQ ID NO: 220. In a particular embodiment, the anti-GAL-3 antibody comprises a HCDR1 amino acid sequence of SEQ ID NO: 10, a HCDR2 amino acid sequence of SEQ ID NO: 31, a HCDR3 amino acid sequence of SEQ ID NO: 52, a LCDR1 amino acid sequence of SEQ ID NO: 178, a LCDR2 amino acid sequence of SEQ ID NO: 199, and a LCDR3 amino acid sequence of SEQ ID NO: 220.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 11, a HCDR2 amino acid sequence at least 70%, at least 75%, at least 80%, at least 85%, at least 90%, or at least 95% identical to SEQ ID NO: 32, and a HCDR3 amino acid sequence at least 70%, at least 75%, at least 80%, at least 85%, at least 90%, or at least 95% identical to SEQ ID NO: 53; and a light chain variable region comprising a LCDR1 amino acid sequence at least 70%, at least 75%, at least 80%, at least 85%, at least 90%, or at least 95% identical to SEQ ID NO: 179, a LCDR2 amino acid sequence at least 70%, at least 75%, at least 80%, at least 85%, at least 90%, or at least 95% identical to SEQ ID NO: 200, and a LCDR3 amino acid sequence at least 70%, at least 75%, at least 80%, at least 85%, at least 90%, or at least 95% identical to SEQ ID NO: 221. In a particular embodiment, the anti-GAL-3 antibody comprises a HCDR1 amino acid sequence of SEQ ID NO: 11, a HCDR2 amino acid sequence of SEQ ID NO: 32, a HCDR3 amino acid sequence of SEQ ID NO: 53, a LCDR1 amino acid sequence of SEQ ID NO: 179, a LCDR2 amino acid sequence of SEQ ID NO: 200, and a LCDR3 amino acid sequence of SEQ ID NO: 221.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 12, a HCDR2 amino acid sequence at least 70%, at least 75%, at least 80%, at least 85%, at least 90%, or at least 95% identical to SEQ ID NO: 33, and a HCDR3 amino acid sequence at least 70%, at least 75%, at least 80%, at least 85%, at least 90%, or at least 95% identical to SEQ ID NO: 54; and a light chain variable region comprising a LCDR1 amino acid sequence at least 70%, at least 75%, at least 80%, at least 85%, at least 90%, or at least 95% identical to SEQ ID NO: 180, a LCDR2 amino acid sequence at least 70%, at least 75%, at least 80%, at least 85%, at least 90%, or at least 95% identical to SEQ ID NO: 201, and a LCDR3 amino acid sequence at least 70%, at least 75%, at least 80%, at least 85%, at least 90%, or at least 95% identical to SEQ ID NO: 222. In a particular embodiment, the anti-GAL-3 antibody comprises a HCDR1 amino acid sequence of SEQ ID NO: 12, a HCDR2 amino acid sequence of SEQ ID NO: 33, a HCDR3 amino acid sequence of SEQ ID NO: 54, a LCDR1 amino acid sequence of SEQ ID NO: 180, a LCDR2 amino acid sequence of SEQ ID NO: 201, and a LCDR3 amino acid sequence of SEQ ID NO: 222.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 13, a HCDR2 amino acid sequence at least 70%, at least 75%, at least 80%, at least 85%, at least 90%, or at least 95% identical to SEQ ID NO: 34, and a HCDR3 amino acid sequence at least 70%, at least 75%, at least 80%, at least 85%, at least 90%, or at least 95% identical to SEQ ID NO: 55; and a light chain variable region comprising a LCDR1 amino acid sequence at least 70%, at least 75%, at least 80%, at least 85%, at least 90%, or at least 95% identical to SEQ ID NO: 181, a LCDR2 amino acid sequence at least 70%, at least 75%, at least 80%, at least 85%, at least 90%, or at least 95% identical to SEQ ID NO: 202, and a LCDR3 amino acid sequence at least 70%, at least 75%, at least 80%, at least 85%, at least 90%, or at least 95% identical to SEQ ID NO: 223. In a particular embodiment, the anti-GAL-3 antibody comprises a HCDR1 amino acid sequence of SEQ ID NO: 13, a HCDR2 amino acid sequence of SEQ ID NO: 34, a HCDR3 amino acid sequence of SEQ ID NO: 55, a LCDR1 amino acid sequence of SEQ ID NO: 181, a LCDR2 amino acid sequence of SEQ ID NO: 202, and a LCDR3 amino acid sequence of SEQ ID NO: 223.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 14, a HCDR2 amino acid sequence at least 70%, at least 75%, at least 80%, at least 85%, at least 90%, or at least 95% identical to SEQ ID NO: 35, and a HCDR3 amino acid sequence at least 70%, at least 75%, at least 80%, at least 85%, at least 90%, or at least 95% identical to SEQ ID NO: 56; and a light chain variable region comprising a LCDR1 amino acid sequence at least 70%, at least 75%, at least 80%, at least 85%, at least 90%, or at least 95% identical to SEQ ID NO: 182, a LCDR2 amino acid sequence at least 70%, at least 75%, at least 80%, at least 85%, at least 90%, or at least 95% identical to SEQ ID NO: 203, and a LCDR3 amino acid sequence at least 70%, at least 75%, at least 80%, at least 85%, at least 90%, or at least 95% identical to SEQ ID NO: 224. In a particular embodiment, the anti-GAL-3 antibody comprises a HCDR1 amino acid sequence of SEQ ID NO: 14, a HCDR2 amino acid sequence of SEQ ID NO: 35, a HCDR3 amino acid sequence of SEQ ID NO: 56, a LCDR1 amino acid sequence of SEQ ID NO: 182, a LCDR2 amino acid sequence of SEQ ID NO: 203, and a LCDR3 amino acid sequence of SEQ ID NO: 224.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 15, a HCDR2 amino acid sequence at least 70%, at least 75%, at least 80%, at least 85%, at least 90%, or at least 95% identical to SEQ ID NO: 36, and a HCDR3 amino acid sequence at least 70%, at least 75%, at least 80%, at least 85%, at least 90%, or at least 95% identical to SEQ ID NO: 57; and a light chain variable region comprising a LCDR1 amino acid sequence at least 70%, at least 75%, at least 80%, at least 85%, at least 90%, or at least 95% identical to SEQ ID NO: 183, a LCDR2 amino acid sequence at least 70%, at least 75%, at least 80%, at least 85%, at least 90%, or at least 95% identical to SEQ ID NO: 204, and a LCDR3 amino acid sequence at least 70%, at least 75%, at least 80%, at least 85%, at least 90%, or at least 95% identical to SEQ ID NO: 225. In a particular embodiment, the anti-GAL-3 antibody comprises a HCDR1 amino acid sequence of SEQ ID NO: 15, a HCDR2 amino acid sequence of SEQ ID NO: 36, a HCDR3 amino acid sequence of SEQ ID NO: 57, a LCDR1 amino acid sequence of SEQ ID NO: 183, a LCDR2 amino acid sequence of SEQ ID NO: 204, and a LCDR3 amino acid sequence of SEQ ID NO: 225.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 16, a HCDR2 amino acid sequence at least 70%, at least 75%, at least 80%, at least 85%, at least 90%, or at least 95% identical to SEQ ID NO: 37, and a HCDR3 amino acid sequence at least 70%, at least 75%, at least 80%, at least 85%, at least 90%, or at least 95% identical to SEQ ID NO: 58; and a light chain variable region comprising a LCDR1 amino acid sequence at least 70%, at least 75%, at least 80%, at least 85%, at least 90%, or at least 95% identical to SEQ ID NO: 184, a LCDR2 amino acid sequence at least 70%, at least 75%, at least 80%, at least 85%, at least 90%, or at least 95% identical to SEQ ID NO: 205, and a LCDR3 amino acid sequence at least 70%, at least 75%, at least 80%, at least 85%, at least 90%, or at least 95% identical to SEQ ID NO: 226. In a particular embodiment, the anti-GAL-3 antibody comprises a HCDR1 amino acid sequence of SEQ ID NO: 16, a HCDR2 amino acid sequence of SEQ ID NO: 37, a HCDR3 amino acid sequence of SEQ ID NO: 58, a LCDR1 amino acid sequence of SEQ ID NO: 184, a LCDR2 amino acid sequence of SEQ ID NO: 205, and a LCDR3 amino acid sequence of SEQ ID NO: 226.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 17, a HCDR2 amino acid sequence at least 70%, at least 75%, at least 80%, at least 85%, at least 90%, or at least 95% identical to SEQ ID NO: 38, and a HCDR3 amino acid sequence at least 70%, at least 75%, at least 80%, at least 85%, at least 90%, or at least 95% identical to SEQ ID NO: 59; and a light chain variable region comprising a LCDR1 amino acid sequence at least 70%, at least 75%, at least 80%, at least 85%, at least 90%, or at least 95% identical to SEQ ID NO: 185, a LCDR2 amino acid sequence at least 70%, at least 75%, at least 80%, at least 85%, at least 90%, or at least 95% identical to SEQ ID NO: 206, and a LCDR3 amino acid sequence at least 70%, at least 75%, at least 80%, at least 85%, at least 90%, or at least 95% identical to SEQ ID NO: 227. In a particular embodiment, the anti-GAL-3 antibody comprises a HCDR1 amino acid sequence of SEQ ID NO: 17, a HCDR2 amino acid sequence of SEQ ID NO: 38, a HCDR3 amino acid sequence of SEQ ID NO: 59, a LCDR1 amino acid sequence of SEQ ID NO: 185, a LCDR2 amino acid sequence of SEQ ID NO: 206, and a LCDR3 amino acid sequence of SEQ ID NO: 227.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 18, a HCDR2 amino acid sequence at least 70%, at least 75%, at least 80%, at least 85%, at least 90%, or at least 95% identical to SEQ ID NO: 39, and a HCDR3 amino acid sequence at least 70%, at least 75%, at least 80%, at least 85%, at least 90%, or at least 95% identical to SEQ ID NO: 60; and a light chain variable region comprising a LCDR1 amino acid sequence at least 70%, at least 75%, at least 80%, at least 85%, at least 90%, or at least 95% identical to SEQ ID NO: 186, a LCDR2 amino acid sequence at least 70%, at least 75%, at least 80%, at least 85%, at least 90%, or at least 95% identical to SEQ ID NO: 207, and a LCDR3 amino acid sequence at least 70%, at least 75%, at least 80%, at least 85%, at least 90%, or at least 95% identical to SEQ ID NO: 228. In a particular embodiment, the anti-GAL-3 antibody comprises a HCDR1 amino acid sequence of SEQ ID NO: 18, a HCDR2 amino acid sequence of SEQ ID NO: 39, a HCDR3 amino acid sequence of SEQ ID NO: 60, a LCDR1 amino acid sequence of SEQ ID NO: 186, a LCDR2 amino acid sequence of SEQ ID NO: 207, and a LCDR3 amino acid sequence of SEQ ID NO: 228.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 19, a HCDR2 amino acid sequence at least 70%, at least 75%, at least 80%, at least 85%, at least 90%, or at least 95% identical to SEQ ID NO: 40, and a HCDR3 amino acid sequence at least 70%, at least 75%, at least 80%, at least 85%, at least 90%, or at least 95% identical to SEQ ID NO: 61; and a light chain variable region comprising a LCDR1 amino acid sequence at least 70%, at least 75%, at least 80%, at least 85%, at least 90%, or at least 95% identical to SEQ ID NO: 187, a LCDR2 amino acid sequence at least 70%, at least 75%, at least 80%, at least 85%, at least 90%, or at least 95% identical to SEQ ID NO: 208, and a LCDR3 amino acid sequence at least 70%, at least 75%, at least 80%, at least 85%, at least 90%, or at least 95% identical to SEQ ID NO: 229. In a particular embodiment, the anti-GAL-3 antibody comprises a HCDR1 amino acid sequence of SEQ ID NO: 19, a HCDR2 amino acid sequence of SEQ ID NO: 40, a HCDR3 amino acid sequence of SEQ ID NO: 61, a LCDR1 amino acid sequence of SEQ ID NO: 187, a LCDR2 amino acid sequence of SEQ ID NO: 208, and a LCDR3 amino acid sequence of SEQ ID NO: 229.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 20, a HCDR2 amino acid sequence at least 70%, at least 75%, at least 80%, at least 85%, at least 90%, or at least 95% identical to SEQ ID NO: 41, and a HCDR3 amino acid sequence at least 70%, at least 75%, at least 80%, at least 85%, at least 90%, or at least 95% identical to SEQ ID NO: 62; and a light chain variable region comprising a LCDR1 amino acid sequence at least 70%, at least 75%, at least 80%, at least 85%, at least 90%, or at least 95% identical to SEQ ID NO: 188, a LCDR2 amino acid sequence at least 70%, at least 75%, at least 80%, at least 85%, at least 90%, or at least 95% identical to SEQ ID NO: 209, and a LCDR3 amino acid sequence at least 70%, at least 75%, at least 80%, at least 85%, at least 90%, or at least 95% identical to SEQ ID NO: 230. In a particular embodiment, the anti-GAL-3 antibody comprises a HCDR1 amino acid sequence of SEQ ID NO: 20, a HCDR2 amino acid sequence of SEQ ID NO: 41, a HCDR3 amino acid sequence of SEQ ID NO: 64, a LCDR1 amino acid sequence of SEQ ID NO: 188, a LCDR2 amino acid sequence of SEQ ID NO: 209, and a LCDR3 amino acid sequence of SEQ ID NO: 230.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 21, a HCDR2 amino acid sequence at least 70%, at least 75%, at least 80%, at least 85%, at least 90%, or at least 95% identical to SEQ ID NO: 42, and a HCDR3 amino acid sequence at least 70%, at least 75%, at least 80%, at least 85%, at least 90%, or at least 95% identical to SEQ ID NO: 63; and a light chain variable region comprising a LCDR1 amino acid sequence at least 70%, at least 75%, at least 80%, at least 85%, at least 90%, or at least 95% identical to SEQ ID NO: 189, a LCDR2 amino acid sequence at least 70%, at least 75%, at least 80%, at least 85%, at least 90%, or at least 95% identical to SEQ ID NO: 220, and a LCDR3 amino acid sequence at least 70%, at least 75%, at least 80%, at least 85%, at least 90%, or at least 95% identical to SEQ ID NO: 231. In a particular embodiment, the anti-GAL-3 antibody comprises a HCDR1 amino acid sequence of SEQ ID NO: 21, a HCDR2 amino acid sequence of SEQ ID NO: 42, a HCDR3 amino acid sequence of SEQ ID NO: 63, a LCDR1 amino acid sequence of SEQ ID NO: 189, a LCDR2 amino acid sequence of SEQ ID NO: 210, and a LCDR3 amino acid sequence of SEQ ID NO: 231.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 370, a HCDR2 amino acid sequence at least 70%, at least 75%, at least 80%, at least 85%, at least 90%, or at least 95% identical to SEQ ID NO: 390, and a HCDR3 amino acid sequence at least 70%, at least 75%, at least 80%, at least 85%, at least 90%, or at least 95% identical to SEQ ID NO: 418; and a light chain variable region comprising a LCDR1 amino acid sequence at least 70%, at least 75%, at least 80%, at least 85%, at least 90%, or at least 95% identical to SEQ ID NO: 440, a LCDR2 amino acid sequence at least 70%, at least 75%, at least 80%, at least 85%, at least 90%, or at least 95% identical to SEQ ID NO: 463, and a LCDR3 amino acid sequence at least 70%, at least 75%, at least 80%, at least 85%, at least 90%, or at least 95% identical to SEQ ID NO: 481. In a particular embodiment, the anti-GAL-3 antibody comprises a HCDR1 amino acid sequence of SEQ ID NO: 370, a HCDR2 amino acid sequence of SEQ ID NO: 390, a HCDR3 amino acid sequence of SEQ ID NO: 418, a LCDR1 amino acid sequence of SEQ ID NO: 440, a LCDR2 amino acid sequence of SEQ ID NO: 463, and a LCDR3 amino acid sequence of SEQ ID NO: 481.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 370, a HCDR2 amino acid sequence at least 70%, at least 75%, at least 80%, at least 85%, at least 90%, or at least 95% identical to SEQ ID NO: 390, and a HCDR3 amino acid sequence at least 70%, at least 75%, at least 80%, at least 85%, at least 90%, or at least 95% identical to SEQ ID NO: 418; and a light chain variable region comprising a LCDR1 amino acid sequence at least 70%, at least 75%, at least 80%, at least 85%, at least 90%, or at least 95% identical to SEQ ID NO: 440, a LCDR2 amino acid sequence at least 70%, at least 75%, at least 80%, at least 85%, at least 90%, or at least 95% identical to SEQ ID NO: 463, and a LCDR3 amino acid sequence at least 70%, at least 75%, at least 80%, at least 85%, at least 90%, or at least 95% identical to SEQ ID NO: 481. In a particular embodiment, the anti-GAL-3 antibody comprises a HCDR1 amino acid sequence of SEQ ID NO: 370, a HCDR2 amino acid sequence of SEQ ID NO: 390, a HCDR3 amino acid sequence of SEQ ID NO: 418, a LCDR1 amino acid sequence of SEQ ID NO: 440, a LCDR2 amino acid sequence of SEQ ID NO: 463, and a LCDR3 amino acid sequence of SEQ ID NO: 481.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 371, a HCDR2 amino acid sequence at least 70%, at least 75%, at least 80%, at least 85%, at least 90%, or at least 95% identical to SEQ ID NO: 391, and a HCDR3 amino acid sequence at least 70%, at least 75%, at least 80%, at least 85%, at least 90%, or at least 95% identical to SEQ ID NO: 418; and a light chain variable region comprising a LCDR1 amino acid sequence at least 70%, at least 75%, at least 80%, at least 85%, at least 90%, or at least 95% identical to SEQ ID NO: 441, a LCDR2 amino acid sequence at least 70%, at least 75%, at least 80%, at least 85%, at least 90%, or at least 95% identical to SEQ ID NO: 464, and a LCDR3 amino acid sequence at least 70%, at least 75%, at least 80%, at least 85%, at least 90%, or at least 95% identical to SEQ ID NO: 482. In a particular embodiment, the anti-GAL-3 antibody comprises a HCDR1 amino acid sequence of SEQ ID NO: 371, a HCDR2 amino acid sequence of SEQ ID NO: 391, a HCDR3 amino acid sequence of SEQ ID NO: 418, a LCDR1 amino acid sequence of SEQ ID NO: 441, a LCDR2 amino acid sequence of SEQ ID NO: 464, and a LCDR3 amino acid sequence of SEQ ID NO: 482.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 372, a HCDR2 amino acid sequence at least 70%, at least 75%, at least 80%, at least 85%, at least 90%, or at least 95% identical to SEQ ID NO: 392, and a HCDR3 amino acid sequence at least 70%, at least 75%, at least 80%, at least 85%, at least 90%, or at least 95% identical to DNL; and a light chain variable region comprising a LCDR1 amino acid sequence at least 70%, at least 75%, at least 80%, at least 85%, at least 90%, or at least 95% identical to SEQ ID NO: 442, a LCDR2 amino acid sequence at least 70%, at least 75%, at least 80%, at least 85%, at least 90%, or at least 95% identical to SEQ ID NO: 465, and a LCDR3 amino acid sequence at least 70%, at least 75%, at least 80%, at least 85%, at least 90%, or at least 95% identical to SEQ ID NO: 483. In a particular embodiment, the anti-GAL-3 antibody comprises a HCDR1 amino acid sequence of SEQ ID NO: 372, a HCDR2 amino acid sequence of SEQ ID NO: 392, a HCDR3 amino acid sequence of DNL, a LCDR1 amino acid sequence of SEQ ID NO: 442, a LCDR2 amino acid sequence of SEQ ID NO: 465, and a LCDR3 amino acid sequence of SEQ ID NO: 483.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 373, a HCDR2 amino acid sequence at least 70%, at least 75%, at least 80%, at least 85%, at least 90%, or at least 95% identical to SEQ ID NO: 393, and a HCDR3 amino acid sequence at least 70%, at least 75%, at least 80%, at least 85%, at least 90%, or at least 95% identical to SEQ ID NO: 419; and a light chain variable region comprising a LCDR1 amino acid sequence at least 70%, at least 75%, at least 80%, at least 85%, at least 90%, or at least 95% identical to SEQ ID NO: 443, a LCDR2 amino acid sequence at least 70%, at least 75%, at least 80%, at least 85%, at least 90%, or at least 95% identical to SEQ ID NO: 466, and a LCDR3 amino acid sequence at least 70%, at least 75%, at least 80%, at least 85%, at least 90%, or at least 95% identical to SEQ ID NO: 484. In a particular embodiment, the anti-GAL-3 antibody comprises a HCDR1 amino acid sequence of SEQ ID NO: 373, a HCDR2 amino acid sequence of SEQ ID NO: 393, a HCDR3 amino acid sequence of SEQ ID NO: 419, a LCDR1 amino acid sequence of SEQ ID NO: 443, a LCDR2 amino acid sequence of SEQ ID NO: 466, and a LCDR3 amino acid sequence of SEQ ID NO: 484.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 374, a HCDR2 amino acid sequence at least 70%, at least 75%, at least 80%, at least 85%, at least 90%, or at least 95% identical to SEQ ID NO: 394, and a HCDR3 amino acid sequence at least 70%, at least 75%, at least 80%, at least 85%, at least 90%, or at least 95% identical to SEQ ID NO: 420; and a light chain variable region comprising a LCDR1 amino acid sequence at least 70%, at least 75%, at least 80%, at least 85%, at least 90%, or at least 95% identical to SEQ ID NO: 444, a LCDR2 amino acid sequence at least 70%, at least 75%, at least 80%, at least 85%, at least 90%, or at least 95% identical to SEQ ID NO: 467, and a LCDR3 amino acid sequence at least 70%, at least 75%, at least 80%, at least 85%, at least 90%, or at least 95% identical to SEQ ID NO: 485. In a particular embodiment, the anti-GAL-3 antibody comprises a HCDR1 amino acid sequence of SEQ ID NO: 374, a HCDR2 amino acid sequence of SEQ ID NO: 394, a HCDR3 amino acid sequence of SEQ ID NO: 420, a LCDR1 amino acid sequence of SEQ ID NO: 444, a LCDR2 amino acid sequence of SEQ ID NO: 467, and a LCDR3 amino acid sequence of SEQ ID NO: 485.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 375, a HCDR2 amino acid sequence at least 70%, at least 75%, at least 80%, at least 85%, at least 90%, or at least 95% identical to SEQ ID NO: 395, and a HCDR3 amino acid sequence at least 70%, at least 75%, at least 80%, at least 85%, at least 90%, or at least 95% identical to SEQ ID NO: 421; and a light chain variable region comprising a LCDR1 amino acid sequence at least 70%, at least 75%, at least 80%, at least 85%, at least 90%, or at least 95% identical to SEQ ID NO: 445, a LCDR2 amino acid sequence at least 70%, at least 75%, at least 80%, at least 85%, at least 90%, or at least 95% identical to SEQ ID NO: 468, and a LCDR3 amino acid sequence at least 70%, at least 75%, at least 80%, at least 85%, at least 90%, or at least 95% identical to SEQ ID NO: 486. In a particular embodiment, the anti-GAL-3 antibody comprises a HCDR1 amino acid sequence of SEQ ID NO: 375, a HCDR2 amino acid sequence of SEQ ID NO: 395, a HCDR3 amino acid sequence of SEQ ID NO: 421, a LCDR1 amino acid sequence of SEQ ID NO: 445, a LCDR2 amino acid sequence of SEQ ID NO: 468, and a LCDR3 amino acid sequence of SEQ ID NO: 486.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 376, a HCDR2 amino acid sequence at least 70%, at least 75%, at least 80%, at least 85%, at least 90%, or at least 95% identical to SEQ ID NO: 396, and a HCDR3 amino acid sequence at least 70%, at least 75%, at least 80%, at least 85%, at least 90%, or at least 95% identical to SEQ ID NO: 422; and a light chain variable region comprising a LCDR1 amino acid sequence at least 70%, at least 75%, at least 80%, at least 85%, at least 90%, or at least 95% identical to SEQ ID NO: 446, a LCDR2 amino acid sequence at least 70%, at least 75%, at least 80%, at least 85%, at least 90%, or at least 95% identical to SEQ ID NO: 469, and a LCDR3 amino acid sequence at least 70%, at least 75%, at least 80%, at least 85%, at least 90%, or at least 95% identical to SEQ ID NO: 487. In a particular embodiment, the anti-GAL-3 antibody comprises a HCDR1 amino acid sequence of SEQ ID NO: 376, a HCDR2 amino acid sequence of SEQ ID NO: 396, a HCDR3 amino acid sequence of SEQ ID NO: 422, a LCDR1 amino acid sequence of SEQ ID NO: 446, a LCDR2 amino acid sequence of SEQ ID NO: 469, and a LCDR3 amino acid sequence of SEQ ID NO: 487.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 373, a HCDR2 amino acid sequence at least 70%, at least 75%, at least 80%, at least 85%, at least 90%, or at least 95% identical to SEQ ID NO: 393, and a HCDR3 amino acid sequence at least 70%, at least 75%, at least 80%, at least 85%, at least 90%, or at least 95% identical to SEQ ID NO: 419; and a light chain variable region comprising a LCDR1 amino acid sequence at least 70%, at least 75%, at least 80%, at least 85%, at least 90%, or at least 95% identical to SEQ ID NO: 447, a LCDR2 amino acid sequence at least 70%, at least 75%, at least 80%, at least 85%, at least 90%, or at least 95% identical to SEQ ID NO: 470, and a LCDR3 amino acid sequence at least 70%, at least 75%, at least 80%, at least 85%, at least 90%, or at least 95% identical to SEQ ID NO: 484. In a particular embodiment, the anti-GAL-3 antibody comprises a HCDR1 amino acid sequence of SEQ ID NO: 373, a HCDR2 amino acid sequence of SEQ ID NO: 393, a HCDR3 amino acid sequence of SEQ ID NO: 419, a LCDR1 amino acid sequence of SEQ ID NO: 447, a LCDR2 amino acid sequence of SEQ ID NO: 470, and a LCDR3 amino acid sequence of SEQ ID NO: 484.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 377, a HCDR2 amino acid sequence at least 70%, at least 75%, at least 80%, at least 85%, at least 90%, or at least 95% identical to SEQ ID NO: 397, and a HCDR3 amino acid sequence at least 70%, at least 75%, at least 80%, at least 85%, at least 90%, or at least 95% identical to SEQ ID NO: 423; and a light chain variable region comprising a LCDR1 amino acid sequence at least 70%, at least 75%, at least 80%, at least 85%, at least 90%, or at least 95% identical to SEQ ID NO: 448, a LCDR2 amino acid sequence at least 70%, at least 75%, at least 80%, at least 85%, at least 90%, or at least 95% identical to SEQ ID NO: 471, and a LCDR3 amino acid sequence at least 70%, at least 75%, at least 80%, at least 85%, at least 90%, or at least 95% identical to SEQ ID NO: 488. In a particular embodiment, the anti-amino acid sequence of SEQ ID NO: 448, a LCDR2 amino acid sequence of SEQ ID NO: 471, and a LCDR3 amino acid sequence of SEQ ID NO: 488.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 377, a HCDR2 amino acid sequence at least 70%, at least 75%, at least 80%, at least 85%, at least 90%, or at least 95% identical to SEQ ID NO: 397, and a HCDR3 amino acid sequence at least 70%, at least 75%, at least 80%, at least 85%, at least 90%, or at least 95% identical to SEQ ID NO: 423; and a light chain variable region comprising a LCDR1 amino acid sequence at least 70%, at least 75%, at least 80%, at least 85%, at least 90%, or at least 95% identical to SEQ ID NO: 449, a LCDR2 amino acid sequence at least 70%, at least 75%, at least 80%, at least 85%, at least 90%, or at least 95% identical to SEQ ID NO: 472, and a LCDR3 amino acid sequence at least 70%, at least 75%, at least 80%, at least 85%, at least 90%, or at least 95% identical to SEQ ID NO: 488. In a particular embodiment, the anti-GAL-3 antibody comprises a HCDR1 amino acid sequence of SEQ ID NO: 377, a HCDR2 amino acid sequence of SEQ ID NO: 397, a HCDR3 amino acid sequence of SEQ ID NO: 423, a LCDR1 amino acid sequence of SEQ ID NO: 449, a LCDR2 amino acid sequence of SEQ ID NO: 472, and a LCDR3 amino acid sequence of SEQ ID NO: 488.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 377, a HCDR2 amino acid sequence at least 70%, at least 75%, at least 80%, at least 85%, at least 90%, or at least 95% identical to SEQ ID NO: 397, and a HCDR3 amino acid sequence at least 70%, at least 75%, at least 80%, at least 85%, at least 90%, or at least 95% identical to SEQ ID NO: 423; and a light chain variable region comprising a LCDR1 amino acid sequence at least 70%, at least 75%, at least 80%, at least 85%, at least 90%, or at least 95% identical to SEQ ID NO: 450, a LCDR2 amino acid sequence at least 70%, at least 75%, at least 80%, at least 85%, at least 90%, or at least 95% identical to SEQ ID NO: 471, and a LCDR3 amino acid sequence at least 70%, at least 75%, at least 80%, at least 85%, at least 90%, or at least 95% identical to SEQ ID NO: 488. In a particular embodiment, the anti-amino acid sequence of SEQ ID NO: 450, a LCDR2 amino acid sequence of SEQ ID NO: 471, and a LCDR3 amino acid sequence of SEQ ID NO: 488.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 377, a HCDR2 amino acid sequence at least 70%, at least 75%, at least 80%, at least 85%, at least 90%, or at least 95% identical to SEQ ID NO: 398, and a HCDR3 amino acid sequence at least 70%, at least 75%, at least 80%, at least 85%, at least 90%, or at least 95% identical to SEQ ID NO: 423; and a light chain variable region comprising a LCDR1 amino acid sequence at least 70%, at least 75%, at least 80%, at least 85%, at least 90%, or at least 95% identical to SEQ ID NO: 448, a LCDR2 amino acid sequence at least 70%, at least 75%, at least 80%, at least 85%, at least 90%, or at least 95% identical to SEQ ID NO: 471, and a LCDR3 amino acid sequence at least 70%, at least 75%, at least 80%, at least 85%, at least 90%, or at least 95% identical to SEQ ID NO: 488. In a particular embodiment, the anti-GAL-3 antibody comprises a HCDR1 amino acid sequence of SEQ ID NO: 377, a HCDR2 amino acid sequence of SEQ ID NO: 398, a HCDR3 amino acid sequence of SEQ ID NO: 423, a LCDR1 amino acid sequence of SEQ ID NO: 448, a LCDR2 amino acid sequence of SEQ ID NO: 471, and a LCDR3 amino acid sequence of SEQ ID NO: 488.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 377, a HCDR2 amino acid sequence at least 70%, at least 75%, at least 80%, at least 85%, at least 90%, or at least 95% identical to SEQ ID NO: 397, and a HCDR3 amino acid sequence at least 70%, at least 75%, at least 80%, at least 85%, at least 90%, or at least 95% identical to SEQ ID NO: 424; and a light chain variable region comprising a LCDR1 amino acid sequence at least 70%, at least 75%, at least 80%, at least 85%, at least 90%, or at least 95% identical to SEQ ID NO: 448, a LCDR2 amino acid sequence at least 70%, at least 75%, at least 80%, at least 85%, at least 90%, or at least 95% identical to SEQ ID NO: 472, and a LCDR3 amino acid sequence at least 70%, at least 75%, at least 80%, at least 85%, at least 90%, or at least 95% identical to SEQ ID NO: 489. In a particular embodiment, the anti-acid sequence of SEQ ID NO: 397, a HCDR3 amino acid sequence of SEQ ID NO: 424, a LCDR1 amino acid sequence of SEQ ID NO: 448, a LCDR2 amino acid sequence of SEQ ID NO: 472, and a LCDR3 amino acid sequence of SEQ ID NO: 489.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 377, a HCDR2 amino acid sequence at least 70%, at least 75%, at least 80%, at least 85%, at least 90%, or at least 95% identical to SEQ ID NO: 397, and a HCDR3 amino acid sequence at least 70%, at least 75%, at least 80%, at least 85%, at least 90%, or at least 95% identical to SEQ ID NO: 423; and a light chain variable region comprising a LCDR1 amino acid sequence at least 70%, at least 75%, at least 80%, at least 85%, at least 90%, or at least 95% identical to SEQ ID NO: 448, a LCDR2 amino acid sequence at least 70%, at least 75%, at least 80%, at least 85%, at least 90%, or at least 95% identical to SEQ ID NO: 472, and a LCDR3 amino acid sequence at least 70%, at least 75%, at least 80%, at least 85%, at least 90%, or at least 95% identical to SEQ ID NO: 488. In a particular embodiment, the anti-GAL-3 antibody comprises a HCDR1 amino acid sequence of SEQ ID NO: 377, a HCDR2 amino acid sequence of SEQ ID NO: 397, a HCDR3 amino acid sequence of SEQ ID NO: 423, a LCDR1 amino acid sequence of SEQ ID NO: 448, a LCDR2 amino acid sequence of SEQ ID NO: 472, and a LCDR3 amino acid sequence of SEQ ID NO: 488.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 377, a HCDR2 amino acid sequence at least 70%, at least 75%, at least 80%, at least 85%, at least 90%, or at least 95% identical to SEQ ID NO: 397, and a HCDR3 amino acid sequence at least 70%, at least 75%, at least 80%, at least 85%, at least 90%, or at least 95% identical to SEQ ID NO: 423; and a light chain variable region comprising a LCDR1 amino acid sequence at least 70%, at least 75%, at least 80%, at least 85%, at least 90%, or at least 95% identical to SEQ ID NO: 448, a LCDR2 amino acid sequence at least 70%, at least 75%, at least 80%, at least 85%, at least 90%, or at least 95% identical to SEQ ID NO: 471, and a LCDR3 amino acid sequence at least 70%, at least 75%, at least 80%, at least 85%, at least 90%, or at least 95% identical to SEQ ID NO: 490. In a particular embodiment, the anti-acid sequence of SEQ ID NO: 397, a HCDR3 amino acid sequence of SEQ ID NO: 423, a LCDR1 amino acid sequence of SEQ ID NO: 448, a LCDR2 amino acid sequence of SEQ ID NO: 471, and a LCDR3 amino acid sequence of SEQ ID NO: 490.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 377, a HCDR2 amino acid sequence at least 70%, at least 75%, at least 80%, at least 85%, at least 90%, or at least 95% identical to SEQ ID NO: 398, and a HCDR3 amino acid sequence at least 70%, at least 75%, at least 80%, at least 85%, at least 90%, or at least 95% identical to SEQ ID NO: 423; and a light chain variable region comprising a LCDR1 amino acid sequence at least 70%, at least 75%, at least 80%, at least 85%, at least 90%, or at least 95% identical to SEQ ID NO: 448, a LCDR2 amino acid sequence at least 70%, at least 75%, at least 80%, at least 85%, at least 90%, or at least 95% identical to SEQ ID NO: 471, and a LCDR3 amino acid sequence at least 70%, at least 75%, at least 80%, at least 85%, at least 90%, or at least 95% identical to SEQ ID NO: 491. In a particular embodiment, the anti-GAL-3 antibody comprises a HCDR1 amino acid sequence of SEQ ID NO: 377, a HCDR2 amino acid sequence of SEQ ID NO: 398, a HCDR3 amino acid sequence of SEQ ID NO: 423, a LCDR1 amino acid sequence of SEQ ID NO: 448, a LCDR2 amino acid sequence of SEQ ID NO: 471, and a LCDR3 amino acid sequence of SEQ ID NO: 491.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 377, a HCDR2 amino acid sequence at least 70%, at least 75%, at least 80%, at least 85%, at least 90%, or at least 95% identical to SEQ ID NO: 399, and a HCDR3 amino acid sequence at least 70%, at least 75%, at least 80%, at least 85%, at least 90%, or at least 95% identical to SEQ ID NO: 424; and a light chain variable region comprising a LCDR1 amino acid sequence at least 70%, at least 75%, at least 80%, at least 85%, at least 90%, or at least 95% identical to SEQ ID NO: 448, a LCDR2 amino acid sequence at least 70%, at least 75%, at least 80%, at least 85%, at least 90%, or at least 95% identical to SEQ ID NO: 472, and a LCDR3 amino acid sequence at least 70%, at least 75%, at least 80%, at least 85%, at least 90%, or at least 95% identical to SEQ ID NO: 489. In a particular embodiment, the anti-acid sequence of SEQ ID NO: 399, a HCDR3 amino acid sequence of SEQ ID NO: 424, a LCDR1 amino acid sequence of SEQ ID NO: 448, a LCDR2 amino acid sequence of SEQ ID NO: 472, and a LCDR3 amino acid sequence of SEQ ID NO: 489.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 377, a HCDR2 amino acid sequence at least 70%, at least 75%, at least 80%, at least 85%, at least 90%, or at least 95% identical to SEQ ID NO: 397, and a HCDR3 amino acid sequence at least 70%, at least 75%, at least 80%, at least 85%, at least 90%, or at least 95% identical to SEQ ID NO: 424; and a light chain variable region comprising a LCDR1 amino acid sequence at least 70%, at least 75%, at least 80%, at least 85%, at least 90%, or at least 95% identical to SEQ ID NO: 448, a LCDR2 amino acid sequence at least 70%, at least 75%, at least 80%, at least 85%, at least 90%, or at least 95% identical to SEQ ID NO: 471, and a LCDR3 amino acid sequence at least 70%, at least 75%, at least 80%, at least 85%, at least 90%, or at least 95% identical to SEQ ID NO: 488. In a particular embodiment, the anti-GAL-3 antibody comprises a HCDR1 amino acid sequence of SEQ ID NO: 377, a HCDR2 amino acid sequence of SEQ ID NO: 397, a HCDR3 amino acid sequence of SEQ ID NO: 424, a LCDR1 amino acid sequence of SEQ ID NO: 448, a LCDR2 amino acid sequence of SEQ ID NO: 471, and a LCDR3 amino acid sequence of SEQ ID NO: 488.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 378, a HCDR2 amino acid sequence at least 70%, at least 75%, at least 80%, at least 85%, at least 90%, or at least 95% identical to SEQ ID NO: 400, and a HCDR3 amino acid sequence at least 70%, at least 75%, at least 80%, at least 85%, at least 90%, or at least 95% identical to SEQ ID NO: 425; and a light chain variable region comprising a LCDR1 amino acid sequence at least 70%, at least 75%, at least 80%, at least 85%, at least 90%, or at least 95% identical to SEQ ID NO: 451, a LCDR2 amino acid sequence at least 70%, at least 75%, at least 80%, at least 85%, at least 90%, or at least 95% identical to SEQ ID NO: 473, and a LCDR3 amino acid sequence at least 70%, at least 75%, at least 80%, at least 85%, at least 90%, or at least 95% identical to SEQ ID NO: 492. In a particular embodiment, the anti-acid sequence of SEQ ID NO: 400, a HCDR3 amino acid sequence of SEQ ID NO: 425, a LCDR1 amino acid sequence of SEQ ID NO: 451, a LCDR2 amino acid sequence of SEQ ID NO: 473, and a LCDR3 amino acid sequence of SEQ ID NO: 492.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 378, a HCDR2 amino acid sequence at least 70%, at least 75%, at least 80%, at least 85%, at least 90%, or at least 95% identical to SEQ ID NO: 401, and a HCDR3 amino acid sequence at least 70%, at least 75%, at least 80%, at least 85%, at least 90%, or at least 95% identical to SEQ ID NO: 425; and a light chain variable region comprising a LCDR1 amino acid sequence at least 70%, at least 75%, at least 80%, at least 85%, at least 90%, or at least 95% identical to SEQ ID NO: 452, a LCDR2 amino acid sequence at least 70%, at least 75%, at least 80%, at least 85%, at least 90%, or at least 95% identical to SEQ ID NO: 473, and a LCDR3 amino acid sequence at least 70%, at least 75%, at least 80%, at least 85%, at least 90%, or at least 95% identical to SEQ ID NO: 493. In a particular embodiment, the anti-GAL-3 antibody comprises a HCDR1 amino acid sequence of SEQ ID NO: 378, a HCDR2 amino acid sequence of SEQ ID NO: 401, a HCDR3 amino acid sequence of SEQ ID NO: 425, a LCDR1 amino acid sequence of SEQ ID NO: 452, a LCDR2 amino acid sequence of SEQ ID NO: 473, and a LCDR3 amino acid sequence of SEQ ID NO: 493.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 378, a HCDR2 amino acid sequence at least 70%, at least 75%, at least 80%, at least 85%, at least 90%, or at least 95% identical to SEQ ID NO: 402, and a HCDR3 amino acid sequence at least 70%, at least 75%, at least 80%, at least 85%, at least 90%, or at least 95% identical to SEQ ID NO: 425; and a light chain variable region comprising a LCDR1 amino acid sequence at least 70%, at least 75%, at least 80%, at least 85%, at least 90%, or at least 95% identical to SEQ ID NO: 452, a LCDR2 amino acid sequence at least 70%, at least 75%, at least 80%, at least 85%, at least 90%, or at least 95% identical to SEQ ID NO: 473, and a LCDR3 amino acid sequence at least 70%, at least 75%, at least 80%, at least 85%, at least 90%, or at least 95% identical to SEQ ID NO: 493. In a particular embodiment, the anti-acid sequence of SEQ ID NO: 402, a HCDR3 amino acid sequence of SEQ ID NO: 425, a LCDR1 amino acid sequence of SEQ ID NO: 452, a LCDR2 amino acid sequence of SEQ ID NO: 473, and a LCDR3 amino acid sequence of SEQ ID NO: 493.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 379, a HCDR2 amino acid sequence at least 70%, at least 75%, at least 80%, at least 85%, at least 90%, or at least 95% identical to SEQ ID NO: 403, and a HCDR3 amino acid sequence at least 70%, at least 75%, at least 80%, at least 85%, at least 90%, or at least 95% identical to SEQ ID NO: 426; and a light chain variable region comprising a LCDR1 amino acid sequence at least 70%, at least 75%, at least 80%, at least 85%, at least 90%, or at least 95% identical to SEQ ID NO: 453, a LCDR2 amino acid sequence at least 70%, at least 75%, at least 80%, at least 85%, at least 90%, or at least 95% identical to SEQ ID NO: 470, and a LCDR3 amino acid sequence at least 70%, at least 75%, at least 80%, at least 85%, at least 90%, or at least 95% identical to SEQ ID NO: 494. In a particular embodiment, the anti-GAL-3 antibody comprises a HCDR1 amino acid sequence of SEQ ID NO: 379, a HCDR2 amino acid sequence of SEQ ID NO: 403, a HCDR3 amino acid sequence of SEQ ID NO: 426, a LCDR1 amino acid sequence of SEQ ID NO: 453, a LCDR2 amino acid sequence of SEQ ID NO: 470, and a LCDR3 amino acid sequence of SEQ ID NO: 494.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 380, a HCDR2 amino acid sequence at least 70%, at least 75%, at least 80%, at least 85%, at least 90%, or at least 95% identical to SEQ ID NO: 403, and a HCDR3 amino acid sequence at least 70%, at least 75%, at least 80%, at least 85%, at least 90%, or at least 95% identical to SEQ ID NO: 426; and a light chain variable region comprising a LCDR1 amino acid sequence at least 70%, at least 75%, at least 80%, at least 85%, at least 90%, or at least 95% identical to SEQ ID NO: 453, a LCDR2 amino acid sequence at least 70%, at least 75%, at least 80%, at least 85%, at least 90%, or at least 95% identical to SEQ ID NO: 470, and a LCDR3 amino acid sequence at least 70%, at least 75%, at least 80%, at least 85%, GAL-3 antibody comprises a HCDR1 amino acid sequence of SEQ ID NO: 380, a HCDR2 amino acid sequence of SEQ ID NO: 403, a HCDR3 amino acid sequence of SEQ ID NO: 426, a LCDR1 and a LCDR3 amino acid sequence of SEQ ID NO: 494.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 380, a HCDR2 amino acid sequence at least 70%, at least 75%, at least 80%, at least 85%, at least 90%, or at least 95% identical to SEQ ID NO: 403, and a HCDR3 amino acid sequence at least 70%, at least 75%, at least 80%, at least 85%, at least 90%, or at least 95% identical to SEQ ID NO: 426; and a light chain variable region comprising a LCDR1 amino acid sequence at least 70%, at least 75%, at least 80%, at least 85%, at least 90%, or at least 95% identical to SEQ ID NO: 453, a LCDR2 amino acid sequence at least 70%, at least 75%, at least 80%, at least 85%, at least 90%, or at least 95% identical to SEQ ID NO: 474, and a LCDR3 amino acid sequence at least 70%, at least 75%, at least 80%, at least 85%, at least 90%, or at least 95% identical to SEQ ID NO: 494. In a particular embodiment, the anti-GAL-3 antibody comprises a HCDR1 amino acid sequence of SEQ ID NO: 380, a HCDR2 amino acid sequence of SEQ ID NO: 403, a HCDR3 amino acid sequence of SEQ ID NO: 426, a LCDR1 amino acid sequence of SEQ ID NO: 453, a LCDR2 amino acid sequence of SEQ ID NO: 474, and a LCDR3 amino acid sequence of SEQ ID NO: 494.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 381, a HCDR2 amino acid sequence at least 70%, at least 75%, at least 80%, at least 85%, at least 90%, or at least 95% identical to SEQ ID NO: 404, and a HCDR3 amino acid sequence at least 70%, at least 75%, at least 80%, at least 85%, at least 90%, or at least 95% identical to SEQ ID NO: 426; and a light chain variable region comprising a LCDR1 amino acid sequence at least 70%, at least 75%, at least 80%, at least 85%, at least 90%, or at least 95% identical to SEQ ID NO: 453, a LCDR2 amino acid sequence at least 70%, at least 75%, at least 80%, at least 85%, at least 90%, or at least 95% identical to SEQ ID NO: 470, and a LCDR3 amino acid sequence at least 70%, at least 75%, at least 80%, at least 85%, acid sequence of SEQ ID NO: 404, a HCDR3 amino acid sequence of SEQ ID NO: 426, a LCDR1 and a LCDR3 amino acid sequence of SEQ ID NO: 494.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 381, a HCDR2 amino acid sequence at least 70%, at least 75%, at least 80%, at least 85%, at least 90%, or at least 95% identical to SEQ ID NO: 404, and a HCDR3 amino acid sequence at least 70%, at least 75%, at least 80%, at least 85%, at least 90%, or at least 95% identical to SEQ ID NO: 426; and a light chain variable region comprising a LCDR1 amino acid sequence at least 70%, at least 75%, at least 80%, at least 85%, at least 90%, or at least 95% identical to SEQ ID NO: 453, a LCDR2 amino acid sequence at least 70%, at least 75%, at least 80%, at least 85%, at least 90%, or at least 95% identical to SEQ ID NO: 470, and a LCDR3 amino acid sequence at least 70%, at least 75%, at least 80%, at least 85%, at least 90%, or at least 95% identical to SEQ ID NO: 495. In a particular embodiment, the anti-GAL-3 antibody comprises a HCDR1 amino acid sequence of SEQ ID NO: 381, a HCDR2 amino acid sequence of SEQ ID NO: 404, a HCDR3 amino acid sequence of SEQ ID NO: 426, a LCDR1 amino acid sequence of SEQ ID NO: 453, a LCDR2 amino acid sequence of SEQ ID NO: 470, and a LCDR3 amino acid sequence of SEQ ID NO: 495.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 381, a HCDR2 amino acid sequence at least 70%, at least 75%, at least 80%, at least 85%, at least 90%, or at least 95% identical to SEQ ID NO: 405, and a HCDR3 amino acid sequence at least 70%, at least 75%, at least 80%, at least 85%, at least 90%, or at least 95% identical to SEQ ID NO: 426; and a light chain variable region comprising a LCDR1 amino acid sequence at least 70%, at least 75%, at least 80%, at least 85%, at least 90%, or at least 95% identical to SEQ ID NO: 453, a LCDR2 amino acid sequence at least 70%, at least 75%, at least 80%, at least 85%, at least 90%, or at least 95% identical to SEQ ID NO: 470, and a LCDR3 amino acid sequence at least 70%, at least 75%, at least 80%, at least 85%, acid sequence of SEQ ID NO: 405, a HCDR3 amino acid sequence of SEQ ID NO: 426, a LCDR1 and a LCDR3 amino acid sequence of SEQ ID NO: 494.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 378, a HCDR2 amino acid sequence at least 70%, at least 75%, at least 80%, at least 85%, at least 90%, or at least 95% identical to SEQ ID NO: 406, and a HCDR3 amino acid sequence at least 70%, at least 75%, at least 80%, at least 85%, at least 90%, or at least 95% identical to SEQ ID NO: 427; and a light chain variable region comprising a LCDR1 amino acid sequence at least 70%, at least 75%, at least 80%, at least 85%, at least 90%, or at least 95% identical to SEQ ID NO: 452, a LCDR2 amino acid sequence at least 70%, at least 75%, at least 80%, at least 85%, at least 90%, or at least 95% identical to SEQ ID NO: 470, and a LCDR3 amino acid sequence at least 70%, at least 75%, at least 80%, at least 85%, at least 90%, or at least 95% identical to SEQ ID NO: 496. In a particular embodiment, the anti-GAL-3 antibody comprises a HCDR1 amino acid sequence of SEQ ID NO: 378, a HCDR2 amino acid sequence of SEQ ID NO: 406, a HCDR3 amino acid sequence of SEQ ID NO: 427, a LCDR1 amino acid sequence of SEQ ID NO: 452, a LCDR2 amino acid sequence of SEQ ID NO: 470, and a LCDR3 amino acid sequence of SEQ ID NO: 496.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 378, a HCDR2 amino acid sequence at least 70%, at least 75%, at least 80%, at least 85%, at least 90%, or at least 95% identical to SEQ ID NO: 407, and a HCDR3 amino acid sequence at least 70%, at least 75%, at least 80%, at least 85%, at least 90%, or at least 95% identical to SEQ ID NO: 425; and a light chain variable region comprising a LCDR1 amino acid sequence at least 70%, at least 75%, at least 80%, at least 85%, at least 90%, or at least 95% identical to SEQ ID NO: 452, a LCDR2 amino acid sequence at least 70%, at least 75%, at least 80%, at least 85%, at least 90%, or at least 95% identical to SEQ ID NO: 475, and a LCDR3 amino acid sequence at least 70%, at least 75%, at least 80%, at least 85%, at least 90%, or at least 95% identical to SEQ ID NO: 496. In a particular embodiment, the anti-acid sequence of SEQ ID NO: 407, a HCDR3 amino acid sequence of SEQ ID NO: 425, a LCDR1 amino acid sequence of SEQ ID NO: 452, a LCDR2 amino acid sequence of SEQ ID NO: 475, and a LCDR3 amino acid sequence of SEQ ID NO: 496.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 382, a HCDR2 amino acid sequence at least 70%, at least 75%, at least 80%, at least 85%, at least 90%, or at least 95% identical to SEQ ID NO: 408, and a HCDR3 amino acid sequence at least 70%, at least 75%, at least 80%, at least 85%, at least 90%, or at least 95% identical to SEQ ID NO: 428; and a light chain variable region comprising a LCDR1 amino acid sequence at least 70%, at least 75%, at least 80%, at least 85%, at least 90%, or at least 95% identical to SEQ ID NO: 454, a LCDR2 amino acid sequence at least 70%, at least 75%, at least 80%, at least 85%, at least 90%, or at least 95% identical to SEQ ID NO: 471, and a LCDR3 amino acid sequence at least 70%, at least 75%, at least 80%, at least 85%, at least 90%, or at least 95% identical to SEQ ID NO: 497. In a particular embodiment, the anti-GAL-3 antibody comprises a HCDR1 amino acid sequence of SEQ ID NO: 382, a HCDR2 amino acid sequence of SEQ ID NO: 408, a HCDR3 amino acid sequence of SEQ ID NO: 428, a LCDR1 amino acid sequence of SEQ ID NO: 454, a LCDR2 amino acid sequence of SEQ ID NO: 471, and a LCDR3 amino acid sequence of SEQ ID NO: 497.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 382, a HCDR2 amino acid sequence at least 70%, at least 75%, at least 80%, at least 85%, at least 90%, or at least 95% identical to SEQ ID NO: 408, and a HCDR3 amino acid sequence at least 70%, at least 75%, at least 80%, at least 85%, at least 90%, or at least 95% identical to SEQ ID NO: 429; and a light chain variable region comprising a LCDR1 amino acid sequence at least 70%, at least 75%, at least 80%, at least 85%, at least 90%, or at least 95% identical to SEQ ID NO: 455, a LCDR2 amino acid sequence at least 70%, at least 75%, at least 80%, at least 85%, at least 90%, or at least 95% identical to SEQ ID NO: 476, and a LCDR3 amino acid sequence at least 70%, at least 75%, at least 80%, at least 85%, at least 90%, or at least 95% identical to SEQ ID NO: 498. In a particular embodiment, the anti-acid sequence of SEQ ID NO: 408, a HCDR3 amino acid sequence of SEQ ID NO: 429, a LCDR1 amino acid sequence of SEQ ID NO: 455, a LCDR2 amino acid sequence of SEQ ID NO: 476, and a LCDR3 amino acid sequence of SEQ ID NO: 498.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 382, a HCDR2 amino acid sequence at least 70%, at least 75%, at least 80%, at least 85%, at least 90%, or at least 95% identical to SEQ ID NO: 408, and a HCDR3 amino acid sequence at least 70%, at least 75%, at least 80%, at least 85%, at least 90%, or at least 95% identical to SEQ ID NO: 429; and a light chain variable region comprising a LCDR1 amino acid sequence at least 70%, at least 75%, at least 80%, at least 85%, at least 90%, or at least 95% identical to SEQ ID NO: 454, a LCDR2 amino acid sequence at least 70%, at least 75%, at least 80%, at least 85%, at least 90%, or at least 95% identical to SEQ ID NO: 468, and a LCDR3 amino acid sequence at least 70%, at least 75%, at least 80%, at least 85%, at least 90%, or at least 95% identical to SEQ ID NO: 497. In a particular embodiment, the anti-GAL-3 antibody comprises a HCDR1 amino acid sequence of SEQ ID NO: 382, a HCDR2 amino acid sequence of SEQ ID NO: 408, a HCDR3 amino acid sequence of SEQ ID NO: 429, a LCDR1 amino acid sequence of SEQ ID NO: 454, a LCDR2 amino acid sequence of SEQ ID NO: 468, and a LCDR3 amino acid sequence of SEQ ID NO: 497.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 382, a HCDR2 amino acid sequence at least 70%, at least 75%, at least 80%, at least 85%, at least 90%, or at least 95% identical to SEQ ID NO: 408, and a HCDR3 amino acid sequence at least 70%, at least 75%, at least 80%, at least 85%, at least 90%, or at least 95% identical to SEQ ID NO: 430; and a light chain variable region comprising a LCDR1 amino acid sequence at least 70%, at least 75%, at least 80%, at least 85%, at least 90%, or at least 95% identical to SEQ ID NO: 454, a LCDR2 amino acid sequence at least 70%, at least 75%, at least 80%, at least 85%, at least 90%, or at least 95% identical to SEQ ID NO: 477, and a LCDR3 amino acid sequence at least 70%, at least 75%, at least 80%, at least 85%, at least 90%, or at least 95% identical to SEQ ID NO: 497. In a particular embodiment, the anti-acid sequence of SEQ ID NO: 408, a HCDR3 amino acid sequence of SEQ ID NO: 430, a LCDR1 amino acid sequence of SEQ ID NO: 454, a LCDR2 amino acid sequence of SEQ ID NO: 477, and a LCDR3 amino acid sequence of SEQ ID NO: 497.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 383, a HCDR2 amino acid sequence at least 70%, at least 75%, at least 80%, at least 85%, at least 90%, or at least 95% identical to SEQ ID NO: 409, and a HCDR3 amino acid sequence at least 70%, at least 75%, at least 80%, at least 85%, at least 90%, or at least 95% identical to SEQ ID NO: 431; and a light chain variable region comprising a LCDR1 amino acid sequence at least 70%, at least 75%, at least 80%, at least 85%, at least 90%, or at least 95% identical to SEQ ID NO: 456, a LCDR2 amino acid sequence at least 70%, at least 75%, at least 80%, at least 85%, at least 90%, or at least 95% identical to SEQ ID NO: 474, and a LCDR3 amino acid sequence at least 70%, at least 75%, at least 80%, at least 85%, at least 90%, or at least 95% identical to SEQ ID NO: 499. In a particular embodiment, the anti-GAL-3 antibody comprises a HCDR1 amino acid sequence of SEQ ID NO: 383, a HCDR2 amino acid sequence of SEQ ID NO: 409, a HCDR3 amino acid sequence of SEQ ID NO: 431, a LCDR1 amino acid sequence of SEQ ID NO: 456, a LCDR2 amino acid sequence of SEQ ID NO: 474, and a LCDR3 amino acid sequence of SEQ ID NO: 499.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 383, a HCDR2 amino acid sequence at least 70%, at least 75%, at least 80%, at least 85%, at least 90%, or at least 95% identical to SEQ ID NO: 409, and a HCDR3 amino acid sequence at least 70%, at least 75%, at least 80%, at least 85%, at least 90%, or at least 95% identical to SEQ ID NO: 431; and a light chain variable region comprising a LCDR1 amino acid sequence at least 70%, at least 75%, at least 80%, at least 85%, at least 90%, or at least 95% identical to SEQ ID NO: 456, a LCDR2 amino acid sequence at least 70%, at least 75%, at least 80%, at least 85%, at least 90%, or at least 95% identical to SEQ ID NO: 478, and a LCDR3 amino acid sequence at least 70%, at least 75%, at least 80%, at least 85%, at least 90%, or at least 95% identical to SEQ ID NO: 499. In a particular embodiment, the anti-GAL-3 antibody comprises a HCDR1 amino acid sequence of SEQ ID NO: 383, a HCDR2 amino acid sequence of SEQ ID NO: 409, a HCDR3 amino acid sequence of SEQ ID NO: 431, a LCDR1 amino acid sequence of SEQ ID NO: 456, a LCDR2 amino acid sequence of SEQ ID NO: 478, and a LCDR3 amino acid sequence of SEQ ID NO: 499.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 384, a HCDR2 amino acid sequence at least 70%, at least 75%, at least 80%, at least 85%, at least 90%, or at least 95% identical to SEQ ID NO: 410, and a HCDR3 amino acid sequence at least 70%, at least 75%, at least 80%, at least 85%, at least 90%, or at least 95% identical to SEQ ID NO: 431; and a light chain variable region comprising a LCDR1 amino acid sequence at least 70%, at least 75%, at least 80%, at least 85%, at least 90%, or at least 95% identical to SEQ ID NO: 457, a LCDR2 amino acid sequence at least 70%, at least 75%, at least 80%, at least 85%, at least 90%, or at least 95% identical to SEQ ID NO: 474, and a LCDR3 amino acid sequence at least 70%, at least 75%, at least 80%, at least 85%, at least 90%, or at least 95% identical to SEQ ID NO: 500. In a particular embodiment, the anti-GAL-3 antibody comprises a HCDR1 amino acid sequence of SEQ ID NO: 384, a HCDR2 amino acid sequence of SEQ ID NO: 410, a HCDR3 amino acid sequence of SEQ ID NO: 431, a LCDR1 amino acid sequence of SEQ ID NO: 457, a LCDR2 amino acid sequence of SEQ ID NO: 474, and a LCDR3 amino acid sequence of SEQ ID NO: 500.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 384, a HCDR2 amino acid sequence at least 70%, at least 75%, at least 80%, at least 85%, at least 90%, or at least 95% identical to SEQ ID NO: 410, and a HCDR3 amino acid sequence at least 70%, at least 75%, at least 80%, at least 85%, at least 90%, or at least 95% identical to SEQ ID NO: 432; and a light chain variable region comprising a LCDR1 amino acid sequence at least 70%, at least 75%, at least 80%, at least 85%, at least 90%, or at least 95% identical to SEQ ID NO: 458, a LCDR2 amino acid sequence at least 70%, at least 75%, at least 80%, at least 85%, at least 90%, or at least 95% identical to SEQ ID NO: 474, and a LCDR3 amino acid sequence at least 70%, at least 75%, at least 80%, at least 85%, at least 90%, or at least 95% identical to SEQ ID NO: 500. In a particular embodiment, the anti-GAL-3 antibody comprises a HCDR1 amino acid sequence of SEQ ID NO: 384, a HCDR2 amino acid sequence of SEQ ID NO: 410, a HCDR3 amino acid sequence of SEQ ID NO: 432, a LCDR1 amino acid sequence of SEQ ID NO: 458, a LCDR2 amino acid sequence of SEQ ID NO: 474, and a LCDR3 amino acid sequence of SEQ ID NO: 500.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 385, a HCDR2 amino acid sequence at least 70%, at least 75%, at least 80%, at least 85%, at least 90%, or at least 95% identical to SEQ ID NO: 411, and a HCDR3 amino acid sequence at least 70%, at least 75%, at least 80%, at least 85%, at least 90%, or at least 95% identical to SEQ ID NO: 433; and a light chain variable region comprising a LCDR1 amino acid sequence at least 70%, at least 75%, at least 80%, at least 85%, at least 90%, or at least 95% identical to SEQ ID NO: 459, a LCDR2 amino acid sequence at least 70%, at least 75%, at least 80%, at least 85%, at least 90%, or at least 95% identical to SEQ ID NO: 470, and a LCDR3 amino acid sequence at least 70%, at least 75%, at least 80%, at least 85%, at least 90%, or at least 95% identical to SEQ ID NO: 501. In a particular embodiment, the anti-GAL-3 antibody comprises a HCDR1 amino acid sequence of SEQ ID NO: 385, a HCDR2 amino acid sequence of SEQ ID NO: 411, a HCDR3 amino acid sequence of SEQ ID NO: 433, a LCDR1 amino acid sequence of SEQ ID NO: 459, a LCDR2 amino acid sequence of SEQ ID NO: 470, and a LCDR3 amino acid sequence of SEQ ID NO: 501.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 386, a HCDR2 amino acid sequence at least 70%, at least 75%, at least 80%, at least 85%, at least 90%, or at least 95% identical to SEQ ID NO: 412, and a HCDR3 amino acid sequence at least 70%, at least 75%, at least 80%, at least 85%, at least 90%, or at least 95% identical to SEQ ID NO: 434; and a light chain variable region comprising a LCDR1 amino acid sequence at least 70%, at least 75%, at least 80%, at least 85%, at least 90%, or at least 95% identical to SEQ ID NO: 460, a LCDR2 amino acid sequence at least 70%, at least 75%, at least 80%, at least 85%, at least 90%, or at least 95% identical to SEQ ID NO: 479, and a LCDR3 amino acid sequence at least 70%, at least 75%, at least 80%, at least 85%, at least 90%, or at least 95% identical to SEQ ID NO: 502. In a particular embodiment, the anti-GAL-3 antibody comprises a HCDR1 amino acid sequence of SEQ ID NO: 386, a HCDR2 amino acid sequence of SEQ ID NO: 412, a HCDR3 amino acid sequence of SEQ ID NO: 434, a LCDR1 amino acid sequence of SEQ ID NO: 460, a LCDR2 amino acid sequence of SEQ ID NO: 479, and a LCDR3 amino acid sequence of SEQ ID NO: 502.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 387, a HCDR2 amino acid sequence at least 70%, at least 75%, at least 80%, at least 85%, at least 90%, or at least 95% identical to SEQ ID NO: 413, and a HCDR3 amino acid sequence at least 70%, at least 75%, at least 80%, at least 85%, at least 90%, or at least 95% identical to SEQ ID NO: 435; and a light chain variable region comprising a LCDR1 amino acid sequence at least 70%, at least 75%, at least 80%, at least 85%, at least 90%, or at least 95% identical to SEQ ID NO: 461, a LCDR2 amino acid sequence at least 70%, at least 75%, at least 80%, at least 85%, at least 90%, or at least 95% identical to SEQ ID NO: 201, and a LCDR3 amino acid sequence at least 70%, at least 75%, at least 80%, at least 85%, at least 90%, or at least 95% identical to SEQ ID NO: 503. In a particular embodiment, the anti-GAL-3 antibody comprises a HCDR1 amino acid sequence of SEQ ID NO: 387, a HCDR2 amino acid sequence of SEQ ID NO: 413, a HCDR3 amino acid sequence of SEQ ID NO: 435, a LCDR1 amino acid sequence of SEQ ID NO: 461, a LCDR2 amino acid sequence of SEQ ID NO: 201, and a LCDR3 amino acid sequence of SEQ ID NO: 503.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 4, a HCDR2 amino acid sequence at least 70%, at least 75%, at least 80%, at least 85%, at least 90%, or at least 95% identical to SEQ ID NO: 414, and a HCDR3 amino acid sequence at least 70%, at least 75%, at least 80%, at least 85%, at least 90%, or at least 95% identical to SEQ ID NO: 436; and a light chain variable region comprising a LCDR1 amino acid sequence at least 70%, at least 75%, at least 80%, at least 85%, at least 90%, or at least 95% identical to SEQ ID NO: 462, a LCDR2 amino acid sequence at least 70%, at least 75%, at least 80%, at least 85%, at least 90%, or at least 95% identical to SEQ ID NO: 480, and a LCDR3 amino acid sequence at least 70%, at least 75%, at least 80%, at least 85%, at least 90%, or at least 95% identical to SEQ ID NO: 504. In a particular embodiment, the anti-acid sequence of SEQ ID NO: 414, a HCDR3 amino acid sequence of SEQ ID NO: 436, a LCDR1 amino acid sequence of SEQ ID NO: 462, a LCDR2 amino acid sequence of SEQ ID NO: 480, and a LCDR3 amino acid sequence of SEQ ID NO: 504.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 388, a HCDR2 amino acid sequence at least 70%, at least 75%, at least 80%, at least 85%, at least 90%, or at least 95% identical to SEQ ID NO: 415, and a HCDR3 amino acid sequence at least 70%, at least 75%, at least 80%, at least 85%, at least 90%, or at least 95% identical to SEQ ID NO: 437; and a light chain variable region comprising a LCDR1 amino acid sequence at least 70%, at least 75%, at least 80%, at least 85%, at least 90%, or at least 95% identical to SEQ ID NO: 461, a LCDR2 amino acid sequence at least 70%, at least 75%, at least 80%, at least 85%, at least 90%, or at least 95% identical to SEQ ID NO: 201, and a LCDR3 amino acid sequence at least 70%, at least 75%, at least 80%, at least 85%, at least 90%, or at least 95% identical to SEQ ID NO: 505. In a particular embodiment, the anti-GAL-3 antibody comprises a HCDR1 amino acid sequence of SEQ ID NO: 388, a HCDR2 amino acid sequence of SEQ ID NO: 415, a HCDR3 amino acid sequence of SEQ ID NO: 437, a LCDR1 amino acid sequence of SEQ ID NO: 461, a LCDR2 amino acid sequence of SEQ ID NO: 201, and a LCDR3 amino acid sequence of SEQ ID NO: 505.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 4, a HCDR2 amino acid sequence at least 70%, at least 75%, at least 80%, at least 85%, at least 90%, or at least 95% identical to SEQ ID NO: 416, and a HCDR3 amino acid sequence at least 70%, at least 75%, at least 80%, at least 85%, at least 90%, or at least 95% identical to SEQ ID NO: 438; and a light chain variable region comprising a LCDR1 amino acid sequence at least 70%, at least 75%, at least 80%, at least 85%, at least 90%, or at least 95% identical to SEQ ID NO: 462, a LCDR2 amino acid sequence at least 70%, at least 75%, at least 80%, at least 85%, at least 90%, or at least 95% identical to SEQ ID NO: 480, and a LCDR3 amino acid sequence at least 70%, at least 75%, at least 80%, at least 85%, at least 90%, or at least 95% identical to SEQ ID NO: 506. In a particular embodiment, the anti-acid sequence of SEQ ID NO: 416, a HCDR3 amino acid sequence of SEQ ID NO: 438, a LCDR1 amino acid sequence of SEQ ID NO: 462, a LCDR2 amino acid sequence of SEQ ID NO: 480, and a LCDR3 amino acid sequence of SEQ ID NO: 506.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 4, a HCDR2 amino acid sequence at least 70%, at least 75%, at least 80%, at least 85%, at least 90%, or at least 95% identical to SEQ ID NO: 414, and a HCDR3 amino acid sequence at least 70%, at least 75%, at least 80%, at least 85%, at least 90%, or at least 95% identical to SEQ ID NO: 436; and a light chain variable region comprising a LCDR1 amino acid sequence at least 70%, at least 75%, at least 80%, at least 85%, at least 90%, or at least 95% identical to SEQ ID NO: 462, a LCDR2 amino acid sequence at least 70%, at least 75%, at least 80%, at least 85%, at least 90%, or at least 95% identical to SEQ ID NO: 480, and a LCDR3 amino acid sequence at least 70%, at least 75%, at least 80%, at least 85%, at least 90%, or at least 95% identical to SEQ ID NO: 507. In a particular embodiment, the anti-GAL-3 antibody comprises a HCDR1 amino acid sequence of SEQ ID NO: 4, a HCDR2 amino acid sequence of SEQ ID NO: 414, a HCDR3 amino acid sequence of SEQ ID NO: 436, a LCDR1 amino acid sequence of SEQ ID NO: 462, a LCDR2 amino acid sequence of SEQ ID NO: 480, and a LCDR3 amino acid sequence of SEQ ID NO: 507.

In some embodiments, the anti-GAL-3 antibody comprises a heavy chain variable region comprising a HCDR1 amino acid sequence at least 70%, at least 75%, at least 80%, at least 85%, at least 90%, or at least 95% identical to SEQ ID NO: 389, a HCDR2 amino acid sequence at least 70%, at least 75%, at least 80%, at least 85%, at least 90%, or at least 95% identical to SEQ ID NO: 417, and a HCDR3 amino acid sequence at least 70%, at least 75%, at least 80%, at least 85%, at least 90%, or at least 95% identical to SEQ ID NO: 439. In a particular embodiment, the anti-GAL-3 antibody comprises a HCDR1 amino acid sequence of SEQ ID NO: 389, a HCDR2 amino acid sequence of SEQ ID NO: 417, a HCDR3 amino acid sequence of SEQ ID NO: 439.

In some embodiments, the anti-GAL-3 antibody comprises all six CDRs of an antibody selected from the group consisting of 1104SBC1068-033, 1104SBC1068-335, 1104SBC1068-365, 1104SBC1068-374, 1104SBC1068-378, 1220SBC1068-022, 1220SBC1068-035, 1220SBC1068-041, 1220SBC1068-056, 1220SBC1068-058, 1220SBC1068-063, 1220SBC1068-097, 1220SBC1068-098, 1220SBC1068-099, 1220SBC1068-129, 1220SBC1068-164, 1220SBC1068-186, 1220SBC1068-197, 1220SBC1068-210, 1220SBC1068-274, 1220SBC1068-281, 20240628SBC1080-002, 20240628SBC1080-003, 20240628SBC1080-004, 20240628SBC1080-005, 20240628SBC1080-006, 20240628SBC1080-007, 20240628SBC1080-008, 20240628SBC1080-009, 20240903SBC1093-015, 20240903SBC1093-020, 20240903SBC1093-021, 20240903SBC1093-025, 20240903SBC1093-030, 20240903SBC1093-182, 20240903SBC1093-183, 20240903SBC1093-184, 20240903SBC1093-185, 20240903SBC1093-230, 20240903SBC1093-033, 20240903SBC1093-034, 20240903SBC1093-035, 20240903SBC1093-044, 20240903SBC1093-054, 20240903SBC1093-055, 20240903SBC1093-056, 20240903SBC1093-057, 20240903SBC1093-059, 20240903SBC1093-219, 20240903SBC1093-228, 20240903SBC1093-023, 20240903SBC1093-036, 20240903SBC1093-037, 20240903SBC1093-220, 20240903SBC1093-074, 20240903SBC1093-075, 20240903SBC1093-193, 20240903SBC1093-194, 20240903SBC1093-099, 20240929SBC1205-022, 20241223SBC1205-061, 20241223SBC1205-063, 20241223SBC1205-119, 20241223SBC1205-130, 20241223SBC1205-131, and ANb1361-hGal-3-LP3R3 P2-69, as the H_{V} and H_{L} CDR amino acid sequences of the antibodies listed in Tables 3 and 4, respectively.

TABLE 3
Heavy chain CDR sequences

| Antibody ID | HCDR1 | HCDR2 | HCDR3 |
|---|---|---|---|
| 1104SBC1068-033 | SDYAWN (SEQ | YISYSGSTSYNPSLKS (SEQ | EGGRGGTEGYYFDY |
| | ID NO: 1) | ID NO: 22) | (SEQ ID NO: 43) |
| 1104SBC1068-335 | NYGMN (SEQ | WINTYSGEPTYVDDFKG | GTVVGFDY (SEQ ID |
| | ID NO: 2) | (SEQ ID NO: 23) | NO: 44) |
| 1104SBC1068-365 | NYGMN (SEQ | WINTNTGEPTYSEEFKG | SSYYAMDY (SEQ ID |
| | ID NO: 3) | (SEQ ID NO: 24) | NO: 45) |
| 1104SBC1068-374 | NYGMN (SEQ | WINTYTGEPTYADDFKG | YAMDY (SEQ ID NO: |
| | ID NO: 4) | (SEQ ID NO: 25) | 46) |
| SIF001 | NYGMN (SEQ | WINTYTGEPTYADDFKG | YAMDY (SEQ ID NO: |
| | ID NO: 4) | (SEQ ID NO: 25) | 46) |
| 1104SBC1068-378 | NYGMN (SEQ | WINTYTGEPTYADDFKG | WDDYDGFAY (SEQ ID |
| | ID NO: 5) | (SEQ ID NO: 26) | NO: 47) |
| 1220SBC1068-022 | DYGMA (SEQ | FISNLAYSVYYADTVTG | DRGLRESMDY (SEQ ID |
| | ID NO: 6) | (SEQ ID NO: 27) | NO: 48) |
| 1220SBC1068-035 | GYTMN (SEQ | LISPYSGGTNYNQRFKG | SGFITTGVPTNFDV |
| | ID NO: 7) | (SEQ ID NO: 28) | (SEQ ID NO: 49) |
| 1220SBC1068-041 | RFWMS (SEQ | EISPDSNTIDLTPSLKD | PYYGYY (SEQ ID NO: |
| | ID NO: 8) | (SEQ ID NO: 29) | 50) |
| SIF002 | RFWMS (SEQ | EISPDSNTIDLTPSLKD | PYYGYY (SEQ ID NO: |
| | ID NO: 8) | (SEQ ID NO: 29) | 50) |
| 1220SBC1068-056 | DYGMA (SEQ | FISNLAYSVYYADTVTG | DRGLRESMDY (SEQ ID |
| | ID NO: 9) | (SEQ ID NO: 30) | NO: 51) |
| 1220SBC1068-058 | DYGMA (SEQ | FISNLAYSVYYADTVTG | DRGLRETMDY (SEQ ID |
| | ID NO: 10) | (SEQ ID NO: 31) | NO: 52) |
| 1220SBC1068-063 | RFWMS (SEQ | EISPNSSTIHQTPSLKD (SEQ | PYYGYY (SEQ ID NO: |
| | ID NO: 11) | ID NO: 32) | 53) |
| 1220SBC1068-097 | RYWMS (SEQ | EINPDSSTIDLTPSLKD | PYHGYY (SEQ ID NO: |
| | ID NO: 12) | (SEQ ID NO: 33) | 54) |
| 1220SBC1068-098 | RYWMS (SEQ | EISPDSSTIDLTPSLKD (SEQ | PYHGYY (SEQ ID NO: |
| | ID NO: 13) | ID NO: 34) | 55) |
| 1220SBC1068-099 | RYWMT (SEQ | EINPHSSTITYTPSLKD | PYYGYY (SEQ ID NO: |
| | ID NO: 14) | (SEQ ID NO: 35) | 56) |
| 1220SBC1068-129 | RFWMS (SEQ | EINPDSSTITYTPSLKD | PYYGYY (SEQ ID NO: |
| | ID NO: 15) | (SEQ ID NO: 36) | 57) |
| 1220SBC1068-164 | RYWMS (SEQ | EISPDSSTINYTASLKD | PYYGYY (SEQ ID NO: |
| | ID NO: 16) | (SEQ ID NO: 37) | 58) |
| 1220SBC1068-186 | GYTMN (SEQ | LISPYSGGTNYNQKFKG | SGFITTGVATNFDV |
| | ID NO: 17) | (SEQ ID NO: 38) | (SEQ ID NO: 59) |
| 1220SBC1068-197 | RFWMS (SEQ | EISPNSSTIHQTPSLKD (SEQ | PYYGYY (SEQ ID NO: |
| | ID NO: 18) | ID NO: 39) | 60) |
| 1220SBC1068-210 | RYWMS (SEQ | EINPDSSTIDLTPSLKD | PYYGYY (SEQ ID NO: |
| | ID NO: 19) | (SEQ ID NO: 40) | 61) |
| 1220SBC1068-274 | RYWMS (SEQ | EISPDSNTIDLTPSLKD | PYYGYY (SEQ ID NO: |
| | ID NO: 20) | (SEQ ID NO: 41) | 62) |
| 1220SBC1068-281 | DFGMA (SEQ | FISNLAYNIYYSDTVTG | DRGLRETMDY (SEQ ID |
| | ID NO: 21) | (SEQ ID NO: 42) | NO: 63) |
| 20240628SBC1080- | NHHIN (SEQ ID | IISASGSAYYANWAKG | ESWMDL(SEQ ID |
| 002 | NO: 370) | (SEQ ID NO: 390) | NO: 418) |
| 20240628SBC1080- | NHHIN (SEQ ID | IISNGGSAYYANWAKG | ESWMDL(SEQ ID |
| 003 | NO: 371) | (SEQ ID NO: 391) | NO: 418) |
| 20240628SBC1080- | NNAMT(SEQ | YIWRGGETYYASWAKG | DNL |
| 004 | ID NO: 372 | (SEQ ID NO: 392) | |
| 20240628SBC1080- | RYGVS(SEQ ID | YIYIGDGSTYYASWVNG | SYLSSGWGVDL(SEQ |
| 005 | NO: 373) | (SEQ ID NO: 393) | ID NO: 419) |
| 20240628SBC1080- | YYAMG(SEQ | IIDTNNVTYYASWAKG | GGLDL(SEQ ID NO: 420) |
| 006 | ID NO: 374) | (SEQ ID NO: 394) | |
| 20240628SBC1080- | HYAMG(SEQ | YITFGGIAYYANWAKG | YADFAYNL(SEQ ID |
| 007 | ID NO: 375) | (SEQ ID NO: 395) | NO: 421) |
| 20240628SBC1080- | NNHMG(SEQ | FINGNGVGSAFYANWAKG | GPGYGCFDL(SEQ ID |
| 008 | ID NO: 376) | (SEQ ID NO: 396) | NO: 422) |
| 20240628SBC1080- | RYGVS(SEQ ID | YIYIGDGSTYYASWVNG | SYLSSGWGVDL(SEQ |
| 009 | NO: 373) | (SEQ ID NO: 393) | ID NO: 419) |
| 20240903SBC1093- | SYWMI(SEQ ID | GISTIDNTYYASWAKG | NFDT(SEQ ID NO: 423) |
| 015 | NO: 377) | (SEQ ID NO: 397) | |
| 20240903SBC1093- | SYWMI(SEQ ID | GISTIDNTYYASWAKG | NFDT(SEQ ID NO: 423) |
| 020 | NO: 377) | (SEQ ID NO: 397) | |
| 20240903SBC1093- | SYWMI(SEQ ID | GISTIDNTYYASWAKG | NFDT(SEQ ID NO: 423) |
| 021 | NO: 377) | (SEQ ID NO: 397) | |
| 20240903SBC1093- | SYWMI(SEQ ID | GISTIDNTYYATWAKG | NFDT(SEQ ID NO: 423) |
| 025 | NO: 377) | (SEQ ID NO: 398) | |
| 20240903SBC1093- | SYWMI(SEQ ID | GISTIDNTYYASWAKG | NFDI(SEQ ID NO: 424) |
| 030 | NO: 377) | (SEQ ID NO: 397) | |
| 20240903SBC1093- | SYWMI(SEQ ID | GISTIDNTYYASWAKG | NFDT(SEQ ID NO: 423) |
| 182 | NO: 377) | (SEQ ID NO: 397) | |
| 20240903SBC1093- | SYWMI(SEQ ID | GISTIDNTYYASWAKG | NEDT(SEQ ID NO: 423) |
| 183 | NO: 377) | (SEQ ID NO: 397) | |
| 20240903SBC1093- | SYWMI(SEQ ID | GISTIDNTYYATWAKG | NFDT(SEQ ID NO: 423) |
| 184 | NO: 377) | (SEQ ID NO: 398) | |
| 20240903SBC1093- | SYWMI(SEQ ID | GISTIDNTYYSTWAKG | NFDI(SEQ ID NO: 424) |
| 185 | NO: 377) | (SEQ ID NO: 399) | |
| 20240903SBC1093- | SYWMI(SEQ ID | GISTIDNTYYASWAKG | NFDI(SEQ ID NO: 424) |
| 230 | NO: 377) | (SEQ ID NO: 397) | |
| 20240903SBC1093- | DYAVF(SEQ ID | FISSIGSTYYASWAKG | WNLDI(SEQ ID NO: 425) |
| 033 | NO: 378) | (SEQ ID NO: 400) | |
| 20240903SBC1093- | DYAVF(SEQ ID | FISSVGTTFYASWAKG | WNLDI(SEQ ID NO: 425) |
| 034 | NO: 378) | (SEQ ID NO: 401) | |
| 20240903SBC1093- | DYAVF(SEQ ID | FISSVGTTFYATWAKG | WNLDI(SEQ ID NO: 425) |
| 035 | NO: 378) | (SEQ ID NO: 402) | |
| 20240903SBC1093- | KYAVN(SEQ | FIDAVGDTYYARWAKG | WNTDI(SEQ ID NO: 426) |
| 044 | ID NO: 379) | (SEQ ID NO: 403) | |
| 20240903SBC1093- | NYAVN(SEQ | FIDAVGDTYYARWAKG | WNTDI(SEQ ID NO: 426) |
| 054 | ID NO: 380) | (SEQ ID NO: 403) | |
| 20240903SBC1093- | NYAVN(SEQ | FIDAVGDTYYARWAKG | WNTDI(SEQ ID NO: 426) |
| 055 | ID NO: 380) | (SEQ ID NO: 403) | |
| 20240903SBC1093- | NYAVY(SEQ | FIDAVGNTYYARWAKG | WNTDI(SEQ ID NO: 426) |
| 056 | ID NO: 381) | (SEQ ID NO: 404) | |
| 20240903SBC1093- | NYAVY(SEQ | FIDAVGNTYYARWAKG | WNTDI(SEQ ID NO: 426) |
| 057 | ID NO: 381) | (SEQ ID NO: 404) | |
| 20240903SBC1093- | NYAVY(SEQ | FIDA VGSTYYARWAKG | WNTDI(SEQ ID NO: 426) |
| 059 | ID NO: 381) | (SEQ ID NO: 405) | |
| 20240903SBC1093- | DYAVF(SEQ ID | FISSVGTKFYASWAKG | WNMDI(SEQ ID NO: 427) |
| 219 | NO: 378) | (SEQ ID NO: 406) | |
| 20240903SBC1093- | DYAVF(SEQ ID | FISSVGTTYYAPWAKG | WNLDI(SEQ ID NO: 425) |
| 228 | NO: 378) | (SEQ ID NO: 407) | |
| 20240903SBC1093- | DYNFQ(SEQ ID | TIGIGTDTYYATWAIG(SEQ | DLYGGDSV(SEQ ID |
| 023 | NO: 382) | ID NO: 408) | NO: 428) |
| 20240903SBC1093- | DYNFQ(SEQ ID | TIGIGTDTYYATWAIG SEQ | DLYGGDSI(SEQ ID |
| 036 | NO: 382) | ID NO: 408) | NO: 429) |
| 20240903SBC1093- | DYNFQ(SEQ ID | TIGIGTDTYYATWAIG SEQ | DLYGGDSI(SEQ ID |
| 037 | NO: 382) | ID NO: 408) | NO: 429) |
| 20240903SBC1093- | DYNFQ(SEQ ID | TIGIGTDTYYATWAIG SEQ | DLYGGDNI(SEQ ID |
| 220 | NO: 382) | ID NO: 408) | NO: 430) |
| 20240903SBC1093- | NYYMN(SEQ | FINTGGSAYYASWAKG | FAVVAGLNI(SEQ ID |
| 074 | ID NO: 383) | (SEQ ID NO: 409) | NO: 431) |
| 20240903SBC1093- | NYYMN(SEQ | FINTGGSAYYASWAKG | FAVVAGLNI(SEQ ID |
| 075 | ID NO: 383) | (SEQ ID NO: 409) | NO: 431) |
| 20240903SBC1093- | SYYMN(SEQ | FIDTGGSAYFASWAKG | FAVVAGLNI(SEQ ID |
| 193 | ID NO: 384) | (SEQ ID NO: 410) | NO: 431) |
| 20240903SBC1093- | SYYMN(SEQ | FIDTGGSAYFASWAK | FAVVGGLNI(SEQ ID |
| 194 | ID NO: 384) | (SEQ ID NO: 410) | NO: 432) |
| 20240903SBC1093- | RYYMS(SEQ ID | SITTGGKGYYASWAKG | WDYLTDFDI(SEQ ID |
| 099 | NO: 385) | (SEQ ID NO: 411) | NO: 433) |
| 20240929SBC1205- | RYVMH(SEQ | YINPYNHDTKYNEKFKG | LDGYYAMDY(SEQ ID |
| 022 | ID NO: 386) | (SEQ ID NO: 412) | NO: 434) |
| 20241223SBC1205- | DYIML(SEQ ID | NINPYYGSTSYNLKFKG | DGNEKYGRFAY(SEQ |
| 061 | NO: 387) | (SEQ ID NO: 413) | ID NO: 435) |
| 20241223SBC1205- | NYGMN(SEQ | WINTYTGEPTYVDDFKG | YGDYYGNSYRGFDY |
| 063 | ID NO: 4) | (SEQ ID NO: 414) | (SEQ ID NO: 436) |
| 20241223SBC1205- | GYFMN(SEQ | RINHYNGDTFYNQKFKG | AEIHYDYDDAMDY |
| 119 | ID NO: 388) | (SEQ ID NO: 415) | (SEQ ID NO: 437) |
| 20241223SBC1205- | NYGMN(SEQ | WINTYTGEPTYSDDFKG | YGDYYGSSYRGFDY |
| 130 | ID NO: 4) | (SEQ ID NO: 416) | (SEQ ID NO: 438) |
| 20241223SBC1205- | NYGMN(SEQ | WINTYTGEPTYVDDFKG | YGDYYGNSYRGFDY |
| 131 | ID NO: 4) | (SEQ ID NO: 414) | (SEQ ID NO: 436) |
| ANb1361-hGal-3- | HYSIG(SEQ ID | CISTSDGSTYYADSVKG | GKGSRCHGDLDA(SEQ |
| LP3R3 P2-69 | NO: 389) | (SEQ ID NO: 417) | ID NO: 439) |

TABLE 4
Light chain CDR sequences

| Antibody ID | LCDR1 | LCDR2 | LCDR3 |
|---|---|---|---|
| 1104SBC1068- | RASESVDSYGNSFMH | LASNLES (SEQ ID NO: | QQNNEDPLT (SEQ ID |
| 033 | (SEQ ID NO: 169) | 190) | NO: 211) |
| 1104SBC1068- | KSSQSLLDSDGKTYLN | LVSKLDS (SEQ ID NO: | WQGTHFPQT (SEQ ID |
| 335 | (SEQ ID NO: 170) | 191) | NO: 212) |
| 1104SBC1068- | KSSQSLLDSDGKTYLN | LVSKLDS (SEQ ID NO: | WQATHFPQT (SEQ ID |
| 365 | (SEQ ID NO: 171) | 192) | NO: 213) |
| 1104SBC1068- | RSSTGAVTTSNYAN | GTSNRAP (SEQ ID | ALWYSTHYV (SEQ ID |
| 374 | (SEQ ID NO: 172) | NO: 193) | NO: 214) |
| SIF001 | RSSTGAVTTSNYAN | GTSNRAP (SEQ ID | ALWYSTHYV (SEQ ID |
| | (SEQ ID NO: 172) | NO: 193) | NO: 214) |
| 1104SBC1068- | RASQSISNNLH (SEQ ID | YASQSIS (SEQ ID NO: | QQSNSWPLT (SEQ ID |
| 378 | NO: 173) | 194) | NO: 215) |
| 1220SBC1068- | RSSKSLLHNNGNTYLC | RMSDLAS (SEQ ID | MQHLEYPYT (SEQ ID |
| 022 | (SEQ ID NO: 174) | NO: 195) | NO: 216) |
| 1220SBC1068- | KASQDVSTAVA (SEQ | SASYRYT (SEQ ID | QQHYTTPRT (SEQ ID |
| 035 | ID NO: 175) | NO: 196) | NO: 217) |
| 1220SBC1068- | RSSQSLFNSTNQKNYL | WASSRES (SEQ ID | QNDYTSPFT (SEQ ID |
| 041 | T (SEQ ID NO: 176) | NO: 197) | NO: 218) |
| SIF002 | RSSQSLFSSTNQKNYLT | WASSRES (SEQ ID | QNDYTSPFT (SEQ ID |
| | (SEQ ID NO: 369) | NO: 197) | NO: 218) |
| 1220SBC1068- | GSSKSLLHKNGNTYLC | RMSNLAS (SEQ ID | MQHLEYPYT (SEQ ID |
| 056 | (SEQ ID NO: 177) | NO: 198) | NO: 219) |
| 1220SBC1068- | RSSKSLLHNNGNTYLC | RMSNLAS (SEQ ID | MQHLEYPYT (SEQ ID |
| 058 | (SEQ ID NO: 178) | NO: 199) | NO: 220) |
| 1220SBC1068- | KSSQSLLSSINQKNYLT | WATSRES (SEQ ID | HNDYSYPFT (SEQ ID |
| 063 | (SEQ ID NO: 179) | NO: 200) | NO: 221) |
| 1220SBC1068- | RSSQSLLNSTNQKNYL | WASTRES (SEQ ID | QNDYTSPFT (SEQ ID |
| 097 | T (SEQ ID NO: 180) | NO: 201) | NO: 222) |
| 1220SBC1068- | RSSQSLLNSTNQKNYL | WASTKES (SEQ ID | QNDYTSPFT (SEQ ID |
| 098 | T (SEQ ID NO: 181) | NO: 202) | NO: 223) |
| 1220SBC1068- | KSSQSLLNSGNQKIYLT | WASIRES (SEQ ID NO: | QNDYSSPFT (SEQ ID |
| 099 | (SEQ ID NO: 182) | 203) | NO: 224) |
| 1220SBC1068- | KSSQSLLNSTNQKNYL | WASTRDS (SEQ ID | HNDYISPFT (SEQ ID |
| 129 | T (SEQ ID NO: 183) | NO: 204) | NO: 225) |
| 1220SBC1068- | KSSQSLFNSGNQKNYL | WAYTRES (SEQ ID | HNDYTSPFT (SEQ ID |
| 164 | T (SEQ ID NO: 184) | NO: 205) | NO: 226) |
| 1220SBC1068- | KASQDVSTAVA (SEQ | SASNRYT (SEQ ID | QQHYSTPRT (SEQ ID |
| 186 | ID NO: 185) | NO: 206) | NO: 227) |
| 1220SBC1068- | KSSQSLLNSINQKNYLT | WATFRES (SEQ ID | HNDYSYPFT (SEQ ID |
| 197 | (SEQ ID NO: 186) | NO: 207) | NO: 228) |
| 1220SBC1068- | RSSQSLLNSTNQKNYL | WASTRES (SEQ ID | QNDYTSPFT (SEQ ID |
| 210 | T (SEQ ID NO: 187) | NO: 208) | NO: 229) |
| 1220SBC1068- | RSSQSLLNSTNQKNYL | WASTRES (SEQ ID | QNDYTSPFT (SEQ ID |
| 274 | T (SEQ ID NO: 188) | NO: 209) | NO: 230) |
| 1220SBC1068- | RSNKSLLHSNGNAYLS | RVSDLAS (SEQ ID | MQHLEFPYT (SEQ ID |
| 281 | (SEQ ID NO: 189) | NO: 210) | NO: 231) |
| 20240628SBC1080- | QASQSVGGNNDLA | KASNLAS(SEQ ID | QGYYPGWIRA(SEQ ID |
| 002 | (SEQ ID NO: 440) | NO: 463) | NO: 481) |
| 20240628SBC1080- | QSSQSVGNNNDLA | RTSNLAP(SEQ ID | LAYYPDWIRG(SEQ ID |
| 003 | (SEQ ID NO: 441) | NO: 464) | NO: 482) |
| 20240628SBC1080- | QSSQRVYNNWLS(SEQ | EASKLAS(SEQ ID | LGTYDCSSADCGA |
| 004 | ID NO: 442) | NO: 465) | (SEQ ID NO: 483) |
| 20240628SBC1080- | QASESIDNALA(SEQ ID | LASTLVP(SEQ ID | QQGYSISNVDNV(SEQ |
| 005 | NO: 443) | NO: 466) | ID NO: 484) |
| 20240628SBC1080- | QSSQSVYNYNWLS | SVSTLAS(SEQ ID | LGIYDCVSADCYG(SEQ |
| 006 | (SEQ ID NO: 444) | NO: 467) | ID NO: 485) |
| 20240628SBC1080- | QASQSVYSDNRLA | RASTLAS(SEQ ID | LGEYSDSGEDT(SEQ ID |
| 007 | (SEQ ID NO: 445) | NO: 468) | NO: 486) |
| 20240628SBC1080- | QSSQSVYNNNLLA | STSTLAS(SEQ ID | QAFYNDDMWV(SEQ |
| 008 | (SEQ ID NO: 446) | NO: 469) | ID NO: 487) |
| 20240628SBC1080- | QANETIGNALA(SEQ ID | LASTLAS(SEQ ID | QQGYSISNVDNV(SEQ |
| 009 | NO: 447) | NO: 470) | ID NO: 484) |
| 20240903SBC1093- | QSSQSVGSNNRLS(SEQ | GASNLAS(SEQ ID | LGSYNCADGDCNA |
| 015 | ID NO: 448) | NO: 471) | (SEQ ID NO: 488) |
| 20240903SBC1093- | QSSHSVGSNNRLS(SEQ | GAFNLAS(SEQ ID | LGSYNCADGDCNA |
| 020 | ID NO: 449) | NO: 472) | (SEQ ID NO: 488) |
| 20240903SBC1093- | QSSQSVGNNKRLS(SEQ | GASNLAS(SEQ ID | LGSYNCADGDCNA |
| 021 | ID NO: 450) | NO: 471) | (SEQ ID NO: 488) |
| 20240903SBC1093- | QSSQSVGSNNRLS(SEQ | GASNLAS(SEQ ID | LGSYNCADGDCNA |
| 025 | ID NO: 448) | NO: 471) | (SEQ ID NO: 488) |
| 20240903SBC1093- | QSSQSVGSNNRLS(SEQ | GAFNLAS(SEQ ID | LGSYNCANGDCNA |
| 030 | ID NO: 448) | NO: 472) | (SEQ ID NO: 489) |
| 20240903SBC1093- | QSSQSVGSNNRLS(SEQ | GAFNLAS(SEQ ID | LGSYNCADGDCNA |
| 182 | ID NO: 448) | NO: 472) | (SEQ ID NO: 488) |
| 20240903SBC1093- | QSSQSVGSNNRLS(SEQ | GASNLAS(SEQ ID | LGSYNCADGDCNT |
| 183 | ID NO: 448) | NO: 471) | (SEQ ID NO: 490) |
| 20240903SBC1093- | QSSQSVGSNNRLS(SEQ | GASNLAS(SEQ ID | LGSYNCAEGDCNA |
| 184 | ID NO: 448) | NO: 471) | (SEQ ID NO: 491) |
| 20240903SBC1093- | QSSQSVGSNNRLS(SEQ | GAFNLAS(SEQ ID | LGSYNCANGDCNA |
| 185 | ID NO: 448) | NO: 472) | (SEQ ID NO: 489) |
| 20240903SBC1093- | QSSQSVGSNNRLS(SEQ | GASNLAS(SEQ ID | ID LGSYNCADGDCNA |
| 230 | ID NO: 448) | NO: 471) | (SEQ ID NO: 488) |
| 20240903SBC1093- | QSSQTIWDNNWLA | LASKLAS(SEQ ID | AGTYSDNIYS(SEQ ID |
| 033 | (SEQ ID NO: 451) | NO: 473) | NO: 492) |
| 20240903SBC1093- | QSSQSIWDNNWLA | LASKLAS(SEQ ID | AGTYSDNVYT(SEQ ID |
| 034 | (SEQ ID NO: 452) | NO: 473) | NO: 493) |
| 20240903SBC1093- | QSSQSIWDNNWLA | LASKLAS(SEQ ID | AGTYSDNVYT(SEQ ID |
| 035 | (SEQ ID NO: 452) | NO: 473) | NO: 493) |
| 20240903SBC1093- | HSSHSIYDNNWLS(SEQ | LASTLAS(SEQ ID | AGTYSYNIYT(SEQ ID |
| 044 | ID NO: 453) | NO: 470) | NO: 494) |
| 20240903SBC1093- | HSSHSIYDNNWLS(SEQ | LASTLAS(SEQ ID | AGTYSYNIYT(SEQ ID |
| 054 | ID NO: 453) | NO: 470) | NO: 494) |
| 20240903SBC1093- | HSSHSIYDNNWLS(SEQ | SASTLAS(SEQ ID | AGTYSYNIYT(SEQ ID |
| 055 | ID NO: 453) | NO: 474) | NO: 494) |
| 20240903SBC1093- | HSSHSIYDNNWLS(SEQ | LASTLAS(SEQ ID | AGTYSYNIYT(SEQ ID |
| 056 | ID NO: 453) | NO: 470) | NO: 494) |
| 20240903SBC1093- | HSSHSIYDNNWLS(SEQ | LASTLAS(SEQ ID | AGTYTYNIYT(SEQ ID |
| 057 | ID NO: 453) | NO: 470) | NO: 495) |
| 20240903SBC1093- | HSSHSIYDNNWLS(SEQ | LASTLAS(SEQ ID | AGTYSYNIYT(SEQ ID |
| 059 | ID NO: 453) | NO: 470) | NO: 494) |
| 20240903SBC1093- | QSSQSIWDNNWLA | LASTLAS(SEQ ID | AGTYSDNIYT(SEQ ID |
| 219 | (SEQ ID NO: 452) | NO: 470) | NO: 496) |
| 20240903SBC1093- | QSSQSIWDNNWLA | LASKLAT(SEQ ID | AGTYSDNIYT(SEQ ID |
| 228 | (SEQ ID NO: 452) | NO: 475) | NO: 496) |
| 20240903SBC1093- | QSSESVWQNNRLS | GASNLAS(SEQ ID | AGLKAWNDDDDG |
| 023 | (SEQ ID NO: 454) | NO: 471) | (SEQ ID NO: 497) |
| 20240903SBC1093- | QASQSVWQNNRLS | TASTLAS(SEQ ID | AGLRAWNDDDDG |
| 036 | (SEQ ID NO: 455) | NO: 476) | (SEQ ID NO: 498) |
| 20240903SBC1093- | QSSESVWQNNRLS | RASTLAS(SEQ ID | AGLKAWNDDDDG |
| 037 | (SEQ ID NO: 454) | NO: 468) | (SEQ ID NO: 497) |
| 20240903SBC1093- | QSSESVWQNNRLS | QASKLAS(SEQ ID | AGLKAWNDDDDG |
| 220 | (SEQ ID NO: 454) | NO: 477) | (SEQ ID NO: 497) |
| 20240903SBC1093- | QSSQSVYNNNWLS | SASTLAS(SEQ ID | AGDYSVGIYT(SEQ ID |
| 093-074 | (SEQ ID NO: 456) | NO: 474) | NO: 499) |
| 20240903SBC1093- | QSSQSVYNNNWLS | YASTLAS(SEQ ID | AGDYSVGIYT(SEQ ID |
| 075 | (SEQ ID NO: 456) | NO: 478) | NO: 499) |
| 20240903SBC1093- | QSSESVYSDWLS(SEQ | SASTLAS(SEQ ID | AGDYSVGIYA(SEQ ID |
| 193 | ID NO: 457) | NO: 474) | NO: 500) |
| 20240903SBC1093- | QSSQSVYSDWLS(SEQ | SASTLAS(SEQ ID | AGDYSVGIYA(SEQ ID |
| 194 | ID NO: 458) | NO: 474) | NO: 500) |
| 20240903SBC1093- | QASESVYNNNFLS(SEQ | LASTLAS(SEQ ID | AGVKSDSTDGIA(SEQ |
| 099 | ID NO: 459) | NO: 470) | ID NO: 501) |
| 20240929SBC1205- | SASQAISNYLN(SEQ ID | YTSSLYS(SEQ ID | QQYIKLPRT(SEQ ID |
| 022 | NO: 460) | NO: 479) | NO: 502) |
| 20241223SBC1205- | KSSQSLLYSSNQKNYL | WASTRES(SEQ ID | QQYYSYPT(SEQ ID |
| 061 | A(SEQ ID NO: 461) | NO: 201) | NO: 503) |
| 20241223SBC1205- | KASQDVGTVVA(SEQ | WASTRHT(SEQ ID | QQYSSYPLT(SEQ ID |
| 063 | ID NO: 462) | NO: 480) | NO: 504) |
| 20241223SBC1205- | KSSQSLLYSSNQKNYL | WASTRES(SEQ ID | QQYYNYPLT(SEQ ID |
| 119 | A(SEQ ID NO: 461) | NO: 201) | NO: 505) |
| 20241223SBC1205- | KASQDVGTVVA(SEQ | WASTRHT(SEQ ID | QQYSRYPLT(SEQ ID |
| 130 | ID NO: 462) | NO: 480) | NO: 506) |
| 20241223SBC1205- | KASQDVGTVVA(SEQ | WASTRHT(SEQ ID | QQYSSFPLT(SEQ ID |
| 131 | ID NO: 462) | NO: 480) | NO: 507) |

In some embodiments, the anti-GAL-3 antibody comprises a heavy chain variable (V_{H}) region further comprising a heavy chain framework 1 (HFR1), HFR2, HFR3, and HFR4 and a light chain variable (V_{L}) region comprising light chain FR 1 (LFR1), LFR2, LFR3, and LFR4. In some embodiments, the V_{H} region comprises a HFR1 amino acid sequence at least 70%, at least 75%, at least 80%, at least 85%, at least 90%, or at least 95% identical to any one of SEQ ID NOS: 64-84, 337, and 341, a HFR2 amino acid sequence at least 70%, at least 75%, at least 80%, at least 85%, at least 90%, or at least 95% identical to any one of SEQ ID NOS: 85-105, 338, and 342, a HFR3 amino acid sequence at least 70%, at least 75%, at least 80%, at least 85%, at least 90%, or at least 95% identical to any one of SEQ ID NOS: 106-126, 339, and 343, and/or a HFR4 amino acid sequence at least 70%, at least 75%, at least 80%, at least 85%, at least 90%, or at least 95% identical to any one of SEQ ID NOS: 137-157, 340, and 344. In some embodiments, the V_{L} region comprises a LFR1 amino acid sequence at least 70%, at least 75%, at least 80%, at least 85%, at least 90%, or at least 95% identical to any one of SEQ ID NOS: 232-252, 345, and 349, a LFR2 amino acid sequence at least 70%, at least 75%, at least 80%, at least 85%, at least 90%, or at least 95% identical to any one of SEQ ID NOS: 253-273, 346, and 350, a LFR3 amino acid sequence at least 70%, at least 75%, at least 80%, at least 85%, at least 90%, or at least 95% identical to any one of SEQ ID NOS: 274-294, 347, and 351, and/or a LFR4 amino acid sequence at least 70%, at least 75%, at least 80%, at least 85%, at least 90%, or at least 95% identical to any one of SEQ ID NOS: 295-315, 348, and 352. The exemplary heavy chain FR and light chain FR amino acid sequences are listed in Tables 5 and 6, respectively.

In some embodiments, the anti-GAL-3 antibody comprises all CDRs and FRs of an antibody selected from the group consisting of 1104SBC1068-033, 1104SBC1068-335, 1104SBC1068-365, 1104SBC1068-374, 1104SBC1068-378, 1220SBC1068-022, 1220SBC1068-035, 1220SBC1068-041, 1220SBC1068-056, 1220SBC1068-058, 1220SBC1068-063, 1220SBC1068-097, 1220SBC1068-098, 1220SBC1068-099, 1220SBC1068-129, 1220SBC1068-164, 1220SBC1068-186, 1220SBC1068-197, 1220SBC1068-210, 1220SBC1068-274, 1220SBC1068-281, SIF-001, and SIF-002. The H_{V} CDR, H_{L} CDR, H_{V} FR, and H_{L} FR amino acid sequences of the antibodies are listed in Tables 3-6, respectively.

TABLE 5
Heavy chain framework sequences

| Antibody | | | | |
|---|---|---|---|---|
| ID | HFR1 | HFR2 | HFR3 | HFR4 |
| 1104SBC1068-033 | DVQLQESGPGLVKPS | WIRQFPGNKLE | RISITRDASKNQFFLQL | WGQGTTLTV |
| | QSLSLTCTVTGYSIT | WMG (SEQ ID | NSVTTEDTATYYCAR | SS (SEQ ID |
| | (SEQ ID NO: 64) | NO: 85) | (SEQ ID NO: 106) | NO: 127) |
| 1104SBC1068-335 | QIQLVQSGPELKKPG | WVKQAPGKGL | RFVFSLETSASTAYLQI | WGQGTTLTV |
| | ETVKISCKASGYIFT | KWMG (SEQ ID | NNLKNEDMATYFCAR | SS (SEQ ID |
| | (SEQ ID NO: 65) | NO: 86) | (SEQ ID NO: 107) | NO: 128) |
| 1104SBC1068-365 | QIQLVQSGPELKKPG | WVKQAPGKGL | RFAFSLETSASTAYLQI | WGQGTSVTV |
| | ETVKISCKASGYTFT | KWMG (SEQ ID | NNLKNEDTATYFCGR | SS (SEQ ID |
| | (SEQ ID NO: 66) | NO: 87) | (SEQ ID NO: 108) | NO: 129) |
| 1104SBC1068-374 | QIQLVQSGPELKKPG | WVKQAPGKGL | RFAFSLETSASTAYLQI | WGQGTSVTV |
| | ETVKISCKASGYTFT | KWMG (SEQ ID | NNLKNEDMATYFCAR | SS (SEQ ID |
| | (SEQ ID NO: 67) | NO: 88) | (SEQ ID NO: 109) | NO: 130) |
| 1104SBC1068-378 | QIQLVQSGPELKKPG | WVKQAPGKGL | RFAFSLETSASTAYLQI | WGQGTLVT |
| | ETVKISCKASGYTFT | KWMG (SEQ ID | NNLKNEDTATYFCAR | VSA (SEQ ID |
| | (SEQ ID NO: 68) | NO: 89) | (SEQ ID NO: 110) | NO: 131) |
| 1220SBC1068-022 | EVKLVESGGGLVQP | WFRQAPGKGP | RFTISRENAKNTLFLEM | WGQGTSVTV |
| | GGSRKLSCAASGFTF | EWVA (SEQ ID | SSLRSEDTAIYYCTR | SS (SEQ ID |
| | S (SEQ ID NO: 69) | NO: 90) | (SEQ ID NO: 111) | NO: 132) |
| 1220SBC1068-035 | EVQLQQSGPELVKPG | WVKQSHGKNL | KATFTVDKSSSTAYME | WGAGTTVT |
| | ASMKISCKASGYAFT | EWIG (SEQ ID | LLSLTSEDSGVYYCAR | VSS (SEQ ID |
| | (SEQ ID NO: 70) | NO: 91) | (SEQ ID NO: 112) | NO: 133) |
| 1220SBC1068-041 | EVKLLESGGGLVQPG | WVRQAPGKGL | NFIISRDNAKNALFLQ | WGQGTLVT |
| | GSLKLSCAASGFDFS | EWIG (SEQ ID | MSKVRSEDTALYYCTR | VSA (SEQ ID |
| | (SEQ ID NO: 71) | NO: 92) | (SEQ ID NO: 113) | NO: 134) |
| 1220SBC1068-056 | EVKLVESGGGLVQP | WFRQAPGKGP | RFTISRENAKNTLYLE | WGQGTSVTV |
| | GGSRKLSCAASGFTF | EWVA (SEQ ID | MSSLRSEDTAIYYCTR | SS (SEQ ID |
| | S (SEQ ID NO: 72) | NO: 93) | (SEQ ID NO: 114) | NO: 135) |
| 1220SBC1068-058 | EVKLVESGGGLVQP | WFRQAPGKGP | RFTISRENAKNTLYLE | WGQGTSVTV |
| | GGSRKLSCAASGFTF | EWVA (SEQ ID | MNSLRSEDTAIYYCTR | SS (SEQ ID |
| | S (SEQ ID NO: 73) | NO: 94) | (SEQ ID NO: 115) | NO: 136) |
| 1220SBC1068-063 | EVNLLESGGGLVQPG | WVRQTPGEGL | RFIISRDNAKNTLFLQM | WGHGTLVT |
| | GSLKLSCEASGFDFS | EWIG (SEQ ID | TKVTSDDSALYYCAR | VSA (SEQ ID |
| | (SEQ ID NO: 74) | NO: 95) | (SEQ ID NO: 116) | NO: 137) |
| 1220SBC1068-097 | EVKLLESGGGLVQPG | WVRQAPGKGL | NFIISRDNAKNALYLQ | WGQGTLVT |
| | GSLKLSCAASGFDFS | EWIG (SEQ ID | MSKVKSEDTALYYCTR | VSA (SEQ ID |
| | (SEQ ID NO: 75) | NO: 96) | (SEQ ID NO: 117) | NO: 138) |
| 1220SBC1068-098 | EVKLLESGGGLVQPG | WVRQAPGKGL | NFIISRDNAKNALYLQ | WGQGTLVT |
| | GSLKLSCAASGFDFS | EWIG (SEQ ID | MSKVKSEDTALYYCTR | VSA (SEQ ID |
| | (SEQ ID NO: 76) | NO: 97) | (SEQ ID NO: 118) | NO: 139) |
| 1220SBC1068-099 | EVKLLESGGGLVQPG | WVRQAPGKGL | KFIISRDNAKNTLYLQ | WGQGTLVT |
| | GSLKLSCAASGFDFS | EWIG (SEQ ID | MSKVSSEDTALYYCAR | VSA (SEQ ID |
| | (SEQ ID NO: 77) | NO: 98) | (SEQ ID NO: 119) | NO: 140) |
| 1220SBC1068-129 | EVKLLESGGDLVQPG | WVRQAPGKGL | KFIISRDNAKSSLYLQM | WGQGTLVT |
| | GSLKLSCAASGFDFS | EWIG (SEQ ID | SKVTSEDTALYYCAR | VSA (SEQ ID |
| | (SEQ ID NO: 78) | NO: 99) | (SEQ ID NO: 120) | NO: 141) |
| 1220SBC1068-164 | EVKLLESGGGLVQPG | WVRQAPGKGL | KFNISRDNAKNTLYLQ | WGQGTLVT |
| | GSLKLSCAASGFDFS | EWIG (SEQ ID | MSKVRSEDTALYYCA | VSE (SEQ ID |
| | (SEQ ID NO: 79) | NO: 100) | R (SEQ ID NO: 121) | NO: 142) |
| 1220SBC1068-186 | EVQLQQSGPELVKPG | WVKQSHGKNL | KATLTVDKSSSTAYME | WGAGTTVT |
| | ASMKMSCKASGYSF | EWIG (SEQ ID | VFSLTSEDSGVYYCAR | VSS (SEQ ID |
| | T (SEQ ID NO: 80) | NO: 101) | (SEQ ID NO: 122) | NO: 143) |
| 1220SBC1068-197 | EVNLLESGGGLVQPG | WVRQTPGEGL | KFFISRDNAKSTLFLQM | WGHGTLVT |
| | GSLKLSCEASGFDFS | EWIG (SEQ ID | TNVTSEDSALYYCAR | VSA (SEQ ID |
| | (SEQ ID NO: 81) | NO: 102) | (SEQ ID NO: 123) | NO: 144) |
| 1220SBC1068-210 | EVKLLESGGGLVQPG | WVRQAPGKGL | NFIISRDNAKNALYLQ | WGQGTLVT |
| | GSLKLSCAASGFDFS | EWIG (SEQ ID | MSKVRSEDTALYYCTR | VSA (SEQ ID |
| | (SEQ ID NO: 82) | NO: 103) | (SEQ ID NO: 124) | NO: 145) |
| 1220SBC1068-274 | EVKLLESGGGLVQPG | WVRQAPGKGL | NFIISRDNAKNALYLQ | WGQGTLVT |
| | GSLKLSCAASGFDFS | EWIG (SEQ ID | MSKLRSEDTALYYCTR | VSA (SEQ ID |
| | (SEQ ID NO: 83) | NO: 104) | (SEQ ID NO: 125) | NO: 146) |
| 1220SBC1068-281 | EVKLVESGGGLVQS | WVRQAPGKGP | RFTISRENAKNTLYLE | WGQGTSVTV |
| | GGSRKLSCAASGFTF | EWVA (SEQ ID | MSSLRSEDTAVYYCTR | SS (SEQ ID |
| | S (SEQ ID NO: 84) | NO: 105) | (SEQ ID NO: 126) | NO: 147) |
| SIF001 | QVQLVQSGSELKKP | WVRQAPGQGL | RFVFSLDTSVSTAYLQI | WGQGTLVT |
| | GASVKVSCKASGYT | EWMG (SEQ ID | SSLKNEDMAVYFCAR | VSS (SEQ ID |
| | FT (SEQ ID NO: 337) | NO: 338) | (SEQ ID NO: 339) | NO: 340) |
| SIF002 | EVQLLESGGGLVQPG | WVRQAPGKGL | RFTISRDNSKNTLYLQ | WGQGTLVT |
| | GSLRLSCAASGFTFS | EWVS (SEQ ID | MNSLRAEDTAVYYCA | VSS (SEQ ID |
| | (SEQ ID NO: 341) | NO: 342) | K (SEQ ID NO: 343) | NO: 344) |

TABLE 6
Light chain framework sequences

| Antibody | | | | |
|---|---|---|---|---|
| ID | LFR1 | LFR2 | LFR3 | LFR4 |
| 1104SBC1068-033 | NIVLTQSPASLAV | WYQQKPGQPP | GVPARFSGSGSRTDFTL | FGAGTKLEL |
| | SLGQRATISC | KLLIY (SEQ ID | TIDPVEADDAATYYC | K (SEQ ID NO: |
| | (SEQ ID NO: 232) | NO: 253) | (SEQ ID NO: 274) | 295) |
| 1104SBC1068-335 | DVVMTQTPLTLS | WLLQRPGQSPK | GVPDRFTGSGSGTDFTL | FGGGTKLEIK |
| | VTIGQPASISC | RLIY (SEQ ID | KISRVEAEDLGVYYC | (SEQ ID NO: |
| | (SEQ ID NO: 233) | NO: 254) | (SEQ ID NO: 275) | 296) |
| 1104SBC1068-365 | DVVMTQTPLTLS | WLLQRPGQSPK | GVPDRFTGSGSGTDFTL | FGGGTKLEIK |
| | VTIGQPASISC | RLIY (SEQ ID | KISRVEAEDLGVYYC | (SEQ ID NO: |
| | (SEQ ID NO: 234) | NO: 255) | (SEQ ID NO: 276) | 297) |
| 1104SBC1068-374 | QAVVTQESALTT | WVQEKPDHLFT | GVPVRFSGSLIGDKAAL | FGGGTKVTV |
| | SPGGTVILTC | GLIG (SEQ ID | TITGAQTEDDAMYFC | L (SEQ ID NO: |
| | (SEQ ID NO: 235) | NO: 256) | (SEQ ID NO: 277) | 298) |
| 1104SBC1068-378 | DIVLTQSPATLSV | WYQQKSHESPR | GIPSRFSGSGSGTDFTLSI | FGAGTKLEL |
| | TPGDSVSLSC | LLIK (SEQ ID | NSVETEDFGMYFC (SEQ | K (SEQ ID NO: |
| | (SEQ ID NO: 236) | NO: 257) | ID NO: 278) | 299) |
| 1220SBC1068-022 | DIVMTQAAPSVP | WFLQRPGQSPQ | GVPDRFSGSGSGTAFTL | FGGGTKLEIK |
| | VTPGESVSISC | LLIY (SEQ ID | RISRVEAEDVGVYYC | (SEQ ID NO: |
| | (SEQ ID NO: 237) | NO: 258) | (SEQ ID NO: 279) | 300) |
| 1220SBC1068-035 | DIVMTQSHKFMS | WYQQKPGQSP | GVPDRFTGSGSGTDFTF | FGGGTKLEIK |
| | TSVGDGVNITC | RLLIY (SEQ ID | TISSMQAEDLAVYYC | (SEQ ID NO: |
| | (SEQ ID NO: 238) | NO: 259) | (SEQ ID NO: 280) | 301) |
| 1220SBC1068-041 | DIVMTQSPSSLTV | WYQQKPGQPP | GVPDRFTGSGSGTDFTL | FGSGTRLEIK |
| | TAGEKVTMNC | KLLIY (SEQ ID | TISTVQTEDLAVYYC | (SEQ ID NO: |
| | (SEQ ID NO: 239) | NO: 260) | (SEQ ID NO: 281) | 302) |
| 1220SBC1068-056 | DIVMTQAAPSVP | WFLQRPGQSPQ | GVPDRFSGSGSGTAFTL | FGGGTKLEIK |
| | VTPGESVSISC | LLIY (SEQ ID | RISSVEAEDVGIYYC | (SEQ ID NO: |
| | (SEQ ID NO: 240) | NO: 261) | (SEQ ID NO: 282) | 303) |
| 1220SBC1068-058 | DIVMTQAAPSVP | WFLQRPGQSPQ | GVPDRFSGSGSGTAFTL | FGGGTKLEIK |
| | VTPGESVSISC | LLIY (SEQ ID | RISRVEAEDVGVYYC | (SEQ ID NO: |
| | (SEQ ID NO: 241) | NO: 262) | (SEQ ID NO: 283) | 304) |
| 1220SBC1068-063 | DIVMTQSPSSLTV | WYQQKPGQPP | GVPDRFTGSGSGTDFTL | FGSGTRLEIK |
| | TAGEKVTMSC | KLLIY (SEQ ID | TISSLQAEDLAVYYC | (SEQ ID NO: |
| | (SEQ ID NO: 242) | NO: 263) | (SEQ ID NO: 284) | 305) |
| 1220SBC1068-097 | DIVMTQSPSSLTV | WYQQKPGQPP | GVPDRFTGSGSGTDFTL | FGSGTKLEIK |
| | TAGEKVTMNC | KLLIY (SEQ ID | TINTVQAEDLAIYYC | (SEQ ID NO: |
| | (SEQ ID NO: 243) | NO: 264) | (SEQ ID NO: 285) | 306) |
| 1220SBC1068-098 | DIVMTQSPSSLTV | WYQQKPGQPP | GVPDRFTGSGSGTDFTL | FGSGTKLEIK |
| | TAGEKVTMNC | KLLIY (SEQ ID | TINTVQAEDLAVYYC | (SEQ ID NO: |
| | (SEQ ID NO: 244) | NO: 265) | (SEQ ID NO: 286) | 307) |
| 1220SBC1068-099 | DIVMTQSPSSLTV | WYQQKPGQPP | GVPDRFTGSGSGTDFTL | FGSGTKLEIK |
| | TAGEKVTMSC | KLLIY (SEQ ID | TISSVQAEDLAVYYC | (SEQ ID NO: |
| | (SEQ ID NO: 245) | NO: 266) | (SEQ ID NO: 287) | 308) |
| 1220SBC1068-129 | DIVMTQSPSSLTV | WYQQKPGQSP | GVPDRFTGSGSGTDFTL | FGSGTKLEIK |
| | TAGKEVTMSC | KLLIY (SEQ ID | TISSVQAEDLAIYYC | (SEQ ID NO: |
| | (SEQ ID NO: 246) | NO: 267) | (SEQ ID NO: 288) | 309) |
| 1220SBC1068-164 | DIVMTQSPSSLTV | WYQQKPGQPP | GVPDRFTGSGSGTDFTL | FGSGTKLEM |
| | TAGEKVTMSC | KLLIY (SEQ ID | TIHGVQAEDLAVYYC | K (SEQ ID NO: |
| | (SEQ ID NO: 247) | NO: 268) | (SEQ ID NO: 289) | 310) |
| 1220SBC1068-186 | DIVMTQSHKFMS | WYQQRPGQSP | GVPDRFTGSGSGTDFTF | FGGGTKLEIK |
| | TSVGDRVSITC | KLLIY (SEQ ID | TISSMQAEDLAIYYC | (SEQ ID NO: |
| | (SEQ ID NO: 248) | NO: 269) | (SEQ ID NO: 290) | 311) |
| 1220SBC1068-197 | DIVMTQSPSSLTV | WYQQKPGQPP | GVPDRFTGSGSGTDFTL | FGSGTRLEIK |
| | TAGEKVTMSC | KLLIY (SEQ ID | TISGLQAEDLAVYYC | (SEQ ID NO: |
| | (SEQ ID NO: 249) | NO: 270) | (SEQ ID NO: 291) | 312) |
| 1220SBC1068-210 | DIVMTQSPSSLTV | WYQQRPGQPP | GVPDRFTGSGSGTDFTL | FGSGTKLEIK |
| | TAGEKVTMNC | KLLIY (SEQ ID | TISTVQAEDLAVYYC | (SEQ ID NO: |
| | (SEQ ID NO: 250) | NO: 271) | (SEQ ID NO: 292) | 313) |
| 1220SBC1068-274 | DIVMTQSPSSLTV | WYQQKPGQPP | GVPDRFTGSGSGSDFTL | FGSGTKLEIK |
| | TAGEKVTMNC | KLLIY (SEQ ID | TISTVQAEDLAVYYC | (SEQ ID NO: |
| | (SEQ ID NO: 251) | NO: 272) | (SEQ ID NO: 293) | 314) |
| 1220SBC1068-281 | DIVMTQAAPSVP | WFLQRPGQSPQ | GVPDRFSGSGSGTAFTL | FGGGTKLEIK |
| | VTPGESVSISC | LLIY (SEQ ID | RISRVEAEDVGIYYC | (SEQ ID NO: |
| | (SEQ ID NO: 252) | NO: 273) | (SEQ ID NO: 294) | 315) |
| SIF001 | EAVVTQEPSLTVS | WVQQKPGQAF | GTPARFSGSLLGDKAAL | FGGGTKLTV |
| | PGGTVTLTC (SEQ | TGLIG (SEQ ID | TLLGAQPEDEAEYFC | L (SEQ ID |
| | ID NO: 345) | NO: 346) | (SEQ ID NO: 347) | NO: 348) |
| SIF002 | DIVMTQSPDSLA | WYQQKPGQPP | GVPDRFSGSGSGTDFTL | FGQGTRLEIK |
| | VSLGERATMNC | KLLIY (SEQ ID | TISSLQAEDVAVYYC | (SEQ ID NO: |
| | (SEQ ID NO: 349) | NO: 350) | (SEQ ID NO: 351) | 352) |

In some embodiments, the anti-GAL-3 antibody comprises a heavy chain variable (V_{H}) region comprising a V_{H} amino acid sequence at least 70%, at least 75%, at least 80%, at least 85%, at least 90%, or at least 95% identical to any one of SEQ ID NOS: 148-168, 353, and 355, and a V_{L} region comprising a V_{L} amino acid sequence at least 70%, at least 75%, at least 80%, at least 85%, at least 90%, or at least 95% identical to any one of SEQ ID NOS: 316-336, 354, and 356.

In some embodiments, the anti-GAL-3 antibody comprises a V_{H} amino acid sequence at least 70%, at least 75%, at least 80%, at least 85%, at least 90%, or at least 95% identity to any one of SEQ ID NO: 148, and a V_{L} amino acid sequence at least 70%, at least 75%, at least 80%, at least 85%, at least 90%, or at least 95% identity to any one of SEQ ID NO: 316. In a particular embodiment, the anti-GAL-3 antibody comprises a V_{H} amino acid sequence of SEQ ID NO: 148 and a V_{L} amino acid sequence of SEQ ID NO: 316 (e.g., the antibody 1104SBC1068-033).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 149, and a V_{L} amino acid sequence at least 70%, at least 75%, at least 80%, at least 85%, at least 90%, or at least 95% identity to any one of SEQ ID NO: 317. In a particular embodiment, the anti-GAL-3 antibody comprises a V_{H} amino acid sequence of SEQ ID NO: 149 and a V_{L} amino acid sequence of SEQ ID NO: 317 (e.g., the antibody 1104SBC1068-035).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 150, and a V_{L} amino acid sequence at least 70%, at least 75%, at least 80%, at least 85%, at least 90%, or at least 95% identity to any one of SEQ ID NO: 318. In a particular embodiment, the anti-GAL-3 antibody comprises a V_{H} amino acid sequence of SEQ ID NO: 150 and a V_{L} amino acid sequence of SEQ ID NO: 318 (e.g., the antibody 1104SBC1068-065).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 151, and a V_{L} amino acid sequence at least 70%, at least 75%, at least 80%, at least 85%, at least 90%, or at least 95% identity to any one of SEQ ID NO: 319. In a particular embodiment, the anti-GAL-3 antibody comprises a V_{H} amino acid sequence of SEQ ID NO: 151 and a V_{L} amino acid sequence of SEQ ID NO: 319 (e.g., the antibody 1104SBC1068-074).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 152, and a V_{L} amino acid sequence at least 70%, at least 75%, at least 80%, at least 85%, at least 90%, or at least 95% identity to any one of SEQ ID NO: 320. In a particular embodiment, the anti-GAL-3 antibody comprises a V_{H} amino acid sequence of SEQ ID NO: 152 and a V_{L} amino acid sequence of SEQ ID NO: 320 (e.g., the antibody 1104SBC1068-078).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 153, and a V_{L} amino acid sequence at least 70%, at least 75%, at least 80%, at least 85%, at least 90%, or at least 95% identity to any one of SEQ ID NO: 321. In a particular embodiment, the anti-GAL-3 antibody comprises a V_{H} amino acid sequence of SEQ ID NO: 153 and a V_{L} amino acid sequence of SEQ ID NO: 321 (e.g., the antibody 1220SBC1068-022).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 154, and a V_{L} amino acid sequence at least 70%, at least 75%, at least 80%, at least 85%, at least 90%, or at least 95% identity to any one of SEQ ID NO: 322. In a particular embodiment, the anti-GAL-3 antibody comprises a V_{H} amino acid sequence of SEQ ID NO: 154 and a V_{L} amino acid sequence of SEQ ID NO: 322 (e.g., the antibody 1220SBC1068-035).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 155, and a V_{L} amino acid sequence at least 70%, at least 75%, at least 80%, at least 85%, at least 90%, or at least 95% identity to any one of SEQ ID NO: 323. In a particular embodiment, the anti-GAL-3 antibody comprises a V_{H} amino acid sequence of SEQ ID NO: 155 and a V_{L} amino acid sequence of SEQ ID NO: 323 (e.g., the antibody 1220SBC1068-041).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 156, and a V_{L} amino acid sequence at least 70%, at least 75%, at least 80%, at least 85%, at least 90%, or at least 95% identity to any one of SEQ ID NO: 324. In a particular embodiment, the anti-GAL-3 antibody comprises a V_{H} amino acid sequence of SEQ ID NO: 156 and a V_{L} amino acid sequence of SEQ ID NO: 324 (e.g., the antibody 1220SBC1068-056).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 157, and a V_{L} amino acid sequence at least 70%, at least 75%, at least 80%, at least 85%, at least 90%, or at least 95% identity to any one of SEQ ID NO: 325. In a particular embodiment, the anti-GAL-3 antibody comprises a V_{H} amino acid sequence of SEQ ID NO: 157 and a V_{L} amino acid sequence of SEQ ID NO: 325 (e.g., the antibody 1220SBC1068-058).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 158, and a V_{L} amino acid sequence at least 70%, at least 75%, at least 80%, at least 85%, at least 90%, or at least 95% identity to any one of SEQ ID NO: 326. In a particular embodiment, the anti-GAL-3 antibody comprises a V_{H} amino acid sequence of SEQ ID NO: 158 and a V_{L} amino acid sequence of SEQ ID NO: 326 (e.g., the antibody 1220SBC1068-063).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 159, and a V_{L} amino acid sequence at least 70%, at least 75%, at least 80%, at least 85%, at least 90%, or at least 95% identity to any one of SEQ ID NO: 327. In a particular embodiment, the anti-GAL-3 antibody comprises a V_{H} amino acid sequence of SEQ ID NO: 159 and a V_{L} amino acid sequence of SEQ ID NO: 327 (e.g., the antibody 1220SBC1068-097).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 160, and a V_{L} amino acid sequence at least 70%, at least 75%, at least 80%, at least 85%, at least 90%, or at least 95% identity to any one of SEQ ID NO: 328. In a particular embodiment, the anti-GAL-3 antibody comprises a V_{H} amino acid sequence of SEQ ID NO: 160 and a V_{L} amino acid sequence of SEQ ID NO: 328 (e.g., the antibody 1220SBC1068-098).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 161, and a V_{L} amino acid sequence at least 70%, at least 75%, at least 80%, at least 85%, at least 90%, or at least 95% identity to any one of SEQ ID NO: 329. In a particular embodiment, the anti-GAL-3 antibody comprises a V_{H} amino acid sequence of SEQ ID NO: 161 and a V_{L} amino acid sequence of SEQ ID NO: 329 (e.g., the antibody 1220SBC1068-099).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 162, and a V_{L} amino acid sequence at least 70%, at least 75%, at least 80%, at least 85%, at least 90%, or at least 95% identity to any one of SEQ ID NO: 330. In a particular embodiment, the anti-GAL-3 antibody comprises a V_{H} amino acid sequence of SEQ ID NO: 162 and a V_{L} amino acid sequence of SEQ ID NO: 330 (e.g., the antibody 1220SBC1068-129).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 163, and a V_{L} amino acid sequence at least 70%, at least 75%, at least 80%, at least 85%, at least 90%, or at least 95% identity to any one of SEQ ID NO: 331. In a particular embodiment, the anti-GAL-3 antibody comprises a V_{H} amino acid sequence of SEQ ID NO: 163 and a V_{L} amino acid sequence of SEQ ID NO: 331 (e.g., the antibody 1220SBC1068-164).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 164, and a V_{L} amino acid sequence at least 70%, at least 75%, at least 80%, at least 85%, at least 90%, or at least 95% identity to any one of SEQ ID NO: 332. In a particular embodiment, the anti-GAL-3 antibody comprises a V_{H} amino acid sequence of SEQ ID NO: 164 and a V_{L} amino acid sequence of SEQ ID NO: 332 (e.g., the antibody 1220SBC1068-186).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 165, and a V_{L} amino acid sequence at least 70%, at least 75%, at least 80%, at least 85%, at least 90%, or at least 95% identity to any one of SEQ ID NO: 333. In a particular embodiment, the anti-GAL-3 antibody comprises a V_{H} amino acid sequence of SEQ ID NO: 165 and a V_{L} amino acid sequence of SEQ ID NO: 333 (e.g., the antibody 1220SBC1068-197).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 166, and a V_{L} amino acid sequence at least 70%, at least 75%, at least 80%, at least 85%, at least 90%, or at least 95% identity to any one of SEQ ID NO: 334. In a particular embodiment, the anti-GAL-3 antibody comprises a V_{H} amino acid sequence of SEQ ID NO: 166 and a V_{L} amino acid sequence of SEQ ID NO: 334 (e.g., the antibody 1220SBC1068-210).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 167, and a V_{L} amino acid sequence at least 70%, at least 75%, at least 80%, at least 85%, at least 90%, or at least 95% identity to any one of SEQ ID NO: 335. In a particular embodiment, the anti-GAL-3 antibody comprises a V_{H} amino acid sequence of SEQ ID NO: 167 and a V_{L} amino acid sequence of SEQ ID NO: 335 (e.g., the antibody 1220SBC1068-274).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 168, and a V_{L} amino acid sequence at least 70%, at least 75%, at least 80%, at least 85%, at least 90%, or at least 95% identity to any one of SEQ ID NO: 336. In a particular embodiment, the anti-GAL-3 antibody comprises a V_{H} amino acid sequence of SEQ ID NO: 168 and a V_{L} amino acid sequence of SEQ ID NO: 336 (e.g., the antibody 1220SBC1068-281).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 353, and a V_{L} amino acid sequence at least 70%, at least 75%, at least 80%, at least 85%, at least 90%, or at least 95% identity to any one of SEQ ID NO: 354. In a particular embodiment, the anti-GAL-3 antibody comprises a V_{H} amino acid sequence of SEQ ID NO: 353 and a V_{L} amino acid sequence of SEQ ID NO: 354 (e.g., the antibody SIF-001).

In some embodiments, the anti-GAL-3 antibody comprises a V_{H} amino acid sequence at least 70%, at least 75%, at least 80%, at least 85%, at least 90%, or at least 95% identity to any one of SEQ ID NO: 355, and a V_{L} amino acid sequence at least 70%, at least 75%, at least 80%, at least 85%, at least 90%, or at least 95% identity to any one of SEQ ID NO: 356. In a particular embodiment, the anti-GAL-3 antibody comprises a V_{H} amino acid sequence of SEQ ID NO: 355 and a V_{L} amino acid sequence of SEQ ID NO: 356 (e.g., the antibody SIF-002).

In some embodiments, the anti-GAL-3 antibody comprises a H_{V} and H_{L} amino acid sequence of an antibody selected from the group consisting of 1104SBC1068-033, 1104SBC1068-335, 1104SBC1068-365, 1104SBC1068-374, 1104SBC1068-378, 1220SBC1068-022, 1220SBC1068-035, 1220SBC1068-041, 1220SBC1068-056, 1220SBC1068-058, 1220SBC1068-063, 1220SBC1068-097, 1220SBC1068-098, 1220SBC1068-099, 1220SBC1068-129, 1220SBC1068-164, 1220SBC1068-186, 1220SBC1068-197, 1220SBC1068-210, 1220SBC1068-274, 1220SBC1068-281, SIF-001, and SIF002. The H_{V} and H_{L} amino acid sequence of the antibodies are listed in Table 7.

TABLE 7
Heavy and Light variable region sequences

| Antibody | | |
|---|---|---|
| ID | HV | LV |
| 1104SBC1068-033 | DVQLQESGPGLVKPSQSLSLTCTVTGYSITS | NIVLTQSPASLAVSLGQRATISCRASES |
| | DYAWNWIRQFPGNKLEWMGYISYSGSTSY | VDSYGNSFMHWYQQKPGQPPKLLIYL |
| | NPSLKSRISITRDASKNQFFLQLNSVTTEDT | ASNLESGVPARFSGSGSRTDFTLTIDPV |
| | ATYYCAREGGRGGTEGYYFDYWGQGTTLT | EADDAATYYCQQNNEDPLTFGAGTKL |
| | VSS (SEQ ID NO: 148) | ELK (SEQ ID NO: 316) |
| 1104SBC1068-335 | QIQLVQSGPELKKPGETVKISCKASGYIFTN | DVVMTQTPLTLSVTIGQPASISCKSSQS |
| | YGMNWVKQAPGKGLKWMGWINTYSGEPT | LLDSDGKTYLNWLLQRPGQSPKRLIYL |
| | YVDDFKGRFVFSLETSASTAYLQINNLKNE | VSKLDSGVPDRFTGSGSGTDFTLKISRV |
| | DMATYFCARGTVVGFDYWGQGTTLTVSS | EAEDLGVYYCWQGTHFPQTFGGGTKL |
| | (SEQ ID NO: 149) | EIK (SEQ ID NO: 317) |
| 1104SBC1068-365 | QIQLVQSGPELKKPGETVKISCKASGYTFTN | DVVMTQTPLTLSVTIGQPASISCKSSQS |
| | YGMNWVKQAPGKGLKWMGWINTNTGEPT | LLDSDGKTYLNWLLQRPGQSPKRLIYL |
| | YSEEFKGRFAFSLETSASTAYLQINNLKNED | VSKLDSGVPDRFTGSGSGTDFTLKISRV |
| | TATYFCGRSSYYAMDYWGQGTSVTVSS | EAEDLGVYYCWQATHFPQTFGGGTKL |
| | (SEQ ID NO: 150) | EIK (SEQ ID NO: 318) |
| 1104SBC1068-374 | QIQLVQSGPELKKPGETVKISCKASGYTFTN | QAVVTQESALTTSPGGTVILTCRSSTGA |
| | YGMNWVKQAPGKGLKWMGWINTYTGEPT | VTTSNYANWVQEKPDHLFTGLIGGTSN |
| | YADDFKGRFAFSLETSASTAYLQINNLKNE | RAPGVPVRESGSLIGDKAALTITGAQTE |
| | DMATYFCARYAMDYWGQGTSVTVSS (SEQ | DDAMYFCALWYSTHYVFGGGTKVTVL |
| | ID NO: 151) | (SEQ ID NO: 319) |
| 1104SBC1068-378 | QIQLVQSGPELKKPGETVKISCKASGYTFTN | DIVLTQSPATLSVTPGDSVSLSCRASQSI |
| | YGMNWVKQAPGKGLKWMGWINTYTGEPT | SNNLHWYQQKSHESPRLLIKYASQSISG |
| | YADDFKGRFAFSLETSASTAYLQINNLKNE | IPSRFSGSGSGTDFTLSINSVETEDFGMY |
| | DTATYFCARWDDYDGFAYWGQGTLVTVS | FCQQSNSWPLTFGAGTKLELK (SEQ ID |
| | A (SEQ ID NO: 152) | NO: 320) |
| 1220SBC1068-022 | EVKLVESGGGLVQPGGSRKLSCAASGFTFS | DIVMTQAAPSVPVTPGESVSISCRSSKS |
| | DYGMAWFRQAPGKGPEWVAFISNLAYSVY | LLHNNGNTYLCWFLQRPGQSPQLLIYR |
| | YADTVTGRFTISRENAKNTLFLEMSSLRSED | MSDLASGVPDRFSGSGSGTAFTLRISRV |
| | TAIYYCTRDRGLRESMDYWGQGTSVTVSS | EAEDVGVYYCMQHLEYPYTFGGGTKL |
| | (SEQ ID NO: 153) | EIK (SEQ ID NO: 321) |
| 1220SBC1068-035 | EVQLQQSGPELVKPGASMKISCKASGYAFT | DIVMTQSHKFMSTSVGDGVNITCKASQ |
| | GYTMNWVKQSHGKNLEWIGLISPYSGGTN | DVSTAVAWYQQKPGQSPRLLIYSASYR |
| | YNQRFKGKATFTVDKSSSTAYMELLSLTSE | YTGVPDRFTGSGSGTDFTFTISSMQAED |
| | DSGVYYCARSGFITTGVPTNFDVWGAGTT | LAVYYCQQHYTTPRTFGGGTKLEIK |
| | VTVSS (SEQ ID NO: 154) | (SEQ ID NO: 322) |
| 1220SBC1068-041 | EVKLLESGGGLVQPGGSLKLSCAASGFDFS | DIVMTQSPSSLTVTAGEKVTMNCRSSQ |
| | RFWMSWVRQAPGKGLEWIGEISPDSNTIDL | SLFNSTNQKNYLTWYQQKPGQPPKLLI |
| | TPSLKDNFIISRDNAKNALFLQMSKVRSEDT | YWASSRESGVPDRFTGSGSGTDFTLTIS |
| | ALYYCTRPYYGYYWGQGTLVTVSA (SEQ | TVQTEDLAVYYCONDYTSPFTFGSGTR |
| | ID NO: 155) | LEIK (SEQ ID NO: 323) |
| 1220SBC1068-056 | EVKLVESGGGLVQPGGSRKLSCAASGFTFS | DIVMTQAAPSVPVTPGESVSISCGSSKS |
| | DYGMAWFRQAPGKGPEWVAFISNLAYSVY | LLHKNGNTYLCWFLQRPGQSPQLLIYR |
| | YADTVTGRFTISRENAKNTLYLEMSSLRSE | MSNLASGVPDRFSGSGSGTAFTLRISSV |
| | DTAIYYCTRDRGLRESMDYWGQGTSVTVS | EAEDVGIYYCMQHLEYPYTFGGGTKLE |
| | S (SEQ ID NO: 156) | IK (SEQ ID NO: 324) |
| 1220SBC1068-058 | EVKLVESGGGLVQPGGSRKLSCAASGFTFS | DIVMTQAAPSVPVTPGESVSISCRSSKS |
| | DYGMAWFRQAPGKGPEWVAFISNLAYSVY | LLHNNGNTYLCWFLQRPGQSPQLLIYR |
| | YADTVTGRFTISRENAKNTLYLEMNSLRSE | MSNLASGVPDRFSGSGSGTAFTLRISRV |
| | DTAIYYCTRDRGLRETMDYWGQGTSVTVS | EAEDVGVYYCMQHLEYPYTFGGGTKL |
| | S (SEQ ID NO: 157) | EIK (SEQ ID NO: 325) |
| 1220SBC1068-063 | EVNLLESGGGLVQPGGSLKLSCEASGFDFS | DIVMTQSPSSLTVTAGEKVTMSCKSSQ |
| | RFWMSWVRQTPGEGLEWIGEISPNSSTIHQ | SLLSSINQKNYLTWYQQKPGQPPKLLIY |
| | TPSLKDRFIISRDNAKNTLFLQMTKVTSDDS | WATSRESGVPDRFTGSGSGTDFTLTISS |
| | ALYYCARPYYGYYWGHGTLVTVSA (SEQ | LQAEDLAVYYCHNDYSYPFTFGSGTRL |
| | ID NO: 158) | EIK (SEQ ID NO: 326) |
| 1220SBC1068-097 | EVKLLESGGGLVQPGGSLKLSCAASGFDFS | DIVMTQSPSSLTVTAGEKVTMNCRSSQ |
| | RYWMSWVRQAPGKGLEWIGEINPDSSTIDL | SLLNSTNQKNYLTWYQQKPGQPPKLLI |
| | TPSLKDNFIISRDNAKNALYLQMSKVKSED | YWASTRESGVPDRFTGSGSGTDFTLTIN |
| | TALYYCTRPYHGYYWGQGTLVTVSA (SEQ | TVQAEDLAIYYCONDYTSPFTFGSGTK |
| | ID NO: 159) | LEIK (SEQ ID NO: 327) |
| 1220SBC1068-098 | EVKLLESGGGLVQPGGSLKLSCAASGFDFS | DIVMTQSPSSLTVTAGEKVTMNCRSSQ |
| | RYWMSWVRQAPGKGLEWIGEISPDSSTIDL | SLLNSTNQKNYLTWYQQKPGQPPKLLI |
| | TPSLKDNFIISRDNAKNALYLQMSKVKSED | YWASTKESGVPDRFTGSGSGTDFTLTIN |
| | TALYYCTRPYHGYYWGQGTLVTVSA (SEQ | TVQAEDLAVYYCONDYTSPFTFGSGTK |
| | ID NO: 160) | LEIK (SEQ ID NO: 328) |
| 1220SBC1068-099 | EVKLLESGGGLVQPGGSLKLSCAASGFDFS | DIVMTQSPSSLTVTAGEKVTMSCKSSQ |
| | RYWMTWVRQAPGKGLEWIGEINPHSSTITY | SLLNSGNQKIYLTWYQQKPGQPPKLLI |
| | TPSLKDKFIISRDNAKNTLYLQMSKVSSEDT | YWASIRESGVPDRFTGSGSGTDFTLTIS |
| | ALYYCARPYYGYYWGQGTLVTVSA (SEQ | SVQAEDLAVYYCONDYSSPFTFGSGTK |
| | ID NO: 161) | LEIK (SEQ ID NO: 329) |
| 1220SBC1068-129 | EVKLLESGGDLVQPGGSLKLSCAASGFDFS | DIVMTQSPSSLTVTAGKEVTMSCKSSQ |
| | RFWMSWVRQAPGKGLEWIGEINPDSSTITY | SLLNSTNQKNYLTWYQQKPGQSPKLLI |
| | TPSLKDKFIISRDNAKSSLYLQMSKVTSEDT | YWASTRDSGVPDRFTGSGSGTDFTLTIS |
| | ALYYCARPYYGYYWGQGTLVTVSA (SEQ | SVQAEDLAIYYCHNDYISPFTFGSGTKL |
| | ID NO: 162) | EIK (SEQ ID NO: 330) |
| 1220SBC1068-164 | EVKLLESGGGLVQPGGSLKLSCAASGFDFS | DIVMTQSPSSLTVTAGEKVTMSCKSSQ |
| | RYWMSWVRQAPGKGLEWIGEISPDSSTINY | SLFNSGNQKNYLTWYQQKPGQPPKLLI |
| | TASLKDKFNISRDNAKNTLYLQMSKVRSED | YWAYTRESGVPDRFTGSGSGTDFTLTI |
| | TALYYCARPYYGYYWGQGTLVTVSE (SEQ | HGVQAEDLAVYYCHNDYTSPFTFGSGT |
| | ID NO: 163) | KLEMK (SEQ ID NO: 331) |
| 1220SBC1068-186 | EVQLQQSGPELVKPGASMKMSCKASGYSF | DIVMTQSHKFMSTSVGDRVSITCKASQ |
| | TGYTMNWVKQSHGKNLEWIGLISPYSGGT | DVSTAVAWYQQRPGQSPKLLIYSASNR |
| | NYNQKFKGKATLTVDKSSSTAYMEVESLTS | YTGVPDRFTGSGSGTDFTFTISSMQAED |
| | EDSGVYYCARSGFITTGVATNFDVWGAGT | LAIYYCQQHYSTPRTFGGGTKLEIK |
| | TVTVSS (SEQ ID NO: 164) | (SEQ ID NO: 332) |
| 1220SBC1068-197 | EVNLLESGGGLVQPGGSLKLSCEASGFDFS | DIVMTQSPSSLTVTAGEKVTMSCKSSQ |
| | RFWMSWVRQTPGEGLEWIGEISPNSSTIHQ | SLLNSINQKNYLTWYQQKPGQPPKLLI |
| | TPSLKDKFFISRDNAKSTLFLQMTNVTSEDS | YWATFRESGVPDRFTGSGSGTDFTLTIS |
| | ALYYCARPYYGYYWGHGTLVTVSA (SEQ | GLQAEDLAVYYCHNDYSYPFTFGSGTR |
| | ID NO: 165) | LEIK (SEQ ID NO: 333) |
| 1220SBC1068-210 | EVKLLESGGGLVQPGGSLKLSCAASGFDFS | DIVMTQSPSSLTVTAGEKVTMNCRSSQ |
| | RYWMSWVRQAPGKGLEWIGEINPDSSTIDL | SLLNSTNQKNYLTWYQQRPGQPPKLLI |
| | TPSLKDNFIISRDNAKNALYLQMSKVRSED | YWASTRESGVPDRFTGSGSGTDFTLTIS |
| | TALYYCTRPYYGYYWGQGTLVTVSA (SEQ | TVQAEDLAVYYCONDYTSPFTFGSGTK |
| | ID NO: 166) | LEIK (SEQ ID NO: 334) |
| 1220SBC1068-274 | EVKLLESGGGLVQPGGSLKLSCAASGFDFS | DIVMTQSPSSLTVTAGEKVTMNCRSSQ |
| | RYWMSWVRQAPGKGLEWIGEISPDSNTIDL | SLLNSTNQKNYLTWYQQKPGQPPKLLI |
| | TPSLKDNFIISRDNAKNALYLQMSKLRSEDT | YWASTRESGVPDRFTGSGSGSDFTLTIS |
| | ALYYCTRPYYGYYWGQGTLVTVSA (SEQ | TVQAEDLAVYYCONDYTSPFTFGSGTK |
| | ID NO: 167) | LEIK (SEQ ID NO: 335) |
| 1220SBC1068-281 | EVKLVESGGGLVQSGGSRKLSCAASGFTFS | DIVMTQAAPSVPVTPGESVSISCRSNKS |
| | DFGMAWVRQAPGKGPEWVAFISNLAYNIY | LLHSNGNAYLSWFLQRPGQSPQLLIYR |
| | YSDTVTGRFTISRENAKNTLYLEMSSLRSED | VSDLASGVPDRESGSGSGTAFTLRISRV |
| | TAVYYCTRDRGLRETMDYWGQGTSVTVSS | EAEDVGIYYCMQHLEFPYTFGGGTKLE |
| | (SEQ ID NO: 168) | IK (SEQ ID NO: 336) |
| SIF001 | QVQLVQSGSELKKPGASVKVSCKASGYTFT | EAVVTQEPSLTVSPGGTVTLTCRSSTGA |
| | NYGMNWVRQAPGQGLEWMGWINTYTGEP | VTTSNYANWVQQKPGQAFTGLIGGTS |
| | TYADDFKGRFVFSLDTSVSTAYLQISSLKNE | NRAPGTPARFSGSLLGDKAALTLLGAQ |
| | DMAVYFCARYAMDYWGQGTLVTVSS | PEDEAEYFCALWYSTHYVFGGGTKLT |
| | (SEQ ID NO: 353) | VL (SEQ ID NO: 354) |
| SIF002 | EVQLLESGGGLVQPGGSLRLSCAASGFTFS | DIVMTQSPDSLAVSLGERATMNCRSSQ |
| | RFWMSWVRQAPGKGLEWVSEISPDSNTIDL | SLFSSTNQKNYLTWYQQKPGQPPKLLI |
| | TPSLKDRFTISRDNSKNTLYLQMNSLRAED | YWASSRESGVPDRFSGSGSGTDFTLTIS |
| | TAVYYCAKPYYGYYWGQGTLVTVSS (SEQ | SLQAEDVAVYYCONDYTSPFTFGQGTR |
| | ID NO: 355) | LEIK (SEQ ID NO: 356) |

In some embodiments, the anti-GAL-3 antibody comprises a heavy chain variable region comprising a HCDR1 comprising a sequence NYGMN (SEQ ID NO: 4), or a variant HCDR1 in which 1, 2, or 3 amino acids are substituted relative to the sequence; a HCDR2 comprising a sequence WINTYTGEPTYADDFKG (SEQ ID NO: 25), or a variant HCDR2 in which 1, 2, or 3 amino acids are substituted relative to the sequence; and a HCDR3 comprising a sequence YAMDY (SEQ ID NO: 46), or a variant HCDR3 in which 1, 2, or 3 amino acids are substituted relative to the sequence. In some embodiments, the anti-GAL-3 antibody further comprises a light chain variable region comprising a LCDR1 comprising a sequence RSSTGAVTTSNYAN (SEQ ID NO: 172), or a variant LCDR1 in which 1 amino acid is substituted relative to the sequence; a LCDR2 comprising a sequence GTSNRAP (SEQ ID NO: 193), or variant LCDR2 in which 1 amino acid is substituted relative to the sequence; and a LCDR3 comprising a sequence ALWYSTHYV (SEQ ID NO: 214), or a variant LCDR3 in which 1 amino acid is substituted relative to the sequence.

In some embodiments, the antibody or the binding fragment thereof that binds to Galectin-3 comprises a heavy chain variable region comprising a HCDR1 comprising a sequence RFWMS (SEQ ID NO: 8), or a variant HCDR1 in which 1, 2, or 3 amino acids are substituted relative to the sequence; a HCDR2 comprising a sequence EISPDSNTIDLTPSLKD (SEQ ID NO: 29), or a variant HCDR2 in which 1, 2, or 3 amino acids are substituted relative to the sequence; and a HCDR3 comprising a sequence PYYGYY (SEQ ID NO: 50), or a variant HCDR3 in which 1, 2, or 3 amino acids are substituted relative to the sequence. In some embodiments, the anti-GAL-3 antibody further comprises a light chain variable region comprising a LCDR1 comprising a sequence RSSQSLFNSTNQKNYLT (SEQ ID NO: 176) or RSSQSLFSSTNQKNYLT (SEQ ID NO: 369), or a variant LCDR1 in which 1 amino acid is substituted relative to the sequence; a LCDR2 comprising a sequence WASSRES (SEQ ID NO: 197), or variant LCDR2 in which 1 amino acid is substituted relative to the sequence; and a LCDR3 comprising a sequence QNDYTSPFT (SEQ ID NO: 218), or a variant LCDR3 in which I amino acid is substituted relative to the sequence.

In some embodiments, the antibody or the binding fragment thereof that binds to Galectin-3 comprises a HCDR1 comprising a sequence of NX₂GMN (SEQ ID NO:357), wherein X₂ is Y, W, or F, wherein the HCDR1 has zero or one aa substitution in rest of the residues relative to the HCDR1 sequence, a HCDR2 comprising a sequence of X₁IX₃TYTGEPTYADDFKG (SEQ ID NO: 358), Where X₁ is W, Y, or F, and wherein X₃ is N or Q, wherein the HCDR2 has zero, one, two, three, or four aa substitutions in rest of the residues relative to the HCDR2 sequence, a HCDR3 comprising a sequence of YAMDY (SEQ ID NO:359), wherein the HCDR3 has zero, one, or two aa substitutions relative to the HCDR3 sequence, a LCDR1 comprising a sequence of RSSTGAVTTSNX₁₂AN (SEQ ID NO:360), wherein X₁₂ is Y, W, or F, wherein the LCDR1 has zero, one, two, three, or four aa substitutions in the rest of the residues relative to the LCDR1 sequence, a LCDR2 comprising a sequence of GTSNRAP (SEQ ID NO: 361), wherein the LCDR2 has zero, one, two, or three aa substitutions in the rest of the residues relative to the LCDR2 sequence, and a LCDR3 comprising a sequence of ALX₃YSTHX₈V (SEQ ID NO: 362), wherein X₃ is Y, W, or F, wherein X₈ is Y, W, or F, wherein the LCDR3 has zero, one, two aa substitutions in the rest of the residues relative to the LCDR3 sequence.

In some embodiments, the antibody or the binding fragment thereof that binds to Galectin-3, wherein the antibody comprises: a HCDR1 comprising a sequence of NX₂GMN (SEQ ID NO: 357), wherein X₂ is Y, W, or F, a HCDR2 comprising a sequence of X₁IX₃TYTGEPTYADDFKG (SEQ ID NO:358), Where X₁ is W, Y, or F, and wherein X₃ is N or Q, a HCDR3 comprising a sequence of YAMDY (SEQ ID NO:359), a LCDR1 comprising a sequence of RSSTGAVTTSNX₁₂AN (SEQ ID NO:360), wherein X₁₂ is Y, W, or F, a LCDR2 comprising a sequence of GTSNRAP (SEQ ID NO: 361), and a LCDR3 comprising a sequence of ALX₃YSTHX₈V (SEQ ID NO: 362), wherein X₃ is Y, W, or F, wherein X₈ is Y, W, or F; wherein the HCDR1 has zero or one aa substitution in rest of the residues relative to the HCDR1 sequence, the HCDR2 has zero, one, two, three, or four aa substitutions in rest of the residues relative to the HCDR2 sequence, the HCDR3 has zero, one, or two aa substitutions relative to the HCDR3 sequence, the LCDR1 has zero, one, two, three, or four aa substitutions in the rest of the residues relative to the LCDR1 sequence, the LCDR2 has zero, one, two, or three aa substitutions in the rest of the residues relative to the LCDR2 sequence, or the LCDR3 has zero, one, two aa substitutions in the rest of the residues relative to the LCDR3 sequence.

In some embodiments, the antibody or the binding fragment thereof that binds to Galectin-3, wherein the antibody comprises: a HCDR1 comprising a sequence of RFX 3 MS (SEQ ID NO: 363), wherein X₃ is Y, W, or F, and wherein the HCDR1 has zero, one, two aa substitutions in the rest of the residues relative to the HCDR1 sequence, a HCDR2 comprising X₁ISPDSNTIDLTPSLKD (SEQ ID NO: 364), wherein X₁ is E or D, and wherein the HCDR2 has zero, one, two, three, or four aa substitutions in the rest of the residues relative to the HCDR2 sequence, a HCDR3 comprising PYYGX₅Y (SEQ ID NO: 365), wherein X₅ is Y, W, or F, wherein the HCDR3 has zero, one, two aa substitutions in the rest of the residues relative to the HCDR3 sequence, a LCDR1 comprising RSSQSLFSSTNQKNX₁₅LT (SEQ ID NO: 366), wherein X₁₅ is Y, W, or F, wherein the LCDR1 has zero, one, two, three, or four aa substitutions in the rest of the residues relative to the LCDR1 sequence, a LCDR2 comprising WASSRES (SEQ ID NO: 367), wherein the LCDR2 has zero, one, two, or three aa substitutions relative to the LCDR2 sequence, and a LCDR3 comprising QNDYTSPFT (SEQ ID NO: 368), wherein the LCDR3 has zero, one, two, three, or four aa substitutions in the rest of the residues relative to the LCDR3 sequence.

In some embodiments, the antibody or the binding fragment thereof that binds to Galectin-3, wherein the antibody comprises: a HCDR1 comprising a sequence of RFX 3 MS (SEQ ID NO: 363), wherein X₃ is Y, W, or F, a HCDR2 comprising X₁ISPDSNTIDLTPSLKD (SEQ ID NO: 364), wherein X₁ is E or D, a HCDR3 comprising PYYGX₅Y (SEQ ID NO: 365), wherein X₅ is Y, W, or F, a LCDR1 comprising RSSQSLFSSTNQKNX₁₅LT (SEQ ID NO: 366), wherein X₁₅ is Y, W, or F, a LCDR2 comprising WASSRES (SEQ ID NO: 367), and a LCDR3 comprising QNDYTSPFT (SEQ ID NO: 368); and wherein the HCDR1 has zero, one, two aa substitutions in the rest of the residues relative to the HCDR1 sequence, the HCDR2 has zero, one, two, three, or four aa substitutions in the rest of the residues relative to the HCDR2 sequence, the HCDR3 has zero, one, two aa substitutions in the rest of the residues relative to the HCDR3 sequence, the LCDR1 has zero, one, two, three, or four aa substitutions in the rest of the residues relative to the LCDR1 sequence, the LCDR2 has zero, one, two, or three aa substitutions relative to the LCDR2 sequence, or the LCDR3 has zero, one, two, three, or four aa substitutions in the rest of the residues relative to the LCDR3 sequence.

In some embodiments, the antibody or the binding fragment thereof that binds to Galectin-3, wherein the antibody comprises a HCDR1 comprising a sequence of NYGMN (SEQ ID NO: 4), a HCDR2 comprising a sequence of WINTYTGEPTYADDFKG (SEQ ID NO: 25), a HCDR3 comprising a sequence of YAMDY (SEQ ID NO: 46), a LCDR1 comprising a sequence of RSSTGAVTTSNYAN (SEQ ID NO: 172), a LCDR2 comprising a sequence of GTSNRAP (SEQ ID NO: 193), and a LCDR3 comprising a sequence of ALWYSTHYV (SEQ ID NO: 214). In some embodiments, the antibody comprises a V_{H} region comprising a sequence of SEQ ID NO: 353, and a V_{L} region comprising a sequence of SEQ ID NO: 354.

In some embodiments, the antibody or the binding fragment thereof that binds to Galectin-3, wherein the antibody comprises a HCDR1 comprising a sequence of RFWMS (SEQ ID NO: 8), a HCDR2 comprising a sequence of EISPDSNTIDLTPSLKD (SEQ ID NO: 29), a HCDR3 comprising a sequence of PYYGYY (SEQ ID NO: 50), a LCDR1 comprising a sequence of RSSQSLFNSTNQKNYLT (SEQ ID NO: 176) or RSSQSLFSSTNQKNYLT (SEQ ID NO: 369), a LCDR2 comprising a sequence of WASSRES (SEQ ID NO: 197), and a LCDR3 comprising a sequence of QNDYTSPFT (SEQ ID NO: 218). In some embodiments, the antibody comprises a V_{H} region comprising a sequence of SEQ ID NO: 355, and a V_{L} region comprising a sequence of SEQ ID NO: 356.

In some embodiments, an anti-GAL-3 antibody disclosed herein comprises i) a V_{H} amino acid sequence and a V_{L} amino acid sequence listed in Table 7, or ii) a V_{H} amino acid sequence with at least 70%, at least 80%, at least 85%, at least 90%, at least 95% identity to the V_{H} amino acid sequence in Table 7 and a V_{L} amino acid sequence with at least 70% identity to the V_{L} amino acid sequence in Table 7, wherein variations as compared to the V_{H} amino acid sequence or the V_{L} amino acid sequence in Table 7 are in the framework regions only.

In some embodiments, an anti-GAL-3 antibody disclosed herein comprises all three heavy chain CDRs of an antibody in Table 3, and the antibody comprises heavy chain framework regions having at least 70%, at least 80%, at least 85%, at least 90%, at least 95% sequence identity to the combined heavy chain framework regions in the same antibody in Table 3. In some embodiments, at least one of the antibodies comprises all three light chain CDRs of an antibody in Table 4, and the antibody comprises light chain framework regions having at least 70%, at least 80%, at least 85%, at least 90%, at least 95% sequence identity to the combined light chain framework regions of the same antibody in Table 4.

The present disclosure also provides an antibody competes for binding to GAL-3 with any one of the antibodies described herein. In some embodiments, the antibody is capable of competing for binding to GAL-3 with an antibody from a community cluster listed in Table 10. In some embodiments, the antibody competes for binding to GAL-3 with antibody 1104SBC1068-033. In some embodiments, the antibody competes for binding to GAL-3 with any one of the antibodies in community cluster 2, including 1220SBC1068-186, 1220SBC1068-205, 1220SBC1068-022, 1220SBC1068-058, 1220SBC1068-056, 20241223SBC1205-119, and 1104SBC1068-160. In some embodiments, the antibody competes for binding to GAL-3 with any one of the antibodies in community cluster 3, including 20240628SBC1080-009, 20240628SBC1080-002, 1104SBC1068-374, 20240929SBC1205-022, 1104SBC1068-335, 20040628SBC1080-006, 20240628SBC1080-008, 1104SBC1068-378, 1104SBC1068-365, ANb1361-G119-5M-LP2R2-58-higg4P, and 20240628SBC1080-007. In some embodiments, the antibody competes for binding to GAL-3 with an antibody in community cluster 4, including the antibody 1220SBC1068-035. In some embodiments, the antibody competes for binding to GAL-3 with any one of the antibodies in community cluster 5, including 1220SBC1068-098, 1220SBC1068-210, 1220SBC1068-041, 1220SBC1068-063, 1220SBC1068-274, 1220SBC1068-129, 1220SBC1068-099, 1220SBC1068-164, and 1220SBC1068-197. In some embodiments, the antibody competes for binding to GAL-3 with an antibody in community cluster 6, including the antibody 1220SBC1068-097. In some embodiments, the antibody competes for binding to GAL-3 with an antibody in community cluster 7, including the antibody 20240628SBC1080-003. In some embodiments, the antibody competes for binding to GAL-3 with the antibody 20240628SBC1080-004. In some embodiments, the antibody competes for binding to GAL-3 with an antibody in community cluster 8, including the antibody 1104SBC1068-033. In some embodiments, the antibody competes for binding to GAL-3 with any one of the antibodies, in community cluster 9, including 20240903SBC1093-015, 20240903SBC1093-020, 20240903SBC1093-023, 20240903SBC1093-030, 20240628SBC1080-005, 20240903SBC1093-183, 20240903SBC1093-220, 20240903SBC1093-230, 20240903SBC1093-185, 20240903SBC1093-021, 20240903SBC1093-037, 20240903SBC1093-036, 20240903SBC1093-099, 20240903SBC1093-025, 20240903SBC1093-182, and 20240903SBC1093-184. In some embodiments, the antibody competes for binding to GAL-3 with an antibody in community cluster 10, including the antibody 20240903SBC1093-033. In some embodiments, the antibody competes for binding to GAL-3 with an antibody in community cluster 11, including the antibody 20240903SBC1093-034. In some embodiments, the antibody competes for binding to GAL-3 with any one of the antibodies, in community cluster 12, including 20240903SBC1093-228, 20240903SBC1093-074, 20240903SBC1093-193, 20240903SBC1093-044, 20240903SBC1093-059, 20240903SBC1093-056, 20240903SBC1093-057, 20240903SBC1093-219, 20240903SBC1093-194, and 20240903SBC1093-035. In some embodiments, the antibody competes for binding to GAL-3 with an antibody in community cluster 13, including antibody 20240903SBC1093-055. In some embodiments, the antibody competes for binding to GAL-3 with an antibody in community cluster 14, including the antibody 20240903SBC1093-075. In some embodiments, the antibody competes for binding to GAL-3 with an antibody in community cluster 15, including the antibody 20241223SBC1205-061. In some embodiments, the antibody competes for binding to GAL-3 with an antibody in community cluster 16, including the antibody 20241223SBC1205-063. In some embodiments, the antibody competes for binding to GAL-3 with an antibody in community cluster 17, including the antibody 20241223SBC1205-130. In some embodiments, the antibody competes for binding to GAL-3 with an antibody in community cluster 18, including the antibody 20241223SBC1205-131.

Based on the differential binding profiles, it is predicted that combinations of the antibodies from the different community clusters described above (for example, any combination of 2 antibodies between Community cluster 1 & Bin 2 would have an improved beneficial therapeutic profile as compared to the therapeutic profile of the individual antibodies. Thus, in some embodiments, the method provides a combination of two or more antibodies from two or more different community clusters for use to treat patients in need. The two or more antibodies may be administered sequentially or simultaneously, for example, as an antibody cocktail.

### Generation of Antibodies

The anti-GAL-3 antibodies can be produced using vectors and recombinant methodology well known in the art (see, e.g., Sambrook & Russell, Molecular Cloning: A Laboratory Manual, Cold Spring Harbor Laboratory Press; Ausubel, Current Protocols in Molecular Biology). Reagents, cloning vectors, and kits for genetic manipulation are available from commercial vendors.

Accordingly, in a further aspect of the disclosure, provided herein are isolated nucleic acids encoding a V_{H} and/or V_{L} region, or fragment thereof, of any of the GAL-3 antibodies as described herein; vectors comprising such nucleic acids and host cells into which the nucleic acids are introduced that are used to replicate the antibody-encoding nucleic acids and/or to express the antibodies. Such nucleic acids may encode an amino acid sequence containing the V_{L} and/or an amino acid sequence containing the V_{H} of the GAL-3 antibody (e.g., the light and/or heavy chains of the antibody).

The present disclosure further provides a polypeptide comprising a V_{H} sequence and/or a V_{L} amino acid sequence of the anti-GAL-3 antibody described herein. In some embodiments, the polypeptide comprises a heavy chain variable (V_{H}) region comprising a V_{H} amino acid sequence at least 70%, at least 75%, at least 80%, at least 85%, at least 90%, or at least 95% identical to any one of SEQ ID NOS: 148-168, 353, and 355. In some embodiments, the polypeptide comprises a V_{L} region comprising a V_{L} amino acid sequence at least 70%, at least 75%, at least 80%, at least 85%, at least 90%, or at least 95% identical to any one of SEQ ID NOS: 316-336, 354, and 356. In some embodiments, the polypeptide comprises a V_{H} amino acid sequence at least 70%, at least 75%, at least 80%, at least 85%, at least 90%, or at least 95% identical to any one of SEQ ID NOS: 148-168, 353, and 355, and a V_{L} amino acid sequence at least 70%, at least 75%, at least 80%, at least 85%, at least 90%, or at least 95% identical to any one of SEQ ID NOS: 316-336, 354, and 356.

In some embodiments, the polypeptide comprising a V_{H} sequence selected from the group consisting of SEQ ID NOS: 148-168, 353, and 355. In some embodiments, the polypeptide comprising a V_{L} sequence selected from the group consisting of SEQ ID NOS: 316-336, 354, and 356. In some embodiments, the polypeptide comprising a V_{H} sequence selected from the group consisting of SEQ ID NOS: 148-168, 353, and 355, and a V_{L} sequence selected from the group consisting of SEQ ID NOS: 316-336, 354, and 356.

In some embodiments, the polypeptide comprising the V_{H} sequence and/or the V_{L} amino acid sequence of the anti-GAL-3 antibody selected from the group consisting of 1104SBC1068-033, 1104SBC1068-335, 1104SBC1068-365, 1104SBC1068-374, 1104SBC1068-378, 1220SBC1068-022, 1220SBC1068-035, 1220SBC1068-041, 1220SBC1068-056, 1220SBC1068-058, 1220SBC1068-063, 1220SBC1068-097, 1220SBC1068-098, 1220SBC1068-099, 1220SBC1068-129, 1220SBC1068-164, 1220SBC1068-186, 1220SBC1068-197, 1220SBC1068-210, 1220SBC1068-274, 1220SBC1068-281, SIF-001, and SIF-002.

The present disclosure further provides a polynucleotide encoding the polypeptide described herein. In some embodiments, the polynucleotide comprising one or more expression cassettes encoding the polypeptide. In some embodiments, the polynucleotide comprising one expression cassettes encoding both V_{H} and V_{L} amino acid sequences of the antibody. Optionally the V_{H} and V_{L} amino acid sequences are linked through a linker. In some embodiments, the polynucleotide comprising one expression cassettes encoding a V_{H} amino acid sequence of the antibody and the other expression cassettes encoding a V_{L} amino acid sequence of the antibody. In some embodiments, the expression cassette comprising a promoter operably linked to a nucleic acid encoding the V_{H} and/or V_{L} amino acid sequences of the antibody.

The present disclosure further provides an expression vector comprising the polynucleotide(s) described herein and a host cell which the polynucleotide(s) are introduced into for antibody expression. In some embodiments, the host cell contains (1) a vector containing a polynucleotide that encodes the V_{L} amino acid sequence and a polynucleotide that encodes the V_{H} amino acid sequence, or (2) a first vector containing a polynucleotide that encodes the V_{L} amino acid sequence and a second vector containing a polynucleotide that encodes the V_{H} amino acid sequence.

The present disclosure further provides a method of making an anti-GAL-3 antibody as described herein. In some embodiments, the method includes culturing a host cell as described in the preceding paragraph under conditions suitable for expression of the antibody. In some embodiments, the antibody is subsequently recovered from the host cell (or host cell culture medium).

Suitable vectors containing polynucleotides encoding antibodies of the present disclosure, or fragments thereof, include cloning vectors and expression vectors. While the cloning vector selected may vary according to the host cell intended to be used, useful cloning vectors generally can self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mp18, mp19, pBR322, pMB9, ColE1 plasmids, pCR1, RP4, phage DNAs, and shuttle vectors. These and many other cloning vectors are available from commercial vendors.

Expression vectors generally are replicable polynucleotide constructs that contain a nucleic acid of the present disclosure. The expression vector can be replicable in the host cells either as episomes or as an integral part of the chromosomal DNA. Suitable expression vectors include but are not limited to plasmids and viral vectors, including adenoviruses, adeno-associated viruses, retroviruses, and any other vector.

Suitable host cells for expressing an anti-GAL-3 antibody as described herein include both prokaryotic and eukaryotic cells. For example, an anti-GAL-3 antibody may be produced in bacteria when glycosylation and Fc effector function are not needed. After expression, the antibody may be isolated from the bacterial cell paste in a soluble fraction and can be further purified. Alternatively, the host cell may be a eukaryotic host cell, including eukaryotic microorganisms, such as filamentous fungi or yeast, including fungi and yeast strains whose glycosylation pathways have been “humanized,” resulting in the production of an antibody with a partially or fully human glycosylation pattern, vertebrate, invertebrate, and plant cells. Examples of invertebrate cells include insect cells. Numerous baculoviral strains have been identified which may be used in conjunction with insect cells. Plant cell cultures can also be utilized as host cells.

In some embodiments, vertebrate host cells are used for producing an anti-GAL-3 antibody of the present disclosure. For example, mammalian cell lines such as a monkey kidney CVI line transformed by SV40 (COS-7); human embryonic kidney line (293 or 293 cells as described, e.g., in Graham et al., J. Gen Virol. 36:59,1977; baby hamster kidney cells (BHK); mouse sertoli cells (TM4 cells as described, e.g., in Mather, Biol. Reprod. 23:243-251, 1980 monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68, 1982; MRC 5 cells; and FS4 cells may be used to express an anti-GAL-3 antibody antibodies. Other useful mammalian host cell lines include Chinese hamster ovary (CHO) cells, including DHFR-CHO cells (Urlaub et al., Proc. Natl. Acad. Sci. USA 77:4216, 1980); and myeloma cell lines such as Y0, NS0 and Sp2/0. Host cells of the present disclosure also include, without limitation, isolated cells, in vitro cultured cells, and ex vivo cultured cells. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268, 2003.

In some embodiments, an anti-GAL-3 antibody of the present invention is produced by a CHO cell line, e.g., the CHO-K1 cell line. One or more expression plasmids can be introduced that encode heavy and light chain sequences. For example, in one embodiment, an expression plasmid encoding a heavy chain disclosed herein, and an expression plasmid encoding a light chain disclosed herein are transfected into host cells. The expression plasmids can be introduced as linearized plasmids at a ratio of 1:1 in the CHO-K1 host cell line using reagents such as Freestyle Max reagent. Fluorescence-activated cell sorting (FACS) coupled with single cell imaging can be used as a cloning method to obtain a production cell line.

A host cell transfected with an expression vector encoding an anti-GAL-3 antibody of the present disclosure, or fragment thereof, can be cultured under appropriate conditions to allow expression of the polypeptide to occur. The polypeptides may be secreted and isolated from a mixture of cells and medium containing the polypeptides. Alternatively, the polypeptide may be retained in the cytoplasm or in a membrane fraction and the cells harvested, lysed, and the polypeptide isolated using a desired method.

In some embodiments, an anti-GAL-3 antibody of the present disclosure can be produced by in vitro synthesis (see, e.g., Sutro Biopharma biochemical protein synthesis platform).

The present disclosure further provides a kit that comprises the antibody, the polypeptide, the polynucleotide, the expression vector, and/or the cell described herein.

### Compositions

In one aspect, the present disclosure provides a composition comprising the antibody described herein. For examples, the composition comprises an anti-GAL-3 antibody comprising a heavy chain variable (V_{H}) region comprising a HCDR1 amino acid sequence at least 70% identical to any one of SEQ ID NOS: 1-21 and 481-507, a HCDR2 amino acid sequence at least 70% identical to any one of SEQ ID NOS: 22-42 and 390-417, and/or a HCDR3 amino acid sequence at least 70% identical to any one of SEQ ID NOS: 43-63 and 418-439, and/or a light chain variable (V_{L}) region comprising: a LCDR1 amino acid sequence at least 70% identical to any one of SEQ ID NOS: 169-189 and 440-462, a LCDR2 amino acid sequence at least 70% identical to any one of SEQ ID NOS: 190-210 and 463-480, and/or a LCDR3 amino acid sequence at least 70% identical to any one of SEQ ID NOS: 211-231 and 481-507. The compositions can additionally contain other therapeutic agents that are suitable for treating or preventing a given disorder.

In another aspect, the present disclosure provides a composition comprising the immunoconjugate described herein, wherein the immunoconjugate comprises an anti-anti-GAL-3 antibody conjugated or linked to therapeutic, imaging/detectable moieties, or enzymes.

In another aspect, the present disclosure provides a pharmaceutical composition. In some embodiments, the pharmaceutical composition comprises any of the compositions described herein and a pharmaceutically acceptable carrier. For example, the pharmaceutical composition may comprise an anti-GAL-3 antibody comprising a heavy chain variable (V_{H}) region comprising a HCDR1 amino acid sequence at least 70% identical to any one of SEQ ID NOS: 1-21 and 481-507, a HCDR2 amino acid sequence at least 70% identical to any one of SEQ ID NOS: 22-42 and 390-417, and/or a HCDR3 amino acid sequence at least 70% identical to any one of SEQ ID NOS: 43-63 and 418-439, and/or a light chain variable (V_{L}) region comprising: a LCDR1 amino acid sequence at least 70% identical to any one of SEQ ID NOS: 169-189 and 440-462, a LCDR2 amino acid sequence at least 70% identical to any one of SEQ ID NOS: 190-210 and 463-480, and/or a LCDR3 amino acid sequence at least 70% identical to any one of SEQ ID NOS: 211-231 and 481-507. The pharmaceutically acceptable carrier can enhance or stabilize the composition, or to facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition as described herein can be administered by a variety of methods known in the art. The route and/or mode of administration vary depending upon the desired results. In some embodiments, the composition is sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohol such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions described herein can be prepared in accordance with methods well known and routinely practiced in the art. Pharmaceutically acceptable carriers are determined in part by the particular composition being administered, as well as by the particular method used to administer the composition. Accordingly, there is a wide variety of suitable formulations of pharmaceutical compositions. Applicable methods for formulating the compositions and determining appropriate dosing and scheduling can be found, for example, in Remington: The Science and Practice of Pharmacy, 21^{st} Ed., University of the Sciences in Philadelphia, Eds., Lippincott Williams & Wilkins (2005); and in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, and Sustained and Controlled Release Drug Delivery Systems, J. R. Robinson, ed., Marcel Dekker, Inc., New York, 1978, each of which are hereby incorporated herein by reference. Pharmaceutical compositions are preferably manufactured under GMP conditions. Typically, a therapeutically effective dose or efficacious dose is employed in the pharmaceutical compositions described herein. The compositions can be formulated into pharmaceutically acceptable dosage forms. Dosage regimens are adjusted to provide the desired response (e.g., a therapeutic response). In determining a therapeutically or prophylactically effective dose, a low dose can be administered and then incrementally increased until a desired response is achieved with minimal or no undesired side effects. For example, a single bolus may be administered, several divided doses may be administered over time or the dose may be proportionally reduced or increased as indicated by the exigencies of the therapeutic situation. It is especially advantageous to formulate parenteral compositions in dosage unit form for case of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

### Method of Treatment

In one aspect, the present disclosure provides a method for preventing or treating epilepsy and/or a neurological disorder in a subject in need thereof, the method comprising administering to the subject an antibody comprising a means for binding to Galectin-3. In some embodiments, the Galectin-3 is human Galectin-3. In some embodiments, the method comprising administering to the subject an anti-GAL-3 antibody, wherein the antibody binds to GAL-3 in the subject thereby preventing or treating epilepsy and/or a neurological disorder. In some embodiments, the neurological disorder is Alzheimer's disease (AD) or Parkinson's disease (PD). In some embodiments, the antibody is administered in combination with an additional therapeutic agent.

In certain embodiments, an antibody described herein is administered to the subject in combination with one or more additional therapeutic agents used to treat epilepsy and/or a neurological disorder, or the side effects or associated symptoms thereof. In certain embodiments, the method comprises administering to a subject a therapeutically effective amount of an antibody as disclosed herein, or a pharmaceutical composition comprising the antibody, in combination with a therapeutically effective amount of one or more additional therapeutic agents. In one embodiment, a method for treating epilepsy and/or a neurological disorder in a subject, the method comprising administering to the subject a therapeutically effective amount of an antibody as disclosed herein, or a pharmaceutical composition comprising the antibody, in combination with a therapeutically effective amount of one or more additional therapeutic agents.

In some embodiments, the antibody or pharmaceutical composition is administered to the subject in combination with one or more additional therapeutic agents that are effective at preventing or treating epilepsy and/or a neurological disorder. In some embodiments, the one or more additional therapeutic agents comprise another anti-Gal-3 antibody that does not compete with the antibody or pharmaceutical composition for binding to Gal-3. In some embodiments, the co-administration includes two or more anti-Gal-3 antibodies disclosed herein, with each antibody selected from a different community cluster listed in Table 10. For example, the antibodies may be selected from any two or more community clusters, including community 1 (Com 1), Com 2, Com 3, Com 4, Com 5, Com 6, Com 7, Com 8, Com 9, Com 10, Com 11, Com 12, Com 13, Com 14, Com 15, Com 16, Com 17, and Com 18 in Table 10. Since each antibody is selected from a different community cluster and binds to a distinct Gal-3 epitope, they do not compete with one another.

### Diseases

The anti-GAL-3 antibodies disclosed herein can be used to prevent or treat epilepsy or neurological disorders. In some embodiments, the neurological disorder is Alzheimer's disease (AD) or Parkinson's disease (PD).

### Epilepsy and Drug-Resistant Epilepsy

In some embodiments, the anti-GAL-3 antibodies disclosed herein can be used to prevent or treat Epilepsy. Epilepsy is a neurological disorder characterized by recurrent unprovoked seizures. It affects approximately 1% of the global population and presents a significant burden of disease worldwide. For about 30-40% of patients, seizures cannot be effectively managed with current drug therapies, leading to a condition known as drug-resistant epilepsy (DRE). Surgical interventions offer relief to some patients, but a substantial number continue to experience seizures even after surgery.

Epileptogenesis is associated with an increased and persistent inflammatory state in the microenvironment of neural tissues, which can lead to the production of cytokines by glial cells and neurons. The anti-Gal-3 antibodies disclosed herein, by inhibiting Gal-3 activity or expression, can mitigate neuroinflammatory responses, potentially alleviating the pathological processes that contribute to epilepsy development and drug resistance. In some embodiments, the anti-Gal-3 antibodies can be used to treat drug-resistant epilepsy.

### Parkinson's Disease

In some embodiments, the anti-GAL-3 antibodies disclosed herein can be used to prevent or treat Parkinson's disease (PD). Parkinson's disease (PD) is the second most prevalent neurodegenerative disease in the world. It is characterized by intense neurodegeneration in the basal ganglia area leading to severe and progressive motor impairment. The presence of Lewy bodies (LBs), described as neuronal intracytoplasmic deposits of α-synuclein (αSYN), is the second hallmark of the disease. While dopaminergic neurons from the substantia nigra (SN) are frequently the most affected cells, neurodegeneration and LB formation commonly appear in other central and enteric nervous system locations even years before than in the SN.

Disease modifying therapies are being developed that work either through targeting αSYN, inflammation, metabolism, vasculature and/or neuroplasticity. Galectin-3 (GAL3) is associated with LB formation and toxicity. As such, an anti-GAL-3 antibody disclosed herein can be used for therapeutical treatment to prevent or slow down PD progression.

### Alzheimer's Disease

In some embodiments, the anti-GAL-3 antibodies disclosed herein can be used to prevent or treat Alzheimer's disease (AD). Alzheimer's disease (AD) is a chronic progressive neurodegenerative disorder that is the leading cause of dementia among older adults. There are two major pathological hallmark proteins associated with toxic changes in the brain that may lead to AD: AB and tau. The disease is characterized by accumulation of extracellular amyloid plaques, intracellular neurofibrillary tangles, and extensive synaptic loss leading to progressive cognitive impairment and eventually dementia. Abnormal tau (hyperphosphorylated tau) accumulates to form neurofibrillary tangles, while Aβ plaques accumulate both extracellular and intracellular. Amyloid plaques and hyperphosphorylated tau in the brain are therefore the major hallmarks of AD.

Gal-3 is highly upregulated in the brains of AD patients. As such, an anti-GAL-3 antibody disclosed herein can be used for therapeutical treatment to prevent or slow down AD progression. In some embodiments, the anti-GAL-3 antibody disclosed herein enhances Aβ oligomerization. In some embodiments, the anti-GAL-3 antibody disclosed herein reduces and/or degrades the formation of neurotoxic conformational oligomers, such as Aβ₄₂ oligomers. In some embodiments, the anti-GAL-3 antibody disclosed herein reduces neuroinflammation and/or AD-related pathology in a subject. In some embodiments, the anti-GAL-3 antibody disclosed herein enhances cognitive memory function in a subject.

In particular embodiments, the method for preventing or treating epilepsy and/or a neurological disorder in a subject, comprises administering to the subject an effective amount of the pharmaceutical composition comprising an antibody which comprises a heavy chain variable (V_{H}) region comprising a HCDR1 amino acid sequence at least 70% identical to any one of SEQ ID NOS: 1-21 and 481-507, a HCDR2 amino acid sequence at least 70% identical to any one of SEQ ID NOS: 22-42 and 390-417, and/or a HCDR3 amino acid sequence at least 70% identical to any one of SEQ ID NOS: 43-63 and 418-439, and/or a light chain variable (V_{L}) region comprising: a LCDR1 amino acid sequence at least 70% identical to any one of SEQ ID NOS: 169-189 and 440-462, a LCDR2 amino acid sequence at least 70% identical to any one of SEQ ID NOS: 190-210 and 463-480, and/or a LCDR3 amino acid sequence at least 70% identical to any one of SEQ ID NOS: 211-231 and 481-507. In some embodiments, the pharmaceutical composition further comprises one or more therapeutic agent(s), wherein the therapeutic agent(s) can be either conjugated to the antibody or in a separate form apart from the antibody.

In another aspect, the disclosure additionally provides methods of identifying subjects who are candidates for treatment with an anti-GAL-3 antibody. Thus, in one embodiment, the disclosure provides a method of identifying a subject who can benefit from treatment with the anti-GAL-3 antibody disclosed herein. In one embodiment, the subject has epilepsy and/or a neurological disorder that overexpresses Galectin-3. Binding of the antibody to Galectin-3 can be measured using any assay, such as immunohistochemistry or flow cytometry. In some embodiments, binding of the antibody to at least 0.2%, 0.5%, or 1%, or at least 5% or 10%, or at least 20%, 30%, or 50%, of Galectin-3 in a sample may be used as a selection criterion for determining a patient to be treated with the anti-GAL-3 antibody as described herein.

In one aspect, methods of the disclosure comprise administering an anti-GAL-3 antibody disclosed herein, or a variant thereof, as a pharmaceutical composition to a patient in a therapeutically effective amount using a dosing regimen suitable for treatment of the epilepsy, the neurological disorder or the cancer. The composition can be formulated for use in a variety of drug delivery systems. One or more physiologically acceptable excipients or carriers can also be included in the compositions for proper formulation. Suitable formulations for use in the present invention are found, e.g., in Remington: The Science and Practice of Pharmacy, 21^{st} Edition, Philadelphia, PA. Lippincott Williams & Wilkins, 2005.

The anti-GAL-3 antibody is provided in a solution suitable for administration to the patient, such as a sterile isotonic aqueous solution for injection. The antibody is dissolved or suspended at a suitable concentration in an acceptable carrier. In some embodiments the carrier is aqueous, e.g., water, saline, phosphate buffered saline, and the like. The compositions may contain auxiliary pharmaceutical substances as required to approximate physiological conditions, such as pH adjusting and buffering agents, tonicity adjusting agents, and the like.

### Administration

The pharmaceutical compositions are administered to a patient in an amount sufficient to cure or at least partially arrest the disease or symptoms of the disease and its complications. An amount adequate to accomplish this is defined as a “therapeutically effective dose.” A therapeutically effective dose is determined by monitoring a patient's response to therapy. Typical benchmarks indicative of a therapeutically effective dose includes the amelioration of symptoms of the disease in the patient. Amounts effective for this use will depend upon the severity of the disease and the general state of the patient's health, including other factors such as age, weight, gender, administration route, and the like Single or multiple administrations of the antibody may be administered depending on the dosage and frequency as required and tolerated by the patient. In any event, the methods provide a sufficient quantity of anti-GAL-3 antibody to effectively treat the patient.

An anti-GAL-3 antibody or the antibody immunoconjugate (e.g., an anti-GAL-3 antibody-drug conjugate) can be administered by any suitable means, including, for example, parenteral, intrapulmonary, and intranasal, administration, as well as local administration, such as intratumor administration. Parenteral infusions include intramuscular, intravenous, intraarterial, intraperitoneal, or subcutaneous administration. In some embodiments, the antibody may be administered by insufflation. In an illustrative embodiment, the antibody or the antibody immunoconjugate (e.g., an anti-GAL-3 antibody-drug conjugate) may be stored at 10 mg/ml in sterile isotonic aqueous saline solution for injection at 4° C. and is diluted in either 100 ml or 200 ml 0.9% sodium chloride for injection prior to administration to the patient. In some embodiments, the antibody or the antibody immunoconjugate (e.g., an anti-GAL-3 antibody-drug conjugate) is administered by intravenous infusion over the course of 1 or 2 hours at a dose of between 0.01 and 100 mg/kg per the subject's body weight. In some embodiments, the antibody or the antibody immunoconjugate (e.g., an anti-GAL-3 antibody-drug conjugate) is administered at a dose range between 0.01-1 mg/kg, between 1-10 mg/kg, between 10⁻⁵⁰ mg/kg, or between 50-100 mg/kg per the subject's body weight. In some embodiments, the antibody or the antibody immunoconjugate (e.g., an anti-GAL-3 antibody-drug conjugate) is administered at a dose of about 10, 20, 30, 40, 50, 60, 70 mg/kg or a dose in a range defined by any two of the aforementioned values per the subject's body weight. In some embodiments, the antibody or the antibody immunoconjugate (e.g., an anti-GAL-3 antibody-drug conjugate) is administered at a dose of about 10, 20, 40, and 70 mg/kg or a dose in a range defined by any two of the aforementioned values per the subject's body weight. In other embodiments, the antibody or the antibody immunoconjugate (e.g., an anti-GAL-3 antibody-drug conjugate) is administered by intravenous infusion over a period of between 15 minutes and 2 hours. In still other embodiments, the administration procedure is via sub-cutaneous bolus injection.

The dose of antibody is chosen to provide effective therapy for the patient and is in the range of less than 0.01 mg/kg body weight to about 100 mg/kg body weight or in the range 1 mg-8 g per patient. Preferably the dose is in the range 0.1-50 mg/kg or approximately 50 mg-5000 mg/patient. The dose may be repeated at an appropriate frequency which may be in the range once per day to once every three months, or every six months, depending on the pharmacokinetics of the antibody (e.g., half-life of the antibody in the circulation) and the pharmacodynamic response (e.g., the duration of the therapeutic effect of the antibody). In some embodiments, the in vivo half-life of between about 7 and about 25 days and antibody dosing is repeated between once per week and once every 3 months or once every 6 months. In other embodiments, the antibody is administered approximately once per month.

In some embodiments, the antibody is administered more than once, such as two, three, four, or more times. In some embodiments, each administration is at least 7 days apart. In some embodiments, each administration is at least 14 days apart. In some embodiments, each administration is at least four weeks apart.

In an illustrative embodiment, the antibody may be stored at 10 mg/ml or 20 mg/ml in a sterile isotonic aqueous solution. The solution can comprise agents such as buffering agents and stabilizing agents. For example, in some embodiments, a buffering agent such as histidine is included to maintain a formulation pH of about 5.5. Additional reagents such as sucrose or alternatives can be added to prevent aggregation and fragmentation in solution and during freezing and thawing. Agents such as polysorbate 80 or an alternative can be included to lower surface tension and stabilizes the antibody against agitation-induced denaturation and air-liquid and ice-liquid surface denaturation. In some embodiments, the solution for injection is stored at 2-8° C. and is diluted in either 100 ml or 200 ml 0.9% sodium chloride for injection prior to administration to the patient. In some embodiments, the antibody is administered as a one to two-hour IV infusion from a diluted saline bag with a total volume of 100, 250, or 500 mL, depending on dose level.

### IV. Examples

The present disclosure will be described in greater detail by way of specific examples. The following examples are offered for illustrative purposes only and are not intended to limit the invention in any manner. Those of skill in the art will readily recognize a variety of noncritical parameters which can be changed or modified to yield essentially the same results.

### Example 1. Cross Species Reactivity of SIF001 Binding with Galectin-3 (GAL-3)

This example illustrates that the in vitro cross-species reactivity of SIF001 against GAL-3 from different species.

SIF001 is a humanized monoclonal antibody raised against the human GAL-3 protein. It has the HCDR sequences of SEQ ID Nos: 4, 25, and 46, and the LCDR sequences of SEQ ID Nos: 172, 193, and 214. The cross-species reactivity of SIF001 against GAL-3 from different species: human, mouse, rat, and cynomolgus monkey (cyno) samples was evaluated in vitro, and the affinity of GAL-3 binding to SIF001 was measured by Surface Plasmon Resonance (SPR). The binding affinity of SIF001 to human, mouse, and rat GAL-3, measured by dissociation constant (KD), is presented in Table 8 below.

TABLE 8
Binding Affinity of SIF001 to GAL-3

| | Human GAL-3 | Mouse GAL-3 | Rat GAL-3 |
|---|---|---|---|
| KD | 7.15 × 10⁻⁸M | 1.09 × 10⁻⁸M | 1.17 × 10⁻⁸M |

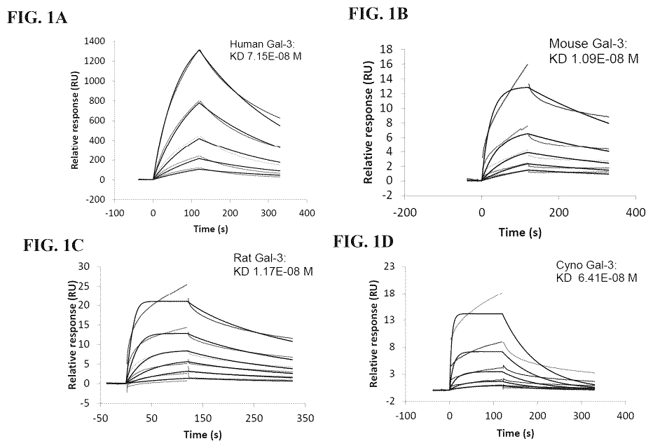

The curve fitting analysis demonstrated strong binding affinity in human, mouse, and rat samples, as evidenced by well-fitted curves (FIGS. 1A-1C). However, the curve fitting did not perform as well in monkey samples (FIG. 1D), suggesting a lack of binding affinity or potential species-specific differences in GAL-3 recognition by SIF001. These results indicate that while the SIF001 effectively binds to GAL-3 in human, mouse, and rat samples, its binding affinity may not extend to monkey GAL-3 or may be reduced in monkeys compared to other species.

### Example 2. SIF001 Reduced Inflammatory Cytokine TNFα Release in LPS-Stimulated Microglia BV2 Cells

This example illustrates the in vitro effect of SIF001 on inflammatory cytokine TNFα release in microglia BV2 cells when stimulated by lipopolysaccharide (LPS).

The inhibitory effects of SIF001 on the release of the inflammatory cytokine TNFα were evaluated in vitro in microglia BV2 cells stimulated by lipopolysaccharide (LPS) to mimic neuroinflammatory processes that are implicated in epileptogenesis and seizure activity. An ELISA assay was performed with the protocol shown below.
1. On the first day, seed BV2 cells into a 96-well plate, with 5,000 cells per well. Incubate at 37° C. with 5% CO2 overnight to allow the cells to adhere to the well.
2. On the second day, use a new 96-well plate with LPS concentration at 30 ug/ml. The 8th row of the 96-well plate serves as the control and contains only complete culture medium without LPS. All other wells contain 30 ug/ml LPS with a volume of 200 ul each.
3. Take two antibodies labeled as Isotype control, mSIF001. Set up 3 replicates for each antibody. The concentration for the first row of antibodies is 100 nM with a volume of 300 ul. Thoroughly mix the replicates containing the antibody, take 100 ul from it and add to the second row, mix well, and then take 100 ul from the second row to the third, continuing this process down to the seventh row, thus performing a 3-fold dilution.
4. After mixing, remove the complete culture medium from the original 96-well plate containing BV2 cells, wash 2-3 times with complete culture medium, 100 ul each time, and transfer all the liquid from step “3” into the BV2 cell plate. Incubate for 14-15 hours in the incubator.
5. After incubation, take 20 ul of culture medium from each well and dilute it 5 times with the sample diluent from a mouse TNF-α ELISA Kit (High-sensitive) (Beyotime, China). Store the remainder at 4° C.
6. Bring the reagents from the kit to room temperature to equilibrate for 20-30 minutes before use. Prepare the wash buffer by diluting the 20× wash buffer with sterile water to 1×.
7. Add 100 μl of the sample to the respective wells, seal the reaction wells with sealing film, and incubate at room temperature for 120 minutes. After incubation, manually wash the plate, adding 200 ul of wash buffer to each well, for a total of 5 washes. Tap dry on thick absorbent paper after the last wash.
8. Add 100 ul of biotinylated antibody to each well, seal the reaction wells with sealing film, and incubate at room temperature for 60 minutes. Repeat the washing step as in step 7.
9. Add 100 ul of Streptavidin-HRP to each well, seal the reaction wells with sealing film, and incubate at room temperature in the dark for 20 minutes. If the room temperature is low (below 25° C.), extend the incubation time as needed. Repeat the washing step as in step 7.
10. Add 100 μl of TMB substrate solution to each well, seal the reaction wells with sealing film, and incubate in the dark at room temperature for 5-10 minutes until a significant color change is observed in the samples. Add 50 ul of stop solution to each well, mix well, and immediately measure the absorbance at 450 nm.

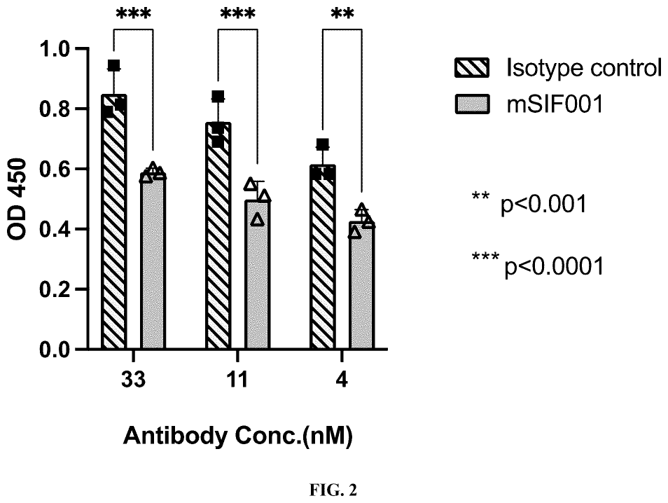

Results demonstrated that SIF001 led to a significant decrease in TNF-α release from LPS-stimulated microglia BV2 cells (FIG. 2). Given the proposed role of neuroinflammation in epilepsy, these findings suggest the potential therapeutic efficacy of SIF001 in mitigating inflammatory responses associated with epileptic pathogenesis, thus highlighting its promise as a treatment for epilepsy.

### Example 3. SIF001 Reduced Inflammation in Kainic Acid (KA)-Stimulated Microglia BV2 Cells

This example illustrates the in vitro effect of SIF001 on inflammation in microglia BV2 cells stimulated by kainic acid (KA).

In this study, the effect of SIF001 on kainic acid (KA)-stimulated microglia BV2 cells was investigated using cell immunofluorescence assays, as the protocol shown below.
1. On the first day, BV2 cells were first planted in three 35 mm glass bottom dishes with 10,000 cells seeded in each dish. The incubator was incubated at 37° C. and cultured overnight with 5% carbon dioxide to make the cells stick to the wall.
2. On the second day, anti-HEL Mouse IgG2A isotype control antibody and SIF001 were taken for 100 nM each, and each antibody was added to a petri dish and incubated at 37° C. in a carbon dioxide incubator for 1 h. The control group was replaced with 1 ml complete medium. After incubation, 200 ug/ml Kainic acid (KA) solution was added to the two treatment group petri dishes. After incubation for 24 h, the cells in 3 glass dishes were stained by immunofluorescence.
3. In a fuming cupboard, brain tissue was fixed at room temperature for 15 min with 4% PFA thawed at room temperature.
4. Transfer the PFA to the special waste liquid bucket, and wash the section tissue with PBS three times, 10 min each time.
5. A blocking solution was prepared and diluted in PBS with 5% goat serum (NGS)+0.5% Triton X-100. After removing PBS, add blocking solution and incubate at room temperature for 1 h.
6. The primary antibody was prepared, diluted in PBS with 5% goat serum+0.1% Triton X-100, added with 1:250 Iba1 primary antibody, and incubated at 4° C. overnight.
7. Wash with PBS three times in the morning of the next day, 10 min each time.
8. The secondary antibody was prepared, diluted in PBS with 5% goat serum+0.1% Triton X-100, added with 1:250 secondary antibody, and incubated at room temperature away from light for 2 h.
9. Discard the secondary antibodies, prepare 5 ug/mL DAPI on PBS for nuclear staining, and incubate at room temperature for 15 min away from light.
10. Dry in a dark place and then add antifade mounting medium with DAPI. Add about 150 uL of the sealing liquid to each slide and cover it with a cover glass to avoid bubbles. After drying in the dark, Liquid blocker super PAP pen can be sealed on all sides and stored at 4° C. in a cassette.

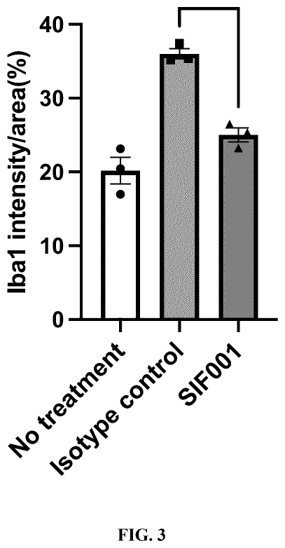

The results from cell immunofluorescence assay (FIG. 3) confirmed that, compared to the vehicle group of microglia BV2 cells without receiving stimulation (no treatment), the proportion of IBA1-stained cells in the KA stimulated microglia cells treated with human HEL (isotype control) significantly increased and the cell morphology enlarged, while the proportion of IBA1-stained cells significantly reduced in the KA stimulated microglia BV2 cells treated with SIF001 (SIF001). These data indicate that SIF001 significantly reduced expression of activation marker IBA1 in in vitro epilepsy model of microglia BV2 cells stimulated by kainic acid (KA). While KA stimulation significantly increased the proportion of IBA1-stained cells and induced morphological changes, indicative of microglial activation, the treatment with SIF001 resulted in a significant reduction in the proportion of IBA1-stained cells in KA-stimulated microglia BV2 cells, suggesting a potential inhibitory effect of SIF001 on microglial activation induced by KA. These findings highlight the therapeutic potential of SIF001 in modulating microglial activation and neuroinflammation, which are implicated in the pathophysiology of conditions such as epilepsy.

### Example 4. SIF001 Reduced Epilepsy in Mouse Model Induced by Kainic Acid (KA)

This example illustrates the effect of SIF001 on epilepsy induced by kainic acid (KA) in C57BL/6J mice.

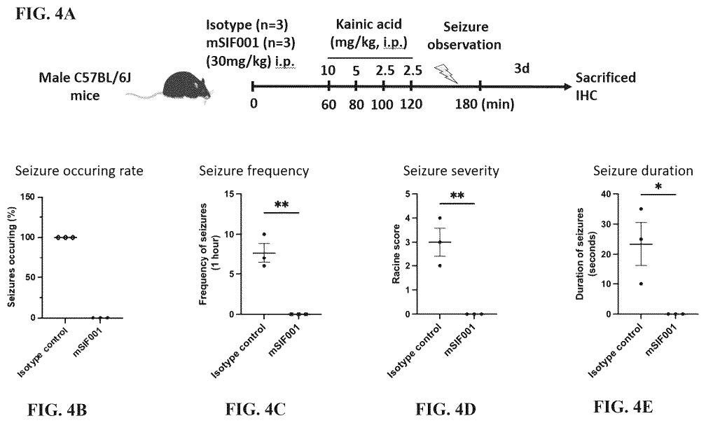

The effect of SIF001 on the reduction of epilepsy symptoms was evaluated in 8-12 weeks male C57BL/6J mice. As illustrated in FIG. 4A, epilepsy was induced in C57BL/6 mice by administering 10 mg/kg, 5 mg/kg, 2.5 mg/kg, and 2.5 mg/kg kainic acid (KA) via i.p. injection at 60 min, 80 min, 100 min, 120 min, respectively. Before KA injection, mice were randomized into two groups to receive 30 mg/kg anti-HEL mouse IgG2A (control group) or receive 30 mg/kg mSIF001 i.p. injection at 0 min (active group). All mice were observed for 72 hours prior to the sacrifice.

Brain tissue processing for staining: Briefly, animals were deeply anesthetized with 50 mg/kg Zoletil (i.p.) and transcardially perfused with phosphate buffered saline (PBS) followed by 4% paraformaldehyde (PFA) in PBS. Whole brains were removed and allowed to post-fix in 4% PFA for 6-8 h at 4° C. before being transferred to a 30% sucrose solution for overnight cryoprotection. Coronal sections containing the medial and ventral hippocampus were sliced on a freezing stage microtome and stored at 4° C. in 0.1M PBS for staining.

Iba1 and GFAP staining was performed as the protocol shown below.
1. In a fuming cupboard, brain tissue was fixed at room temperature for 15 min with 4% PFA thawed at room temperature.
2. Transfer the PFA to the special waste liquid bucket, and wash the section tissue with PBS three times, 10 min each time.
3. A blocking solution was prepared and diluted in PBS with 5% goat serum (NGS)+0.5% Triton X-100. After removing PBS, add blocking solution and incubate at room temperature for 1 h.
4. The primary antibody was prepared, diluted in PBS with 5% goat serum+0.1% Triton X-100, added with 1:250 Iba1 primary antibody, and incubated at 4° C. overnight.
5. Wash with PBS three times in the morning of the next day, 10 min each time.
6. The secondary antibody was prepared, diluted in PBS with 5% goat serum+0.1% Triton X-100, added with 1:250 secondary antibody, and incubated at room temperature away from light for 2 h.
7. Discard the secondary antibodies, prepare 5 ug/mL DAPI in PBS for nuclear staining, and incubate at room temperature for 15 min away from light.
8. Dry in a dark place and then add antifade mounting medium with DAPI. Add about 150 uL of the sealing liquid to each slide and cover it with a cover glass to avoid bubbles. After drying in the dark, Liquid blocker super PAP pen can be sealed on all sides and stored at 4° C. in a cassette.

Bioanalysis: Three brain tissue sections were taken from each sample for staining, and the hippocampus region was selected for 10× photography with Olympus FV3000. For analyses of microglia and astrogliosis, Iba1 and GFAP staining intensities, a one-way ANOVA design was utilized with a Dunnett's multiple comparison to controls. A p-value below 0.05 was considered significant in post-hoc testing. All statistical analyses were performed using Prism 9 (GraphPad, La Jolla, CA).

Therapeutic effects of SIF001 on the epilepsy model were evaluated in terms of seizure occurring rate (FIG. 4B) among the test animals in each group, seizure frequency (FIG. 4C) in individual mouse in each group, seizure severity (FIG. 4D) per Racine score and seizure duration (FIG. 4E) in individual mouse in each group. As shown in FIGS. 4A-4D, significant inhibition of epilepsy occurrence has been observed in the active group, in which no mice treated with SIF001 showed seizure symptoms, while all mice in the control group suffered frequently occurred seizures, an average severity Racine score as of 3, and commonly with more than 20 seconds seizure duration.

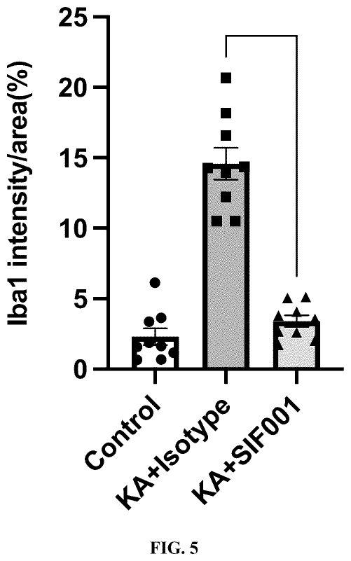

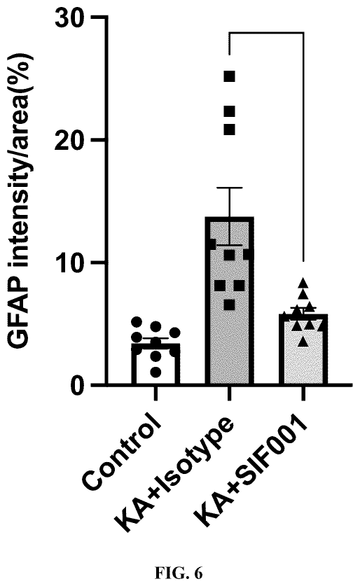

To further explore any pathological changes underlying epilepsy, brain tissues from isotype control and active groups, and a group without any treatment, were immune-fluorescent stained with IBA1 (ionized calcium-binding adapter molecule 1) and GFAP (glial fibrillary acidic protein), biomarkers for activation of microglia and astrocytes, respectively. As shown in FIGS. 5 and 6, SIF001 significantly reduced activation of microglia (FIG. 5), and astrocytes (FIG. 6), indicating that SIF001 inhibits epilepsy through disease modification. The results suggest that treatment with SIF001 led to a potential modulation of glial activation and neuroinflammation, which may contribute to the regulation of epileptic activity and neuroprotection in the hippocampus.

The results showed that, in KA-induced epilepsy model, treatment with SIF001 was able to significantly inhibit the occurrence of seizure in terms of Racine Score, frequency of seizures, duration of seizures, and significantly reduced neuroinflammatory activation of microglia and astrocytes.

### Example 5. SIF001 and SIF002 Sequence Analysis

This example illustrates alanine scanning and mutagenesis assays to evaluate essential amino acid residues of the anti-GAL-3 antibodies SIF001 and SIF002 for antigen binding affinity.

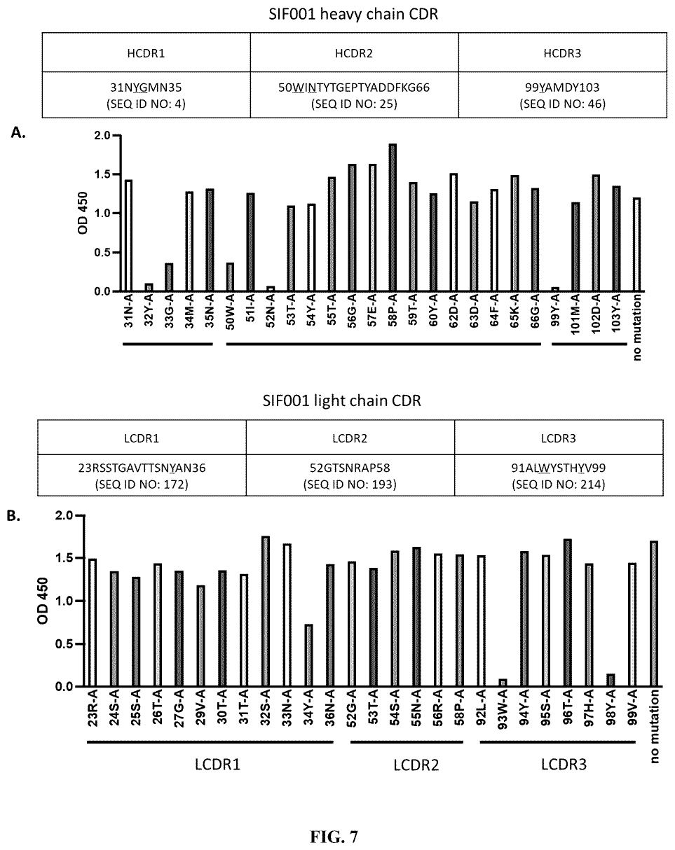

First, we utilized the Kabat numbering system to annotate the heavy chain CDRs of SIF001. As indicated in FIG. 7A, the HCDR1 (SEQ ID NO: 4) of SIF001 starts at position 31 and ends at position 35, the HCDR2 (SEQ ID NO: 25) starts at position 50 and ends at position 66, and the HCDR3 (SEQ ID NO: 46) starts at position 99 and ends at position 103. As indicated in FIG. 7B, the LCDR1 (SEQ ID NO: 172) of SIF001 starts at position 23 and ends at position 36, the LCDR2 (SEQ ID NO: 193) starts at position 52 and ends at position 58, and the LCDR3 (SEQ ID NO: 214) starts at position 91 and ends at position 99.

To determine which amino acids of the antibody are critical for antigen binding, we performed an alanine scanning assay in which each amino acid within the heavy and light chain CDR regions was individually replaced with an alanine and antigen binding affinity of each antibody variants were tested. We introduced an alanine mutation individually to each amino acid within both the heavy and light chain CDRs of SIF001, and then spliced the complete sequence of the human IgG4P subtype. We inserted the designed sequence into the pcDNA3.4 vector (Amp), extracted an ample amount of plasmid, and used an appropriate quantity of plasmid (concentration 500 ng/ul) for electroporation transfection into CHOK1 cells. The transfected cells were incubated at 37° C., 270 rpm, in an 8% CO2 environment, ensuring timely addition of supplements during the 4-day culture period. Subsequently, the cell supernatant was collected for protein expression assessment using Gator.

We evaluated the binding affinity of the SIF001 variants with an alanine mutation to human Galectin-3 through ELISA. As shown in FIG. 7A, a replacement of any one of the following amino acid residues of SIF001: Y32 and G33 in HCDR1, W50 and N52 in HCDR2; and Y99 in HCDR3 to an alanine resulted in a significant reduction of the antigen binding affinity, indicating that these amino acid residues are essential to the antibody SIF001 binding to the antigen GAL-3. As showed in FIG. 7B, a replacement of any one of the following amino acid residues of SIF001: Y34 in LCDR1, W93 and Y98 in LCDR3 to an alanine resulted in a significant reduction of the antigen binding affinity, indicating that these amino acid residues are essential to the antibody SIF001 binding to the antigen GAL-3.

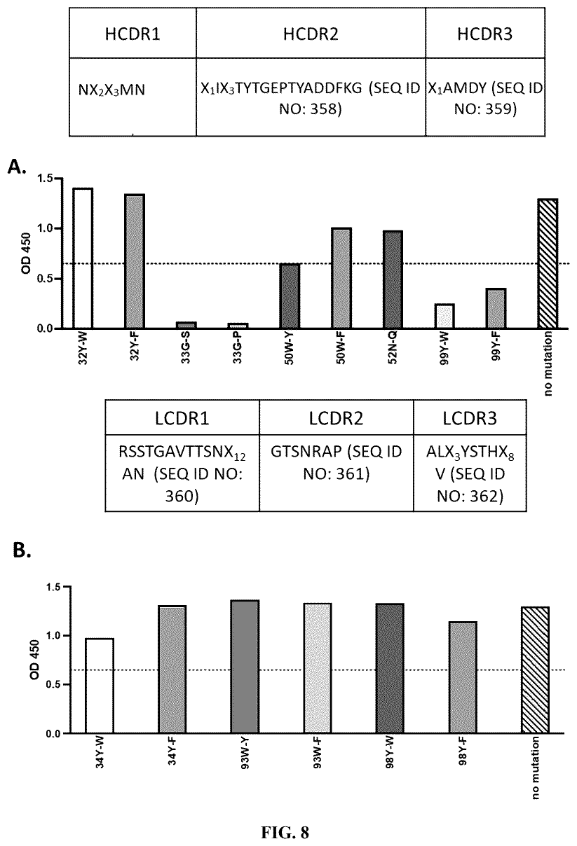

We further mutated these essential amino acids: Y32, G33, W50, N52, Y99 in the HCDR regions of SIF001 and Y34, W93, and Y98 in the LCDR regions of SIF001 to different amino acid residues, and concluded that a mutation of Y32W, Y32F, W50Y, W50F, or N52Q in the HCDR regions of SIF001 (FIG. 8A), or a mutation of Y34W, Y34F, W93Y, W93F, Y98W, or Y98F in the LCDR regions of SIF001 (FIG. 8B) did not impact antibody binding to the antigen GAL-3.

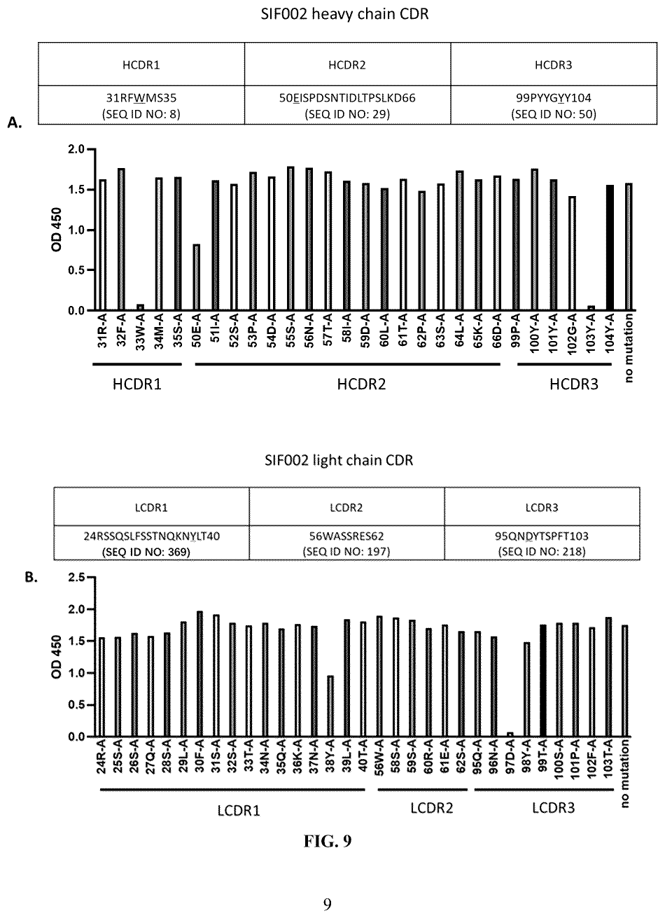

Similarly, we utilized the Kabat numbering system to annotate the heavy chain CDRs of SIF002. As indicated in the top table of FIG. 9, the HCDR1 (SEQ ID NO: 8) of SIF002 starts at position 31 and ends at position 35, the HCDR2 (SEQ ID NO: 29) starts at position 50 and ends at position 66, and the HCDR3 (SEQ ID NO: 50) starts at position 99 and ends at position 104. As indicated in the lower table of FIG. 9, the LCDR1 (SEQ ID NO: 369) of SIF002 starts at position 24 and ends at position 40, the LCDR2 (SEQ ID NO: 197) starts at position 56 and ends at position 62, and the LCDR3 (SEQ ID NO: 218) starts at position 95 and ends at position 103.

We evaluated the binding affinity of the SIF002 variants with an alanine mutation to human Galectin-3 through ELISA. As shown in FIG. 9A, a replacement of any one of the following amino acid residues of SIF002: Y33 in HCDR1, E50 in HCDR2; and Y103 in HCDR3 to an alanine resulted in a significant reduction of the antigen binding affinity, indicating that these amino acid residues are essential to the antibody SIF002's binding to the antigen GAL-3. As showed in FIG. 9B, a replacement of any one of the following amino acid residues of SIF002: Y38 in LCDR1 and D97 in LCDR3 to an alanine resulted in a significant reduction of the antigen binding affinity, indicating that these amino acid residues are essential to the antibody SIF002 binding to the antigen GAL-3.

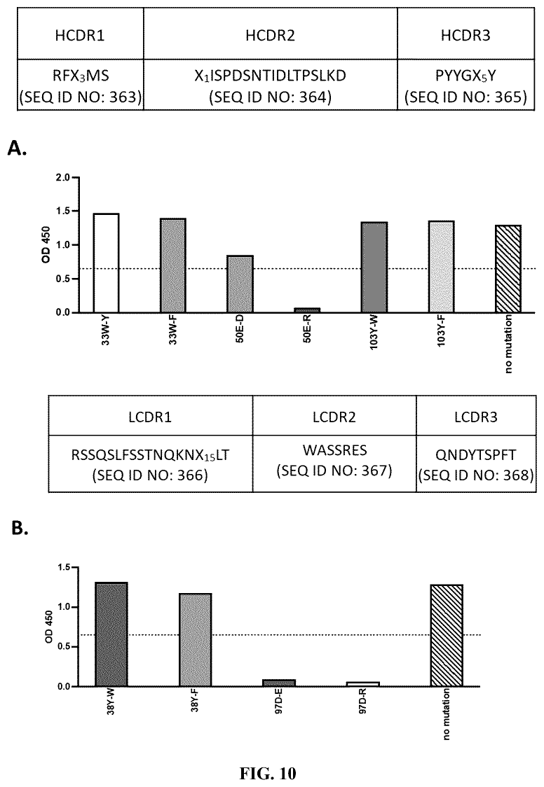

We further mutated these essential amino acids: Y33, E50, Y103 in the HCDR regions of SIF002 and Y38 and D97 in the LCDR regions of SIF002 to different amino acid residues, and concluded that a mutation of W33Y, W33F, E50D, Y103W, or Y103F in the HCDR regions of SIF002 (FIG. 10A), or a mutation of Y38W or Y38F in the LCDR regions of SIF002 (FIG. 10B) did not impact antibody binding to the antigen GAL-3.

### Example 6. SIF001 Prevented Seizure Occurring in a Dose-Dependent Manner

This example illustrates that the anti-GAL-3 antibody SIF001 can prevent seizure occurring in a dose-dependent manner in mice.

Using the same methods described in Example 4, the effects of SIF001 on preventing seizure were evaluated in 8-12 weeks male C57BL/6J mice. As measured in seizure frequency (FIG. 11A), seizure severity (FIG. 11B), and seizure duration (FIG. 11C), 3 mg/kg of SIF001 can significantly reduce seizure occurring in mice, where 10 mg/kg of SIF001 showed even better effect, indicating that anti-GAL-3 antibody SIF001 can prevent seizure occurring in a dose-dependent manner in mice.

### Example 7. Structure Determination of Galectin3-e (Gal3-e) and Fab Complex

This example illustrates utilizing cryogenic electron microscopy (Cryo-EM) to capture a high-resolution structure of a Gal3-e and Fab complex.

### Summary

The objective of this research is to obtain a high-resolution structure of the Gal3-e and Fab complex. The Gal3-e is a Gal-3 peptide with an amino acid sequence of GAPGAYPGAPAPGVYPGPPSGPGAYPSSG (SEQ ID NO: 508). X-ray diffraction and Cryo-EM Single particle analysis (SPA) are the most powerful biophysical methods to build the high-resolution structure of protein or protein complex. Initially, the X-ray diffraction technique was employed to elucidate the structure. However, no diffraction patterns were observed in the optimized large crystals. Consequently, cryogenic electron microscopy (Cryo-EM) was selected as the alternative approach to determine the structure of the complex.

For X-ray diffraction, first we developed a homogeneous Gal3-e-Fab complex through Size Exclusion Chromatography (SEC) and analyzed the complex through crystallization screening and optimization. However, no diffraction patterns but only large block- and rod-shaped crystals were observed by multi-rounds of optimization. Screened commercial kits included crystal screens, structure screens, Index, JCSG plus, PEG/Ion1&2, ProPlex screens, and additive screens.

For Cryo-EM, multiple Cryo-EM sample screening experiments were conducted, employing various grids and detergents to identify optimal conditions. We obtained the optimal Cryo-EM samples by using FGG grid exhibiting particles with high contrast and diverse orientations. Next, we collected more than 5 datasets on Talos Glacios 200 kV and produced a preliminary density map with a resolution of 5.2 Å. To further enhance the resolution, we collected a dataset, including 8, 447 movies, on Titan Krios 300 kV equipped with a Falcon 4i camera and an energy filter. This dataset enabled the resolution of the Gal3-Fab complex density map to 3.17 Å. We further performed initial model building and model refinement against the 3.17 Å density map. Eventually, the 15 amino residues of the Gal3-e peptide were traced within the density map, and 9 of them were identified.

### Methods

### Assembly of Gal3-e and Fab

Excess Gal3-e were incubated with the Fab on ice for 30 minutes. After the incubation, Gal3-e-Fab complex was isolated from the mixture using SEC (Column: Superdex 75 increase 10/300).

### Co-Crystallization

For the production of crystals, a solution of purified Gal3-e-Fab complex (15-30 mg/mL) in buffer containing 20 mM Tris pH7.5 and 150 mM NaCl was used. Crystals of the complex were grown at 18° C. using the sitting drop vapor diffusion technique by mixing equal amounts of protein solution and reservoir buffer containing the precipitating agents.

### Cryo-EM

### 1. Cryo-EM Sample Preparation

Apply 3 μL protein sample on the grid treated with Pelco EasiGlow discharge, with subsequent blotting leaving only a thin liquid film on the grid by using Vitrobot. The thin film is then vitrified, typically by plunging into liquid ethane.

### 2. Cryo-EM Sample Screening & Optimization

Proceed to load the Cryo-EM samples onto the Talos Glacios 200 kV and assess the sample behavior, specifically focusing on the contrast and orientations of particle. Typically, it is anticipated that several iterations of optimization will be requisite to obtain optimal Cryo-EM samples.

### 3. Data Collection on Talos Glacios 200 kV

Acquire datasets on optimal Cryo-EM samples, followed by image processing. Further evaluate the quality of datasets. If possible, obtaining a density map at medium resolution.

### 4. Data Collection on Titan Krios 300 kV

To increase the resolution of structure by using a 300 kV TEM with higher performance.

### 5. Image Processing & Model Building

Image processing is a multi-stage processing workflow, which encompassed initial pre-processing, followed by particle selection, 2D classification (two-dimensional classification), 3D classification (three-dimensional classification), 3D refinement (three-dimensional refinement), and subsequent local refinement. Multiple iterations of model construction and refinement were executed in against the acquired density map.

### Results

### Protein Assembly

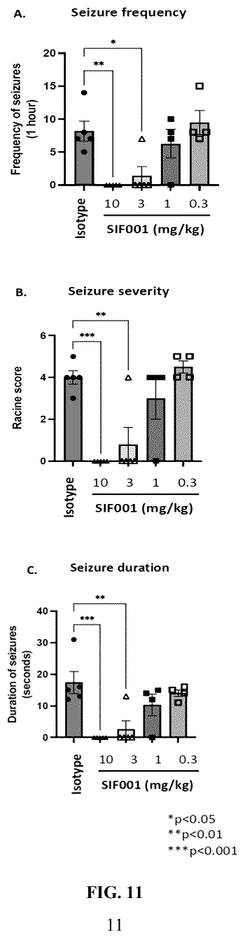

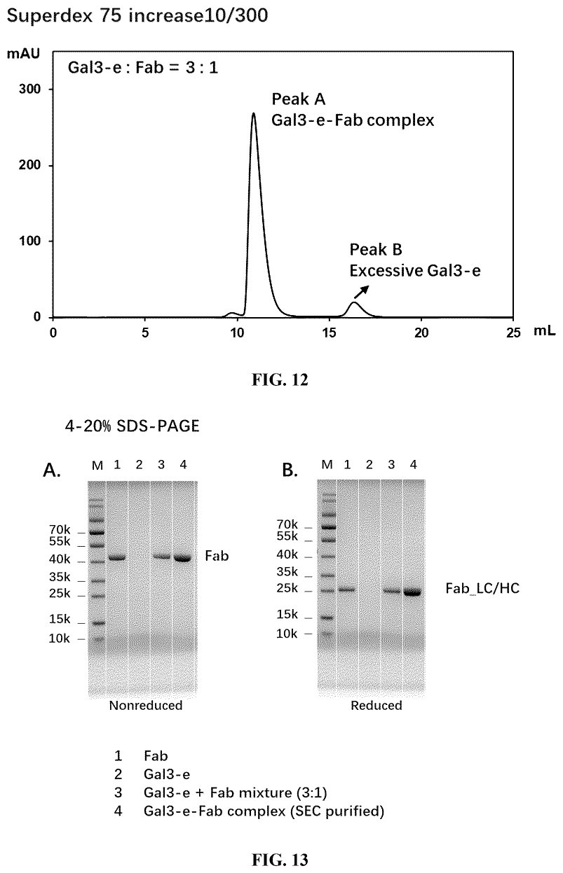

The results of SEC demonstrated that Gal3-e and Fab were successfully assembled into homogeneous binary complex (FIG. 12). It is hard to detect the Gal3-e peptide by using regular SDS-PAGE due to its small molecular weight (3 kDa) (FIGS. 13A and 13B).

We have performed multi-rounds of co-crystallization screening and optimization of Gal3-e-Fab complex, including co-crystallized with different concentrations of samples, screening about 700 conditions (Kits). The large block-shaped and rod-shaped crystals were observed by multi-rounds of optimization, but no diffraction was detected by using internal XRD or at SSRF (Shanghai Synchrotron Radiation Facility).

### Cryo-EM

To obtain optimal Cryo-EM samples, we have undertaken multi-rounds of sample screening and optimization, including adding types of detergent to increase diversity of particle orientation and using types of grids. Finally, we found that utilization of FGG grid (treated with Graphene) for sample preparation exhibits the best behaviors of particle with high contrast and diverse orientations. Then we collected 5 datasets of optimal Cryo-EM samples on Talos Glacios 200 kV. Obtained a density map of 5.7 Å (angstroms). The local density map of Gal3-e still could not be well identified due to the low resolution.

To further increase resolution, we prepared another batch of Cryo-EM samples and collected a dataset (8, 447 images) on Titan Krios 300 kV equipped with Falcon 4i and energy filter. Finally, we obtained a 3.17 Å map through delicate image processing.

### Model Building

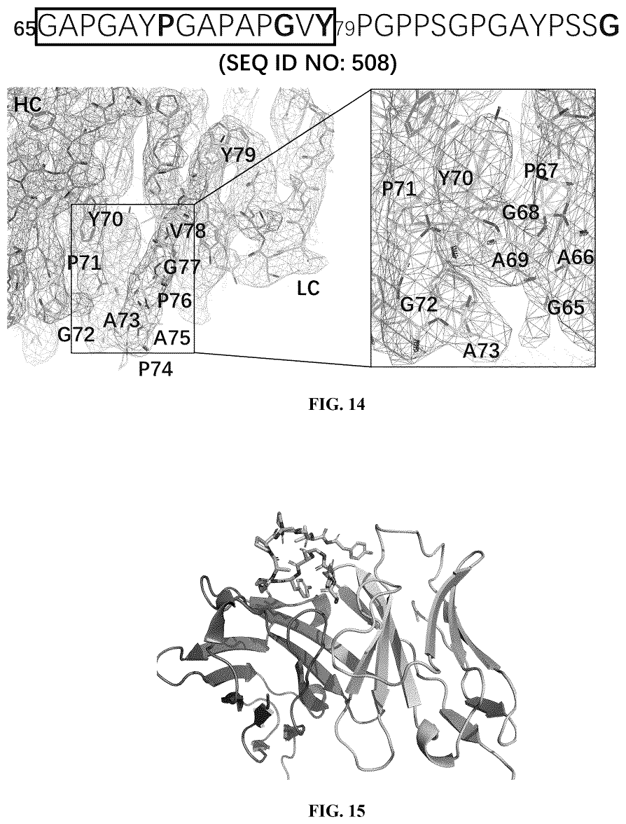

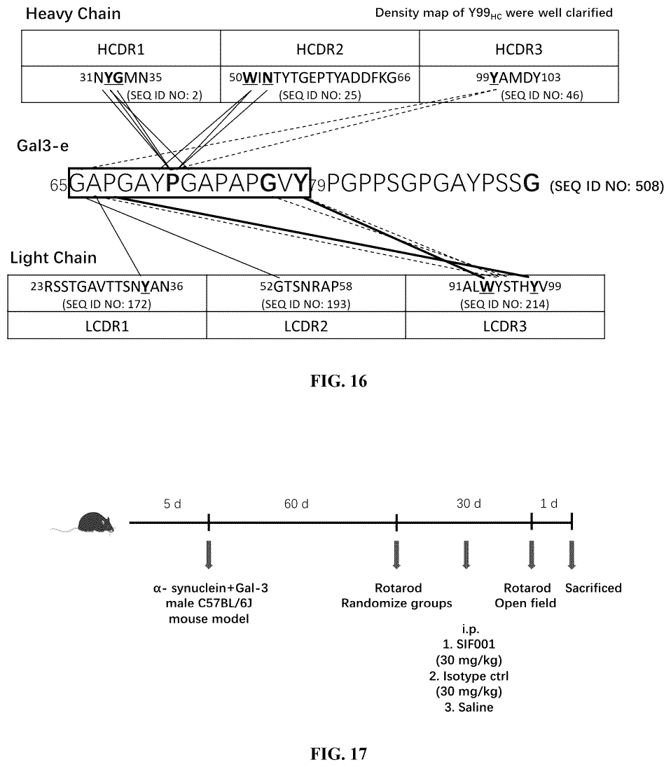

Two preliminary structural models of the Fab fragment were constructed utilizing the Swiss-Model (https://swissmodel.expasy.org) and Alphafold3 (https://alphafoldserver.com), respectively. Subsequently, these models were fitted into the electron density map at a resolution of 3.17 Å. Comparative analysis revealed that the model generated by Swiss-Model exhibited markedly higher precision. To enhance the accuracy of the model, iterative rounds of refinement were conducted against the density map employing the software tools Coot and Phenix. Within the density map, 15 amino residues of Gal3-e could be traced in the density map, 9 of these could be identified (FIG. 14). The interface and interactions between Gal3-e and Fab are shown in FIGS. 15 and 16.

### Example 8. SIF001 for Parkinson's Disease Treatment

This example illustrates therapeutic effects of SIF001 in a Parkinson's disease mimic mice model.

### Summary

The primary objective of this in vivo study is to assess the therapeutic effect of anti-Galectin-3 antibody (mSIF001) in reducing neuroinflammation and PD pathology in 10-week-old C57BL/6 male mice injected with αSYN oligomers. Amyloid protein conformational oligomers are neurotoxic in PD. In vitro, we found that Gal-3 intrinsically promotes oligomerization and that SF001 reduced or degraded αSYN oligomer formation. In order to mimic the PD pathology, αSYN oligomers intrinsically promoted by Galectin-3 was injected into the parenchyma of one hemisphere under stereotactic control. In order to investigate the effect of αSYN oligomers, mice under went the locomotor function test using rotarod. After 3 months, mice produced a statistically significant dysfunction as compared to age match control mice which is one of the phenotypic feature in PD. Mice were dosed with 30 mg/kg anti-HEL mouse IgG2A (isotype control group) or received 30 mg/kg mSIF001 (active group) via i.p. injection four weeks (2 doses/week). Treatment with anti-Galectin-3 antibody showed improvement in locomotor function and was statistically significant as compared to mice treated with Isotype control Ab. Mice were sacrificed for brain neuroinflammation analysis by immunohistochemistry (IHC) immunofluorescence (IF) staining. Results showed that, compared to the isotype antibody treatment, mSIF001 treatment significantly reduced underlying neuroinflammatory activation of microglia. In addition, mice dosed with mSF001 shows significant decrease in αSYN aggregates as compared to mice dosed with Isotype-control.

Parkinson's disease (PD) is the second most prevalent neurodegenerative disease in the world. It is characterized by intense neurodegeneration in the basal ganglia area leading to severe and progressive motor impairment. The presence of Lewy bodies (LBs), described as neuronal intracytoplasmic deposits of α-synuclein (αSYN), is the second hallmark of the disease. While dopaminergic neurons from the substantia nigra (SN) are frequently the most affected cells, neurodegeneration and LB formation commonly appear in other central and enteric nervous system locations even years before than in the SN. Disease modifying therapies are being developed that work either through targeting αSYN, inflammation, metabolism, vasculature and/or neuroplasticity. Of all the clinical trials conducted to date, none of these treatments have met clinical efficacy endpoints for PD. Thus, there is an urgent unmet medical need for treatments that can either stop or reverse the progression of PD. It has been observed prominent endogenous role for galectin-3 (GAL3) in pathological αSYN strains associated with LB formation and toxicity, thus pointing to GAL3 inhibition as a potential future therapeutical strategy to prevent or slow down PD progression

Galectin-3 (Gal-3) plays a pivotal role in microglia-mediated neuroinflammation causing many neurological disorders. A mouse model of PD showing an increase in αSYN aggregates and neuroinflammation was used to assess the effect of mSIF001, an anti-Gal-3 monoclonal antibody, on the severity of neuroinflammation and the pathological changes underlying the PD pathology.

### Methods and Experiments

Study Design. Study design is outlined in FIG. 17.

Preparation of Alpha synuclein oligomers. It has been observed Galectin-3 intrinsically promotes oligomers of amyloid prooteins. Alpha synuclein oligomers were prepared by incubating monomeric α-synuclein (300 μM) with and without Galectin-3 (100 ug/ml) in phosphate buffer pH 7.4 under stirring condition with a stir bar for 0-5 hrs. Alpha synuclein oligomers were characterized by dot blot and western blot using conformational oligomer specific antibody A11 and Alpha synuclein Ab Syn1.

Intrastriatal injection of recombinant human α-synuclein oligomers. At 10 weeks of age, male mice were deeply anesthetized with vaporized isoflurane on a stereotactic frame. Animals were bilaterally injected with 2 μL (per side) of 300 μM oligomeric α-synuclein intrinsically promoted by Galectin-3, or phosphate-buffered saline (PBS) as controls. Solutions were injected at a constant rate of 0.5 L/min and the needle left in place for 5 min followed by slowly with—drawing the needle. Coordinates for the striatum were +0.2 mm AP, ±2.0 mm ML, −2.6 mm DV. Two separate cohorts were utilized in this study. The first cohort had an experimental group injected with Alpha synuclein+Galectin-3 and a control group with bur hole only.

Behavior testing. Locomotor function test was conducted before and every month after injection of oligomers. Mice were handled for 3 days before testing began and habituated to testing room for 1 h at the start of each testing day. The order of test will be designed to minimize stress to the animals, such that low-stress tasks will be completed before high-stress tasks. Additionally, mice received at least one day rest between each test.

To further test motor activity, mice were placed on an accelerating rotarod apparatus, and time taken to fall was measured. Mice were trained on the apparatus over 2 days, with the rotarod speed increasing from 4 rpm to 40 rpm over 300 s. If mice had not fallen by 120 s on the testing trial, the rotation was halted, the mouse was removed, and the time was recorded as 120 s. Mice underwent three trials per day, with an inter-trial rest time of at least 30 min.

Animals and Husbandry. 10 weeks old C57BL/6J male mice (n=30) were used for experiments. Mice were on a 12-h light/dark cycle and had ad libitum access to food and water. Animals were housed in pairs in a controlled temperature environment under a 12-h light/dark cycle, with access to chow and water ad libitum. All animals were provided at least 72 h to acclimate following arrival at the facility, with periodic cage changes, before surgery.

Administration and Sample Collection. 10-week old C57BL/6J male mice mimicking PD pathology after stereotactic injection of αSYN oligomers were first administered with antibody treatments via intraperitoneal (i.p.) injection. Mice were dosed with 30 mg/kg anti-HEL mouse IgG2A (isotype control group) or received 30 mg/kg mSIF001 (active group) via i.p. injection four weeks (2 doses/week). After four weeks, locomotor function test was conducted to evaluate the efficacy of mSIF001Ab. Mice were sacrificed, and brain tissues were processed for histological staining.

Brain tissue processing. Briefly, animals were deeply anesthetized with 50 mg/kg Zoletil (i.p.) and transcardially perfused with phosphate buffered saline (PBS) followed by 4% paraformaldehyde (PFA) in PBS. Brain was then post-fix in 4% PFA for 24 hrs at 4° C. before being transferred to a 30% sucrose solution for overnight cryoprotection. Coronal sections containing the medial and ventral hippocampus were sliced into 25 μm thick slices on a freezing stage cryostat and stored at 4° C. in 0.1M PBS for staining.

Iba1 and αSYN Immunofluorescence staining. Brain sections were washed with PBS for 3 times, 10 min each time. Sections were then incubated with a blocking solution (PBS with 5% goat serum, 0.5% Triton X-100) at room temperature for 1 h. Tissues were incubated overnight with, IBA-1, and αSYN antibodies (1:250 diluted with the blocking solution) overnight at 4° C. After being washing with PBS three times in the morning of the next day, sections were incubated with the blocking solution containing 1:250 diluted secondary antibody for 2h. Brain sections were incubated with 5 μg/mL DAPI in PBS for nuclear staining for 15 minutes and mounted on to microscope slides to dry in a dark place and then were added with antifade mounting medium with DAPI for further analysis

Bioanalysis. Five brain tissue slices were taken from each mouse for staining, and the hippocampus region was selected for Nikon ECLIPSE Ti2-U. To analyze the staining intensities of Iba1, the biomarkers of microglia, and αSYN, the antibody for αSYN aggregates, respectively, ordinary one-way ANOVA was used to compare active antibody group with isotype control antibody group. A p-value below 0.05 was considered significant in post-hoc testing (*p<0.05; ***p<0.01; **p<0.001). A p≥0.05 was considered not significant (ns). All statistical analyses were performed using Prism 9 macbook (GraphPad, La Jolla, CA).

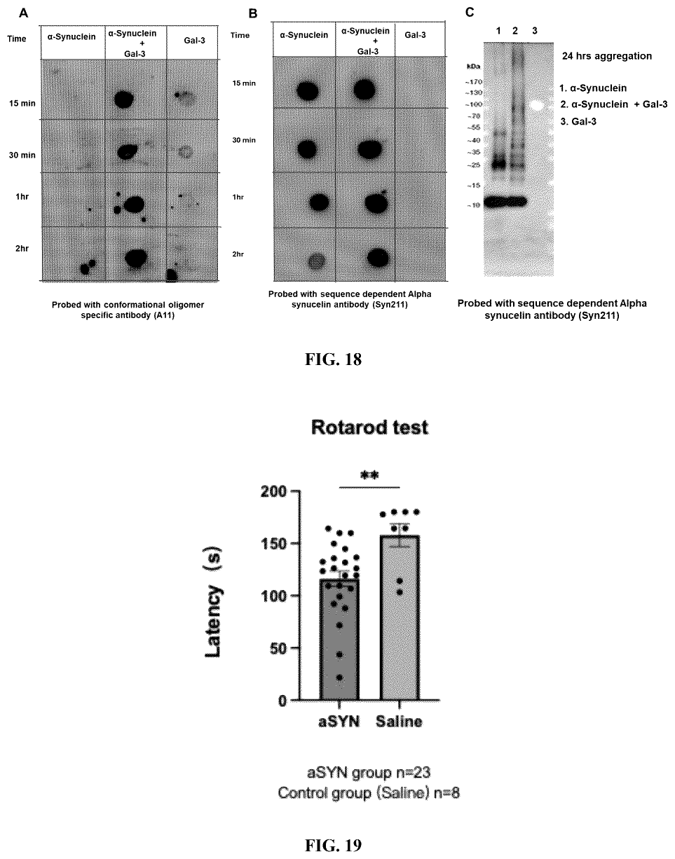

Results. It has been observed Galectin-3 intrinsically promotes oligomers of amyloid proteins. αSYN oligomers will be prepared by incubating monomeric α-synuclein (300 μM) with and without Galectin-3 (100 ug/ml) in phosphate buffer pH 7.4 under stirring condition with a stir bar for 0-5 hrs. Alpha synuclein oligomers were characterized by western blot using conformational oligomer specific antibody A11 and αSYN Ab Syn1. As shown in FIGS. 18A and 18B, the first two lanes were loaded with equal amounts of αSYN, as confirmed by the anti-αSYN antibody Syn211 (FIG. 18B), only αSYN loaded with Gal-3 formed oligomers that were detected using the conformational oligomer specific antibody A11 (FIG. 18A). There finding indicates that Galectin-3 intrinsically promotes αSYN oligomers. After 4 hrs of incubation with Galectin-3, αSYN form LMW and HMW oligomers characterized by Western blot as shown in FIG. 18C.

Animals will be bilaterally injected with 2 μL (per side) of 300 μM oligomeric α-synuclein intrinsically promoted by Galectin-3, or phosphate-buffered saline (Saline) as controls. Solutions will be injected at a constant rate of 0.5 L/min and the needle left in place for 5 min followed by slowly with—drawing the needle. Coordinates for the striatum were +0.2 mm AP, ±2.0 mm ML, −2.6 mm DV. After 2 months, animals under locomotor function test by rotarod and it was observed animals stereotactically injected with α-synuclein oligomer shows statistically significant deficit in locomotor function test in terms of latency as compared to age matched saline treated controls as shown in FIG. 19.

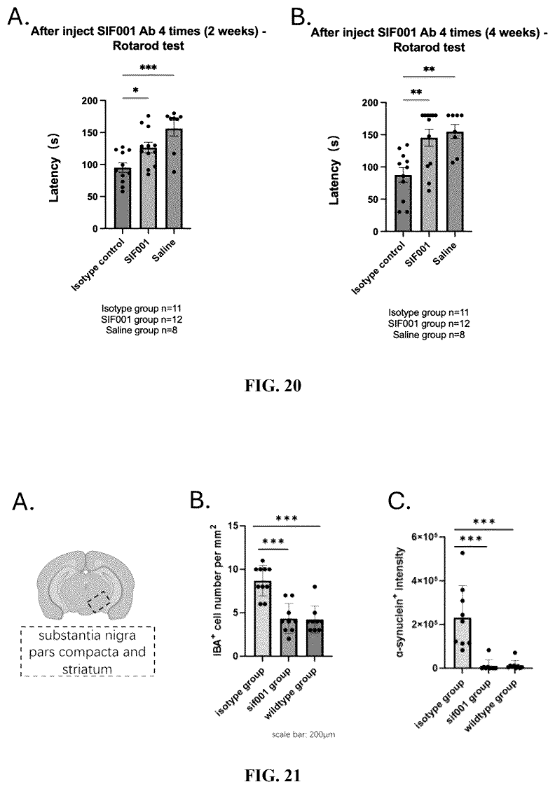

We investigated the effect of mSIF001 on the reduction of neuroinflamation and α-Synuclein pathology induced by toxic α-Synuclein oligomers. After Induction of locomotor dysfunction by α-Synuclein oligomers, mice were dosed with 30 mg/kg anti-HEL mouse IgG2A (isotype control group) or received 30 mg/kg mSIF001 (active group) via i.p. injection for four weeks (2 doses/week). In order to investigate the effect of antibodies, mice underwent the locomotor function test using rotarod. It was observed that, treatment with anti-Galectin-3 antibody shows improvement in locomotor function and the latency time was statistically significant as compared to mice treated with isotype control Ab. As shown in FIGS. 20A and 20B, significant improvement in locomotor function in terms in increase in latency time was statistically significant in mice dosed with mSIF001 Ab as compared to age matched isotype-control treated mice both after 2 and 4 weeks of treatment.

To further explore any pathological changes underlying neuroinflammation, brain tissues from isotype control and active groups, and a group without any treatment, were immuno histologically stained with IBA1 biomarkers for activation of microglia. FIG. 21A indicates the region to be chosen for analysis and it is within pars compacta of the substantia nigra and striatum. As shown in FIG. 21B, mSIF001 significantly reduced activation of microglia indicating that mSIF001 significantly reduced the activated microglia in PD mimic model through disease modification.

In Parkinson's disease, alpha-synuclein mis-folds and aggregates into clumps called Lewy Bodies. It is hypothesized that these may be toxic and the clumps of α-Synuclein get passed from one neuron to another, possibly causing the spread of the disease through the brain. In order to investigate the toxic α-Synuclein aggregates underlying the PD pathology brain sections were stained with α-Synuclein antibody Syn211. By immunofluorescence, it was observed that mSIF001 significantly reduced α-Synuclein aggregated as compared to Isotype control mice indicating that mSIF001 significantly reduced the aggregated α-Synuclein in PD mimic model through disease modification, as shown in FIG. 21C.

### Conclusions

In Parkinson's disease mice model, treatment with mSIF001 was able to significantly improves locomotor function test and significantly reduced neuroinflammation and reduced the aggregated α-Synuclein.

### Example 9. SIF001 for Alzheimer's Disease Treatment

This example illustrates therapeutic effects of SIF001 in a Alzheimer's disease mimic mice model.

### Summary

Alzheimer's disease (AD) is a chronic progressive neurodegenerative disorder that is the leading cause of dementia among older adults. In the United States, AD is one of the leading causes of death, ranking 6th among US adults and 5th among adults aged 65 years or older (CDC, 2020). AD is hypothesized to be caused by toxic changes in the brain involving 2 major pathological hallmark proteins, Aβ and tau. The disease is characterized by accumulation of extracellular amyloid plaques, intracellular neurofibrillary tangles, and extensive synaptic loss leading to progressive cognitive impairment and eventually dementia. Abnormal tau (hyperphosphorylated tau) accumulates to form neurofibrillary tangles, while Aβ plaques accumulate both extracellular and intracellular. Amyloid plaques and hyperphosphorylated tau in the brain are therefore the major hallmarks of AD. Gal3 is highly upregulated in the brains of AD patients and transgenic mouse models of AD (Alzheimer's Association. 2018 Alzheimer's disease facts and figures. Alzheimer's Dement. 201814:367-429). It has been associated with aggregation of Aβ plaques in AD mouse models. Injection of Aβ in Gal3 knockout mice demonstrated reduction in AB oligomerization, whereas injection of Aβ in mice overexpressing Gal3 demonstrated enhanced AB oligomerization (Tao, C, Cheng, K, Ma, Y, et al. Galectin-3 promotes Aβ oligomerization and Aβ toxicity in a mouse model of Alzheimer's disease. Cell Death Differ. 2020; 27:192-209). The primary objective of this in vivo study is to assess the dose range therapeutic effect of anti-Galectin-3 antibody (mSIF001) on hippocampal dependent spatial memory test and reducing neuroinflammation as well as AD pathology in APP/PSI mice (transgenic mouse model of AD). Based on different conformational states as monomers, oligomers and fibril of amyloid protein, conformational oligomers are neurotoxic in AD. In vitro, we found that Gal-3 intrinsically promotes Aβ oligomerization and that SF001 reduced or degraded Aβ42 oligomer formation. APP/PS1 mice were dosed with 30 mg/kg anti-HEL mouse IgG2A (isotype control group) or received dose range of 3, 10 and 30 mg/kg mSIF001 (active group) via intraperitoneal (i.p.) injection four weeks (2 doses/week). Treatment with anti-Galectin-3 antibody of 30 mg/Kg shows improvement in hippocampal dependent memory test (Morris water maze) and was statistically significant as compared to mice treated with Isotype control Ab.

Alzheimer's disease (AD) is one of the most common devastating, irreversible, neurodegenerative disorders related to age, that eventually result in death. Amyloid beta (Aβ) plaque and neurofibrillary tangles deposition are two pathological indicators of AD. So far, there are seven FDA approved prescription drugs to treat AD. However, these drugs can only relieve symptoms of the disease temporarily and none one of them has proven the ability to cure or stop the progression of the disease. The newest FDA-approved drug lecanemab only works in the stage of mild cognitive impairment (MCI) or very early AD (Redzic, Z. Molecular biology of the blood-brain and the bloodcerebrospinal fluid barriers: Similarities and differences. Fluids Barriers CNS. 2011, 8:3). Galectin-3 (Gal-3) plays a pivotal role in microglia-mediated neuroinflammation causing many neurological disorders. In vitro, we found that Gal-3 intrinsically promotes oligomerization and that SF001 reduced or degraded Aβ42 oligomer formation. A transgenic mouse model of AD shows increasing in Aβ aggregates and neuroinflammation and it was used to assess the dose range effect of mSIF001, an anti-Gal-3 monoclonal antibody on the spatial memory hippocampal dependent function test.

### Methods and Experiments

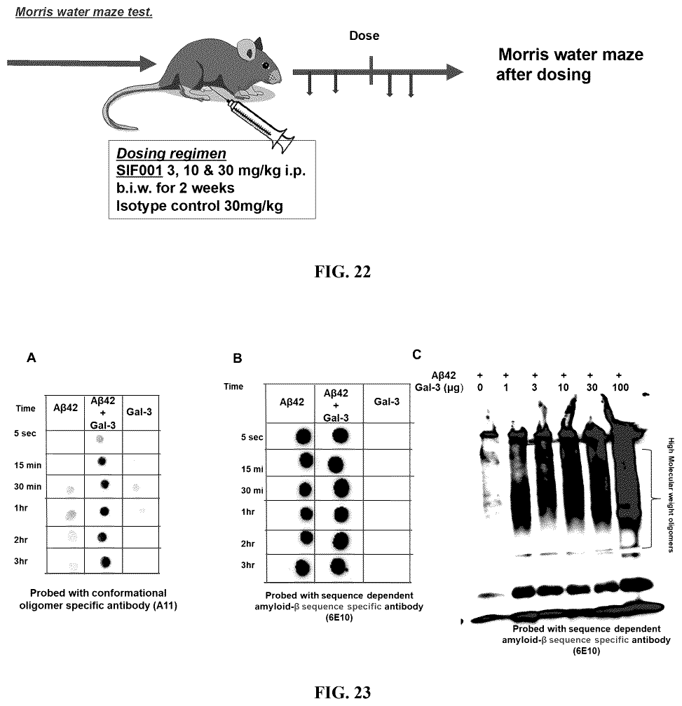

Study Design. Study design is outlined in FIG. 22.

### Preparation of Aβ42 Oligomers.

It has been observed Galectin-3 intrinsically promotes oligomers of amyloid proteins. Aβ₄₂ oligomers were prepared by incubating monomeric Aβ₄₂ (100 ug) with and without Galectin-3 (100 μg/ml) in phosphate buffer pH 7.4 under stirring condition with a stir bar for 0-3 hrs. The solution was continuously stirred using a stir bar while incubating at room temperature. At various time points after mixing, samples were taken and probed on a Whatman nitrocellulose membrane for dot blotting. Different concentrations of SIF001 and isotype control (1-100 μg/ml) antibodies were added to Gal-3-induced Aβ₄₂ oligomers. After 1 hour of incubation, 2 μl of each sample was pipetted onto a dot blot. Aβ₄₂ oligomers were characterized by dot blotting and western blotting using conformational oligomer specific antibody A11 and Aβ sequence-dependent antibody 6E10. Similar procedure was used for the aggregation of Tau, Phospho-Tau, APOE3 & APOE4.

### Dot Blotting

Dot blot membrane was incubated in 10% nonfat dried milk in TBS-T for 1 hour at room temperature to block nonspecific binding. The blot was then incubated with A11 conformational oligomer-specific antibody (Invitrogen) and Aβ sequence dependent antibody 6E10 (BioLegend) overnight at 4° C. After three washes in TBS-T, the membrane was incubated with the appropriate secondary antibody (goat anti-rabbit IgG H&L [HRP] or goat anti-mouse IgG H&L [HRP], Abcam) for 1 hour at room temperature. Following three washes in TBS-T, the membrane was incubated by mixing equal amounts of chemiluminescent detection reagents (Advansta) for 1-5 sec. The dot blot images of the results were obtained using imager (Bio-Rad Chemi doc).

### 5.2.2 Western Blotting

Fifteen μl of each sample were mixed with 4× loading buffer, and the resulting 20-ul mixtures were loaded onto a 4-12% Criterion™ XT Bis-Tris Protein Gel (Bio-Rad). The gel was run at 100 V in sodium dodecyl sulfate-polyacrylamide gel electrophoresis (SDS-PAGE) apparatus (Bio-Rad), and the resolved proteins were transferred onto a nitrocellulose membrane at 300 mA for 60 min. The membrane was thereafter processed as described above. Blots were incubated with 6E10 antibody.

### Animals

APPPS1 mice contain human transgenes for both APP bearing the Swedish mutation and PSEN1 containing an L166P mutation, both under the control of the Thy l promoter. In these mice, expression of the human APP transgene is approximately 3-fold higher than endogenous murine APP. Human Aβ₄₂ is preferentially generated over Aβ₄₀, but levels of both increase with age. AB deposition begins at 6 weeks of age in the cortex and 3-4 months of age in the hippocampus. Six months old APPSwe mice were purchased from Huachuang Sino, Ltd. For SIF001 and Iso-control treatment, mice were administered intraperitoneally (IP) with doses of 3, 10 and 30 mg/kg of SIF001 and isotype control antibody of 30 mg/kg.

### Hippocampal Dependent Spatial Memory Test

Morris water maze test was conducted before and after receiving the different doses of SIF001 antibody. Mice were handled for 3 days before testing and were habituated to testing room for 1 h at the start of each testing day. The order of test will be designed to minimize stress to the animals, such that low-stress tasks will be completed before high-stress tasks. Additionally, mice received at least one day rest between each test.

Morris water maze is a special memory task related to hippocampus. The apparatus used for all water maze tasks was a circular aluminum tank (1.5 m diameter) painted white and filled with water maintained at 26° C.-29° C. The maze was located in a room containing simple visual, extra-maze cues. To reduce stress, mice were placed on the platform in both the hidden and cued versions of the task for 15 sec. prior to the first training trial. Mice were trained to swim to a circular clear Plexiglas platform (14 cm diameter) submerged 1.5 cm beneath the surface of the water and invisible to the mice while swimming. The platform location was selected randomly, but was kept the same for each individual mouse throughout training. On each trial, the mouse was placed into the tank at one of four designated start points in a pseudorandom order. Mice were allowed 60 sec to find the submerged platform. If a mouse failed to find the platform at 60 sec, it was manually guided to the platform and allowed to remain there for 15 sec. After this, each mouse was placed into a holding cage under a warming lamp for 30 s before beginning the next trial. To ensure that memory differences were not due to lack of task learning, mice were given four trials a day for as many days as required to train the APP/PSI mice to reach the criterion (<20 sec.). To control for overtraining, probe trials were run for each group, both as soon as they reached group criterion and after all groups had reached criterion. We trained the APP/PSI mice for 8 days. Retention of the spatial training was tested 1.5 hr and again 24 hr after the last training trial. Both probe trials consisted of a 60s free swim in the pool without the platform. Mice were monitored by a camera mounted in the ceiling directly above the pool to record the 1.5 hr and 24 hr test. The parameters measured during the probe trial included initial latency to cross the platform location, number of platform location crosses, and time spent in the quadrant opposite to the one containing the platform during training

### Animals and Treatment Groups

6 months old APP/PS1 (n=30) and age matched wild type C57BL/6J mice (n=8) were used for experiments. Animals were housed in pairs in a controlled temperature environment under a 12-h light/dark cycle, with access to chow and water ad libitum. All animals were provided at least 72 h to acclimate following arrival at the facility, with periodic cage changes, before surgery. All mice were divided to three treatment groups as shown in Table 9.

TABLE 9
Treatment groups

| Treatment Group | Animal | Dose | Animal No. | Route |
|---|---|---|---|---|
| Isotype control antibody (Anti-HEL antibody) | APP/PS1 | 30 mg/kg | 4 | Intraperitoneal |
| Active (mSIF001 in mouse IgG2A backbone) | APP/PS1 | 3, 10 and 30 mg/kg | 5 per dose | Intraperitoneal |
| Wild type C57BL/6J mice | C57BL/6J | No dose | 10 | |

### Bioanalysis

To analyze the behavior memory data, ordinary one-way ANOVA was used to compare active antibody group with isotype control antibody group. A p-value below 0.05 was considered significant in post-hoc testing (*p<0.05; **p<0.01; ***p<0.001). A p≥0.05 was considered not significant (ns). All statistical analyses were performed using Prism 9 (GraphPad, La Jolla, CA).

### Results

It is widely accepted that Aβ oligomers rather than monomers and fibrils play a critical role in AD progression. Aβ₄₂ oligomers can trigger a series of toxic reactions in neurons, such as receptor disability, mitochondrial damage, Ca²⁺ homeostasis dysregulation and abnormal Tau phosphorylation. Targeting the most toxic oligomers may be an effective treatment for AD by preventing aggregation of Aβ₄₂. Gal-3 was reported to be involved directly or indirectly in the oligomerization of AB in in-vivo studies. It has been observed Galectin-3 intrinsically promotes oligomers of amyloid proteins. Aβ₄₂ oligomers were prepared by incubating monomeric Aβ₄₂ (100 μg) with and without Galectin-3 (100 ug/ml) in 100 mM phosphate buffer pH 7.4 under stirring condition with a stir bar for 0-3 hrs. Aβ₄₂ oligomers were characterized by dot blotting using conformational oligomer specific antibody A11 (FIG. 23A) and total Aβ₄₂ was demonstrated by Aβ₄₂ sequence-dependent antibody 6E10 (FIG. 23B). Gal-3 oligomerized Aβ₄₂ peptide into high molecular weight oligomers in a dose dependent manner as shown in Western blot probed with Aβ sequence dependent antibody 6E10 (FIG. 23C). As shown in FIG. 23C, the sample without Gal-3 exhibits significantly fewer high molecular weight oligomers, while with the increasing amount of Gal-3, the intensity of high molecular weight oligomers also increases, indicating that Gal-3 dose-dependently enhances the oligomerization.

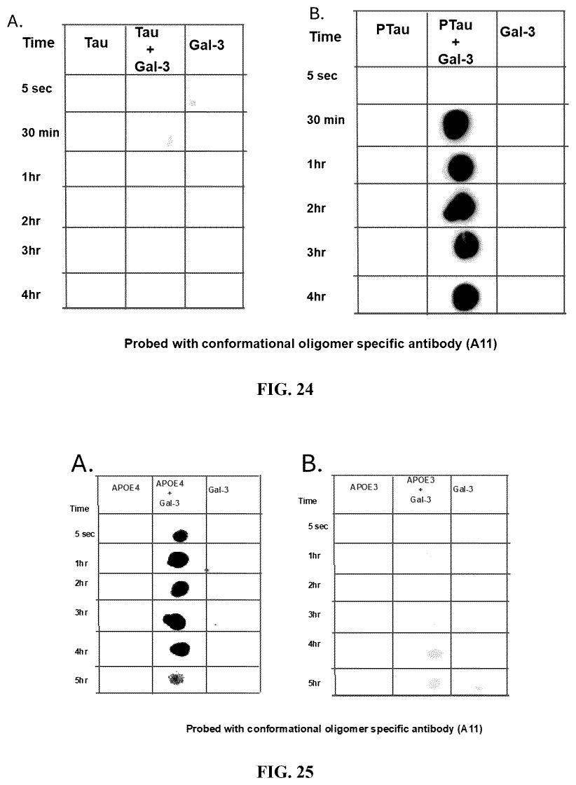

Abnormal tau (hyperphosphorylated tau) is another pathological hallmark of AD. In addition to Aβ, Gal-3 intrinsically promotes aggregation of phosphorylated tau (FIG. 24B), but not normal tau (FIG. 24A), probed with conformational oligomer antibody A11.

AD is the most common cause of dementia worldwide, and its prevalence is rapidly increasing due to extended lifespans. Among the increasing number of genetic risk factors identified, the apolipoprotein E (APOE) gene remains the strongest and most prevalent, impacting more than half of all AD cases. While the ε4 allele of the APOE gene significantly increases AD risk. Genome-wide association studies revealed that the ε4 allele, but not the ε3 allele, of the apolipoprotein E (APOE4) gene, is the strongest genetic risk factor for AD and Lewy body dementia (LBD), signifying an important role of APOE4 in both amyloid-β (Aβ) and α-Synuclein pathogenesis. Here we showed Gal-3 intrinsically promotes APOE4 but not APOE3 as shown in FIGS. 25A and 25B.

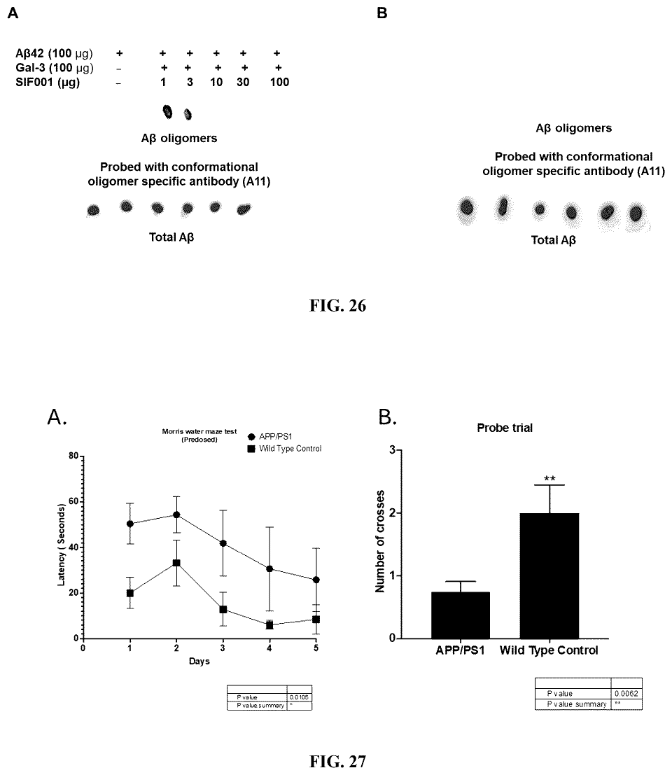

Next, we tested if Gal-3 blocking antibodies can dissolve Aβ₄₂ oligomers induced by Gal-3. As shown in FIGS. 26A and 26B, SIF001 dissolved Aβ₄₂ aggregates in a dose-dependent manner, while an iso-type control antibody had no effect.

We further investigated the hippocampal dependent memory function test in AD transgenic mice model. APP/PSI mice demonstrated significant deficits in cognition in terms of latency to reach the platform and number of crosses in the Morris water maze as compared to age matched wild type mice, as shown in FIGS. 27A and 27B.

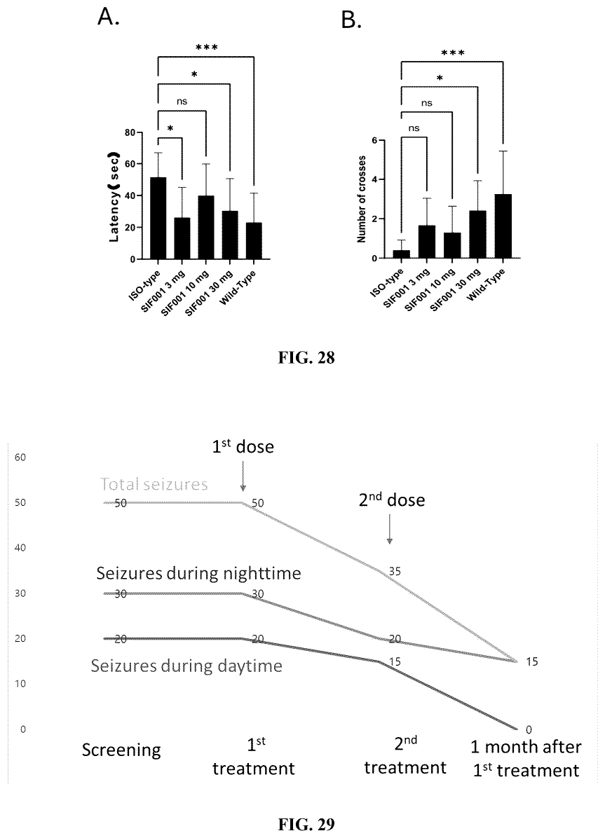

To explore the effect of SIF001 Ab (3-30 mg/kg) on spatial memory test, Morris water maze test was conducted. As shown in FIGS. 28A and 28B, mice dosed with 10 and 30 mg/kg significantly decreased latency to reach platform (FIG. 28A) and mice with 30 mg/kg significantly increased the number of crosses of the platform (FIG. 28B), as compared to the isotype control antibody treated mice.

### Conclusions

Galectin-3 intrinsically promotes oligomerization of Aβ₄₂, Phospho tau and APOE4 but not normal tau. Anti galectin-3 antibody SIF001 dissolved Aβ₄₂ aggregates in a dose-dependent manner, while an iso-type control antibody had no effect. In APP/PS1 transgenic mouse model of AD, treatment with mSIF001 of 30 mg/kg was able to show statistically significant improvement in cognitive function in terms of memory as compared to the isotype antibody treated mice.

### Example 10. SIF001 Investigator-Initiated Trial (IIT) for Treating Drug-Resistant Epilepsy

This example illustrates a pilot study of a monoclonal antibody (SIF001) for the treatment of drug-resistant epilepsy.

The exemplary study is a multicenter, prospective, open-label study to explore the efficacy and safety of the monoclonal antibody SIF001 in the treatment of drug-resistant epilepsy. A total of 6 patients with drug-resistant epilepsy who could not undergo respective surgery were enrolled (3 patients in each center). The effectiveness of the monoclonal antibody SIF001 was evaluated by recording epilepsy diaries and electroencephalogram epileptic discharges, and the safety was evaluated by recording adverse reactions.

### SIF001 Product

SIF001 is a monoclonal antibody drug (IgG4) targeting Gal-3. It is a colorless transparent solution stored in a disposable sterile glass bottle for injection, sealed with a rubber stopper and an aluminum flat flip cap. Each bottle contains 10 mL, with a concentration of 20 mg/mL and an optimal storage temperature of 2-8 degrees Celsius.

### SIF001 Drug Safety

Monoclonal antibodies based on the same target and the same region have similar safety profiles, so the safety profile of SIF001 should be similar to that of TB006, a similar product that has completed clinical trials. In a Phase 1 single-dose escalation study of healthy volunteers completed in the United States, the monoclonal antibody TB006 showed good tolerability and safety even at a dose of up to 5000 mg. In a Phase 2 clinical trial of TB006 for Alzheimer's disease that has been completed, only 4 drug-related adverse reactions were reported among 63 patients who received the drug, including one dizziness (1/63, 1.59%), one phlebitis (1/63, 1.59%), and two infusion reactions (2/63, 3.17%), all of which were mild, sporadic and self-limiting, and the drug had good safety.

Based on the above conditions, the potential benefits of SIF001 treatment to patients far outweigh the possible risks, so an exploratory pilot study is planned to evaluate the safety and efficacy of this product as an adjunctive treatment for drug-resistant epilepsy. Referring to the dose of TB006, this study uses a 50 mg/kg intravenous injection for two times.

### Study Design

### 1. Overall Design and Determination Basis

The purpose of this trial is to preliminarily verify the safety and efficacy of monoclonal antibody SIF001 in the treatment of drug-resistant epilepsy. This project is an exploratory clinical trial. After enrollment, the subjects need to receive a single intravenous injection of monoclonal antibody SIF001. The trial adopts a multicenter, exploratory pilot design. This study will be conducted in two centers in China, and a total of 6 subjects are planned to be included. After the subjects sign the informed consent form (ICF) approved by the Ethics Committee, they will enter the screening procedure to screen subjects who meet the inclusion criteria and do not meet any of the exclusion criteria, and assign subject numbers in order of enrollment time. After completing all follow-up visits for each case, the next case will be enrolled.

Subjects were treated with intravenous monoclonal antibody SIF001 on the 1st and 15th days (±1 day) after enrollment. On the 28th day after enrollment, the number of SIF001 injections could be increased according to the patient's specific situation. The frequency of subsequent additional treatment was limited to no more than once every 28 days, and continued for 1 year after treatment. The clinical evaluation time nodes include 7 days after the first treatment, 14 days after treatment (±1 day), 21 days after treatment (±1 day), 28 days after treatment (±3 days), and 84 days after treatment (±7 days). If additional treatment is given, the follow-up point should also include 28 days (±3 days) after each subsequent treatment. Laboratory tests, scale evaluations, etc. are performed at the specified nodes to verify the treatment effect and safety.

### 2. Selection of Subjects

Subjects were selected using the following inclusion criteria:
a) Diagnosed with drug-resistant epilepsy;
b) Aged ≥10 and ≤50 years old, regardless of gender;
c) The subject is considered unable to benefit from respective surgery through preoperative evaluation of epilepsy surgery or the subject refuses the treatment;
d) The subject is not suitable for or refuses implantable vagus nerve stimulation or deep brain stimulation treatment;
e) Epileptic seizures ≥8 times per month, including focal seizures and/or generalized seizures;
f) Not participated in other clinical trials within three months;
g) The subjects and their families have reasonable expectations that the subjects can keep an epileptic seizure diary alone or with the help of their family members;
h) Volunteer to participate in this trial and sign the written informed consent;
i) No severe immune deficiency;
j) Weight ≥50 kg, body mass index (BMI) between 18-35.

### 3. Enrollment Time and Study Duration

This feasibility trial was conducted in a hospital in China, and the enrollment time of the feasibility trial is expected to be 3 months. The expected overall duration of the feasibility study and the reasons for its determination

The feasibility trial plan was formulated in 1 month, and the ethical review and approval is expected to take 1-2 months. The enrollment is planned to start in August 2024, and it will take 12-24 months from the start of enrollment to the completion of the trial and data collection. The overall duration of this trial is 15-27 months from the ethics application to the end of the trial.

The subjects are expected to participate in the trial for about 3 months, including screening (−14 to +7 days), informed consent, drug treatment (+1 day, +15 days), 7 days after treatment, 14 days after treatment (±1 day), 21 days after treatment (±1 day), 28 days after treatment (±3 days), and 84 days after treatment (±7 days). If additional treatment is given, the follow-up point should also include 28 days (±3 days) after each subsequent treatment.

### 4. Evaluation Methods

Based on previous clinical research and treatment practices, this trial requires clinical evaluation of the subjects enrolled at screening (−14˜+7 days, 0 day for enrollment), drug treatment day (+1 day, +15 day), 7 days after treatment, 14 days after treatment (±1 day), 21 days after treatment (±1 day), 28 days after treatment (±3 days), and 84 days after treatment (±7 days). If additional treatment is given, the follow-up point should also include 28 days (+3 days) after each subsequent treatment. Laboratory tests, scale evaluations, etc. are performed at specified nodes to verify the treatment effect and safety.

### A. Effectiveness Evaluation

### Main Evaluation Indicators

The main evaluation indicator set in this trial is the reduction rate of epileptic seizure frequency 28 days after drug treatment compared with the baseline, which is used to evaluate the effectiveness of treating epilepsy.

Evaluation of epilepsy treatment efficacy: The subjects or their family members record the type, number, and duration of epileptic seizures every day. The researchers count the frequency of epileptic seizures on a weekly basis. The treatment is considered effective if the frequency of epileptic seizures in the subjects is reduced by 50% or more compared to the baseline. The treatment is considered “significantly effective” if the frequency of epileptic seizures is reduced by more than 90%. The treatment is considered “effective” if the frequency of epileptic seizures is reduced by 50%-89% compared with before. The treatment is considered “ineffective” if the frequency of epileptic seizures decreased by 49% or less compared with the previous period.

Total effective=significant effect+effective

Effectiveness=(total number of effective subjects/total number of subjects participating in clinical trials)×100%

Inefficiency=(total number of ineffective subjects/total number of subjects participating in clinical trials)×100%

Evaluation time points: screening period, 7 days after treatment, 14 days (±1 day), 21 days (±1 day), 28 days (±3 days), 84 days (±7 days). If additional treatment is given, the follow-up point should also include 28 days (±3 days) after each subsequent treatment.

### Secondary Evaluation Indicators

Secondary evaluation indicators include the following three changes.
\textcircled{1} Changes in the number of EEG epileptiform discharges relative to the baseline, and compare with the baseline to evaluate the improvement of EEG.

Evaluation method: The number of epileptiform discharges per hour recorded by EEG.

Evaluation time points: screening period, 7 days after treatment, 28 days (±3 days), 84 days (±7 days). If additional treatment is given, the follow-up point should also include 28 days (±3 days) after each subsequent treatment.
\textcircled{2} Changes from baseline in the Quality of Life in Epilepsy Scale-31 (QOLIE-31) after treatment.

Evaluation method: The patients were evaluated according to the Quality of Life in Epilepsy Scale-31 (QOLIE-31).

Evaluation time points: screening period, 28 days (±3 days), and 84 days (±7 days) after treatment. If additional treatment is given, the follow-up point should also include 28 days (±3 days) after each subsequent treatment.
\textcircled{3} Changes in the Hamilton Depression Rating Scale and Hamilton Anxiety Rating Scale of epilepsy patients relative to baseline after treatment.

Evaluation method: Evaluation according to the Hamilton Depression Rating Scale and the Hamilton Anxiety Rating Scale.

Evaluation time points: screening period, 28 days (±3 days), and 84 days (±7 days) after treatment. If additional treatment is given, the follow-up point should also include 28 days (±3 days) after each subsequent treatment.

### B. Safety Evaluation

Safety evaluation includes the following six criteria.
\textcircled{1} Collect and analyze blood biochemistry (alanine aminotransferase, aspartate aminotransferase, creatine kinase, albumin, creatinine, direct bilirubin, glucose, high-density lipoprotein, low-density lipoprotein, total bilirubin, triglycerides, urea nitrogen, calcium ion, potassium ion, sodium ion, uric acid);
\textcircled{2} Collect and analyze routine blood test;
\textcircled{4} Collect and analyze glycosylated hemoglobin;
\textcircled{5} Collect and analyze urine routine test;
\textcircled{6} Collect vital signs (blood pressure, respiration, body temperature, pulse).

Evaluation time points: screening period, 7 days, 28 days (±3 days), and 84 days (±7 days) after treatment. If additional treatment is given, the follow-up point should also include 28 days (±3 days) after each subsequent treatment.

### C. Test Process

Test process includes screening period and treatment period.

(1) Screening Period (−14˜+7 days)
\textcircled{1} Sign the informed consent form;
\textcircled{2} Collect medical history, demographic and other information;
\textcircled{3} Vital signs and laboratory tests (blood routine, biochemistry, glycosylated hemoglobin, urine routine, HIV antibody, Gal-3 (tested by Kangxu Biotechnology Co., Ltd.), electrocardiogram);
\textcircled{4} Anti-epileptic drug records;
\textcircled{5} Record of epileptic seizure frequency (daily);
\textcircled{6} Scales (Hamilton Depression Rating Scale, Hamilton Anxiety Rating Scale, Quality of Life in Epilepsy Scale-31 (QOLIE-31));
\textcircled{7} The number of epileptic discharges per hour recorded by EEG.

### (2) Treatment Period

Treatment period includes (drug treatment day (+1 day, +15 day), 7 days after treatment, 14 days after treatment (±1 day), 21 days after treatment (±1 day), 28 days after treatment (±3 days), 84 days after treatment (±7 days). If additional treatment is given, the follow-up point should also include 28 days after each subsequent treatment (±3 days)).
\textcircled{1} Drug treatment days (+1 day, +15 days): medication process records, vital signs, adverse event records, seizure frequency, anti-epileptic drug records and combined treatments.
\textcircled{2} 7 days after treatment: vital signs, adverse event records, seizure frequency, anti-epileptic drug records and concomitant treatment, electroencephalogram, laboratory tests (blood routine, biochemistry, urine routine, electrocardiogram).
\textcircled{3} 14 days after treatment (±1 day): vital signs, adverse event records, seizure frequency, anti-epileptic drug records and concomitant treatments.
\textcircled{4} 21 days after treatment (±1 day): vital signs, adverse event records, seizure frequency, anti-epileptic drug records and concomitant treatments.
\textcircled{5} 28 days after treatment (±3 days): vital signs, adverse event records, seizure frequency, anti-epileptic drug records and concomitant treatment, electroencephalogram, laboratory tests (blood routine, biochemistry, urine routine, Gal-3 (tested by Kangxu Biotechnology Co., Ltd.), electrocardiogram), Hamilton Depression Rating Scale, Hamilton Anxiety Rating Scale, Quality of Life Assessment Scale for Epilepsy-31 (QOLIE-31).
\textcircled{6} 84 days after treatment (±7 days): vital signs, adverse event records, seizure frequency, anti-epileptic drug records and concomitant treatment, electroencephalogram, laboratory tests (blood routine, biochemistry, glycosylated hemoglobin, urine routine, Gal-3 (tested by Antenruilin (Shanghai) Biotechnology Co., Ltd.), electrocardiogram), Hamilton Depression Rating Scale, Hamilton Anxiety Rating Scale, Quality of Life Assessment Scale for Epilepsy-31 (QOLIE-31).
\textcircled{7} If additional treatment is given, the follow-up point should also include 28 days (±3 days) after each subsequent treatment: vital signs, adverse event records, seizure frequency, anti-epileptic drug records and concomitant treatment, electroencephalogram, laboratory tests (blood routine, biochemistry, glycosylated hemoglobin, urine routine, Gal-3 (tested by Kangxu Biotechnology Co., Ltd.), electrocardiogram), Hamilton Depression Rating Scale, Hamilton Anxiety Rating Scale, Quality of Life Assessment Scale for Epilepsy-31 (QOLIE-31).

### D. Drug Treatment Specifications

Infusion procedures were performed by epilepsy specialist nurses. Drug storage: 4-degree refrigerator, epilepsy ward. Before administration, the dose was calculated according to the patient's weight (50 mg/kg), and the SIF001 drug (20 mg/mL) was diluted to 500 mL with sterile saline, and the infusion was completed within 1 or 2 hours. The drug was infused on the 1st and 15th days respectively. On the 28th day after enrollment, the number of SIF001 injections can be increased as appropriate according to the patient's specific situation, and the subsequent treatment frequency is limited to no more than 1 time every 28 days, and continues for 1 year after treatment.

### E. Statistical Considerations

This trial was an exploratory pilot trial and only included 6 subjects. Therefore, no statistical analysis was performed. Only information such as the subjects' demographic data, medical history, and trial conditions were described.

### F. Data Management

This feasibility trial uses a case report form, which is filled out by the researcher or his/her authorized personnel. The case report form for each selected case must be completed.

### Study Results

### Patient 1

Patient 1 was 10 years and 4 months old when enrolled to the trail. The disease started at the age of 4. The possibility of Rasmussen's encephalitis was considered high, and the family refused surgery. When the patient was enrolled in December 2024, his left muscle strength was grade 4 and the right muscle strength was grade 3. The patient had mixed aphasia, could occasionally answer single words, and had poor intelligence. Seizures occurred once every 10⁻³⁰ minutes, and >50 attacks per day. The specific number of attacks could not be counted in detail, with more frequent attacks during sleep, occurring once every 5-10 minutes. The patient had one seizure pattern: looking to the right with head and shaking of right limbs, which lasts for 5-10 seconds and is relieved. The patient appeared poor consciousness, drowsiness, bedridden, and nasogastric feeding.

One month after using the monoclonal antibody SIF001, there were almost no attacks during daytime, and the attacks at night were significantly reduced to no more than 15 attacks during nighttime, as shown in FIG. 29. The patient appeared in good mental condition and conscious. The left muscle strength changed to grade 5 and the right muscle strength up to grade 4. The patient can get out of bed and eat independently.

### Patient 2

Patient 2 was 13 years and 2 months old when enrolled to the trail, with an epilepsy history of 7 years since 2017 Sep. 14. Commonly used anti-epileptic drugs are ineffective to the patient. The patient did vagus nerve stimulation (VNS) in 2018. Before treatment, the patient experienced more than 10 attacks per month and in a cognitive state of dementia.

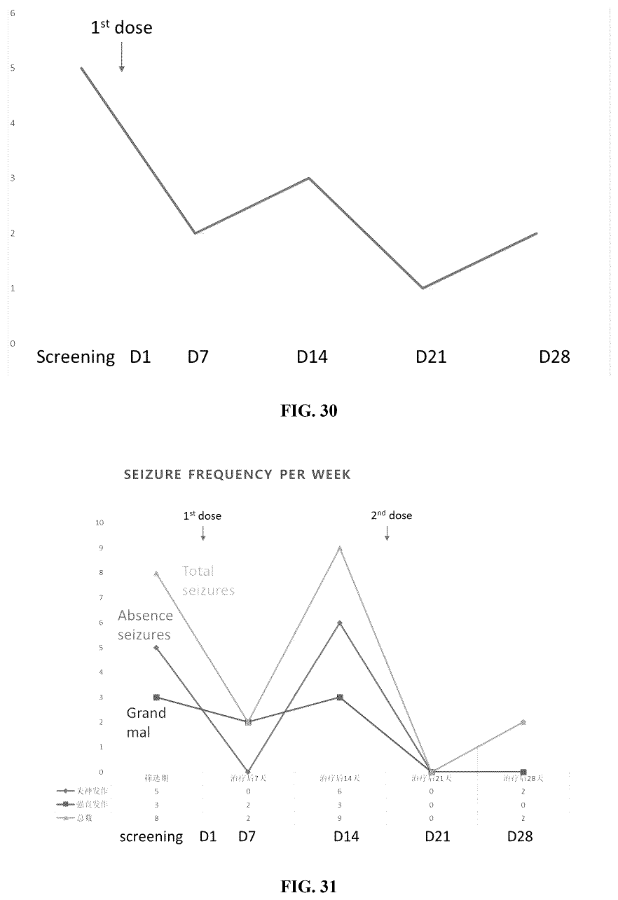

Four weeks after using the monoclonal antibody SIF001, seizure frequency of the patient reduced from 5 times per week to 2 times per week, as shown in FIG. 30.

### Patient 3

Patient 3 was 12 years old when enrolled to the trail, male, episodic limb twitching, consciousness disorder for 7 years. The disease started at the age of 5, with 2 types of attacks: Episodic stupor and Episodic limb twitching, disturbance of consciousness, flexion of the upper limbs, straightening of the lower limbs and shaking. Before treatment, the average number of attacks was 8-10 times a day. Commonly used anti-epileptic drugs are ineffective to the patient. hormone shock, intravenous immunoglobulin (IVIG) treatments and low-temperature plasma (LTP) stimulation had certain effects to the patient. Transcranial magnetic stimulation and electrical stimulation treatments had poor results on the patient. The patient had cognition worse than peers but still can take care of himself. The patient was diagnosed with post-encephalitic epilepsy.

Four weeks after treating with the monoclonal antibody SIF001 for twice, the grand mal seizure frequency of the patient reduced from 3 times per week to zero; the absence seizure frequency of the patient reduced from 5 times per week to 2 times per week; and the total seizure frequency of the patient reduced from 8 times per week to 2 times per week, as shown in FIG. 31. Caregiver reported that patient's language expression and motor abilities improved significantly after taking the medicine.

### Patient 4

Patient 4 was 13 years when enrolled to the trail, male, with episodic loss of consciousness and twitching of limbs for more than 10 years. The disease onset at the age of 3, with 2 seizure patterns: 1. Attack while awake, loss of consciousness, falling to the ground with limb twitching, obvious twitching of the right upper limb, left limb stiffness, and head turning to the right; 2. Attack while sleeping, sitting up, right corner of mouth twitching, left upper limb flexion and stiffness. The patient experienced average of 15-20 attacks per day before treatment. The patient was diagnosed with genetically related epilepsy. Gene testing results indicated that MAN2B2 heterozygous mutation and STK26 hemizygous missense mutation. Commonly used anti-epileptic drugs are ineffective to the patient. Effects of both resection of the epileptic focus in the left central functional area performed in 2022 and VNS implantation in 2024 were not satisfactory. Cognitive impairment was significant, and schooling had been discontinued.

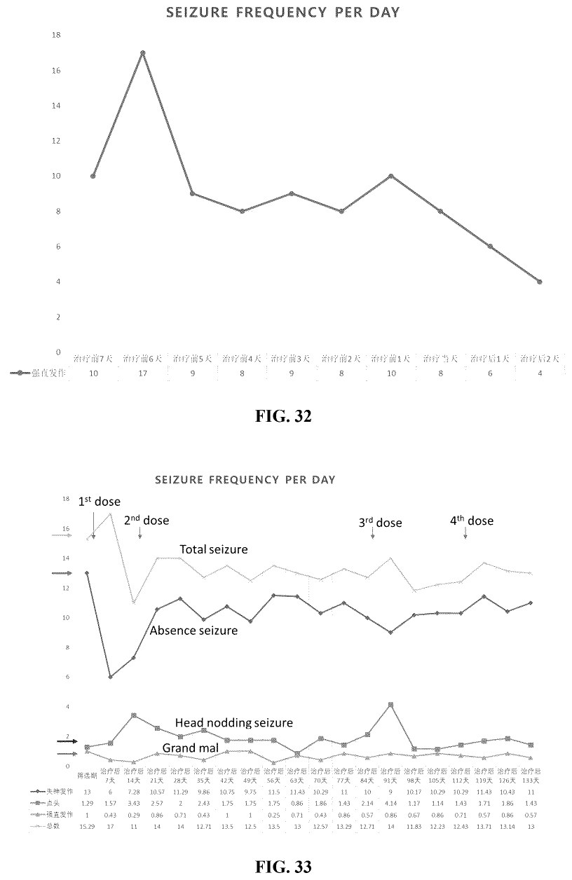

Two days after using the monoclonal antibody SIF001, seizure frequency of the patient reduced from 8-17 times per day before the treatment to 4 times per day post the treatment, as shown in FIG. 32.

### Patient 5

Patient 5 was 30 years old when enrolled to the trail, male, with episodic stupor, limb rigidity and loss of consciousness for 27 years. The disease onset at the age of 3, with 3 seizure patterns: 1. Paroxysmal stupor, lasting for dozens of seconds; 2. Paroxysmal nodding, lasting for 1-2 seconds; 3. Loss of consciousness with limb rigidity, lasting for 1-2 minutes and relieved. The patient experienced average >20 seizures a day before treatment. The patient was diagnosed with Lennox-Gastaut Syndrome (LGS).

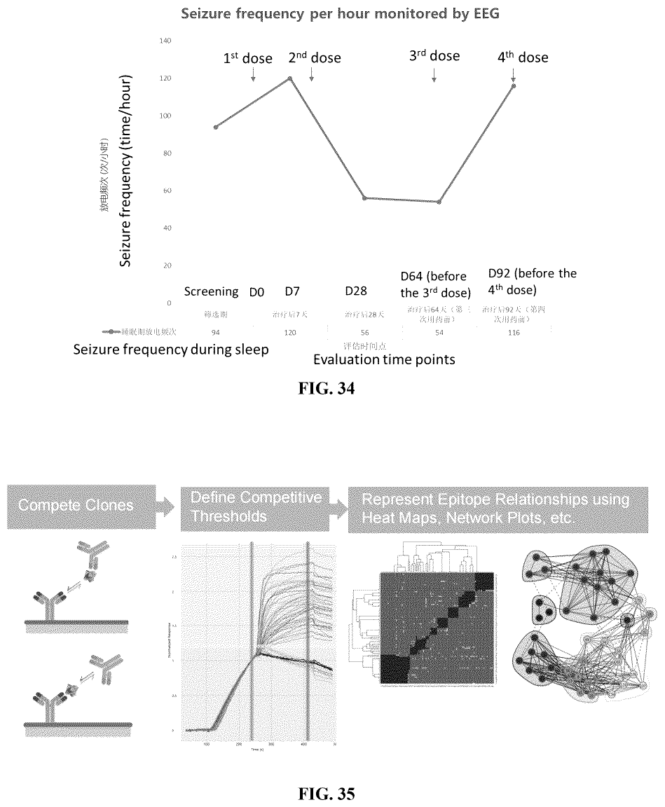

The patient was administrated with the SIF001 four times. As shown in FIG. 33, 133 days after the first treatment with the monoclonal antibody SIF001, the absence seizure frequency of the patient reduced from 13 times per day before the treatment to 6-11 times per day; and the total seizure frequency of the patient reduced from 15.29 times per day before the treatment to 11-13 times per day post the treatment, as shown in FIG. 33. We also monitored the patient's seizure frequence during sleep through electroencephalogram (EEG). As shown in FIG. 34, the patient's seizure frequence during sleep was reduced from 94 times per hour before the treatment to 54-56 times per hour post the treatment, while some fluctuations were observed.

### Example 11. SIF001 Phase I Study in Healthy Subjects and in a Patient Cohort with Epilepsy

This example illustrates a Phase 1, double-blind, placebo-controlled, randomized, single and multiple ascending dose escalation study to assess the safety, tolerability, pharmacokinetics and pharmacodynamics of SIF001 in healthy subjects and in a patient cohort with epilepsy.

### Study Rationale

SIF001 is an investigational humanized IgG4 (S228P) antibody against galectin-3 (Gal-3), developed for the treatment of neurological disorders such as epilepsy. In order to evaluate the safety, tolerability, and pharmacokinetics of SIF001, a Phase 1, randomized, double-blind, placebo-controlled, single and multiple ascending dose escalation study will be conducted in healthy volunteers.

The single-ascending dose (SAD) stage will be conducted in healthy volunteers to evaluate the dose levels to be further investigated in the multiple-ascending dose (MAD) stage. The MAD stage will then evaluate the safety of 3 doses of SIF001 administered every 2 weeks (Q2W) in healthy volunteers.

Given the unmet medical need of patients with drug-resistant epilepsy, an additional cohort in the MAD stage is planned to enroll patients with epilepsy, who have not responded on prior treatment of antiepileptic drugs (AEDs). This cohort will help assess the safety and pharmacokinetics (PK) of SIF001-001 in the target patient population and generate preliminary efficacy data in a relatively small group of patients before exposing larger groups of patients to SIF001.

A recommended phase 2 dose (RP2D) will then be selected after evaluation of the SAD and MAD stages for further proof-of-concept evaluation of SIF001 in patients with epilepsy.

### Study Design

This is a Phase 1, randomized, double-blind, placebo-controlled, single and multiple ascending dose escalation study to evaluate the safety, tolerability, and pharmacokinetics of SIF001 in healthy volunteers and one cohort of patients with epilepsy. The total study duration for each subject will be up to approximately 11 weeks.

This study consists of two stages:

### Stage I: Phase 1a Single-Ascending Dose (SAD) Study of SIF001 in Healthy Volunteers

Subjects in this study stage will receive a single administration of SIF001 or placebo on Day 1 of the study.

Dose groups will be enrolled sequentially. The starting dose in humans will be 10 mg/kg. The subsequent planned doses are 20 mg/kg, 40 mg/kg and 70 mg/kg. The planned doses for each group following the initial dose group are subject to change (increase or decrease) based on the safety, tolerability and pharmacokinetic (if available) data from the previous groups. In addition, dose groups may be added or removed from the planned design based on emerging data. All doses are infused intravenously over 1 or 2 hours.

Eight subjects will be enrolled into each dose group; 6 subjects in each group will be randomized to active SIF001 treatment and 2 subjects to placebo. A total of approximately 24 subjects will be randomized in this stage, with 18 receiving SIF001 treatment at different dose levels and 6 receiving the placebo.

Within each dose group, a sentinel dosing schedule will be used as follows:
On Day 1, 2 subjects (1 active SIF001; 1 placebo) will be dosed and observed.
On Day 3, if safety and tolerability from subjects dosed on Day 1 is satisfactory, the remaining subjects will be dosed and observed. As an additional precaution, the remaining subjects will not all be dosed simultaneously, but be staggered with two subjects being dosed at a time.

Dose escalation decisions will use emerging safety and tolerability data through 7 days after the last subject in the current dose group was dosed (Day 8). Data will be reviewed by a blinded Safety Review Committee (SRC). For dose levels higher than 20 mg/kg, available clinical data will also be presented to the US FDA for review.

The initiation of enrollment at the 40 mg/kg dose level will only be initiated upon the review of the clinical data of the 10 and 20 mg/kg dose levels by the FDA. The Sponsor will submit to the FDA all available safety and tolerability data (e.g., AEs, clinical laboratory tests, vital signs, 12-lead safety electrocardiograms [ECGs], and physical examinations) for all subjects in the 10 mg/kg SAD dose group and all available data through at least 7 days for at least 6 out of 8 subjects in the 20 mg/kg SAD dose group.

Similarly, the initiation of enrollment at the 70 mg/kg dose level will only be initiated upon the review of the clinical data of the 10, 20, and 40 mg/kg dose levels by the FDA. The Sponsor will submit to the FDA all available safety and tolerability data for all subjects in the 10 and 20 mg/kg SAD dose groups and all available data through at least 7 days for at least 6 out of 8 subjects in the 40 mg/kg SAD dose group.

Study assessments will be conducted, and subjects will be dosed as scheduled. Subjects will be admitted to the clinical unit 1 day prior to their scheduled dosing (Day-1). Subjects will fast overnight prior to dosing. Subjects will remain at the clinical unit for at least 2 days following their dosing for safety assessments and pharmacokinetics (PK) sampling. Subjects will be discharged from the clinical unit at the discretion of the principal investigator following completion of the assessments on Day 2 provided there are no safety concerns identified from review of the clinical data. Subjects will return to the clinical unit on pre-specified days outlined in the Schedule of Assessments for safety assessments and PK/pharmacodynamics (PD) sampling for 75 days. If a subject reports any adverse events (AEs), they may be required to return to the clinical unit at the discretion of the principal investigator for additional assessments. All AEs must be followed to adequate resolution.

### Stage II: Phase 1b Multiple-Ascending Dose (MAD) Study of SIF001 in Healthy Volunteers, Followed by One Patient Cohort

Subjects in this study stage will receive repeated administrations of SIF001 or placebo on Days 1, 15, and 29 of the study.

Stage II enrollment for the first dose group will be initiated at least 15 days after the last subject of Group 2 of Stage I has received their dose of SIF001 and there are no safety concerns identified from review of the clinical data. The other dose groups will also have the same rule applied.

The provisional planned doses are 10 mg/kg, 20 mg/kg, 40 mg/kg and 70 mg/kg. The dose escalation design is identical with Stage I, including sentinel dosing of the first dose and randomization of 6 subjects to SIF001 and 2 subjects to placebo, with the exception that the SRC will convene at least 14 days after the last subject in the dose group has received their last dose of SIF001 (Day 43).

Within each dose group, a sentinel dosing schedule for the first dose administration will be used as follows:
On Day 1, 2 subjects (1 active SIF001; 1 placebo) will be dosed and observed.
On Day 3, if safety and tolerability from subjects dosed on Day 1 is satisfactory, the remaining subjects will be dosed and observed. As an additional precaution, the remaining subjects will not all be dosed simultaneously but be staggered with two subjects being dosed at a time.

After all subjects in the highest provisional dose level have completed their second dose (D15) or after a lower dose level is declared as the highest tolerated dose, one patient cohort of patients with epilepsy is planned to be enrolled at the highest tolerated dose. Forty (40) subjects will be randomized 3:2 to receive SIF001 or placebo.

### Dose Escalation and Stopping Rules:

Dose escalation decisions will be made based on all available safety and tolerability data (e.g., AEs, clinical laboratory tests, vital signs, 12-lead safety electrocardiograms [ECGs], and physical examinations) through 7 days after all subjects in the current dose group have received their first dose of SIF001 (SAD) and through 14 days after all subjects in the current dose group received their last dose of SIF001 (MAD). Data will be reviewed by a blinded SRC consisting of the Principal Investigator and sponsor representatives including the medical monitor, pharmacokineticist (if PK data is available), and others as deemed necessary.

The following variables (at a minimum) will be taken into account by the SRC at the protocol-specified time points for dose escalation decisions, and when events arise during the course of study treatment. AEs will be graded by the Investigator or qualified designee as noted.
Number and frequency of AEs characterized as moderate or severe in severity
Number and frequency of serious adverse events (SAEs)
Discontinuation due to AEs
Emergent cardiovascular events, such as corrected QT interval (QTc) increases or vital sign changes
Liver function enzyme increases

Interim PK data from previous cohort(s) will not be needed for a dose escalation decision but may be used to guide the dose escalation decision, as data becomes available. Any PK data will be presented to the SRC in an anonymized format to preserve the blind. A decision will be made by the group to either continue dosing as planned, modify dosing of the next and subsequent groups, or discontinue the study.

### Scientific Rationale for Study Design

The two stages of the proposed clinical trial evaluate the single- and multiple-dose safety and tolerability of SIF001 in healthy volunteers. In the SAD (Stage I) and MAD (Stage II) portions of the study, 8 healthy volunteers will be randomized 3:1 to receive active SIF001 treatment or placebo. Within each dose group, a sentinel dosing schedule will be applied to SAD and MAD stages mitigate potential safety risks.

Dose escalation decisions will be based on all available safety and tolerability data through 7 days after at least 6 out of 8 subjects in the current dose group have received their first dose of SIF001. Data will be reviewed by a blinded SRC consisting of the Principal Investigator and sponsor representatives including the medical monitor, pharmacokineticist (if PK data is available), statistician, and others as deemed necessary. A decision will be made by the group to either continue dosing as planned, modify dosing of the next and subsequent groups, or discontinue the study.

Given the unmet medical need of patients with drug-resistant epilepsy, an additional cohort in the MAD stage is planned to enroll patients with epilepsy, who have not responded on prior treatment of AEDs. This cohort will help assess the safety and PK of SIF001-001 in the target patient population and generate preliminary efficacy data in a relatively small group of patients before exposing larger groups of patients to SIF001. Forty (40) patients with epilepsy will be enrolled, randomizing 24 patients to SIF001 and 16 patients to placebo (3:2). Assuming an early dropout rate of approximately 12.5%, 35 patients randomized 3:2 to active or placebo will provide 80% power to detect a difference of 30% in median percent reduction in seizure frequency between an active dosage group and placebo group, at a 2-sided alpha level of 0.05, assuming a standard deviation of 30%.

Upon the determination of an RP2D from the SAD and MAD evaluation, further studies of SIF001 may be conducted at the RP2D in the clinical development of SIF001.

### Justification for Dose

Nonclinical toxicology evaluation of SIF001 was conducted in Sprague Dawley rats and beagle dogs. The no observed adverse effect level (NOAEL) from the repeat-dose Good Laboratory Practice-compliant toxicology studies was 200 mg/kg (Q2W×3 doses) in both rats and beagle dogs. The human equivalent dose (HED) of the NOAEL calculated based on body surface area conversion is as follows:
In rats: 200 mg/kg/6.2=32.3 mg/kg
In dogs: 200 mg/kg/1.8=111.1 mg/kg

Assuming beagle dogs as the most appropriate species for calculation of the initial clinical dose, and applying a safety factor of 10, the maximum recommended starting dose (MRSD) is: 111.1 mg/kg (HED)×1/10=11.1 mg/kg. Thus, the starting dose for the proposed Investigational New Drug (IND)-opening study is conservatively set at 10 mg/kg.

The high-dose selection of 200 mg/kg for the pivotal GLP toxicology studies in rats and dogs was partially due to the maximum allowable volume of intravenous fluids that may be administered to healthy adult animals according to Institutional Animal Care and Use Committee (IACUC) guidelines, which is 10.0 ml/kg for both rats and dogs [12]. Because the formulation of the SIF001 drug substance used for the toxicology studies was 20 mg/mL, the maximum feasible dose that could be tested in rats and dogs was 200 mg/kg.

According to ICH S6 (R1) Addendum (May 2012), the high-dose selection should reflect an approximately 10-fold exposure multiple over the maximum exposure to be achieved in the clinic unless there is a justification for using a lower dose (e.g., maximum feasible dose) [13]. Since dose levels above 200 mg/kg could not be tested in rats and dogs, a review of the available clinical data by US FDA will be planned for dose escalations above 20 mg/kg (1/10 of the maximum feasible dose tested in nonclinical studies). The proposed dose levels for the SAD and MAD stages of this trial are 10, 20, 40, and 70 mg/kg.

### Population Size

A total of 104 subjects are tentatively planned to be enrolled in the dose escalation study. Stage I (10⁻⁷⁰ mg/kg): approximately 32 subjects. Stage II (10⁻⁷⁰ mg/kg): approximately 72 subjects including 32 healthy volunteers and 40 patients with epilepsy

### Inclusion Criteria

Participants are eligible to be included in the study only if they meet the following criteria:

### Healthy Volunteers (Stage I and II (Phase 1a and 1b)):

1. Male or female 18 to 55 years of age at the time of signing the informed consent.
2. In good health as determined by the Investigator, based on medical history and screening evaluations.
3. Body weight of ≥50 kg and BMI within the range 18-30 kg/m² (inclusive)
   Patients with Epilepsy (Stage II (Phase 1b)):
4. Male or female 18 to 70 years of age at the time of signing the informed consent.
5. Clinical diagnosis of epilepsy. Subjects having either partial or generalized epilepsy with motor seizures or seizures with clear alteration of awareness are eligible for enrollment.
6. Has a minimum of 4 seizures per 4-week period while taking 1 to 3 anti-seizure medications
7. All medications and epilepsy interventions must be stable for 8 weeks before screening and are expected to remain stable during the study

### All Subjects:

8. Negative serum pregnancy test at screening and urine pregnancy test on Day −1 before starting study treatment in all pre-menopausal women and women <12 months after the onset of menopause.
9. Female participants of child-bearing potential and male participants must agree to use adequate contraception for the duration of the protocol.
10. Able to sign informed consent and comply with the protocol.

### Storage, Preparation, and Administration of Study Drug

The study drug, SIF001 Injection or placebo, is supplied as a 20 mg/mL solution, with 8 mL per vial. The study drug should be stored at 2˜8° C.

Subjects will be randomly assigned to receive SIF001 or placebo. Investigators will remain blinded to each subject's assigned study treatment throughout the course of the study. In order to maintain this blind, an unblinded pharmacist will be responsible for the reconstitution and dispensation of the study drug.

SIF001 will be administered based on body weight. The provisional dose levels planned for SAD stage are 10, 20, 40, and 70 mg/kg on Day 1, and the provisional dose levels planned for the MAD stage are 10, 20, 40, and 70 mg/kg on Days 1, 15, and 29.

The test drug for this study is SIF001 and the control drug is placebo. Study drug will be administered as a one to two-hour IV infusion from a diluted saline bag with a total volume of 100, 250, or 500 mL, depending on dose level.

### Sample Size

In the SAD and MAD healthy volunteer cohorts, subjects will be recruited sequentially in separate increasing dose groups, with each group randomizing 6 subjects to a specific dose of SIF001 and 2 randomized to placebo (3:1). No formal sample size calculations have been performed for the SAD and MAD healthy volunteer cohorts. The number of subjects is based on feasibility and is considered sufficient to meet the study objectives.

In the MAD patient cohort, 40 patients with epilepsy will be enrolled and dosed, randomizing 24 patients to SIF001 and 16 patients to placebo (3:2). Assuming an early dropout rate of approximately 12.5%, 35 patients randomized 3:2 to active or placebo will provide 80% power to detect a difference of 30% in mean percent reduction in seizure frequency between an active dosage group and placebo group, at a 2-sided alpha level of 0.05, assuming a standard deviation of 30%.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, may be referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure and are to be included within the scope of this application.

While particular alternatives of the present disclosure have been disclosed, it is to be understood that various modifications and combinations are possible and are contemplated within the scope of the appended claims. There is no intention, therefore, of limitations to the exact abstract and disclosure herein presented.

### Example 12. In Vitro Epitope Binning of Antibodies Binding with Human Galectin-3 (hGal-3)

This example illustrates utilizing competitive binning to determine the anti-Gal-3 antibodies compete to bind to human Gal-3.

### Summary

The purpose of this study is to determine the epitope binning of antibodies against human Gal-3. Binning assay was conducted to characterize epitopes of the anti-Gal-3 antibodies using a Carterra LSA instrument. All the antibodies were constructed in hIgG4 backbone. Antibodies as ligands were covalently linked to a HC200M chip. The antigen hGal-3 as the analyte was injected to flow cells to bind to the captured ligands. Competitive binning was programmed to determine if two mAbs can bind simultaneously to the same antigen. The experiment showed 66 antibodies of the dendrogram with a cut height that created 18 well differentiated branches.

Carterra LSA is a monoclonal antibody (mAb) characterization platform that combines patented flow printing microfluidics with high throughput surface plasmon resonance (SPR) detection. In this study, Carterra LSA was used to measure the affinity of Gal-3 binding to antibody with surface plasmon resonance (SPR) and epitope binning. The LSA Epitope Binning workflow is illustrated in FIG. 35.

Epitope binning assay procedure. An array of antibodies as ligand were coupled to the chip surface HC200M using the Multichannel PH with the running buffer (25 mM MES buffer). The NHS/EDC activation time and coupling time was set at 5 min and 15 min respectively. The SFC then docked onto the chip, and in each cycle, antigen human Galectin-3 was injected across the entire Ab array, with association time of 5 min, followed by a single antibody as analyte with association time of 5 min. After 1 min dissociation, regeneration buffer (pH 2.0, 10 mM Glycine) was applied for 20 s for three times. The data were analyzed by Caterra Epitope Data Analysis Software.

An overview of the epitope binning results is displayed as follows.

Community clusters. A combined dendrogram with a cut height creating 18 well differentiated branches, which correspond to 18 epitope clusters. The individual community clusters with their exemplary antibodies are listed in Table 10. It is understood that additional antibodies may be classified into one of the community clusters based on results of epitope binning experiments as described above.

TABLE 10
18 epitope clusters

| | | | |
|---|---|---|---|
| Com 1 | 1104SBC1068-033 | Com 9 | 20240903SBC1093-015 |
| Com 2 | 1220SBC1068-186 | | 20240903SBC1093-020 |
| | 1220SBC1068-205 | | 20240903SBC1093-023 |
| | 1220SBC1068-022 | | 20240903SBC1093-030 |
| | 1220SBC1068-058 | | 20240628SBC1080-005 |
| | 1220SBC1068-056 | | 20240903SBC1093-183 |
| | 20241223SBC1205-119 | | 20240903SBC1093-220 |
| | 1104SBC1068-160 | | 20240903SBC1093-230 |
| Com 3 | 20240628SBC1080-009 | | 20240903SBC1093-185 |
| | 20240628SBC1080-002 | | 20240903SBC1093-021 |
| | 1104SBC1068-374 | | 20240903SBC1093-037 |
| | 20240929SBC1205-022 | | 20240903SBC1093-036 |
| | 1104SBC1068-335 | | 20240903SBC1093-099 |
| | 20040628SBC1080-006 | | 20240903SBC1093-025 |
| | 20240628SBC1080-008 | | 20240903SBC1093-182 |
| | 1104SBC1068-378 | | 20240903SBC1093-184 |
| | 1104SBC1068-365 | Com 10 | 20240903SBC1093-033 |
| | ANb1361-G119-5M- | Com 11 | 20240903SBC1093-034 |
| | LP2R2-58-higg4P | Com 12 | 20240903SBC1093-228 |
| | 20240628SBC1080-007 | | 20240903SBC1093-074 |
| Com 4 | 1220SBC1068-035 | | 20240903SBC1093-193 |
| Com 5 | 1220SBC1068-098 | | 20240903SBC1093-044 |
| | 1220SBC1068-210 | | 20240903SBC1093-059 |
| | 1220SBC1068-041 | | 20240903SBC1093-056 |
| | 1220SBC1068-063 | | 20240903SBC1093-057 |
| | 1220SBC1068-274 | | 20240903SBC1093-219 |
| | 1220SBC1068-129 | | 20240903SBC1093-194 |
| | 1220SBC1068-099 | | 20240903SBC1093-035 |
| | 1220SBC1068-164 | Com 13 | 20240903SBC1093-055 |
| | 1220SBC1068-197 | Com 14 | 20240903SBC1093-075 |
| Com 6 | 1220SBC1068-097 | Com 15 | 20241223SBC1205-061 |
| Com 7 | 20240628SBC1080-003 | Com 16 | 20241223SBC1205-063 |
| Com 8 | 20240628SBC1080-004 | Com 17 | 20241223SBC1205-130 |
| Com 18 | 20241223SBC1205-131 | | |

The term “Com” stands for community cluster.

### Conclusions

The epitope identification data of the 66 antibodies demonstrate a diverse range of epitopes.

### REFERENCES

1. Ahmed, H.; Du, S.-J.; O'Leary, N.; Vasta, G. R. Biochemical and molecular characterization of galectins from zebrafish (Danio rerio): Notochord-specific expression of a prototype galectin during early embryogenesis. Glycobiology 2003, 14, 219-232.
2. Nakahara, S.; Raz, A. Regulation of cancer-related gene expression by galectin-3 and the molecular mechanism of its nuclear import pathway. Cancer Metastasis Rev. 2007, 26, 605-610.
3. Mariño, K. V.; Cagnoni, A. J.; Croci, D. O.; Rabinovich, G. A. Targeting galectin-driven regulatory circuits in cancer and fibrosis. Nat. Rev. Drug. Discov. 2023, 22, 295-316.
4. Nakahara, S.; Raz, A. Biological modulation by lectins and their ligands in tumor progression and metastasis. Anticancer. Agents Med. Chem. 2008, 8, 22-36.
5. Vasta, G. R. Roles of galectins in infection. Nat. Rev. Genet. 2009, 7, 424-438.
6. Henderson, N. C.; Mackinnon, A. C.; Farnworth, S. L.; Poirier, F.; Russo, F. P.; Iredale, J. P.; Haslett, C.; Simpson, K. J.; Sethi, T. Galectin-3 regulates myofibroblast activation and hepatic fibrosis. Proc. Natl. Acad. Sci. USA 2006, 103, 5060-5065.
7. Nishi, Y.; Sano, H.; Kawashima, T.; Okada, T.; Kuroda, T.; Kikkawa, K.; Kawashima, S.; Tanabe, M.; Goto, T.; Matsuzawa, Y.; et al. Role of Galectin-3 in Human Pulmonary Fibrosis. Allergol. Int. 2007, 56, 57-65.
8. Juniantito, V.; Izawa, T.; Yamamoto, E.; Murai, F.; Kuwamura, M.; Yamate, J. Heterogeneity of Macrophage Populations and Expression of Galectin-3 in Cutaneous Wound Healing in Rats. J. Comp. Pathol. 2011, 145, 378-389.
9. Henderson, N. C.; Mackinnon, A. C.; Farnworth, S. L.; Kipari, T.; Haslett, C.; Iredale, J. P.; Liu, F.-T.; Hughes, J.; Sethi, T. Galectin-3 Expression and Secretion Links Macrophages to the Promotion of Renal Fibrosis. Am. J. Pathol. 2008, 172, 288-298.
10. Ho, J. E.; Liu, C.; Lyass, A.; Courchesne, P.; Pencina, M. J.; Vasan, R. S.; Larson, M. G.; Levy, D. Galectin-3, a Marker of Cardiac Fibrosis, Predicts Incident Heart Failure in the Community. J. Am. Coll. Cardiol. 2012, 60, 1249-1256.
11. Li, P.; Liu, S.; Lu, M.; Bandyopadhyay, G.; Oh, D.; Imamura, T.; Johnson, A. M. F.; Scars, D.; Shen, Z.; Cui, B.; et al. Hematopoietic Derived Galectin-3 Causes Cellular and Systemic Insulin Resistance. Cell 2016, 167, 973-984.e12.
12. Pejnovic, N. Galectin-3 In Obesity And Type 2 Diabetes. Serb. J. Exp. Clin. Res. 2015, 16, 273-280.
13. Bobronnikova, L. Galectin-3 as a potential biomarker of metabolic disorders and cardiovascular remodeling in patients with hypertension and type 2 diabetes. Vessel Plus
14. Yilmaz, H.; Cakmak, M.; Inan, O.; Darcin, T.; Akcay, A. Increased levels of galectin-3 were associated with prediabetes and diabetes: New risk factor? J. Endocrinol. Investig. 2015, 38, 527-533.
15. Siwicki, M.; Engblom, C.; Pittet, M. J. Gal3 Links Inflammation and Insulin Resistance. Cell Metab. 2016, 24, 655-656.
16. Mensah-Brown, E.; Al Rabesi, Z.; Shahin, A.; Al Shamsi, M.; Arsenijevic, N.; Hsu, D.; Liu, F.-T.; Lukic, M. Targeted disruption of the galectin-3 gene results in decreased susceptibility to multiple low dose streptozotocin-induced diabetes in mice. Clin. Immunol. 2009, 130, 83-88.
17. Elola, M. T.; Wolfenstein-Todel, C.; Troncoso, M. F.; Vasta, G. R.; Rabinovich, G. A. Galectins: Matricellular glycan-binding proteins linking cell adhesion, migration, and survival. Cell. Mol. Life Sci. 2007, 64, 1679-1700.
18. Nakahara, S.; Oka, N.; Raz, A. On the role of galectin-3 in cancer apoptosis. Apoptosis. 2005, 10, 267-275.
19. Dumic, J.; Dabelic, S.; Flögel, M. Galectin-3: An open-ended story. Biochim. Biophys. Acta (BBA) Gen. Subj. 2006, 1760, 616-635.
20. Henderson, N. C.; Sethi, T. The regulation of inflammation by galectin-3. Immunol. Rev. 2009, 230, 160-171.
21. Ahmed, H.; AlSadek, D. M. M. Galectin-3 as a Potential Target to Prevent Cancer Metastasis. Clin. Med. Insights Oncol. 2015, 9, 113-121.
22. Bian, C.-F.; Zhang, Y.; Sun, H.; Li, D.-F.; Wang, D.-C. Structural Basis for Distinct Binding Properties of the Human Galectins to Thomsen-Friedenreich Antigen. PloS ONE 2011, 6, e25007.
23. Liao, D. I.; Kapadia, G.; Ahmed, H.; Vasta, G. R.; Herzberg, O. Structure of S-lectin, a developmentally regulated vertebrate beta-galactoside binding protein. Proc. Natl. Acad. Sci. USA 1994, 91, 1428-1432.
24. Bianchet, M. A.; Ahmed, H.; Vasta, G. R.; Amzel, L. M. A soluble β-galactosyl-binding lectin (galectin) from toad (Bufo arenarum Hensel) ovary: Crystallographic studies of two protein-sugar complexes. Proteins. 2000, 40, 378-388.
25. Hirabayashi, J.; Kasai, K. The family of metazoan metal-independent β-galactoside-binding lectins: Structure, function and molecular evolution. Glycobiology. 1993, 3, 297-304.
26. Cooper, D. N.; Barondes, S. H. Evidence for export of a muscle lectin from cytosol to extracellular matrix and for a novel secretory mechanism. J. Cell Biol. 1990, 110, 1681-1691.
27. Cho, M.; Cummings, R. D. Galectin-1, a β-galactoside-binding lectin in Chinese hamster ovary cells, II. Localization and biosynthesis. J. Biol. Chem. 1995, 270, 5207-5212.
28. Barboni, E. A.; Bawumia, S.; Hughes, R. C. Kinetic measurements of binding of galectin 3 to a laminin substratum. Glycoconj. J. 1999, 16, 365-373.
29. Kariya, Y.; Kawamura, C.; Tabci, T.; Gu, J. Bisecting GlcNAc Residues on Laminin-332 Down-regulate Galectin-3-dependent Keratinocyte Motility. J. Biol. Chem. 2010, 285, 3330-3340.
30. Ochieng, J.; Leite-Browning, M. L.; Warfield, P. Regulation of Cellular Adhesion to Extracellular Matrix Proteins by Galectin-3. Biochem. Biophys. Res. Commun. 1998, 246, 788-791.
31. Fukumori, T.; Takenaka, Y.; Yoshii, T.; Kim, H.-R. C.; Hogan, V.; Inohara, H.; Kagawa, S.; Raz, A. CD29 and CD7 mediate galectin-3-induced type II T-cell apoptosis. Cancer Res. 2003, 63, 8302-8311.
32. Feuk-Lagerstedt, E.; Jordan, E. T.; Leffler, H.; Dahlgren, C.; Karlsson, A. Identification of CD66a and CD66b as the major galectin-3 receptor candidates in human neutrophils. J. Immunol. 1999, 163, 5592-5598.
33. Rosenberg, I.; Cherayil, B. J.; Isselbacher, K. J.; Pillai, S. Mac-2-binding glycoproteins. Putative ligands for a cytosolic betagalactoside lectin. J. Biol. Chem. 1991, 266, 18731-18736.
34. Park, J. W.; Voss, P. G.; Grabski, S.; Wang, J. L.; Patterson, R. J. Association of galectin-1 and galectin-3 with Gemin4 in complexes containing the SMN protein. Nucleic Acids Res. 2001, 29, 3595-3602.
35. Liu, L.; Sakai, T.; Sano, N.; Fukui, K. Nucling mediates apoptosis by inhibiting expression of galectin-3 through interference with nuclear factor kappaB signalling. Biochem. J. 2004, 380, 31-41.
36. Yu, F.; Finley, R. L., Jr.; Raz, A.; Kim, H. R. Galectin-3 translocates to the perinuclear membranes and inhibits cytochrome c release from the mitochondria. A role for synexin in galectin-3 translocation. J. Biol. Chem. 2002, 277, 15819-15827.
37. Shimura, T.; Takenaka, Y.; Tsutsumi, S.; Hogan, V.; Kikuchi, A.; Raz, A. Galectin-3, a novel binding partner of beta-catenin. Cancer Res. 2004, 64, 6363-6367.
38. Song, S.; Mazurek, N.; Liu, C.; Sun, Y.; Ding, Q. Q.; Liu, K.; Hung, M. C.; Bresalier, R. S. Galectin-3 mediates nuclear beta-catenin accumulation and Wnt signaling in human colon cancer cells by regulation of glycogen synthase kinase-3beta activity. Cancer Res. 2009, 69, 1343-1349.
39. Hencka M T, Golenbock D T, Latz E. Innate immunity in Alzheimer's disease. Nat Immunol. 2015; 16 (3): 229-36.
40. Cai Z, Hussain M D, Yan L J. Microglia, neuroinflammation, and beta-amyloid protein in Alzheimer's disease. Int J Neurosci. 2014; 124 (5): 307-21.
41. Yang R Y, Rabinovich G A, Liu F T. Galectins: structure, function and therapeutic potential. Expert Rev Mol Med. 2008; 10: e17.
42. Ramírez Hernández E, et al. The therapeutic potential of galectin-1 and galectin-3 in the treatment of neurodegenerative diseases. Expert Rev Neurother. 2020; 20 (5): 439-48.
43. Mendonca H R, et al. Neuroinflammation and oxidative stress act in concert to promote neurodegeneration in the diabetic retina and optic nerve: galectin-3 participation. Neural Regen Res. 2020; 15 (4): 625-35.
44. Dong R, et al. Galectin-3 as a novel biomarker for disease diagnosis and a target for therapy (Review). Int J Mol Med. 2018; 41 (2): 599-614.
45. Shin T. The pleiotropic effects of galectin-3 in neuroinflammation: a review. Acta Histochem. 2013; 115 (5): 407-11.
46. Yoo H I, et al. Neuroanatomical distribution of galectin-3 in the adult rat brain. J Mol Histol. 2017; 48 (2): 133-46.
47. Song L, et al. Annexin A7 and its binding protein galectin-3 influence mouse hepatocellular carcinoma cell line in vitro. Biomed Pharmacother. 2014; 68 (3): 377-84.
48. Li Q, Barres B A. Microglia and macrophages in brain homeostasis and disease. Nat Rev Immunol. 2018; 18 (4): 225-42.
49. Xu L, He D, Bai Y. Microglia-mediated inflammation and neurodegenerative disease. Mol Neurobiol. 2016; 53 (10): 6709-15.
50. Tang Y, Le W. Differential roles of M1 and M2 microglia in neurodegenerative diseases. Mol Neurobiol. 2016; 53 (2): 1181-94.
51. Jeon S B, et al. Galectin-3 exerts cytokine-like regulatory actions through the JAK-STAT pathway. J Immunol. 2010; 185 (11): 7037-46.
52. Rahimian R, Beland L C, Kriz J. Galectin-3: mediator of microglia responses in injured brain. Drug Discov Today. 2018; 23 (2): 375-81.
53. Siew J J, Chern Y. Microglial lectins in health and neurological diseases. Front Mol Neurosci. 2018; 11:158.
54. Rahimian R, et al. Delayed galectin-3-mediated reprogramming of microglia after stroke is protective. Mol Neurobiol. 2019; 56 (9): 6371-85.
55. Yip P K, et al. Galectin-3 released in response to traumatic brain injury acts as an alarmin orchestrating brain immune response and promoting neurodegeneration. Sci Rep. 2017; 7:41689.
56. Itabashi T, et al. Cell- and stage-specific localization of galectin-3, a beta-galactoside-binding lectin, in a mouse model of experimental autoimmune encephalomyelitis. Neurochem Int. 2018; 118:176-84.
57. Ahmed, R.; Anam, K.; Ahmed, H. Development of Galectin-3 Targeting Drugs for Therapeutic Applications in Various Diseases. Int. J. Mol. Sci. 2023, 24, 8116.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. An antibody or a binding fragment thereof that binds to Galectin-3, wherein the antibody comprises:
a heavy chain variable (V_{H}) region comprising:
a HCDR1 amino acid sequence at least 70% identical to any one of SEQ ID NOS: 1-21 and 370-389, a HCDR2 amino acid sequence at least 70% identical to any one of SEQ ID NOS: 22-42 and 390-417, and/or a HCDR3 amino acid sequence at least 70% identical to any one of SEQ ID NOS: 43-63 and 418-439, and/or
a light chain variable (V_{L}) region comprising:
a LCDR1 amino acid sequence at least 70% identical to any one of SEQ ID NOS: 169-189 and 440-462, a LCDR2 amino acid sequence at least 70% identical to any one of SEQ ID NOS: 190-210 and 463-480, and/or a LCDR3 amino acid sequence at least 70% identical to any one of SEQ ID NOS: 211-231 and 481-507,
optionally wherein the antibody or the binding fragment thereof is for use in the treatment of epilepsy or a related neurological disorder in a subject.

2. The antibody or the binding fragment thereof of claim 1, wherein the antibody comprises:
a heavy chain variable region comprising a HCDR1 amino acid sequence at least 70% identical to SEQ ID NO: 1, a HCDR2 amino acid sequence at least 70% identical to SEQ ID NO: 22, and a HCDR3 amino acid sequence at least 70% identical to SEQ ID NO: 43; and a light chain variable region comprising a LCDR1 amino acid sequence at least 70% identical to SEQ ID NO: 169, a LCDR2 amino acid sequence at least 70% identical to SEQ ID NO: 190, and a LCDR3 amino acid sequence at least 70% identical to SEQ ID NO: 211, or
a heavy chain variable region comprising a HCDR1 amino acid sequence at least 70% identical to SEQ ID NO: 2, a HCDR2 amino acid sequence at least 70% identical to SEQ ID NO: 23, and a HCDR3 amino acid sequence at least 70% identical to SEQ ID NO: 44; and a light chain variable region comprising a LCDR1 amino acid sequence at least 70% identical to SEQ ID NO: 170, a LCDR2 amino acid sequence at least 70% identical to SEQ ID NO: 191, and a LCDR3 amino acid sequence at least 70% identical to SEQ ID NO: 212, or
a heavy chain variable region comprising a HCDR1 amino acid sequence at least 70% identical to SEQ ID NO: 3, a HCDR2 amino acid sequence at least 70% identical to SEQ ID NO: 24, and a HCDR3 amino acid sequence at least 70% identical to SEQ ID NO: 45; and a light chain variable region comprising a LCDR1 amino acid sequence at least 70% identical to SEQ ID NO: 171, a LCDR2 amino acid sequence at least 70% identical to SEQ ID NO: 192, and a LCDR3 amino acid sequence at least 70% identical to SEQ ID NO: 213, or
a heavy chain variable region comprising a HCDR1 amino acid sequence at least 70% identical to SEQ ID NO: 4, a HCDR2 amino acid sequence at least 70% identical to SEQ ID NO: 25, and a HCDR3 amino acid sequence at least 70% identical to SEQ ID NO: 46; and a light chain variable region comprising a LCDR1 amino acid sequence at least 70% identical to SEQ ID NO: 172, a LCDR2 amino acid sequence at least 70% identical to SEQ ID NO: 193, and a LCDR3 amino acid sequence at least 70% identical to SEQ ID NO: 214, or
a heavy chain variable region comprising a HCDR1 amino acid sequence at least 70% identical to SEQ ID NO: 5, a HCDR2 amino acid sequence at least 70% identical to SEQ ID NO: 26, and a HCDR3 amino acid sequence at least 70% identical to SEQ ID NO: 47; and a light chain variable region comprising a LCDR1 amino acid sequence at least 70% identical to SEQ ID NO: 173, a LCDR2 amino acid sequence at least 70% identical to SEQ ID NO: 194, and a LCDR3 amino acid sequence at least 70% identical to SEQ ID NO: 215, or
a heavy chain variable region comprising a HCDR1 amino acid sequence at least 70% identical to SEQ ID NO: 6, a HCDR2 amino acid sequence at least 70% identical to SEQ ID NO: 27, and a HCDR3 amino acid sequence at least 70% identical to SEQ ID NO: 48; and a light chain variable region comprising a LCDR1 amino acid sequence at least 70% identical to SEQ ID NO: 174, a LCDR2 amino acid sequence at least 70% identical to SEQ ID NO: 195, and a LCDR3 amino acid sequence at least 70% identical to SEQ ID NO: 216, or
a heavy chain variable region comprising a HCDR1 amino acid sequence at least 70% identical to SEQ ID NO: 7, a HCDR2 amino acid sequence at least 70% identical to SEQ ID NO: 28, and a HCDR3 amino acid sequence at least 70% identical to SEQ ID NO: 49; and a light chain variable region comprising a LCDR1 amino acid sequence at least 70% identical to SEQ ID NO: 175, a LCDR2 amino acid sequence at least 70% identical to SEQ ID NO: 196, and a LCDR3 amino acid sequence at least 70% identical to SEQ ID NO: 217, or
a heavy chain variable region comprising a HCDR1 amino acid sequence at least 70% identical to SEQ ID NO: 8, a HCDR2 amino acid sequence at least 70% identical to SEQ ID NO: 29, and a HCDR3 amino acid sequence at least 70% identical to SEQ ID NO: 50; and a light chain variable region comprising a LCDR1 amino acid sequence at least 70% identical to SEQ ID NO: 176, a LCDR2 amino acid sequence at least 70% identical to SEQ ID NO: 197, and a LCDR3 amino acid sequence at least 70% identical to SEQ ID NO: 218, or
a heavy chain variable region comprising a HCDR1 amino acid sequence at least 70% identical to SEQ ID NO: 9, a HCDR2 amino acid sequence at least 70% identical to SEQ ID NO: 30, and a HCDR3 amino acid sequence at least 70% identical to SEQ ID NO: 51; and a light chain variable region comprising a LCDR1 amino acid sequence at least 70% identical to SEQ ID NO: 177, a LCDR2 amino acid sequence at least 70% identical to SEQ ID NO: 198, and a LCDR3 amino acid sequence at least 70% identical to SEQ ID NO: 219, or
a heavy chain variable region comprising a HCDR1 amino acid sequence at least 70% identical to SEQ ID NO: 10, a HCDR2 amino acid sequence at least 70% identical to SEQ ID NO: 31, and a HCDR3 amino acid sequence at least 70% identical to SEQ ID NO: 52; and a light chain variable region comprising a LCDR1 amino acid sequence at least 70% identical to SEQ ID NO: 178, a LCDR2 amino acid sequence at least 70% identical to SEQ ID NO: 199, and a LCDR3 amino acid sequence at least 70% identical to SEQ ID NO: 220, or
a heavy chain variable region comprising a HCDR1 amino acid sequence at least 70% identical to SEQ ID NO: 11, a HCDR2 amino acid sequence at least 70% identical to SEQ ID NO: 32, and a HCDR3 amino acid sequence at least 70% identical to SEQ ID NO: 53; and a light chain variable region comprising a LCDR1 amino acid sequence at least 70% identical to SEQ ID NO: 179, a LCDR2 amino acid sequence at least 70% identical to SEQ ID NO: 200, and a LCDR3 amino acid sequence at least 70% identical to SEQ ID NO: 221, or
a heavy chain variable region comprising a HCDR1 amino acid sequence at least 70% identical to SEQ ID NO: 12, a HCDR2 amino acid sequence at least 70% identical to SEQ ID NO: 33, and a HCDR3 amino acid sequence at least 70% identical to SEQ ID NO: 54; and a light chain variable region comprising a LCDR1 amino acid sequence at least 70% identical to SEQ ID NO: 180, a LCDR2 amino acid sequence at least 70% identical to SEQ ID NO: 201, and a LCDR3 amino acid sequence at least 70% identical to SEQ ID NO: 222, or
a heavy chain variable region comprising a HCDR1 amino acid sequence at least 70% identical to SEQ ID NO: 13, a HCDR2 amino acid sequence at least 70% identical to SEQ ID NO: 34, and a HCDR3 amino acid sequence at least 70% identical to SEQ ID NO: 55; and a light chain variable region comprising a LCDR1 amino acid sequence at least 70% identical to SEQ ID NO: 181, a LCDR2 amino acid sequence at least 70% identical to SEQ ID NO: 202, and a LCDR3 amino acid sequence at least 70% identical to SEQ ID NO: 223, or
a heavy chain variable region comprising a HCDR1 amino acid sequence at least 70% identical to SEQ ID NO: 14, a HCDR2 amino acid sequence at least 70% identical to SEQ ID NO: 35, and a HCDR3 amino acid sequence at least 70% identical to SEQ ID NO: 56; and a light chain variable region comprising a LCDR1 amino acid sequence at least 70% identical to SEQ ID NO: 182, a LCDR2 amino acid sequence at least 70% identical to SEQ ID NO: 203, and a LCDR3 amino acid sequence at least 70% identical to SEQ ID NO: 224, or
a heavy chain variable region comprising a HCDR1 amino acid sequence at least 70% identical to SEQ ID NO: 15, a HCDR2 amino acid sequence at least 70% identical to SEQ ID NO: 36, and a HCDR3 amino acid sequence at least 70% identical to SEQ ID NO: 57; and a light chain variable region comprising a LCDR1 amino acid sequence at least 70% identical to SEQ ID NO: 183, a LCDR2 amino acid sequence at least 70% identical to SEQ ID NO: 204, and a LCDR3 amino acid sequence at least 70% identical to SEQ ID NO: 225, or
a heavy chain variable region comprising a HCDR1 amino acid sequence at least 70% identical to SEQ ID NO: 16, a HCDR2 amino acid sequence at least 70% identical to SEQ ID NO: 37, and a HCDR3 amino acid sequence at least 70% identical to SEQ ID NO: 58; and a light chain variable region comprising a LCDR1 amino acid sequence at least 70% identical to SEQ ID NO: 184, a LCDR2 amino acid sequence at least 70% identical to SEQ ID NO: 205, and a LCDR3 amino acid sequence at least 70% identical to SEQ ID NO: 226, or
a heavy chain variable region comprising a HCDR1 amino acid sequence at least 70% identical to SEQ ID NO: 17, a HCDR2 amino acid sequence at least 70% identical to SEQ ID NO: 38, and a HCDR3 amino acid sequence at least 70% identical to SEQ ID NO: 59; and a light chain variable region comprising a LCDR1 amino acid sequence at least 70% identical to SEQ ID NO: 185, a LCDR2 amino acid sequence at least 70% identical to SEQ ID NO: 206, and a LCDR3 amino acid sequence at least 70% identical to SEQ ID NO: 227, or
a heavy chain variable region comprising a HCDR1 amino acid sequence at least 70% identical to SEQ ID NO: 18, a HCDR2 amino acid sequence at least 70% identical to SEQ ID NO: 39, and a HCDR3 amino acid sequence at least 70% identical to SEQ ID NO: 60; and a light chain variable region comprising a LCDR1 amino acid sequence at least 70% identical to SEQ ID NO: 186, a LCDR2 amino acid sequence at least 70% identical to SEQ ID NO: 207, and a LCDR3 amino acid sequence at least 70% identical to SEQ ID NO: 228, or
a heavy chain variable region comprising a HCDR1 amino acid sequence at least 70% identical to SEQ ID NO: 19, a HCDR2 amino acid sequence at least 70% identical to SEQ ID NO: 40, and a HCDR3 amino acid sequence at least 70% identical to SEQ ID NO: 61; and a light chain variable region comprising a LCDR1 amino acid sequence at least 70% identical to SEQ ID NO: 187, a LCDR2 amino acid sequence at least 70% identical to SEQ ID NO: 208, and a LCDR3 amino acid sequence at least 70% identical to SEQ ID NO: 229, or
a heavy chain variable region comprising a HCDR1 amino acid sequence at least 70% identical to SEQ ID NO: 20, a HCDR2 amino acid sequence at least 70% identical to SEQ ID NO: 41, and a HCDR3 amino acid sequence at least 70% identical to SEQ ID NO: 62; and a light chain variable region comprising a LCDR1 amino acid sequence at least 70% identical to SEQ ID NO: 188, a LCDR2 amino acid sequence at least 70% identical to SEQ ID NO: 209, and a LCDR3 amino acid sequence at least 70% identical to SEQ ID NO: 230, or
a heavy chain variable region comprising a HCDR1 amino acid sequence at least 70% identical to SEQ ID NO: 21, a HCDR2 amino acid sequence at least 70% identical to SEQ ID NO: 42, and a HCDR3 amino acid sequence at least 70% identical to SEQ ID NO: 63; and a light chain variable region comprising a LCDR1 amino acid sequence at least 70% identical to SEQ ID NO: 189, a LCDR2 amino acid sequence at least 70% identical to SEQ ID NO: 210, and a LCDR3 amino acid sequence at least 70% identical to SEQ ID NO: 231.

3. The antibody or the binding fragment thereof of claim 1, wherein the antibody comprises:
a heavy chain variable region comprising a HCDR1 amino acid sequence at least 70% identical to SEQ ID NO: 370, a HCDR2 amino acid sequence at least 70% identical to SEQ ID NO: 390, and a HCDR3 amino acid sequence at least 70% identical to SEQ ID NO: 418; and a light chain variable region comprising a LCDR1 amino acid sequence at least 70% identical to SEQ ID NO: 440, a LCDR2 amino acid sequence at least 70% identical to SEQ ID NO: 463, and a LCDR3 amino acid sequence at least 70% identical to SEQ ID NO: 481, or
a heavy chain variable region comprising a HCDR1 amino acid sequence at least 70% identical to SEQ ID NO: 371, a HCDR2 amino acid sequence at least 70% identical to SEQ ID NO: 391, and a HCDR3 amino acid sequence at least 70% identical to SEQ ID NO: 418; and a light chain variable region comprising a LCDR1 amino acid sequence at least 70% identical to SEQ ID NO: 441, a LCDR2 amino acid sequence at least 70% identical to SEQ ID NO: 464, and a LCDR3 amino acid sequence at least 70% identical to SEQ ID NO: 482, or
a heavy chain variable region comprising a HCDR1 amino acid sequence at least 70% identical to SEQ ID NO: 372, a HCDR2 amino acid sequence at least 70% identical to SEQ ID NO: 392, and a HCDR3 amino acid sequence at least 70% identical to DNL; and a light chain variable region comprising a LCDR1 amino acid sequence at least 70% identical to SEQ ID NO: 442, a LCDR2 amino acid sequence at least 70% identical to SEQ ID NO: 465, and a LCDR3 amino acid sequence at least 70% identical to SEQ ID NO: 483, or
a heavy chain variable region comprising a HCDR1 amino acid sequence at least 70% identical to SEQ ID NO: 373, a HCDR2 amino acid sequence at least 70% identical to SEQ ID NO: 393, and a HCDR3 amino acid sequence at least 70% identical to SEQ ID NO: 419; and a light chain variable region comprising a LCDR1 amino acid sequence at least 70% identical to SEQ ID NO: 443, a LCDR2 amino acid sequence at least 70% identical to SEQ ID NO: 466, and a LCDR3 amino acid sequence at least 70% identical to SEQ ID NO: 484, or
a heavy chain variable region comprising a HCDR1 amino acid sequence at least 70% identical to SEQ ID NO: 374, a HCDR2 amino acid sequence at least 70% identical to SEQ ID NO: 394, and a HCDR3 amino acid sequence at least 70% identical to SEQ ID NO: 420; and a light chain variable region comprising a LCDR1 amino acid sequence at least 70% identical to SEQ ID NO: 444, a LCDR2 amino acid sequence at least 70% identical to SEQ ID NO: 467, and a LCDR3 amino acid sequence at least 70% identical to SEQ ID NO: 485, or
a heavy chain variable region comprising a HCDR1 amino acid sequence at least 70% identical to SEQ ID NO: 375, a HCDR2 amino acid sequence at least 70% identical to SEQ ID NO: 395, and a HCDR3 amino acid sequence at least 70% identical to SEQ ID NO: 421; and a light chain variable region comprising a LCDR1 amino acid sequence at least 70% identical to SEQ ID NO: 445, a LCDR2 amino acid sequence at least 70% identical to SEQ ID NO: 468, and a LCDR3 amino acid sequence at least 70% identical to SEQ ID NO: 486, or
a heavy chain variable region comprising a HCDR1 amino acid sequence at least 70% identical to SEQ ID NO: 376, a HCDR2 amino acid sequence at least 70% identical to SEQ ID NO: 396, and a HCDR3 amino acid sequence at least 70% identical to SEQ ID NO: 422; and a light chain variable region comprising a LCDR1 amino acid sequence at least 70% identical to SEQ ID NO: 446, a LCDR2 amino acid sequence at least 70% identical to SEQ ID NO: 469, and a LCDR3 amino acid sequence at least 70% identical to SEQ ID NO: 487, or
a heavy chain variable region comprising a HCDR1 amino acid sequence at least 70% identical to SEQ ID NO: 373, a HCDR2 amino acid sequence at least 70% identical to SEQ ID NO: 393, and a HCDR3 amino acid sequence at least 70% identical to SEQ ID NO: 419; and a light chain variable region comprising a LCDR1 amino acid sequence at least 70% identical to SEQ ID NO: 447, a LCDR2 amino acid sequence at least 70% identical to SEQ ID NO: 470, and a LCDR3 amino acid sequence at least 70% identical to SEQ ID NO: 484, or
a heavy chain variable region comprising a HCDR1 amino acid sequence at least 70% identical to SEQ ID NO: 377, a HCDR2 amino acid sequence at least 70% identical to SEQ ID NO: 397, and a HCDR3 amino acid sequence at least 70% identical to SEQ ID NO: 423; and a light chain variable region comprising a LCDR1 amino acid sequence at least 70% identical to SEQ ID NO: 448, a LCDR2 amino acid sequence at least 70% identical to SEQ ID NO: 471, and a LCDR3 amino acid sequence at least 70% identical to SEQ ID NO: 488, or
a heavy chain variable region comprising a HCDR1 amino acid sequence at least 70% identical to SEQ ID NO: 377, a HCDR2 amino acid sequence at least 70% identical to SEQ ID NO: 397, and a HCDR3 amino acid sequence at least 70% identical to SEQ ID NO: 423; and a light chain variable region comprising a LCDR1 amino acid sequence at least 70% identical to SEQ ID NO: 449, a LCDR2 amino acid sequence at least 70% identical to SEQ ID NO: 472, and a LCDR3 amino acid sequence at least 70% identical to SEQ ID NO: 488, or
a heavy chain variable region comprising a HCDR1 amino acid sequence at least 70% identical to SEQ ID NO: 377, a HCDR2 amino acid sequence at least 70% identical to SEQ ID NO: 397, and a HCDR3 amino acid sequence at least 70% identical to SEQ ID NO: 423; and a light chain variable region comprising a LCDR1 amino acid sequence at least 70% identical to SEQ ID NO: 450, a LCDR2 amino acid sequence at least 70% identical to SEQ ID NO: 471, and a LCDR3 amino acid sequence at least 70% identical to SEQ ID NO: 488, or
a heavy chain variable region comprising a HCDR1 amino acid sequence at least 70% identical to SEQ ID NO: 377, a HCDR2 amino acid sequence at least 70% identical to SEQ ID NO: 398, and a HCDR3 amino acid sequence at least 70% identical to SEQ ID NO: 423; and a light chain variable region comprising a LCDR1 amino acid sequence at least 70% identical to SEQ ID NO: 448, a LCDR2 amino acid sequence at least 70% identical to SEQ ID NO: 471, and a LCDR3 amino acid sequence at least 70% identical to SEQ ID NO: 488, or
a heavy chain variable region comprising a HCDR1 amino acid sequence at least 70% identical to SEQ ID NO: 377, a HCDR2 amino acid sequence at least 70% identical to SEQ ID NO: 397, and a HCDR3 amino acid sequence at least 70% identical to SEQ ID NO: 424; and a light chain variable region comprising a LCDR1 amino acid sequence at least 70% identical to SEQ ID NO: 448, a LCDR2 amino acid sequence at least 70% identical to SEQ ID NO: 472, and a LCDR3 amino acid sequence at least 70% identical to SEQ ID NO: 489, or
a heavy chain variable region comprising a HCDR1 amino acid sequence at least 70% identical to SEQ ID NO: 377, a HCDR2 amino acid sequence at least 70% identical to SEQ ID NO: 397, and a HCDR3 amino acid sequence at least 70% identical to SEQ ID NO: 423; and a light chain variable region comprising a LCDR1 amino acid sequence at least 70% identical to SEQ ID NO: 448, a LCDR2 amino acid sequence at least 70% identical to SEQ ID NO: 472, and a LCDR3 amino acid sequence at least 70% identical to SEQ ID NO: 488, or
a heavy chain variable region comprising a HCDR1 amino acid sequence at least 70% identical to SEQ ID NO: 377, a HCDR2 amino acid sequence at least 70% identical to SEQ ID NO: 397, and a HCDR3 amino acid sequence at least 70% identical to SEQ ID NO: 423; and a light chain variable region comprising a LCDR1 amino acid sequence at least 70% identical to SEQ ID NO: 448, a LCDR2 amino acid sequence at least 70% identical to SEQ ID NO: 471, and a LCDR3 amino acid sequence at least 70% identical to SEQ ID NO: 490, or
a heavy chain variable region comprising a HCDR1 amino acid sequence at least 70% identical to SEQ ID NO: 377, a HCDR2 amino acid sequence at least 70% identical to SEQ ID NO: 398, and a HCDR3 amino acid sequence at least 70% identical to SEQ ID NO: 423; and a light chain variable region comprising a LCDR1 amino acid sequence at least 70% identical to SEQ ID NO: 448, a LCDR2 amino acid sequence at least 70% identical to SEQ ID NO: 471, and a LCDR3 amino acid sequence at least 70% identical to SEQ ID NO: 491, or
a heavy chain variable region comprising a HCDR1 amino acid sequence at least 70% identical to SEQ ID NO: 377, a HCDR2 amino acid sequence at least 70% identical to SEQ ID NO: 399, and a HCDR3 amino acid sequence at least 70% identical to SEQ ID NO: 424; and a light chain variable region comprising a LCDR1 amino acid sequence at least 70% identical to SEQ ID NO: 448, a LCDR2 amino acid sequence at least 70% identical to SEQ ID NO: 472, and a LCDR3 amino acid sequence at least 70% identical to SEQ ID NO: 489, or
a heavy chain variable region comprising a HCDR1 amino acid sequence at least 70% identical to SEQ ID NO: 377, a HCDR2 amino acid sequence at least 70% identical to SEQ ID NO: 397, and a HCDR3 amino acid sequence at least 70% identical to SEQ ID NO: 424; and a light chain variable region comprising a LCDR1 amino acid sequence at least 70% identical to SEQ ID NO: 448, a LCDR2 amino acid sequence at least 70% identical to SEQ ID NO: 471, and a LCDR3 amino acid sequence at least 70% identical to SEQ ID NO: 488, or
a heavy chain variable region comprising a HCDR1 amino acid sequence at least 70% identical to SEQ ID NO: 378, a HCDR2 amino acid sequence at least 70% identical to SEQ ID NO: 400, and a HCDR3 amino acid sequence at least 70% identical to SEQ ID NO: 425; and a light chain variable region comprising a LCDR1 amino acid sequence at least 70% identical to SEQ ID NO: 451, a LCDR2 amino acid sequence at least 70% identical to SEQ ID NO: 473, and a LCDR3 amino acid sequence at least 70% identical to SEQ ID NO: 492, or
a heavy chain variable region comprising a HCDR1 amino acid sequence at least 70% identical to SEQ ID NO: 378, a HCDR2 amino acid sequence at least 70% identical to SEQ ID NO: 401, and a HCDR3 amino acid sequence at least 70% identical to SEQ ID NO: 425; and a light chain variable region comprising a LCDR1 amino acid sequence at least 70% identical to SEQ ID NO: 452, a LCDR2 amino acid sequence at least 70% identical to SEQ ID NO: 473, and a LCDR3 amino acid sequence at least 70% identical to SEQ ID NO: 493, or
a heavy chain variable region comprising a HCDR1 amino acid sequence at least 70% identical to SEQ ID NO: 378, a HCDR2 amino acid sequence at least 70% identical to SEQ ID NO: 402, and a HCDR3 amino acid sequence at least 70% identical to SEQ ID NO: 425; and a light chain variable region comprising a LCDR1 amino acid sequence at least 70% identical to SEQ ID NO: 452, a LCDR2 amino acid sequence at least 70% identical to SEQ ID NO: 473, and a LCDR3 amino acid sequence at least 70% identical to SEQ ID NO: 493, or
a heavy chain variable region comprising a HCDR1 amino acid sequence at least 70% identical to SEQ ID NO: 379, a HCDR2 amino acid sequence at least 70% identical to SEQ ID NO: 403, and a HCDR3 amino acid sequence at least 70% identical to SEQ ID NO: 426; and a light chain variable region comprising a LCDR1 amino acid sequence at least 70% identical to SEQ ID NO: 453, a LCDR2 amino acid sequence at least 70% identical to SEQ ID NO: 470, and a LCDR3 amino acid sequence at least 70% identical to SEQ ID NO: 494, or
a heavy chain variable region comprising a HCDR1 amino acid sequence at least 70% identical to SEQ ID NO: 380, a HCDR2 amino acid sequence at least 70% identical to SEQ ID NO: 403, and a HCDR3 amino acid sequence at least 70% identical to SEQ ID NO: 426; and a light chain variable region comprising a LCDR1 amino acid sequence at least 70% identical to SEQ ID NO: 453, a LCDR2 amino acid sequence at least 70% identical to SEQ ID NO: 470, and a LCDR3 amino acid sequence at least 70% identical to SEQ ID NO: 494, or
a heavy chain variable region comprising a HCDR1 amino acid sequence at least 70% identical to SEQ ID NO: 380, a HCDR2 amino acid sequence at least 70% identical to SEQ ID NO: 403, and a HCDR3 amino acid sequence at least 70% identical to SEQ ID NO: 426; and a light chain variable region comprising a LCDR1 amino acid sequence at least 70% identical to SEQ ID NO: 453, a LCDR2 amino acid sequence at least 70% identical to SEQ ID NO: 474, and a LCDR3 amino acid sequence at least 70% identical to SEQ ID NO: 494, or
a heavy chain variable region comprising a HCDR1 amino acid sequence at least 70% identical to SEQ ID NO: 381, a HCDR2 amino acid sequence at least 70% identical to SEQ ID NO: 404, and a HCDR3 amino acid sequence at least 70% identical to SEQ ID NO: 426; and a light chain variable region comprising a LCDR1 amino acid sequence at least 70% identical to SEQ ID NO: 453, a LCDR2 amino acid sequence at least 70% identical to SEQ ID NO: 470, and a LCDR3 amino acid sequence at least 70% identical to SEQ ID NO: 494, or
a heavy chain variable region comprising a HCDR1 amino acid sequence at least 70% identical to SEQ ID NO: 381, a HCDR2 amino acid sequence at least 70% identical to SEQ ID NO: 404, and a HCDR3 amino acid sequence at least 70% identical to SEQ ID NO: 426; and a light chain variable region comprising a LCDR1 amino acid sequence at least 70% identical to SEQ ID NO: 453, a LCDR2 amino acid sequence at least 70% identical to SEQ ID NO: 470, and a LCDR3 amino acid sequence at least 70% identical to SEQ ID NO: 495, or
a heavy chain variable region comprising a HCDR1 amino acid sequence at least 70% identical to SEQ ID NO: 381, a HCDR2 amino acid sequence at least 70% identical to SEQ ID NO: 405, and a HCDR3 amino acid sequence at least 70% identical to SEQ ID NO: 426; and a light chain variable region comprising a LCDR1 amino acid sequence at least 70% identical to SEQ ID NO: 453, a LCDR2 amino acid sequence at least 70% identical to SEQ ID NO: 470, and a LCDR3 amino acid sequence at least 70% identical to SEQ ID NO: 494, or
a heavy chain variable region comprising a HCDR1 amino acid sequence at least 70% identical to SEQ ID NO: 378, a HCDR2 amino acid sequence at least 70% identical to SEQ ID NO: 406, and a HCDR3 amino acid sequence at least 70% identical to SEQ ID NO: 427; and a light chain variable region comprising a LCDR1 amino acid sequence at least 70% identical to SEQ ID NO: 452, a LCDR2 amino acid sequence at least 70% identical to SEQ ID NO: 470, and a LCDR3 amino acid sequence at least 70% identical to SEQ ID NO: 496, or
a heavy chain variable region comprising a HCDR1 amino acid sequence at least 70% identical to SEQ ID NO: 378, a HCDR2 amino acid sequence at least 70% identical to SEQ ID NO: 407, and a HCDR3 amino acid sequence at least 70% identical to SEQ ID NO: 425; and a light chain variable region comprising a LCDR1 amino acid sequence at least 70% identical to SEQ ID NO: 452, a LCDR2 amino acid sequence at least 70% identical to SEQ ID NO: 475, and a LCDR3 amino acid sequence at least 70% identical to SEQ ID NO: 496, or
a heavy chain variable region comprising a HCDR1 amino acid sequence at least 70% identical to SEQ ID NO: 382, a HCDR2 amino acid sequence at least 70% identical to SEQ ID NO: 408, and a HCDR3 amino acid sequence at least 70% identical to SEQ ID NO: 428; and a light chain variable region comprising a LCDR1 amino acid sequence at least 70% identical to SEQ ID NO: 454, a LCDR2 amino acid sequence at least 70% identical to SEQ ID NO: 471, and a LCDR3 amino acid sequence at least 70% identical to SEQ ID NO: 497, or
a heavy chain variable region comprising a HCDR1 amino acid sequence at least 70% identical to SEQ ID NO: 382, a HCDR2 amino acid sequence at least 70% identical to SEQ ID NO: 408, and a HCDR3 amino acid sequence at least 70% identical to SEQ ID NO: 429; and a light chain variable region comprising a LCDR1 amino acid sequence at least 70% identical to SEQ ID NO: 455, a LCDR2 amino acid sequence at least 70% identical to SEQ ID NO: 476, and a LCDR3 amino acid sequence at least 70% identical to SEQ ID NO: 498, or
a heavy chain variable region comprising a HCDR1 amino acid sequence at least 70% identical to SEQ ID NO: 382, a HCDR2 amino acid sequence at least 70% identical to SEQ ID NO: 408, and a HCDR3 amino acid sequence at least 70% identical to SEQ ID NO: 429; and a light chain variable region comprising a LCDR1 amino acid sequence at least 70% identical to SEQ ID NO: 454, a LCDR2 amino acid sequence at least 70% identical to SEQ ID NO: 468, and a LCDR3 amino acid sequence at least 70% identical to SEQ ID NO: 497, or
a heavy chain variable region comprising a HCDR1 amino acid sequence at least 70% identical to SEQ ID NO: 382, a HCDR2 amino acid sequence at least 70% identical to SEQ ID NO: 408, and a HCDR3 amino acid sequence at least 70% identical to SEQ ID NO: 430; and a light chain variable region comprising a LCDR1 amino acid sequence at least 70% identical to SEQ ID NO: 454, a LCDR2 amino acid sequence at least 70% identical to SEQ ID NO: 477, and a LCDR3 amino acid sequence at least 70% identical to SEQ ID NO: 497, or
a heavy chain variable region comprising a HCDR1 amino acid sequence at least 70% identical to SEQ ID NO: 383, a HCDR2 amino acid sequence at least 70% identical to SEQ ID NO: 409, and a HCDR3 amino acid sequence at least 70% identical to SEQ ID NO: 431; and a light chain variable region comprising a LCDR1 amino acid sequence at least 70% identical to SEQ ID NO: 456, a LCDR2 amino acid sequence at least 70% identical to SEQ ID NO: 474, and a LCDR3 amino acid sequence at least 70% identical to SEQ ID NO: 499, or
a heavy chain variable region comprising a HCDR1 amino acid sequence at least 70% identical to SEQ ID NO: 383, a HCDR2 amino acid sequence at least 70% identical to SEQ ID NO: 409, and a HCDR3 amino acid sequence at least 70% identical to SEQ ID NO: 431; and a light chain variable region comprising a LCDR1 amino acid sequence at least 70% identical to SEQ ID NO: 456, a LCDR2 amino acid sequence at least 70% identical to SEQ ID NO: 478, and a LCDR3 amino acid sequence at least 70% identical to SEQ ID NO: 499, or
a heavy chain variable region comprising a HCDR1 amino acid sequence at least 70% identical to SEQ ID NO: 384, a HCDR2 amino acid sequence at least 70% identical to SEQ ID NO: 410, and a HCDR3 amino acid sequence at least 70% identical to SEQ ID NO: 431; and a light chain variable region comprising a LCDR1 amino acid sequence at least 70% identical to SEQ ID NO: 457, a LCDR2 amino acid sequence at least 70% identical to SEQ ID NO: 474, and a LCDR3 amino acid sequence at least 70% identical to SEQ ID NO: 500, or
a heavy chain variable region comprising a HCDR1 amino acid sequence at least 70% identical to SEQ ID NO: 384, a HCDR2 amino acid sequence at least 70% identical to SEQ ID NO: 410, and a HCDR3 amino acid sequence at least 70% identical to SEQ ID NO: 432; and a light chain variable region comprising a LCDR1 amino acid sequence at least 70% identical to SEQ ID NO: 458, a LCDR2 amino acid sequence at least 70% identical to SEQ ID NO: 474, and a LCDR3 amino acid sequence at least 70% identical to SEQ ID NO: 500, or
a heavy chain variable region comprising a HCDR1 amino acid sequence at least 70% identical to SEQ ID NO: 385, a HCDR2 amino acid sequence at least 70% identical to SEQ ID NO: 411, and a HCDR3 amino acid sequence at least 70% identical to SEQ ID NO: 433; and a light chain variable region comprising a LCDR1 amino acid sequence at least 70% identical to SEQ ID NO: 459, a LCDR2 amino acid sequence at least 70% identical to SEQ ID NO: 470, and a LCDR3 amino acid sequence at least 70% identical to SEQ ID NO: 501.

4. The antibody or the binding fragment thereof of claim 1, wherein the antibody comprises:
a heavy chain variable region comprising a HCDR1 amino acid sequence at least 70% identical to SEQ ID NO: 386, a HCDR2 amino acid sequence at least 70% identical to SEQ ID NO: 412, and a HCDR3 amino acid sequence at least 70% identical to SEQ ID NO: 434; and a light chain variable region comprising a LCDR1 amino acid sequence at least 70% identical to SEQ ID NO: 460, a LCDR2 amino acid sequence at least 70% identical to SEQ ID NO: 479, and a LCDR3 amino acid sequence at least 70% identical to SEQ ID NO: 502, or
a heavy chain variable region comprising a HCDR1 amino acid sequence at least 70% identical to SEQ ID NO: 387, a HCDR2 amino acid sequence at least 70% identical to SEQ ID NO: 413, and a HCDR3 amino acid sequence at least 70% identical to SEQ ID NO: 435; and a light chain variable region comprising a LCDR1 amino acid sequence at least 70% identical to SEQ ID NO: 461, a LCDR2 amino acid sequence at least 70% identical to SEQ ID NO: 201, and a LCDR3 amino acid sequence at least 70% identical to SEQ ID NO: 503, or
a heavy chain variable region comprising a HCDR1 amino acid sequence at least 70% identical to SEQ ID NO: 4, a HCDR2 amino acid sequence at least 70% identical to SEQ ID NO: 414, and a HCDR3 amino acid sequence at least 70% identical to SEQ ID NO: 436; and a light chain variable region comprising a LCDR1 amino acid sequence at least 70% identical to SEQ ID NO: 462, a LCDR2 amino acid sequence at least 70% identical to SEQ ID NO: 480, and a LCDR3 amino acid sequence at least 70% identical to SEQ ID NO: 504, or
a heavy chain variable region comprising a HCDR1 amino acid sequence at least 70% identical to SEQ ID NO: 388, a HCDR2 amino acid sequence at least 70% identical to SEQ ID NO: 415, and a HCDR3 amino acid sequence at least 70% identical to SEQ ID NO: 437; and a light chain variable region comprising a LCDR1 amino acid sequence at least 70% identical to SEQ ID NO: 461, a LCDR2 amino acid sequence at least 70% identical to SEQ ID NO: 201, and a LCDR3 amino acid sequence at least 70% identical to SEQ ID NO: 505, or
a heavy chain variable region comprising a HCDR1 amino acid sequence at least 70% identical to SEQ ID NO: 4, a HCDR2 amino acid sequence at least 70% identical to SEQ ID NO: 416, and a HCDR3 amino acid sequence at least 70% identical to SEQ ID NO: 438; and a light chain variable region comprising a LCDR1 amino acid sequence at least 70% identical to SEQ ID NO: 462, a LCDR2 amino acid sequence at least 70% identical to SEQ ID NO: 480, and a LCDR3 amino acid sequence at least 70% identical to SEQ ID NO: 506, or
a heavy chain variable region comprising a HCDR1 amino acid sequence at least 70% identical to SEQ ID NO: 4, a HCDR2 amino acid sequence at least 70% identical to SEQ ID NO: 414, and a HCDR3 amino acid sequence at least 70% identical to SEQ ID NO: 436; and a light chain variable region comprising a LCDR1 amino acid sequence at least 70% identical to SEQ ID NO: 462, a LCDR2 amino acid sequence at least 70% identical to SEQ ID NO: 480, and a LCDR3 amino acid sequence at least 70% identical to SEQ ID NO: 507.

5. The antibody or the binding fragment thereof of claim 1, wherein the antibody comprises:
a heavy chain variable region comprising a HCDR1 amino acid sequence at least 70% identical to SEQ ID NO: 389, a HCDR2 amino acid sequence at least 70% identical to SEQ ID NO: 417, and a HCDR3 amino acid sequence at least 70% identical to SEQ ID NO: 439.

6. The antibody or the binding fragment thereof of claim 1, wherein the antibody comprises all six CDRs of an antibody selected from the group consisting of 1104SBC1068-033, 1104SBC1068-335, 1104SBC1068-365, 1104SBC1068-374, 1104SBC1068-378, 1220SBC1068-022, 1220SBC1068-035, 1220SBC1068-041, 1220SBC1068-056, 1220SBC1068-058, 1220SBC1068-063, 1220SBC1068-097, 1220SBC1068-098, 1220SBC1068-099, 1220SBC1068-129, 1220SBC1068-164, 1220SBC1068-186, 1220SBC1068-197, 1220SBC1068-210, 1220SBC1068-274, 1220SBC1068-281, 20240628SBC1080-002, 20240628SBC1080-003, 20240628SBC1080-004, 20240628SBC1080-005, 20240628SBC1080-006, 20240628SBC1080-007, 20240628SBC1080-008, 20240628SBC1080-009, 20240903SBC1093-015, 20240903SBC1093-020, 20240903SBC1093-021, 20240903SBC1093-025, 20240903SBC1093-030, 20240903SBC1093-182, 20240903SBC1093-183, 20240903SBC1093-184, 20240903SBC1093-185, 20240903SBC1093-230, 20240903SBC1093-033, 20240903SBC1093-034, 20240903SBC1093-035, 20240903SBC1093-044, 20240903SBC1093-054, 20240903SBC1093-055, 20240903SBC1093-056, 20240903SBC1093-057, 20240903SBC1093-059, 20240903SBC1093-219, 20240903SBC1093-228, 20240903SBC1093-023, 20240903SBC1093-036, 20240903SBC1093-037, 20240903SBC1093-220, 20240903SBC1093-074, 20240903SBC1093-075, 20240903SBC1093-193, 20240903SBC1093-194, 20240903SBC1093-099, 20240929SBC1205-022, 20241223SBC1205-061, 20241223SBC1205-063, 20241223SBC1205-119, 20241223SBC1205-130, 20241223SBC1205-131, and ANb1361-hGal-3-LP3R3 P2-69.

7. The antibody or the binding fragment thereof of claim 1, wherein the antibody comprises:
a V_{H} region comprising a V_{H} amino acid sequence at least 70% identity to any one of SEQ ID NOS: 148-168, 353, and 355; and/or
a V_{L} region comprising a V_{L} amino acid sequence at least 70% identity to any one of SEQ ID NOS: 316-336, 354, and 356.

8. The antibody or the binding fragment thereof of claim 1, wherein the antibody comprises:
a V_{H} region comprising a V_{H} amino acid sequence at least 70% identity to SEQ ID NO: 148, and a V_{L} region comprising a V_{L} amino acid sequence at least 70% identity to SEQ ID NO: 316; or
a V_{H} region comprising a V_{H} amino acid sequence at least 70% identity to SEQ ID NO: 149, and a V_{L} region comprising a V_{L} amino acid sequence at least 70% identity to SEQ ID NO: 317; or
a V_{H} region comprising a V_{H} amino acid sequence at least 70% identity to SEQ ID NO: 150, and a V_{L} region comprising a V_{L} amino acid sequence at least 70% identity to SEQ ID NO: 318; or
a V_{H} region comprising a V_{H} amino acid sequence at least 70% identity to SEQ ID NO: 151, and a V_{L} region comprising a V_{L} amino acid sequence at least 70% identity to SEQ ID NO: 319; or
a V_{H} region comprising a V_{H} amino acid sequence at least 70% identity to SEQ ID NO: 152, and a V_{L} region comprising a V_{L} amino acid sequence at least 70% identity to SEQ ID NO: 320; or
a V_{H} region comprising a V_{H} amino acid sequence at least 70% identity to SEQ ID NO: 153, and a V_{L} region comprising a V_{L} amino acid sequence at least 70% identity to SEQ ID NO: 321; or
a V_{H} region comprising a V_{H} amino acid sequence at least 70% identity to SEQ ID NO: 154, and a V_{L} region comprising a V_{L} amino acid sequence at least 70% identity to SEQ ID NO: 322; or
a V_{H} region comprising a V_{H} amino acid sequence at least 70% identity to SEQ ID NO: 155, and a V_{L} region comprising a V_{L} amino acid sequence at least 70% identity to SEQ ID NO: 323; or
a V_{H} region comprising a V_{H} amino acid sequence at least 70% identity to SEQ ID NO: 156, and a V_{L} region comprising a V_{L} amino acid sequence at least 70% identity to SEQ ID NO: 324; or
a V_{H} region comprising a V_{H} amino acid sequence at least 70% identity to SEQ ID NO: 157, and a V_{L} region comprising a V_{L} amino acid sequence at least 70% identity to SEQ ID NO: 325; or
a V_{H} region comprising a V_{H} amino acid sequence at least 70% identity to SEQ ID NO: 158, and a V_{L} region comprising a V_{L} amino acid sequence at least 70% identity to SEQ ID NO: 326; or
a V_{H} region comprising a V_{H} amino acid sequence at least 70% identity to SEQ ID NO: 159, and a V_{L} region comprising a V_{L} amino acid sequence at least 70% identity to SEQ ID NO: 327; or
a V_{H} region comprising a V_{H} amino acid sequence at least 70% identity to SEQ ID NO: 160, and a V_{L} region comprising a V_{L} amino acid sequence at least 70% identity to SEQ ID NO: 328; or
a V_{H} region comprising a V_{H} amino acid sequence at least 70% identity to SEQ ID NO: 161, and a V_{L} region comprising a V_{L} amino acid sequence at least 70% identity to SEQ ID NO: 329; or
a V_{H} region comprising a V_{H} amino acid sequence at least 70% identity to SEQ ID NO: 162, and a V_{L} region comprising a V_{L} amino acid sequence at least 70% identity to SEQ ID NO: 330; or
a V_{H} region comprising a V_{H} amino acid sequence at least 70% identity to SEQ ID NO: 163, and a V_{L} region comprising a V_{L} amino acid sequence at least 70% identity to SEQ ID NO: 331; or
a V_{H} region comprising a V_{H} amino acid sequence at least 70% identity to SEQ ID NO: 164, and a V_{L} region comprising a V_{L} amino acid sequence at least 70% identity to SEQ ID NO: 332; or
a V_{H} region comprising a V_{H} amino acid sequence at least 70% identity to SEQ ID NO: 165, and a V_{L} region comprising a V_{L} amino acid sequence at least 70% identity to SEQ ID NO: 333; or
a V_{H} region comprising a V_{H} amino acid sequence at least 70% identity to SEQ ID NO: 166, and a V_{L} region comprising a V_{L} amino acid sequence at least 70% identity to SEQ ID NO: 334; or
a V_{H} region comprising a V_{H} amino acid sequence at least 70% identity to SEQ ID NO: 167, and a V_{L} region comprising a V_{L} amino acid sequence at least 70% identity to SEQ ID NO: 335; or
a V_{H} region comprising a V_{H} amino acid sequence at least 70% identity to SEQ ID NO: 168, and a V_{L} region comprising a V_{L} amino acid sequence at least 70% identity to SEQ ID NO: 336; or
a V_{H} region comprising a V_{H} amino acid sequence at least 70% identity to SEQ ID NO: 353, and a V_{L} region comprising a V_{L} amino acid sequence at least 70% identity to SEQ ID NO: 354; or
a V_{H} region comprising a V_{H} amino acid sequence at least 70% identity to SEQ ID NO: 355, and a V_{L} region comprising a V_{L} amino acid sequence at least 70% identity to SEQ ID NO: 356.

9. The antibody or the binding fragment thereof of claim 1, wherein the antibody comprises both the V_{H} and V_{L} of an antibody selected from the group consisting of 1104SBC1068-033, 1104SBC1068-335, 1104SBC1068-365, 1104SBC1068-374, 1104SBC1068-378, 1220SBC1068-022, 1220SBC1068-035, 1220SBC1068-041, 1220SBC1068-056, 1220SBC1068-058, 1220SBC1068-063, 1220SBC1068-097, 1220SBC1068-098, 1220SBC1068-099, 1220SBC1068-129, 1220SBC1068-164, 1220SBC1068-186, 1220SBC1068-197, 1220SBC1068-210, 1220SBC1068-274, 1220SBC1068-281, SIF-001, SIF-002, and a variant thereof.

10. The antibody of claim 1, wherein the antibody is a humanized, chimeric, or human antibody.

11. A polypeptide comprising a V_{H} sequence and/or a V_{L} sequence of the antibody of claim 1.

12. A polynucleotide encoding the polypeptide of claim 11.

13. A cell that comprises the polypeptide of claim 11.

14. An immunoconjugate comprising the antibody of claim 1 and a cytotoxic agent.

15. A pharmaceutical composition comprising the antibody of claim 1 and a pharmaceutically acceptable carrier.

16. A method of treating epilepsy or a related neurological disorder in a subject in need thereof, comprising administering the subject an effective amount of the pharmaceutical composition of claim 15.

17. The method of claim 16, wherein the effective amount is about 5-100 mg/kg of the antibody or the immunoconjugate per the subject's body weight, optionally wherein the effective amount is about 10, 20, 40, and 70 mg/kg of the antibody or the immunoconjugate per the subject's body weight, wherein optionally, the pharmaceutical composition is administered intravenously into the subject, and
optionally wherein the pharmaceutical composition is administered more than once, optionally wherein each administration is at least 14 days apart.

18. The antibody or the binding fragment thereof of claim 1, wherein the antibody comprises: a heavy chain variable region comprising:
a HCDR1 comprising a sequence NYGMN (SEQ ID NO: 4), or a variant HCDR1 in which 1, 2, or 3 amino acids are substituted relative to the sequence;
a HCDR2 comprising a sequence WINTYTGEPTYADDFKG (SEQ ID NO: 25), or a variant HCDR2 in which 1, 2, or 3 amino acids are substituted relative to the sequence; and
a HCDR3 comprising a sequence YAMDY (SEQ ID NO: 46), or a variant HCDR3 in which 1, 2, or 3 amino acids are substituted relative to the sequence; and
a light chain variable region comprising:
a LCDR1 comprising a sequence RSSTGAVTTSNYAN (SEQ ID NO: 172), or a variant LCDR1 in which 1 amino acid is substituted relative to the sequence;
a LCDR2 comprising a sequence GTSNRAP (SEQ ID NO: 193), or variant LCDR2 in which 1 amino acid is substituted relative to the sequence; and
a LCDR3 comprising a sequence ALWYSTHYV (SEQ ID NO: 214), or a variant LCDR3 in which 1 amino acid is substituted relative to the sequence;
optionally wherein the substitution in HCDR1 is in any of the residue #1, and #3-#5, wherein the substitution in HCDR2 is in any of the residue #2, #4-#17, wherein the substitution in HCDR3 is in any of the residues #2-#4, wherein the substitution in LCDR1 is in any of the residues #1-#11 and #13-#14, wherein the substitution in LCDR3 is in any of the residues #1-#2, #4-#7, and #9;
further optionally wherein the aa at position #2 in HCDR1 is Y, W, or F, wherein the aa at position #1 in HCDR2 is W, Y, or F, and aa at position #3 residue in HCDR1 is N or Q, wherein the aa at position #1 in HCDR3 is Y, wherein the aa at position #12 in LCDR1 is Y, W, or F, wherein the aa at position #3 in LCDR3 is Y, W, or F, and aa at position #8 in LCDR3 is Y, W, or F;
or
a heavy chain variable region comprising:
a HCDR1 comprising a sequence RFWMS (SEQ ID NO: 8), or a variant HCDR1 in which 1, 2, or 3 amino acids are substituted relative to the sequence;
a HCDR2 comprising a sequence EISPDSNTIDLTPSLKD (SEQ ID NO: 29), or a variant HCDR2 in which 1, 2, or 3 amino acids are substituted relative to the sequence; and
a HCDR3 comprising a sequence PYYGYY (SEQ ID NO: 50), or a variant HCDR3 in which 1, 2, or 3 amino acids are substituted relative to the sequence; and
a light chain variable region comprising:
a LCDR1 comprising a sequence RSSQSLFNSTNQKNYLT (SEQ ID NO: 176) or RSSQSLFSSTNQKNYLT (SEQ ID NO: 369), or a variant LCDR1 in which 1 amino acid is substituted relative to the sequence;
a LCDR2 comprising a sequence WASSRES (SEQ ID NO: 197), or variant LCDR2 in which 1 amino acid is substituted relative to the sequence; and
a LCDR3 comprising a sequence QNDYTSPFT (SEQ ID NO: 218), or a variant LCDR3 in which 1 amino acid is substituted relative to the sequence;
optionally wherein the substitution in HCDR1 is in any of the positions #1-#2, and #4-#5 in HCDR1, wherein the substitution in HCDR2 is in any of the positions #2-#17 in HCDR2, wherein the substitution in HCDR3 is in any of the residues #1-#4 and #6 in HCDR3, wherein the substitution in LCDR1 is in any of the residues #1-#14 and #16-#17 in LCDR1, wherein the substitution in LCDR3 is in any of the residues #1-#2, #4-#9 in LCDR3;
further optionally wherein the aa at position #3 in HCDR1 is Y, W, or F, wherein the aa at position #1 in HCDR2 is E or D, wherein the aa at position #5 in HCDR3 is Y, W, or F, wherein the aa at position #15 in LCDR1 is Y, W, or F, wherein the aa at position #3 in LCDR3 is Y.

19. An antibody or a binding fragment thereof that binds to Galectin-3, wherein the antibody comprises:
a HCDR1 comprising a HCDR1 sequence of NX₂GMN (SEQ ID NO:357), wherein X₂ is Y, W, or F, wherein the HCDR1 has zero or one aa substitution relative to the HCDR1 sequence,
a HCDR2 comprising a HCDR2 sequence of X₁IX₃TYTGEPTYADDFKG (SEQ ID NO: 358), Where X₁ is W, Y, or F, and wherein X₃ is N or Q, wherein the HCDR2 has zero, one, two, three, or four aa substitutions relative to the HCDR2 sequence,
a HCDR3 comprising a HCDR3 sequence of YAMDY (SEQ ID NO:359), wherein the HCDR3 has zero, one, or two aa substitutions relative to the HCDR3 sequence,
a LCDR1 comprising a LCDR1 sequence of RSSTGAVTTSNX₁₂AN (SEQ ID NO: 360), wherein X₁₂ is Y, W, or F, wherein the LCDR1 has zero, one, two, three, or four aa substitutions in the rest of the residues relative to the LCDR1 sequence,
a LCDR2 comprising a LCDR2 sequence of GTSNRAP (SEQ ID NO: 361), wherein the LCDR2 has zero, one, two, or three aa substitutions in the rest of the residues relative to the LCDR2 sequence, and
a LCDR3 comprising a LCDR3 sequence of ALX₃YSTHX₈V (SEQ ID NO: 362), wherein X₃ is Y, W, or F, wherein X₈ is Y, W, or F, wherein the LCDR3 has zero, one, two aa substitutions in the rest of the residues relative to the LCDR3 sequence;
or
a HCDR1 comprising a HCDR1 sequence of RFX₃MS (SEQ ID NO: 363), wherein X₃ is Y, W, or F, and wherein the HCDR1 has zero, one, two aa substitutions relative to the HCDR1 sequence,
a HCDR2 comprising a HCDR2 sequence of X₁ISPDSNTIDLTPSLKD (SEQ ID NO: 364), wherein X₁ is E or D, and wherein the HCDR2 has zero, one, two, three, or four aa substitutions relative to the HCDR2 sequence,
a HCDR3 comprising a HCDR3 sequence of PYYGX₅Y (SEQ ID NO: 365), wherein X₅ is Y, W, or F, wherein the HCDR3 has zero, one, two aa substitutions relative to the HCDR3 sequence,
a LCDR1 comprising a LCDR1 sequence of RSSQSLFSSTNQKNX₁₅LT (SEQ ID NO: 366), wherein X₁₅ is Y, W, or F, wherein the LCDR1 has zero, one, two, three, or four aa substitutions relative to the LCDR1 sequence,
a LCDR2 comprising a LCDR2 sequence of WASSRES (SEQ ID NO: 367), wherein the LCDR2 has zero, one, two, or three aa substitutions relative to the LCDR2 sequence, and/or
a LCDR3 comprising a LCDR3 sequence of QNDYTSPFT (SEQ ID NO: 368), wherein the LCDR3 has zero, one, two, three, or four aa substitutions relative to the LCDR3 sequence.

20. An antibody or a binding fragment thereof that binds to Galectin-3, wherein the antibody comprises:
a HCDR1 comprising a HCDR1 sequence of NX₂GMN (SEQ ID NO:357), wherein X₂ is Y, W, or F,
a HCDR2 comprising a HCDR2 sequence of X₁IX₃TYTGEPTYADDFKG (SEQ ID NO: 358), Where X₁ is W, Y, or F, and wherein X₃ is N or Q,
a HCDR3 comprising a HCDR3 sequence of YAMDY (SEQ ID NO:359),
a LCDR1 comprising a LCDR1 sequence of RSSTGAVTTSNX₁₂AN (SEQ ID NO: 360), wherein X₁₂ is Y, W, or F,
a LCDR2 comprising a LCDR2 sequence of GTSNRAP (SEQ ID NO: 361), and
a LCDR3 comprising a LCDR3 sequence of ALX₃YSTHX₈V (SEQ ID NO: 362), wherein X₃ is Y, W, or F, wherein X₈ is Y, W, or F;
wherein
the HCDR1 has zero or one aa substitution relative to the HCDR1 sequence,
the HCDR2 has zero, one, two, three, or four aa substitutions relative to the HCDR2 sequence,
the HCDR3 has zero, one, or two aa substitutions relative to the HCDR3 sequence, the LCDR1 has zero, one, two, three, or four aa substitutions relative to the LCDR1 sequence,
the LCDR2 has zero, one, two, or three aa substitutions relative to the LCDR2 sequence, and/or
the LCDR3 has zero, one, two aa substitutions relative to the LCDR3 sequence; or
a HCDR1 comprising a sequence of RFX₃MS (SEQ ID NO: 363), wherein X₃ is Y, W, or F,
a HCDR2 comprising X₁ISPDSNTIDLTPSLKD (SEQ ID NO: 364), wherein X₁ is E or D,
a HCDR3 comprising PYYGX₅Y (SEQ ID NO: 365), wherein X₅ is Y, W, or F,
a LCDR1 comprising RSSQSLFSSTNQKNX₁₅LT (SEQ ID NO: 366), wherein X₁₅ is Y, W, or F,
a LCDR2 comprising WASSRES (SEQ ID NO: 367), and
a LCDR3 comprising QNDYTSPFT (SEQ ID NO: 368);
wherein
the HCDR1 has zero, one, two aa substitutions relative to the HCDR1 sequence,
the HCDR2 has zero, one, two, three, or four aa substitutions relative to the HCDR2 sequence,
the HCDR3 has zero, one, two aa substitutions relative to the HCDR3 sequence, the LCDR1 has zero, one, two, three, or four aa substitutions relative to the LCDR1 sequence,
the LCDR2 has zero, one, two, or three aa substitutions relative to the LCDR2 sequence, and/or
the LCDR3 has zero, one, two, three, or four aa substitutions relative to the LCDR3 sequence.

21. The antibody or the binding fragment thereof of claim 1, wherein the antibody comprises
a HCDR1 comprising a sequence of NYGMN (SEQ ID NO: 4), a HCDR2 comprising a sequence of WINTYTGEPTYADDFKG (SEQ ID NO: 25), a HCDR3 comprising a sequence of YAMDY (SEQ ID NO: 46), a LCDR1 comprising a sequence of RSSTGAVTTSNYAN (SEQ ID NO: 172), a LCDR2 comprising a sequence of GTSNRAP (SEQ ID NO: 193), and a LCDR3 comprising a sequence of ALWYSTHYV (SEQ ID NO: 214); optionally wherein the antibody comprises a V_{H} region comprising a sequence of SEQ ID NO: 353, and a V_{L} region comprising a sequence of SEQ ID NO: 354,
or
a HCDR1 comprising a sequence of RFWMS (SEQ ID NO: 8), a HCDR2 comprising a sequence of EISPDSNTIDLTPSLKD (SEQ ID NO: 29), a HCDR3 comprising a sequence of PYYGYY (SEQ ID NO: 50), a LCDR1 comprising a sequence of RSSQSLFNSTNQKNYLT (SEQ ID NO: 176) or RSSQSLFSSTNQKNYLT (SEQ ID NO: 369), a LCDR2 comprising a sequence of WASSRES (SEQ ID NO: 197), and a LCDR3 comprising a sequence of QNDYTSPFT (SEQ ID NO: 218), optionally wherein the antibody comprises a V_{H} region comprising a sequence of SEQ ID NO: 355, and a V_{L} region comprising a sequence of SEQ ID NO: 356.
